(19) United States
(12) Patent Application Publication
(10) Pub. No.: US 2026/0193254 A1
(43) Pub. Date: Jul. 9, 2026

(54) 2-OXOQUINAZOLINE CRYSTALLINE FORMS

(71) Applicant: IDEAYA Biosciences, Inc., South San Francisco, CA (US)

(72) Inventor: Xin LINGHU, South San Francisco, CA (US)

(21) Appl. No.: 19/134,059

(22) PCT Filed: Nov. 30, 2022
(86) PCT No.: PCT/US2022/051396

§ 371 Date: May 29, 2025

(51) Int. Cl. C07D 471/04; A61K 31/519

(52) U.S. Cl. CPC C07D 471/04; A61K 31/519

(57) ABSTRACT

The present disclosure provides crystalline forms of the compound having Formula (I), wherein the crystalline forms are crystalline Forms A to U. The crystalline Forms A to U are characterized by an X-ray powder diffraction (XRPD) pattern. Selected crystalline forms are further characterized by a differential scanning calorimetry (DSC), a thermal gravimetric analysis (TGA), and/or a polarized light microscope (PLM) profile. The present disclosure also provides a substantially amorphous form of the compound having Formula (I), which is prepared by grinding any one of crystalline Forms A to U, in particular Form T. The present disclosure also provides methods for preparing crystalline forms, in particular Forms A, C, D, E, and I. Also disclosed herein are solid state forms of a compound of Formula (I) and methods of making the same. The present disclosure further provides methods of treating a disease mediated by MAT2A, for example, cancer, using the crystalline forms of the disclosure or a pharmaceutical composition thereof.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cancer is a leading cause of death throughout the world. A limitation of prevailing therapeutic approaches, e.g. chemotherapy and immunotherapy is that their cytotoxic effects are not restricted to cancer cells and adverse side effects can occur within normal tissues. Consequently, novel strategies are needed to better target cancer cells.

Synthetic lethality arises when a combination of deficiencies in the expression of two or more genes leads to cell death, whereas a deficiency in only one of these genes does not. The concept of synthetic lethality originates from studies in drosophila model systems in which a combination of mutations in two or more separate genes leads to cell death (in contrast to viability, which occurs when only one of the genes is mutated or deleted). More recently, a multitude of studies have explored maladaptive genetic changes in cancer cells that render them vulnerable to synthetic-lethality approaches. These tumor-specific genetic defects lead to the use of targeted agents that induce the death of tumor cells while sparing normal cells.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA and proteins. One methylase that utilizes SAM as a methyl donor, is protein arginine N-methyltransferase 5 (PRMT5). While SAM is required for PRMT5 activity, PRMT5 is competitively inhibited by 5′methylthioadenosine (MTA). Since MTA is part of the methionine salvage pathway, cellular MTA levels stay low in a process initiated by methylthioadenosine phosphorylase (MTAP).

MTAP is in a locus on chromosome 9 that is often deleted in cells of patients with cancers from several tissues of origin including central nervous system, pancreas, esophageal, bladder and lung (cBioPortal database). Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted cells compare to MTAP WT cells (see McDonald et. al. 2017 Cell 170, 577-592). Furthermore, inducible knockdown of MAT2A protein decreased tumor growth in vivo (see Marjon et. al., 2016 Cell Reports 15(3), 574-587). These results indicate that MAT2A inhibitors and their polymorphic forms may provide a useful therapy for cancer patients including those with MTAP-deleted tumors.

### BRIEF SUMMARY OF THE INVENTION

In a first aspect, the present disclosure provides a crystalline form of a compound having Formula (I):

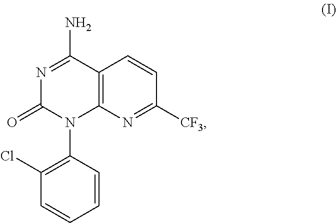 [SMILES: Nc1nc(=O)n(-c2ccccc2Cl)c2nc(C(F)(F)F)ccc12]

wherein the crystalline form is any one of crystalline Forms A to U, each of which is characterized by an X-ray powder diffraction (XRPD) pattern as described herein.

In a second aspect, the present disclosure provides solid state forms of a compound having Formula (I)

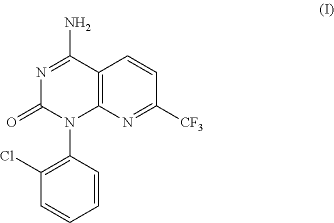 [SMILES: Nc1nc(=O)n(-c2ccccc2Cl)c2nc(C(F)(F)F)ccc12]

or a pharmaceutically acceptable salt thereof. In some embodiments the solid state form of Formula (I) is solid state Form V. In some embodiments, solid state Form V of Formula (I) is substantially crystalline.

In a third aspect, the present disclosure provides a method for preparing crystalline Form A of a compound having Formula (I). The method includes:
a) forming a first mixture comprising a crude compound of Formula (I), ACN, and water;
b) solvent exchanging with water at a temperature of no more than about 65° C. to form a second mixture;
c) cooling the second mixture and stirring to form a third precipitate;
d) isolating the third precipitate;
e) forming a slurry comprising the third precipitate, methyl ethyl ketone (MEK), and water;
f) isolating a fourth precipitate from step e);
g) drying the fourth precipitate to provide the crystalline Form A of Formula (I).

In a fourth aspect, the present disclosure provides a method for preparing a crystalline Form of a compound having Formula (I), including:
a) forming a slurry comprising solid state Form V of Formula (I) and a solvent;
b) stirring the slurry for a period of at least a day; and
c) isolating a precipitate; and
d) drying the precipitate to provide the crystalline Form of Formula (I),
wherein the crystalline Form is crystalline Form C and the solvent is methanol, Form D and the solvent is water, Form E and the solvent is methanol/water, optionally in a 1:1 ratio, Form I and the solvent is acetone/water, optionally in a 1:1 ratio, or Form A and the solvent is acetonitrile/water, optionally in a 1:1 ratio.

In a fifth aspect, the present disclosure provides a method for treating a disease mediated by MAT2A in a patient, the method including administering to the patient a therapeutically effective amount of a crystalline form of the compound of Formula (I) as described herein or a pharmaceutical composition thereof.

In a sixth aspect, the present disclosure provides a method of treating a MTAP null cancer in a patient, the method including administering to the patient a therapeutically effective amount of a crystalline form of the compound of Formula (I) as described herein or a pharmaceutical composition thereof.

In an seventh aspect, the present disclosure provides a method for treating a cancer in a patient, wherein the cancer is characterized by a reduction or absence of MTAP gene expression, the absence of the MTAP gene, reduced level of MTAP protein, reduced function of MTAP protein, absence of MTAP protein, or a combination thereof, the method includes administering to the subject a therapeutically effective amount of a crystalline form of the compound of Formula (I) as described herein or a pharmaceutical composition thereof.

### BRIEF DESCRIPTION OF THE DRAWINGS

FIG. 1 shows an X-ray powder diffraction pattern of solid state Form V.

FIG. 2 shows a differential scanning calorimetry (DSC) thermogram of solid state Form V.

FIG. 3 shows a thermal gravimetric analysis (TGA) thermogram of solid state Form V.

FIG. 4 shows a polarized light microscope (PLM) profile of solid state Form V.

FIG. 5 shows a ¹H NMR spectrum of solid state Form V.

FIG. 6 shows an X-ray powder diffraction pattern of crystalline Form A.

FIG. 7 shows a differential scanning calorimetry (DSC) thermogram of crystalline Form A.

FIG. 8 shows a thermal gravimetric analysis (TGA) thermogram of crystalline Form A.

FIG. 9 shows a polarized light microscope (PLM) profile of crystalline Form A.

FIGS. 10A and B shows a dynamic vapor sorption (DVS) profile of crystalline Form A.

FIG. 11 shows a ¹H NMR spectrum of crystalline Form A.

FIG. 12 shows an X-ray powder diffraction pattern of crystalline Form B.

FIG. 13 shows an X-ray powder diffraction pattern of crystalline Form C.

FIG. 14 shows a differential scanning calorimetry (DSC) thermogram of crystalline Form C.

FIG. 15 shows a thermal gravimetric analysis (TGA) thermogram of crystalline Form C.

FIG. 16 shows a polarized light microscope (PLM) profile of crystalline Form C.

FIG. 17 shows a ¹H NMR spectrum of crystalline Form C.

FIGS. 18A and B shows a dynamic vapor sorption (DVS) profile of crystalline Form C.

FIG. 19 shows an X-ray powder diffraction pattern of crystalline Form D.

FIG. 20 shows a differential scanning calorimetry (DSC) thermogram of crystalline Form D.

FIG. 21 shows a thermal gravimetric analysis (TGA) thermogram of crystalline Form D.

FIG. 22 shows a polarized light microscope (PLM) profile of crystalline Form D.

FIG. 23 shows a ¹H NMR spectrum of crystalline Form D.

FIGS. 24A and B shows a dynamic vapor sorption (DVS) profile of crystalline Form D.

FIG. 25 shows an X-ray powder diffraction pattern of crystalline Form E.

FIG. 26 shows a differential scanning calorimetry (DSC) thermogram of crystalline Form E.

FIG. 27 shows a thermal gravimetric analysis (TGA) thermogram of crystalline Form E.

FIG. 28 shows a polarized light microscope (PLM) profile of crystalline Form E.

FIG. 29 shows a ¹H NMR spectrum of crystalline Form E.

FIG. 30 shows an X-ray powder diffraction pattern of crystalline Form F.

FIG. 31 shows a differential scanning calorimetry (DSC) thermogram of crystalline Form F.

FIG. 32 shows a thermal gravimetric analysis (TGA) thermogram of crystalline Form F.

FIG. 33 shows an X-ray powder diffraction pattern of crystalline Form G.

FIG. 34 shows a differential scanning calorimetry (DSC) thermogram of crystalline Form G.

FIG. 35 shows a thermal gravimetric analysis (TGA) thermogram of crystalline Form G.

FIG. 36 shows a ¹H NMR spectrum of crystalline Form G.

FIG. 37 shows an X-ray powder diffraction pattern of crystalline Form H.

FIG. 38 shows a differential scanning calorimetry (DSC) thermogram of crystalline Form H.

FIG. 39 shows a thermal gravimetric analysis (TGA) thermogram of crystalline Form H.

FIG. 40 shows a ¹H NMR spectrum of crystalline Form H.

FIG. 41 shows an X-ray powder diffraction pattern of crystalline Form I.

FIG. 42 shows a differential scanning calorimetry (DSC) thermogram of crystalline Form I.

FIG. 43 shows a thermal gravimetric analysis (TGA) thermogram of crystalline Form I.

FIG. 44 shows a polarized light microscope (PLM) profile of crystalline Form I.

FIG. 45 shows a ¹H NMR spectrum of crystalline Form I.

FIG. 46 shows an X-ray powder diffraction pattern of crystalline Form J.

FIG. 47 shows a differential scanning calorimetry (DSC) thermogram of crystalline Form J.

FIG. 48 shows a thermal gravimetric analysis (TGA) thermogram of crystalline Form J.

FIG. 49 shows a ¹H NMR spectrum of crystalline Form J.

FIG. 50 shows an X-ray powder diffraction pattern of crystalline Form K.

FIG. 51 shows an X-ray powder diffraction pattern of crystalline Form L.

FIG. 52 shows a differential scanning calorimetry (DSC) thermogram of crystalline Form L.

FIG. 53 shows a thermal gravimetric analysis (TGA) thermogram of crystalline Form L.

FIG. 54 shows an X-ray powder diffraction pattern of crystalline Form M.

FIG. 55 shows an X-ray powder diffraction pattern of crystalline Form N.

FIG. 56 shows an X-ray powder diffraction pattern of crystalline Form O.

FIG. 57 shows an X-ray powder diffraction pattern of crystalline Form P.

FIG. 58 shows an X-ray powder diffraction pattern of crystalline Form Q.

FIG. 59 shows an X-ray powder diffraction pattern of crystalline Form R.

FIG. 60 shows an X-ray powder diffraction pattern of crystalline Form S.

FIG. 61 shows an X-ray powder diffraction pattern of crystalline Form T.

FIG. 62 shows a differential scanning calorimetry (DSC) thermogram of crystalline Form T.

FIG. 63 shows a thermal gravimetric analysis (TGA) thermogram of crystalline Form T.

FIG. 64 shows a polarized light microscope (PLM) profile of crystalline Form T.

FIG. 65 shows a ¹H NMR spectrum of crystalline Form T.

FIGS. 66A and B shows a dynamic vapor sorption (DVS) profile of crystalline Form T.

FIG. 67 shows an X-ray powder diffraction pattern of crystalline Form U.

FIG. 68 shows an X-ray powder diffraction pattern of a substantially amorphous form obtained from dry grinding Form T.

### DETAILED DESCRIPTION OF THE INVENTION

### I. General

The present disclosure provides crystalline forms of the compound having Formula (I), wherein the crystalline forms are crystalline Forms A to U. The crystalline Forms A to U are characterized by an X-ray powder diffraction (XRPD) pattern. Selected crystalline forms are further characterized by a differential scanning calorimetry (DSC), a thermal gravimetric analysis (TGA), and/or a polarized light microscope (PLM) profile. The present disclosure also provides methods for preparing crystalline forms, in particular Forms A, C, D, E, and I. Also disclosed herein are solid state forms of a compound of Formula (I) and methods of making the same. In some embodiments, the solid state form is solid state Form V. The present disclosure further provides methods of treating a disease mediated by MAT2A, in particular cancer using the crystalline forms of the disclosure or a pharmaceutical composition thereof. The present disclosure is useful for the treatment of a variety of cancers, including solid tumors. The present disclosure is useful for the treatment of a variety of diseases or disorders treatable by inhibiting MAT2A. The present disclosure is also useful for the treating MTAP-deficient tumors.

### II. Definitions

“Substantially free” refers to that an amount of 10% or less of another form is present in a particular desired form, preferably 9%, 8.5%, 8%, 7.55, 7%, 6.5%, 6%, 5.5%, 5%, 4.5%, 4%, 3.5%, 3%, 2.5%, 2%, 1.5%, 1%, 0.5%, or less of another form.

“Crude” refers to a mixture including a desired compound (e.g., the compound of Formula (I)) and at least one other species (e.g., a solvent, a reagent such as an acid or base, a starting material, or a byproduct of a reaction giving rise to the desired compound).

“Alkyl alcohol” refers to an alkyl group having a hydroxy group attached to a carbon of the alkyl group, wherein the alkyl group is defined as a straight or branched, saturated, aliphatic radical having the number of carbon atoms indicated (i.e., C₁₋₄ means one to four carbons). For example, C₁₋₄ alkyl alcohol includes methanol, ethanol, n-propanol, isopropanol, n-butanol, sec-butanol, isobutanol, and tert-butanol. Alkyl alcohols useful in the present invention are fully saturated. One of skill in the art will appreciate that other alcohols are useful in the present invention.

“Solvate” refers to a compound provided herein or a salt thereof, that binds to a stoichiometric or non-stoichiometric amount of solvent by non-covalent intermolecular forces. Where the solvent is water, the solvate is a hydrate.

“Hydrate” refers to a compound that is complexed to a stoichiometric or non-stoichiometric amount of water. The compounds of the present invention can be complexed with from ½ or 1 to 10 water molecules. For example, the compounds of the present invention can be complexed with ½ water molecule, the compounds of the present invention can be complexed with 1 water molecule, or the compounds of the present invention can be complexed with 2 water molecules.

“Crystalline form” refers to a solid form of a compound wherein the constituent molecules are packed in a regularly ordered, repeating pattern. A crystalline form can include triclinic, monoclinic, orthorhombic, tetragonal, trigonal, hexagonal, and cubic crystal geometries. A crystalline form can include one or more regions, i.e., grains, with distinct crystal boundaries. A crystalline solid can include two or more crystal geometries.

“Amorphous form” refers to a solid form of a compound having no definite crystal structure, i.e., lacking a regularly ordered, repeating pattern of constituent molecules.

“FeSSIF” stands for Fed State Simulated Intestinal Fluid. “FaSSIF” stands for Fasted State Simulated Intestinal Fluid. “SGF” stands for Simulated Gastric Fluid.

“About” means a range of values including the specified value, which a person of ordinary skill in the art would consider reasonably similar to the specified value. In some embodiments, the term “about” means within a standard deviation using measurements generally acceptable in the art. In some embodiments, “about” means a range extending to +/−10% of the specified value.

“Pharmaceutically acceptable salts” as used herein is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

“Solid state form” refers to any crystalline and/or amorphous solid phase of a compound having Formula (I) or a pharmaceutically acceptable salt thereof. This includes mixtures of crystalline or amorphous solid phases. Solid state forms include anhydrous, hydrate, and solvate solid phase forms of a compound having Formula (I) or a pharmaceutically acceptable salt thereof. Solid state forms described herein can include at least 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 99 wt. % of a particular crystalline form. In some embodiments the particular crystalline form is Form A.

### III. Crystalline Forms

In a first aspect, the present disclosure provides crystalline forms of a compound having Formula (I):

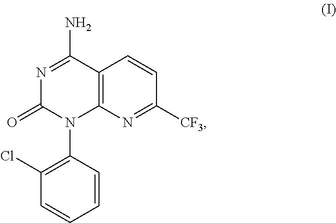 [SMILES: Nc1nc(=O)n(-c2ccccc2Cl)c2nc(C(F)(F)F)ccc12]

wherein the crystalline form is any one of crystalline Forms A to U, each of which is characterized by an X-ray powder diffraction (XRPD) pattern as described herein. Certain crystalline forms may include pharmaceutically acceptable salts of the compound of Formula (I). Certain crystalline forms may be hydrates, solvates, or anhydrous forms of the compound of Formula (I). This disclosure also provides solid state forms of a compound having Formula (I) or a pharmaceutically acceptable salt thereof. In some embodiments the solid state form of Formula (I) is solid state Form V. In some embodiments, solid state Form V of Formula (I) is substantially crystalline.

The compound having Formula (I) shows complicated polymorphic behaviors. Three anhydrous forms, Forms A, I, and T, and three hydrate forms, Forms C, D, and E, were identified. In addition, other crystalline forms and a substantially amorphous form were identified.

Methods for collection of XRPD data are known in the art, and any such methods can be used for characterizing the crystalline forms of the compound of Formula (I). For example, the X-ray powder diffraction patterns described herein can be generated using Cu Kul radiation.

In some embodiments, the crystalline form described herein is further characterized by a differential scanning calorimetry (DSC) thermogram. In some embodiments, a DSC thermogram is recorded using a sample weight of about 1-2 mg, which is subjected to temperatures ranging from 30° C. to 350° C. using a ramp of 10° C./min.

In some embodiments, the crystalline form described herein is further characterized by a thermal gravimetric analysis (TGA). In some embodiments, a TGA thermogram is recorded using a sample weight of about 2-10 mg, which is subjected to temperatures ranging from 30° C. to 300° C. using a ramp of 10° C./min.

In some embodiments, the crystalline form described herein is further characterized by a polarized light microscope (PLM) profile. In some embodiments, a polarized light microscope (PLM) is recorded by using a crossed polarizer.

In some embodiments, the crystalline form described herein is further characterized by is further characterized by a dynamic vapor sorption (DVS) profile. In some embodiments, a DVS is recorded according to the method as described herein with a cycle of 40-0-95-0-40% RH at 25° C.

### III-1. Crystalline Form A

In one embodiment, the present disclosure provides crystalline Form A of the compound having Formula (I). In some embodiments, the X-ray powder diffraction pattern includes peaks at 6.1, 11.1, and 16.6 degrees 2θ (±0.2 degrees 2θ). In some embodiments, the X-ray powder diffraction pattern includes peaks at 6.1, 11.1, 12.1, and 16.6 degrees 2θ (±0.2 degrees 2θ). In some embodiments, the X-ray powder diffraction pattern includes peaks at 6.1, 6.6, 11.1, 12.1, and 16.6 degrees 2θ (±0.2 degrees 2θ).

In one embodiment, the present disclosure provides crystalline Form A of the compound having Formula (I), characterized by an X-ray powder diffraction (XRPD) pattern including peaks at 6.1, 11.1, 12.1, 16.6, and 27.7 degrees 2θ (±0.2 degrees 2θ). In some embodiments, crystalline Form A of the compound having Formula (I), characterized by the X-ray powder diffraction pattern further includes peaks at 6.6, 15.6, 22.4, 27.4, and 28.4 degrees 2θ (±0.2 degrees 2θ). In some embodiments, crystalline Form A of the compound having Formula (I), characterized by the X-ray powder diffraction pattern further includes peaks at 10.0, 18.5, 20.8, 25.3, and 25.7 degrees 2θ (±0.2 degrees 2θ). In some embodiments, crystalline Form A of the compound having Formula (I), characterized by an X-ray powder diffraction (XRPD) pattern including peaks (i.e., the first 10 peaks ranked according to relative peak intensity %) at 6.1, 6.6, 11.1, 12.1, 15.6, 16.6, 22.4, 27.4, 27.7, and 28.4 degrees 2θ (±0.2 degrees 2θ). In some embodiments, crystalline Form A of the compound having Formula (I), characterized by an X-ray powder diffraction (XRPD) pattern including peaks at 6.1, 6.6, 10.0, 11.1, 12.1, 15.6, and 16.6 degrees 2θ (±0.2 degrees 2θ). In some embodiments, crystalline Form A of the compound having Formula (I), characterized by an X-ray powder diffraction (XRPD) pattern comprising at least any of the two peaks selected from 6.1, 6.6, 10.0, 11.1, 12.1, 15.6, and 16.6 degrees 2θ (±0.2 degrees 2θ). In some embodiments, crystalline Form A of the compound having Formula (I), characterized by an X-ray powder diffraction (XRPD) pattern comprising at least any of the three peaks selected from 6.1, 6.6, 10.0, 11.1, 12.1, 15.6, and 16.6 degrees 2θ (±0.2 degrees 2θ). In some embodiments, crystalline Form A of the compound having Formula (I), characterized by an X-ray powder diffraction (XRPD) pattern comprising at least any of the four peaks selected from 6.1, 6.6, 10.0, 11.1, 12.1, 15.6, and 16.6 degrees 2θ (±0.2 degrees 2θ). In some embodiments, crystalline Form A of the compound having Formula (I), characterized by an X-ray powder diffraction (XRPD) pattern comprising at least any of the five peaks selected from 6.1, 6.6, 10.0, 11.1, 12.1, 15.6, and 16.6 degrees 2θ (±0.2 degrees 2θ). In some embodiments, Form A is characterized by an X-ray powder diffraction (XRPD) pattern including one, two, three, four, five or more peaks listed in Table 4. In some embodiments, Form A is characterized by an X-ray powder diffraction (XRPD) pattern including at least five peaks listed in Table 4. In some embodiments, Form A is characterized by an X-ray powder diffraction (XRPD) pattern including at least four peaks listed in Table 4. In some embodiments, Form A is characterized by an X-ray powder diffraction (XRPD) pattern including at least three peaks listed in Table 4.

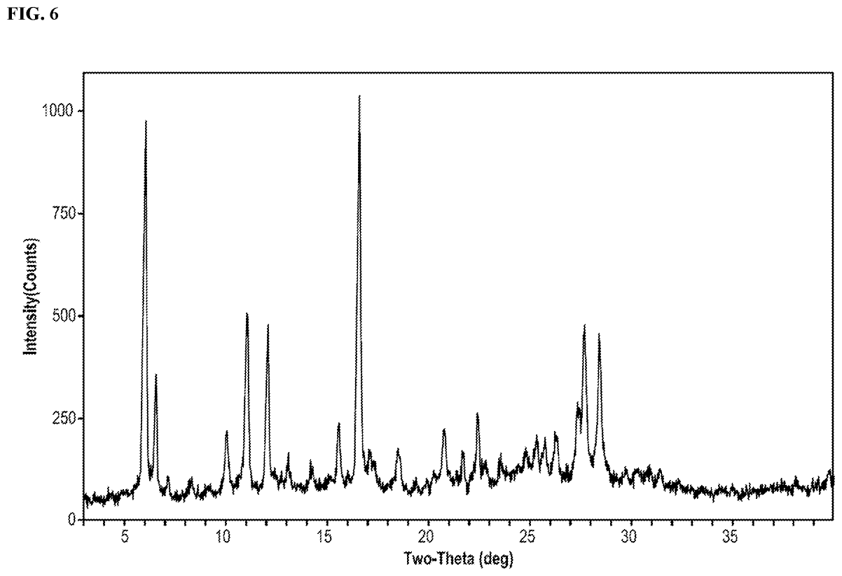

In some embodiments, crystalline Form A of the compound having Formula (I) is characterized by an X-ray powder diffraction (XRPD) pattern substantially in accordance with FIG. 6.

In some embodiments, the crystalline Form A is substantially free of other crystalline or amorphous forms of the compound having Formula (I).

In some embodiments, crystalline Form A is further characterized by a differential scanning calorimetry (DSC) thermogram including an endothermic peak at about 323.5° C. In some embodiments, crystalline Form A is further characterized by a differential scanning calorimetry (DSC) thermogram including an onset temperature of about 318.2° C. and an endothermic peak at about 323.5° C.

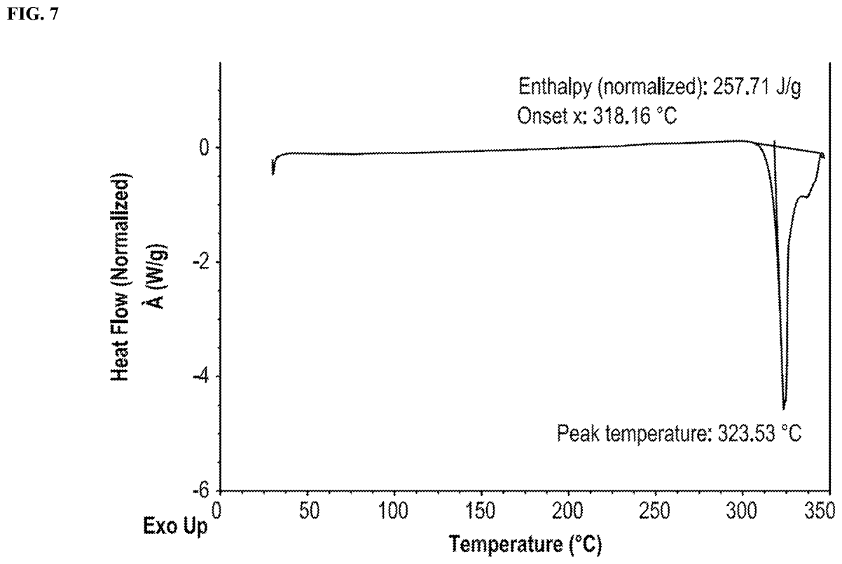

In some embodiments, crystalline Form A is further characterized by a differential scanning calorimetry (DSC) thermogram substantially in accordance with FIG. 7.

In some embodiments, crystalline Form A is further characterized by a weight percent loss of about 1.0% upon heating to about 220° C., as measured by a thermal gravimetric analysis (TGA).

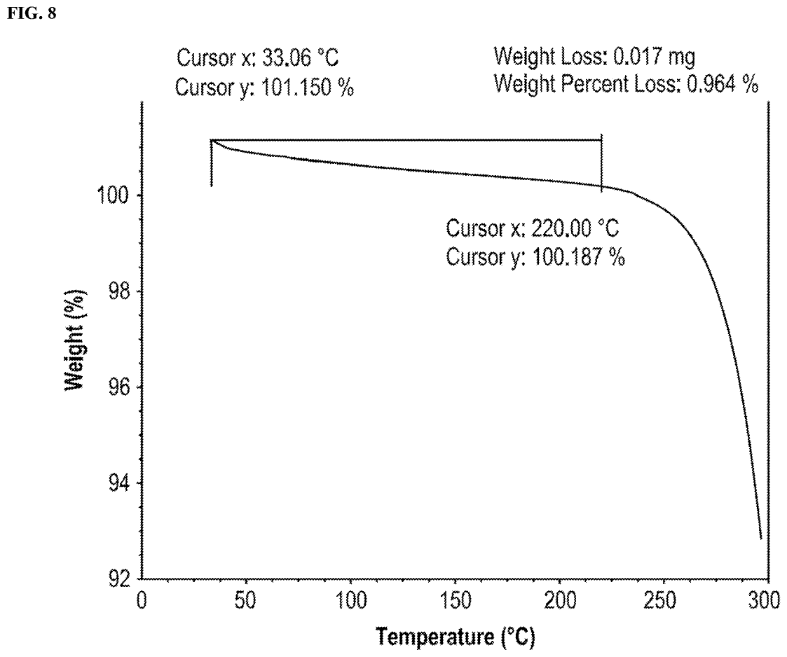

In some embodiments, crystalline Form A is further characterized by a thermal gravimetric analysis (TGA) thermogram substantially in accordance with FIG. 8.

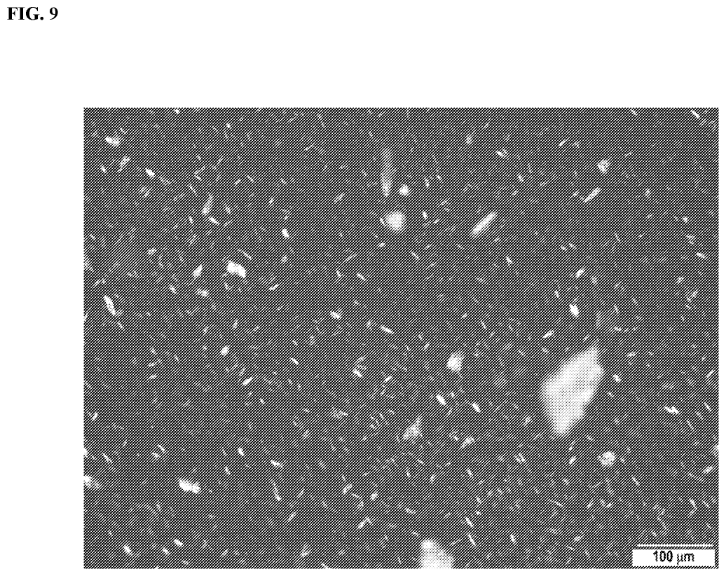

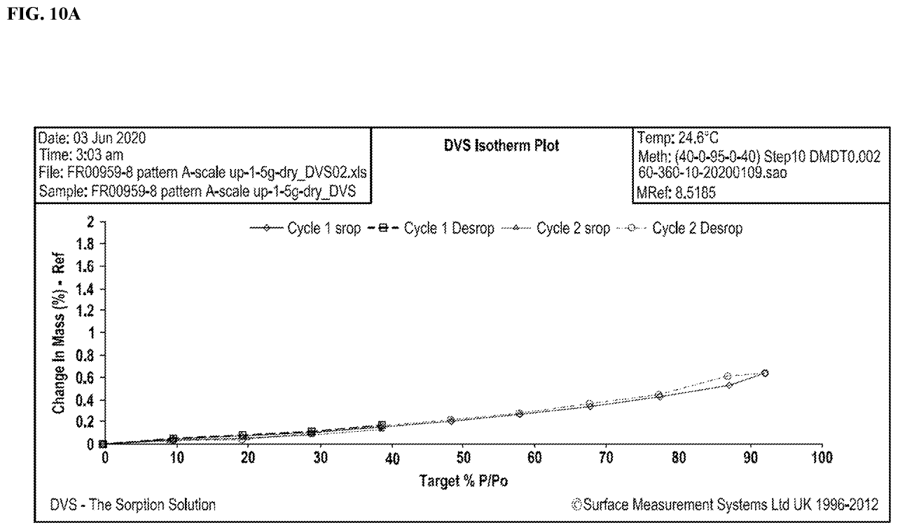

In some embodiments, crystalline Form A is further characterized by a polarized light microscope (PLM) profile substantially as shown in FIG. 9.

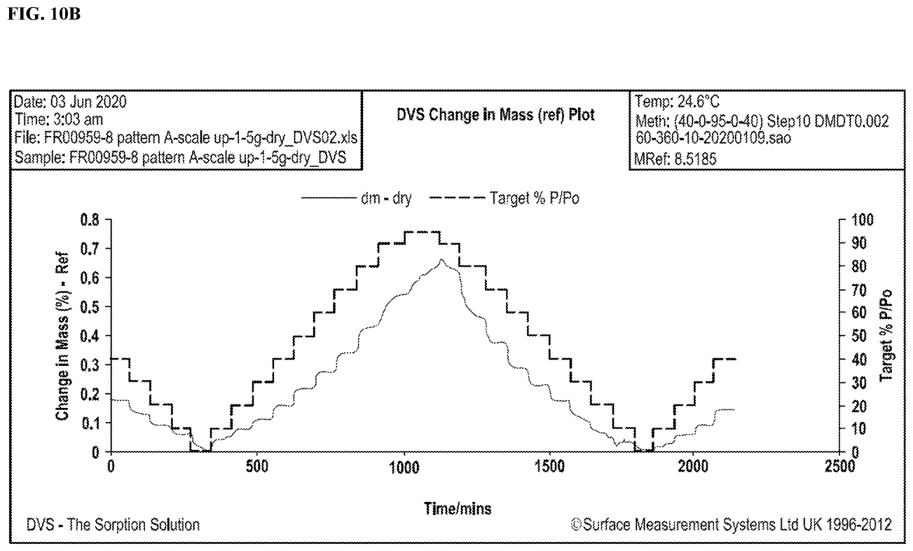

In some embodiments, crystalline Form A is further characterized by a dynamic vapor sorption (DVS) profile substantially as shown in FIG. 10.

Crystalline Form A is in an anhydrous form.

In some embodiments, crystalline Form A is characterized by an X-ray powder diffraction pattern substantially in accordance with FIG. 6; and is further characterized by a differential scanning calorimetry (DSC) thermogram substantially in accordance with FIG. 7. In some embodiments, crystalline Form A is characterized by an X-ray powder diffraction pattern substantially in accordance with FIG. 6; is further characterized by a differential scanning calorimetry (DSC) thermogram substantially in accordance with FIG. 7; and is further characterized by a thermal gravimetric analysis (TGA) thermogram substantially in accordance with FIG. 8.

### III-2. Crystalline Form B

In one embodiment, the present disclosure provides crystalline Form B of the compound having Formula (I). In some embodiments, the X-ray powder diffraction pattern includes peaks at 10.6, 16.6, and 18.1 degrees 2θ (±0.2 degrees 2θ). In some embodiments, the X-ray powder diffraction pattern includes peaks at 10.6, 11.8, 16.6, and 18.1 degrees 2θ (±0.2 degrees 2θ). In some embodiments, the X-ray powder diffraction pattern includes peaks at 10.6, 11.5, 11.8, 16.6, and 18.1 degrees 2θ (±0.2 degrees 2θ).

In one embodiment, the present disclosure provides crystalline Form B of the compound having Formula (I), characterized by an X-ray powder diffraction (XRPD) pattern including peaks at 10.6, 16.6, 18.1, 26.6, and 27.3 degrees 2θ (±0.2 degrees 2θ). In some embodiments, the X-ray powder diffraction pattern further includes peaks at 11.5, 11.8, 12.0, 19.7, and 28.3 degrees 2θ (±0.2 degrees 2θ). In some embodiments, the X-ray powder diffraction pattern further includes peaks at 17.3, 20.3, 22.1, 24.2, and 29.6 degrees 2θ (±0.2 degrees 2θ). In some embodiments, crystalline Form B of the compound having Formula (I), characterized by an X-ray powder diffraction (XRPD) pattern including peaks (i.e., the first 10 peaks ranked according to relative peak intensity %) at 10.6, 11.5, 11.8, 12.0, 16.6, 18.1, 19.7, 26.6, 27.3, and 28.3 degrees 2θ (±0.2 degrees 2θ). In some embodiments, Form B is characterized by an X-ray powder diffraction (XRPD) pattern including one, two, three, four, five or more peaks listed in Table 5. In some embodiments, Form B is characterized by an X-ray powder diffraction (XRPD) pattern including at least five peaks listed in Table 5. In some embodiments, Form B is characterized by an X-ray powder diffraction (XRPD) pattern including at least four peaks listed in Table 5.

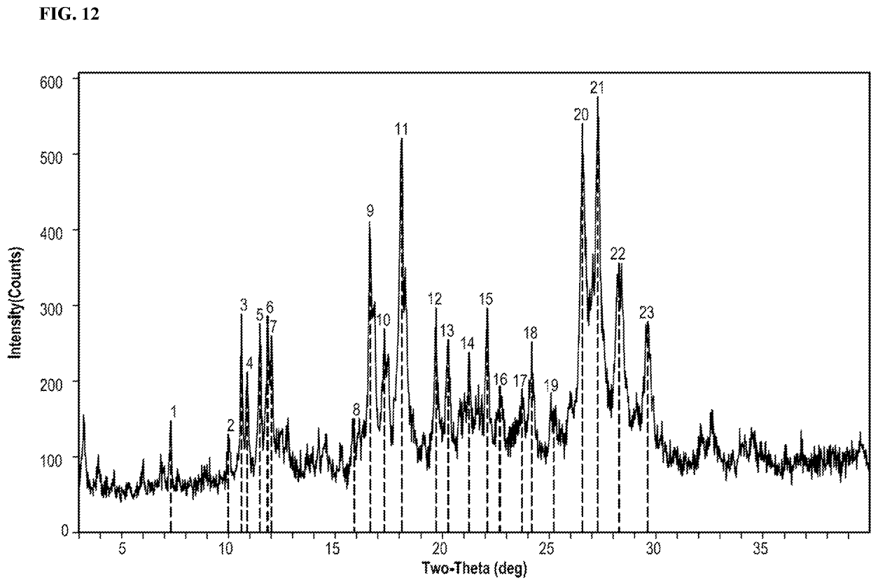

In some embodiments, crystalline Form B of the compound having Formula (I) is characterized by an X-ray powder diffraction (XRPD) pattern substantially in accordance with FIG. 12.

In some embodiments, crystalline Form B is substantially free of other crystalline or amorphous forms of the compound having Formula (I).

### III-3. Crystalline Form C

In one embodiment, the present disclosure provides crystalline Form C of a compound having Formula (I). In some embodiments, the X-ray powder diffraction pattern includes peaks at 11.8, 16.6, and 17.5 degrees 2θ (±0.2 degrees 2θ). In some embodiments, the X-ray powder diffraction pattern includes peaks at 10.9, 11.8, 16.6, and 17.5 degrees 2θ (±0.2 degrees 2θ). In some embodiments, the X-ray powder diffraction pattern includes peaks at 10.9, 11.8, 16.6, 17.5, and 17.8 degrees 2θ (±0.2 degrees 2θ).

In one embodiment, the present disclosure provides crystalline Form C of a compound having Formula (I), characterized by an X-ray powder diffraction (XRPD) pattern including peaks at 11.8, 16.6, 17.5, 27.2, and 28.2 degrees 2θ (±0.2 degrees 2θ). In some embodiments, the X-ray powder diffraction pattern further includes peaks at 19.7, 20.3, 23.7, 24.5, and 29.8 degrees 2θ (±0.2 degrees 2θ). In some embodiments, the X-ray powder diffraction pattern further includes peaks at 10.9, 17.8, 21.8, 26.0, and 26.6 degrees 2θ (±0.2 degrees 2θ). In some embodiments, crystalline Form C of a compound having Formula (I), characterized by an X-ray powder diffraction (XRPD) pattern including peaks (i.e., the first 10 peaks ranked according to relative peak intensity %) at 1.8, 16.6, 17.5, 19.7, 20.3, 23.7, 24.5, 27.2, 28.2, and 29.8 degrees 2θ (±0.2 degrees 2θ). In some embodiments, crystalline Form C of the compound having Formula (I), characterized by an X-ray powder diffraction (XRPD) pattern including peaks at 10.9, 11.8, 16.6, 17.5, and 17.8 degrees 2θ (±0.2 degrees 2θ). In some embodiments, crystalline Form C of the compound having Formula (I), characterized by an X-ray powder diffraction (XRPD) pattern comprising at least any of the two peaks selected from 10.9, 11.8, 16.6, 17.5, and 17.8 degrees 2θ (±0.2 degrees 2θ). In some embodiments, crystalline Form C of the compound having Formula (I), characterized by an X-ray powder diffraction (XRPD) pattern comprising at least any of the three peaks selected from 10.9, 11.8, 16.6, 17.5, and 17.8 degrees 2θ (±0.2 degrees 2θ). In some embodiments, crystalline Form C of the compound having Formula (I), characterized by an X-ray powder diffraction (XRPD) pattern comprising at least any of the four peaks selected from 10.9, 11.8, 16.6, 17.5, and 17.8 degrees 2θ (±0.2 degrees 2θ). In some embodiments, Form C is characterized by an X-ray powder diffraction (XRPD) pattern including one, two, three, four, five or more peaks listed in Table 7. In some embodiments, Form C is characterized by an X-ray powder diffraction (XRPD) pattern including at least five peaks listed in Table 7. In some embodiments, Form C is characterized by an X-ray powder diffraction (XRPD) pattern including at least four peaks listed in Table 7.

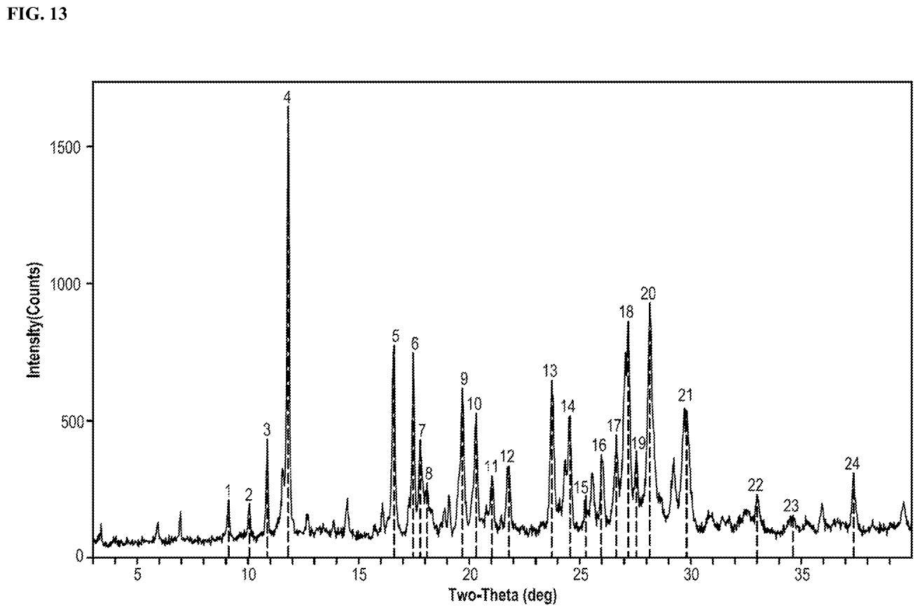

In some embodiments, crystalline Form C of a compound having Formula (I), is characterized by an X-ray powder diffraction pattern substantially in accordance with FIG. 13.

In some embodiments, crystalline Form C is substantially free of other crystalline or amorphous forms of the compound having Formula (I).

In some embodiments, crystalline Form C is further characterized by a differential scanning calorimetry (DSC) thermogram including one or more endothermic peaks at about 69.5° C., about 197.5° C., and about 326.6° C. In some embodiments, crystalline Form C is further characterized by a differential scanning calorimetry (DSC) thermogram including an endothermic peak at about 69.5° C. In some embodiments, crystalline Form C is further characterized by a differential scanning calorimetry (DSC) thermogram including an endothermic peak at about 197.5° C. In some embodiments, crystalline Form C is further characterized by a differential scanning calorimetry (DSC) thermogram including an endothermic peak at about 326.6° C. In some embodiments, crystalline Form C is further characterized by a differential scanning calorimetry (DSC) thermogram including an onset temperature of about 43.8° C. and an endothermic peak at about 69.5° C. In some embodiments, crystalline Form C is further characterized by a differential scanning calorimetry (DSC) thermogram including an onset temperature of about 184.5° C. and an endothermic peak at about 197.5° C. In some embodiments, crystalline Form C is further characterized by a differential scanning calorimetry (DSC) thermogram including an onset temperature of about 321.3° C. and an endothermic peak at about 326.6° C.

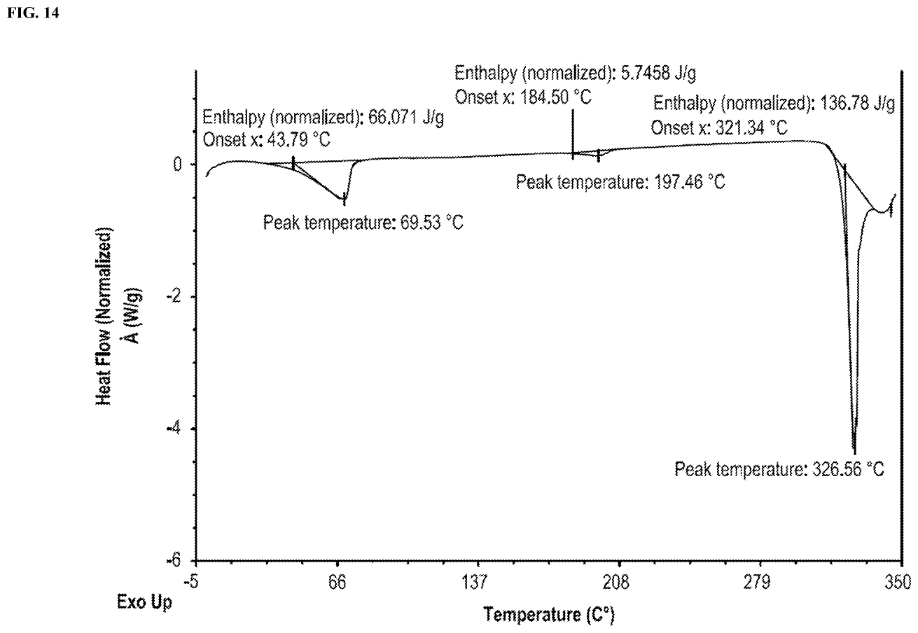

In some embodiments, crystalline Form C is further characterized by a differential scanning calorimetry (DSC) thermogram substantially in accordance with FIG. 14.

In some embodiments, crystalline Form C is further characterized by a weight percent loss of about 3.9% upon heating to about 220° C., as measured by a thermal gravimetric analysis (TGA).

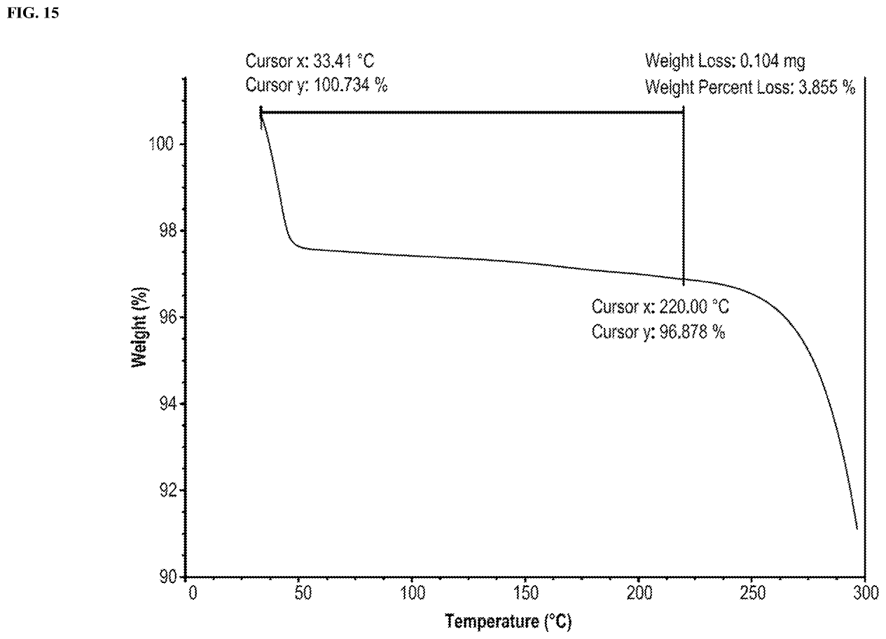

In some embodiments, crystalline Form C is further characterized by a thermal gravimetric analysis (TGA) thermogram substantially in accordance with FIG. 15.

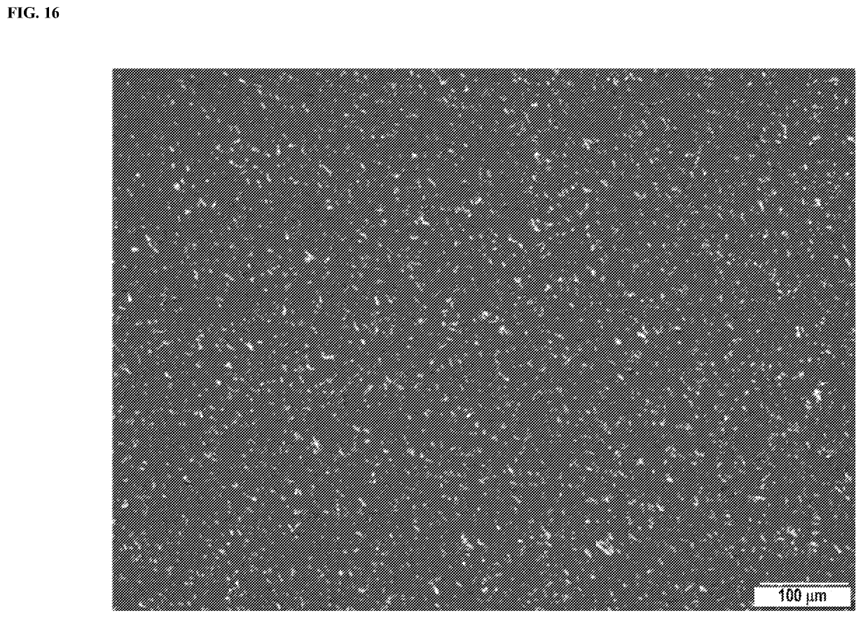

In some embodiments, crystalline Form C is further characterized by a polarized light microscope (PLM) profile substantially as shown in FIG. 16. In some embodiments, crystalline Form C is in an irregular particle characterized by a polarized light microscope (PLM) profile substantially as shown in FIG. 16.

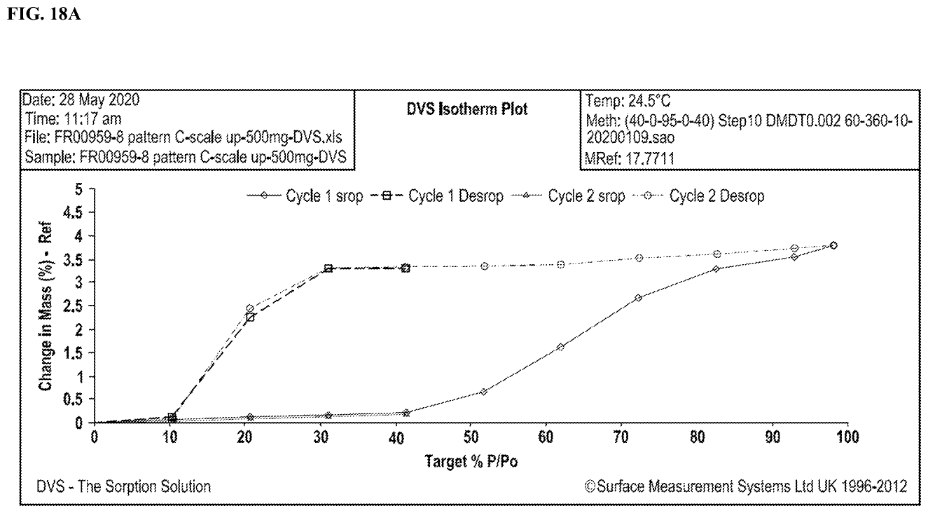

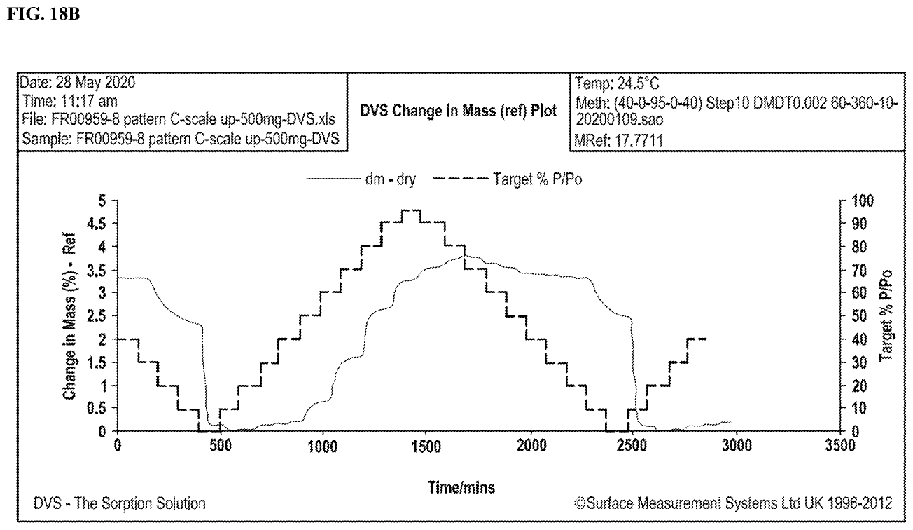

In some embodiments, crystalline Form C is further characterized by a dynamic vapor sorption (DVS) profile substantially as shown in FIG. 18.

Crystalline Form C is in a hydrate form.

In some embodiments, crystalline Form C is characterized by an X-ray powder diffraction pattern substantially in accordance with FIG. 13; and is further characterized by a differential scanning calorimetry (DSC) thermogram substantially in accordance with FIG. 14. In some embodiments, crystalline Form C is characterized by an X-ray powder diffraction pattern substantially in accordance with FIG. 13; is further characterized by a differential scanning calorimetry (DSC) thermogram substantially in accordance with FIG. 14; and is further characterized by a thermal gravimetric analysis (TGA) thermogram substantially in accordance with FIG. 15.

### III-4. Crystalline Form D

In one embodiment, the present disclosure provides crystalline Form D of a compound having Formula (I). In some embodiments, the X-ray powder diffraction pattern includes peaks at 6.1, 12.4, and 16.5 degrees 2θ (±0.2 degrees 2θ). In some embodiments, the X-ray powder diffraction pattern includes peaks at 6.1, 12.4, 13.7, and 16.5 degrees 2θ (±0.2 degrees 2θ). In some embodiments, the X-ray powder diffraction pattern includes peaks at 6.1, 10.9 12.4, 13.7, and 16.5 degrees 2θ (±0.2 degrees 2θ).

In one embodiment, the present disclosure provides crystalline Form D of a compound having Formula (I), characterized by an X-ray powder diffraction (XRPD) pattern including peaks at 6.1, 12.4, 13.7, 16.5, and 27.6 degrees 2θ (±0.2 degrees 2θ). In some embodiments, the X-ray powder diffraction pattern further includes peaks at 10.9, 14.8, 25.2, 26.7, and 27.9 degrees 2θ (±0.2 degrees 2θ). In some embodiments, the X-ray powder diffraction pattern further includes peaks at 10.3, 19.3, 21.2, 24.1, and 29.9 degrees 2θ (±0.2 degrees 2θ). In some embodiments, crystalline Form D of a compound having Formula (I), characterized by an X-ray powder diffraction (XRPD) pattern including peaks (i.e., the first 10 peaks ranked according to relative peak intensity %) at 6.1, 10.9, 12.4, 13.7, 14.8, 16.5, 25.2, 26.7, 27.6, and 27.9 degrees 2θ (±0.2 degrees 2θ). In some embodiments, crystalline Form D of the compound having Formula (I), characterized by an X-ray powder diffraction (XRPD) pattern including peaks at 6.1, 10.9, 12.4, 13.7, 14.8 and 16.5 degrees 2θ (±0.2 degrees 2θ). In some embodiments, crystalline Form D of the compound having Formula (I), characterized by an X-ray powder diffraction (XRPD) pattern comprising at least any of the two peaks selected from 6.1, 10.9, 12.4, 13.7, 14.8, and 16.5 degrees 2θ (±0.2 degrees 2θ). In some embodiments, crystalline Form D of the compound having Formula (I), characterized by an X-ray powder diffraction (XRPD) pattern comprising at least any of the three peaks selected from 6.1, 10.9, 12.4, 13.7, 14.8, and 16.5 degrees 2θ (±0.2 degrees 2θ). In some embodiments, crystalline Form D of the compound having Formula (I), characterized by an X-ray powder diffraction (XRPD) pattern comprising at least any of the four peaks selected from 6.1, 10.9, 12.4, 13.7, 14.8, and 16.5 degrees 2θ (±0.2 degrees 2θ). In some embodiments, crystalline Form D of the compound having Formula (I), characterized by an X-ray powder diffraction (XRPD) pattern comprising at least any of the five peaks selected from 6.1, 10.9, 12.4, 13.7, 14.8, and 16.5 degrees 2θ (±0.2 degrees 2θ). In some embodiments, Form D is characterized by an X-ray powder diffraction (XRPD) pattern including one, two, three, four, five or more peaks listed in Table 8. In some embodiments, Form D is characterized by an X-ray powder diffraction (XRPD) pattern including at least five peaks listed in Table 8. In some embodiments, Form D is characterized by an X-ray powder diffraction (XRPD) pattern including at least four peaks listed in Table 8.

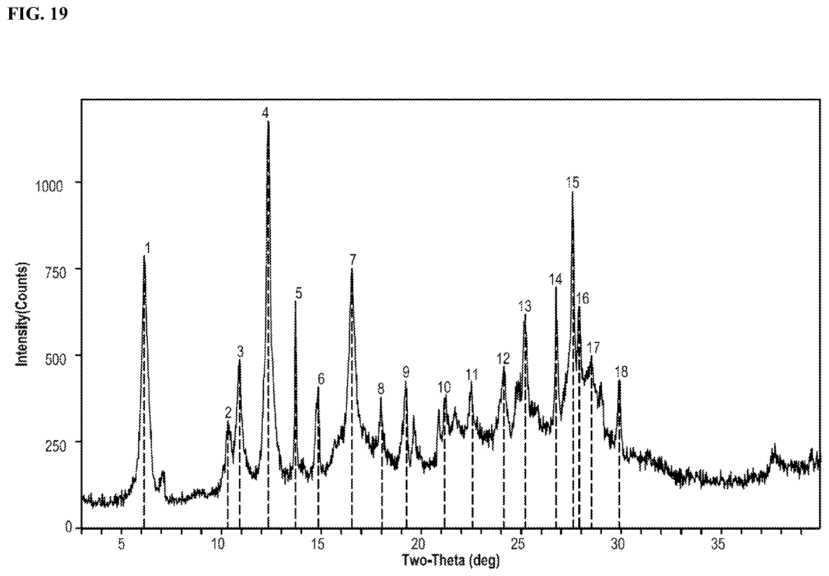

In some embodiments, crystalline Form D of a compound having Formula (I), is characterized by an X-ray powder diffraction pattern substantially in accordance with FIG. 19.

In some embodiments, crystalline Form D is substantially free of other crystalline or amorphous forms of the compound having Formula (I).

In some embodiments, crystalline Form D is further characterized by a differential scanning calorimetry (DSC) thermogram including one or more endothermic peaks at about 66.8° C. and about 322.0° C. In some embodiments, crystalline Form D is further characterized by a differential scanning calorimetry (DSC) thermogram including an endothermic peak at about 66.8° C. In some embodiments, crystalline Form D is further characterized by a differential scanning calorimetry (DSC) thermogram including an endothermic peak at about 322.0° C. In some embodiments, crystalline Form D is further characterized by a differential scanning calorimetry (DSC) thermogram including an onset temperature of about 45.8° C. and an endothermic peak at about 66.8° C. In some embodiments, crystalline Form D is further characterized by a differential scanning calorimetry (DSC) thermogram including an onset temperature of about 317.1° C. and an endothermic peak at about 322.0° C.

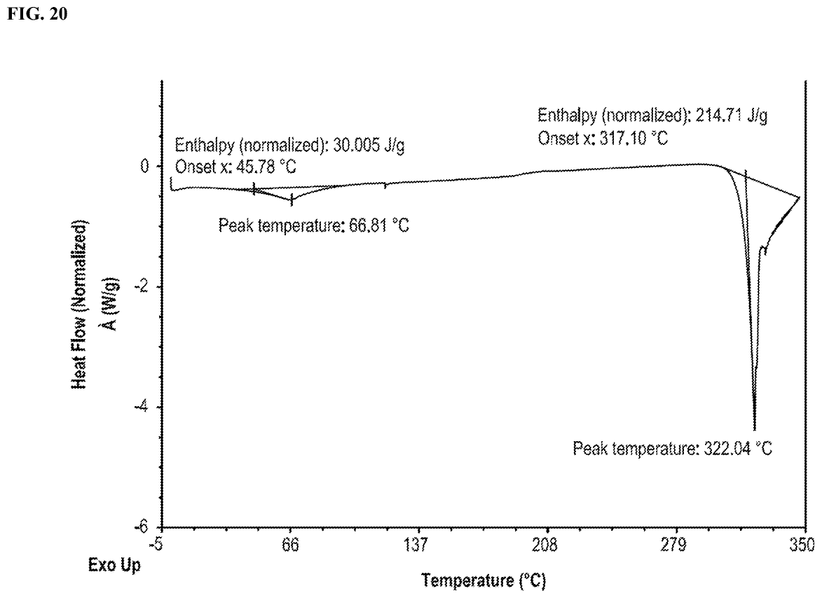

In some embodiments, crystalline Form D is further characterized by a differential scanning calorimetry (DSC) thermogram substantially in accordance with FIG. 20.

In some embodiments, crystalline Form D is further characterized by a weight percent loss of about 2.10% upon heating to about 220° C., as measured by a thermal gravimetric analysis (TGA).

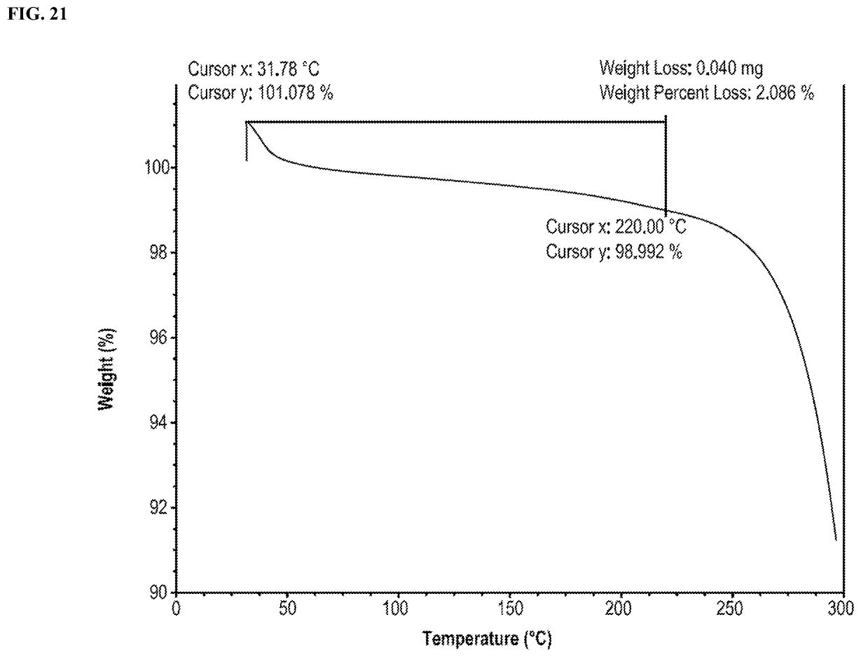

In some embodiments, crystalline Form D is further characterized by a thermal gravimetric analysis (TGA) thermogram substantially in accordance with FIG. 21.

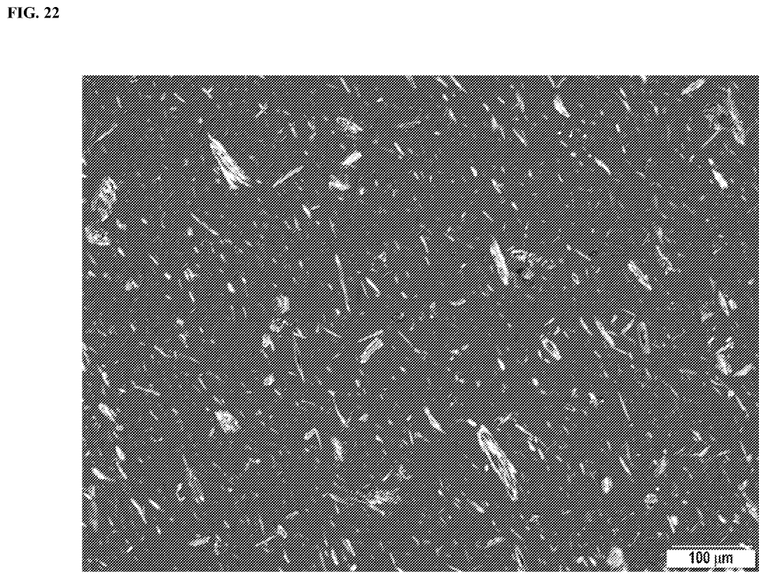

In some embodiments, crystalline Form D is further characterized by a polarized light microscope (PLM) profile substantially as shown in FIG. 22. In some embodiments, crystalline Form D is in an irregular particle characterized by a polarized light microscope (PLM) profile substantially as shown in FIG. 22.

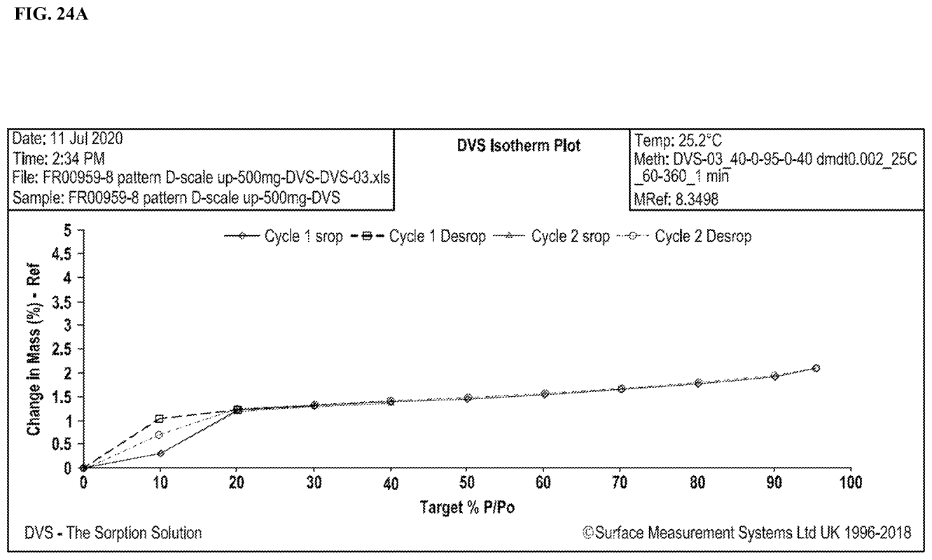

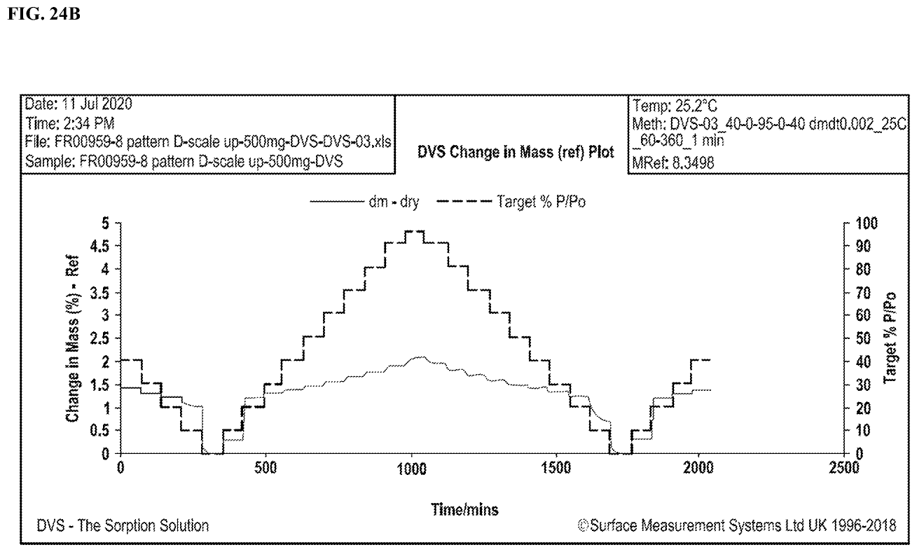

In some embodiments, crystalline Form C is further characterized by a dynamic vapor sorption (DVS) profile substantially as shown in FIG. 24.

Crystalline Form D is in a hydrate form.

In some embodiments, crystalline Form D is characterized by an X-ray powder diffraction pattern substantially in accordance with FIG. 19; and is further characterized by a differential scanning calorimetry (DSC) thermogram substantially in accordance with FIG. 20. In some embodiments, crystalline Form D is characterized by an X-ray powder diffraction pattern substantially in accordance with FIG. 19; is further characterized by a differential scanning calorimetry (DSC) thermogram substantially in accordance with FIG. 20; and is further characterized by a thermal gravimetric analysis (TGA) thermogram substantially in accordance with FIG. 21.

### III-5. Crystalline Form E

In one embodiment, the present disclosure provides crystalline Form E of a compound having Formula (I). In some embodiments, the X-ray powder diffraction pattern includes peaks at 12.3, 13.7, and 19.4 degrees 2θ (±0.2 degrees 2θ). In some embodiments, the X-ray powder diffraction pattern includes peaks at 12.3, 13.7, 15.0, and 19.4 degrees 2θ (±0.2 degrees 2θ). In some embodiments, the X-ray powder diffraction pattern includes peaks at 12.3, 13.7, 14.6, 15.0, and 19.4 degrees 2θ (±0.2 degrees 2θ).

In one embodiment, the present disclosure provides crystalline Form E of a compound having Formula (I), characterized by an X-ray powder diffraction (XRPD) pattern including peaks at 12.3, 13.7, 19.4, 26.7, and 27.6 degrees 2θ (±0.2 degrees 2θ). In some embodiments, the X-ray powder diffraction pattern further includes peaks at 20.8, 24.5, 25.3, 28.1, and 30.0 degrees 2θ (±0.2 degrees 2θ). In some embodiments, the X-ray powder diffraction pattern further includes peaks at 7.2, 14.6, 14.9, 18.2, and 22.5 degrees 2θ (±0.2 degrees 2θ). In some embodiments, crystalline Form E of a compound having Formula (I), characterized by an X-ray powder diffraction (XRPD) pattern including peaks (i.e., the first 10 peaks ranked according to relative peak intensity %) at 12.3, 13.7, 19.4, 20.8, 24.5, 25.3, 26.7, 27.6, 28.1, and 30.0 degrees 2θ (±0.2 degrees 2θ). In some embodiments, crystalline Form E of the compound having Formula (I), characterized by an X-ray powder diffraction (XRPD) pattern including peaks at 7.2, 12.3, 13.7, 14.6, 14.9, 18.2 and 19.4 degrees 2θ (±0.2 degrees 2θ). In some embodiments, crystalline Form E of the compound having Formula (I), characterized by an X-ray powder diffraction (XRPD) pattern comprising at least any of the two peaks selected from 7.2, 12.3, 13.7, 14.6, 14.9, 18.2 and 19.4 degrees 2θ (±0.2 degrees 2θ). In some embodiments, crystalline Form E of the compound having Formula (I), characterized by an X-ray powder diffraction (XRPD) pattern comprising at least any of the three peaks selected from 7.2, 12.3, 13.7, 14.6, 14.9, 18.2 and 19.4 degrees 2θ (±0.2 degrees 2θ). In some embodiments, crystalline Form E of the compound having Formula (I), characterized by an X-ray powder diffraction (XRPD) pattern comprising at least any of the four peaks selected from 7.2, 12.3, 13.7, 14.6, 14.9, 18.2 and 19.4 degrees 2θ (±0.2 degrees 2θ). In some embodiments, crystalline Form E of the compound having Formula (I), characterized by an X-ray powder diffraction (XRPD) pattern comprising at least any of the five peaks selected from 7.2, 12.3, 13.7, 14.6, 14.9, 18.2 and 19.4 degrees 2θ (±0.2 degrees 2θ). In some embodiments, crystalline Form E of the compound having Formula (I), characterized by an X-ray powder diffraction (XRPD) pattern comprising at least any of the six peaks selected from 7.2, 12.3, 13.7, 14.6, 14.9, 18.2 and 19.4 degrees 2θ (±0.2 degrees 2θ). In some embodiments, Form E is characterized by an X-ray powder diffraction (XRPD) pattern including one, two, three, four, five or more peaks listed in Table 10. In some embodiments, Form E is characterized by an X-ray powder diffraction (XRPD) pattern including at least five peaks listed in Table 10. In some embodiments, Form E is characterized by an X-ray powder diffraction (XRPD) pattern including at least four peaks listed in Table 10.

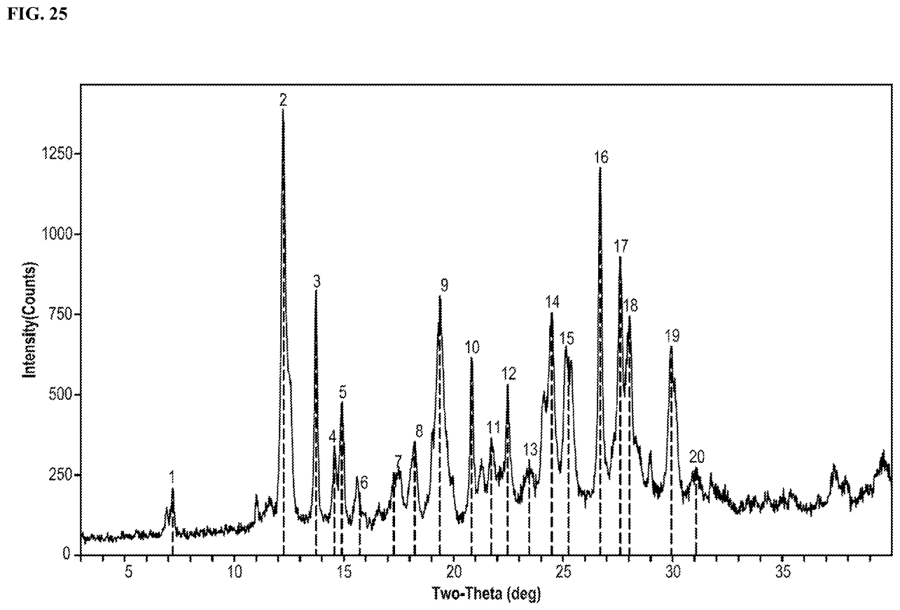

In some embodiments, crystalline Form E of a compound having Formula (I), is characterized by an X-ray powder diffraction pattern substantially in accordance with FIG. 25.

In some embodiments, crystalline Form E is substantially free of other crystalline or amorphous forms of the compound having Formula (I).

In some embodiments, crystalline Form E is further characterized by a differential scanning calorimetry (DSC) thermogram including one or more endothermic peaks at about 85.9° C. and about 325.0° C. In some embodiments, the differential scanning calorimetry (DSC) thermogram further includes an exothermic peak at 199.6° C. In some embodiments, crystalline Form E is further characterized by a differential scanning calorimetry (DSC) thermogram including an endothermic peak at about 85.9° C. In some embodiments, crystalline Form E is further characterized by a differential scanning calorimetry (DSC) thermogram including an endothermic peak at about 325.0° C. In some embodiments, crystalline Form E is further characterized by a differential scanning calorimetry (DSC) thermogram including an onset temperature of about 58.9° C. and an endothermic peak at about 85.9° C. In some embodiments, crystalline Form E is further characterized by a differential scanning calorimetry (DSC) thermogram including an onset temperature of about 195.4° C. and an exothermic peak at about 199.6° C. In some embodiments, crystalline Form E is further characterized by a differential scanning calorimetry (DSC) thermogram including an onset temperature of about 318.3° C. and an endothermic peak at about 325.0° C.

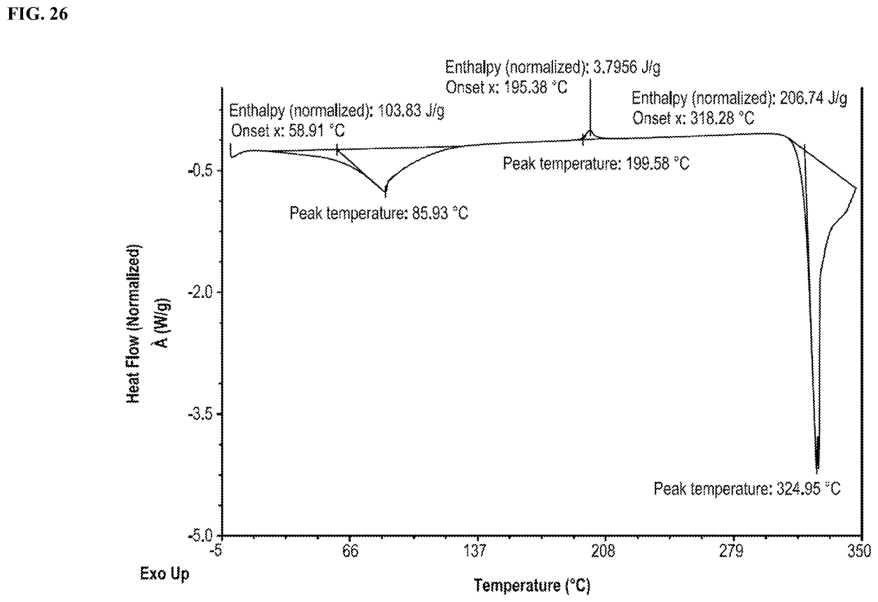

In some embodiments, crystalline Form E is further characterized by a differential scanning calorimetry (DSC) thermogram substantially in accordance with FIG. 26.

In some embodiments, crystalline Form E is further characterized by a weight percent loss of about 4.3% upon heating to about 195° C., as measured by a thermal gravimetric analysis (TGA).

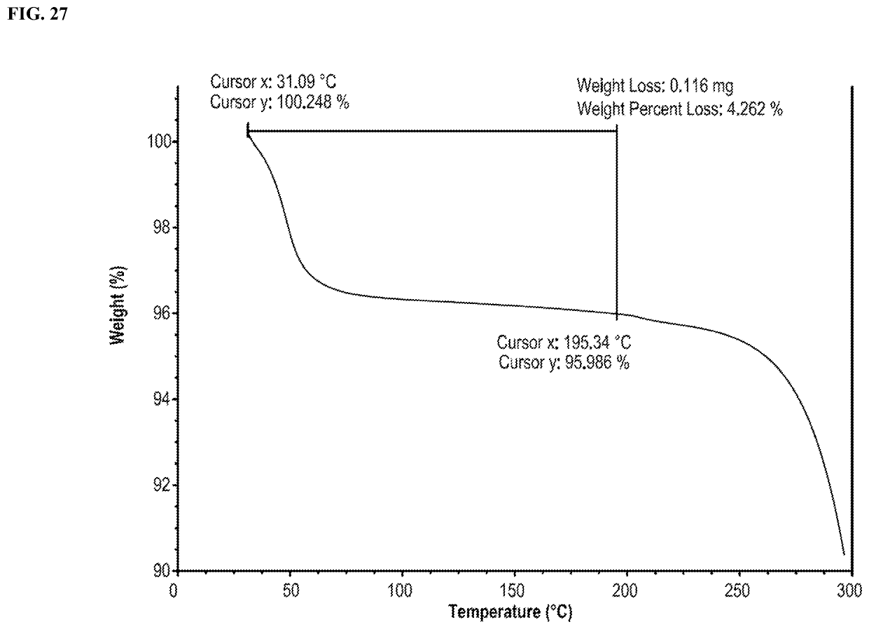

In some embodiments, crystalline Form E is further characterized by a thermal gravimetric analysis (TGA) thermogram substantially in accordance with FIG. 27.

In some embodiments, crystalline Form E is further characterized by a polarized light microscope (PLM) profile substantially as shown in FIG. 28. In some embodiments, crystalline Form E is in an irregular particle characterized by a polarized light microscope (PLM) profile substantially as shown in FIG. 28.

Crystalline Form E is in a hydrate form.

In some embodiments, crystalline Form E is characterized by an X-ray powder diffraction pattern substantially in accordance with FIG. 25; and is further characterized by a differential scanning calorimetry (DSC) thermogram substantially in accordance with FIG. 26. In some embodiments, crystalline Form E is characterized by an X-ray powder diffraction pattern substantially in accordance with FIG. 25; is further characterized by a differential scanning calorimetry (DSC) thermogram substantially in accordance with FIG. 26; and is further characterized by a thermal gravimetric analysis (TGA) thermogram substantially in accordance with FIG. 27.

### III-6. Crystalline Form F

In one embodiment, the present disclosure provides crystalline Form F of a compound having Formula (I). In some embodiments, the X-ray powder diffraction pattern includes peaks at 7.8 and 18.2 degrees 2θ (±0.2 degrees 2θ). In some embodiments, the X-ray powder diffraction pattern includes peaks at 7.8, 17.8 and 18.2 degrees 2θ (±0.2 degrees 2θ). In some embodiments, the X-ray powder diffraction pattern includes peaks at 7.8, 14.7, 17.8 and 18.2 degrees 2θ (±0.2 degrees 2θ). In some embodiments, the X-ray powder diffraction pattern includes peaks at 7.8, 14.7, 15.7, 17.8 and 18.2 degrees 2θ (±0.2 degrees 2θ).

In one embodiment, the present disclosure provides crystalline Form F of a compound having Formula (I), characterized by an X-ray powder diffraction (XRPD) pattern including peaks at 7.8, 18.2, 23.8, 27.5, and 27.8 degrees 2θ (±0.2 degrees 2θ). In some embodiments, the X-ray powder diffraction pattern further includes peaks at 14.7, 15.7, 17.8, 24.9, and 25.4 degrees 2θ (±0.2 degrees 2θ). In some embodiments, the X-ray powder diffraction pattern further includes peaks at 12.4, 13.1, 17.3, 19.7, and 21.3 degrees 2θ (±0.2 degrees 2θ). In some embodiments, crystalline Form F of a compound having Formula (I), characterized by an X-ray powder diffraction (XRPD) pattern including peaks (i.e., the first 10 peaks ranked according to relative peak intensity %) at 7.8, 14.7, 15.7, 17.8, 18.2, 23.8, 24.9, 25.4, 27.5, and 27.8 degrees 2θ (±0.2 degrees 2θ). In some embodiments, Form F is characterized by an X-ray powder diffraction (XRPD) pattern including one, two, three, four, five or more peaks listed in Table 11. In some embodiments, Form F is characterized by an X-ray powder diffraction (XRPD) pattern including at least five peaks listed in Table 11. In some embodiments, Form F is characterized by an X-ray powder diffraction (XRPD) pattern including at least four peaks listed in Table 11.

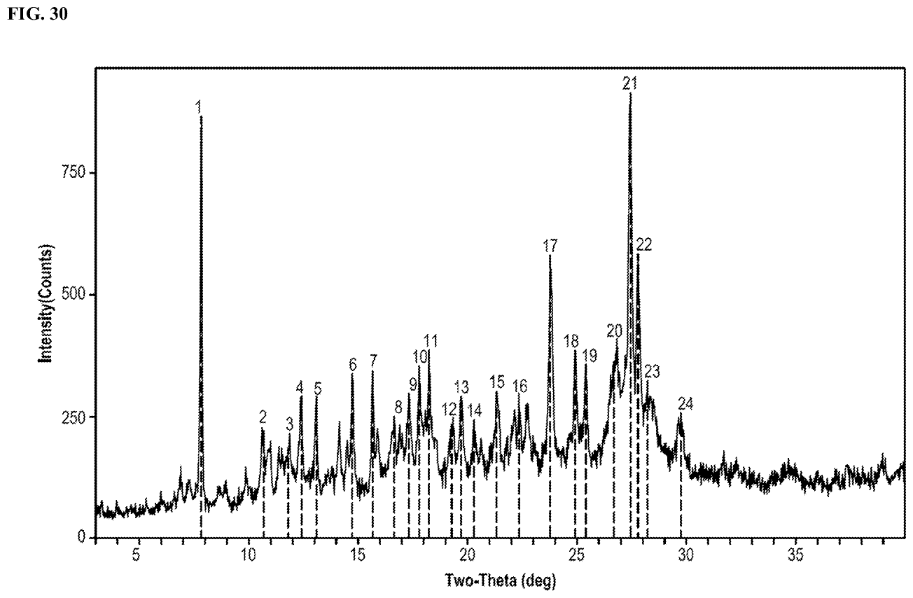

In some embodiments, crystalline Form F of a compound having Formula (I), is characterized by an X-ray powder diffraction pattern substantially in accordance with FIG. 30.

In some embodiments, crystalline Form F is substantially free of other crystalline or amorphous forms of the compound having Formula (I).

In some embodiments, crystalline Form F is further characterized by a differential scanning calorimetry (DSC) thermogram including one or more endothermic peaks at about 74.9° C., 212.0° C., and about 325.8° C. In some embodiments, crystalline Form F is further characterized by a differential scanning calorimetry (DSC) thermogram including an endothermic peak at about 74.9° C. In some embodiments, crystalline Form F is further characterized by a differential scanning calorimetry (DSC) thermogram including an endothermic peak at about 212.0° C. In some embodiments, crystalline Form F is further characterized by a differential scanning calorimetry (DSC) thermogram including an endothermic peak at about 325.0° C. In some embodiments, crystalline Form F is further characterized by a differential scanning calorimetry (DSC) thermogram including an onset temperature of about 45.2° C. and an endothermic peak at about 74.9° C. In some embodiments, crystalline Form F is further characterized by a differential scanning calorimetry (DSC) thermogram including an onset temperature of about 205.2° C. and an endothermic peak at about 212.0° C. In some embodiments, crystalline Form F is further characterized by a differential scanning calorimetry (DSC) thermogram including an onset temperature of about 319.7° C. and an endothermic peak at about 325.8° C.

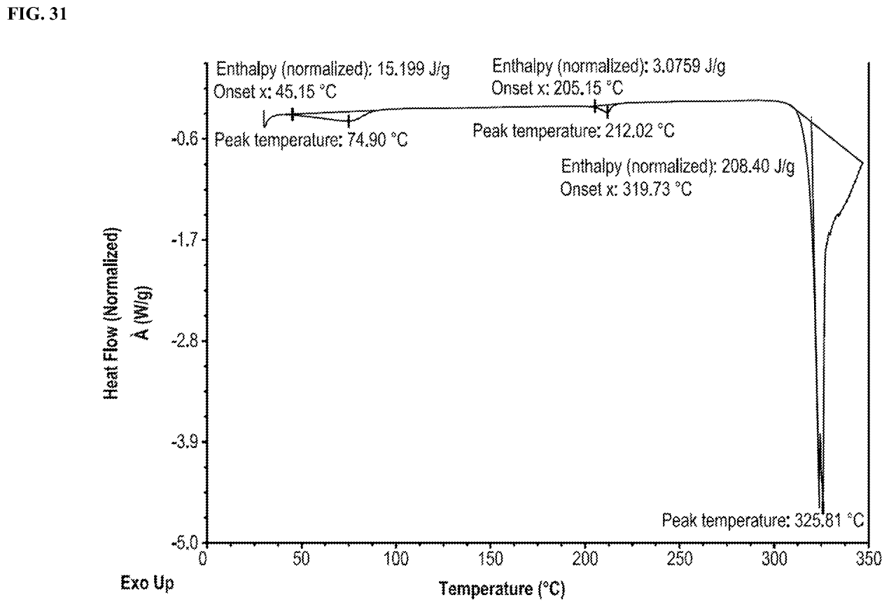

In some embodiments, crystalline Form F is further characterized by a differential scanning calorimetry (DSC) thermogram substantially in accordance with FIG. 31.

In some embodiments, crystalline Form F is further characterized by a weight percent loss of about 3.2% upon heating to about 200° C., as measured by a thermal gravimetric analysis (TGA).

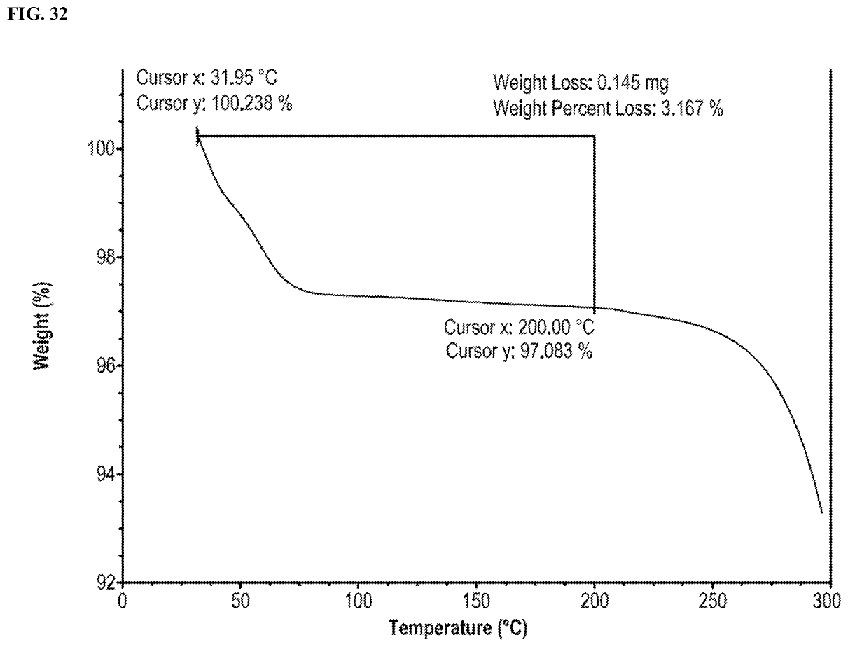

In some embodiments, crystalline Form F is further characterized by a thermal gravimetric analysis (TGA) thermogram substantially in accordance with FIG. 32.

Crystalline Form F is in a hydrate form.

In some embodiments, crystalline Form F is characterized by an X-ray powder diffraction pattern substantially in accordance with FIG. 30; and is further characterized by a differential scanning calorimetry (DSC) thermogram substantially in accordance with FIG. 31. In some embodiments, crystalline Form F is characterized by an X-ray powder diffraction pattern substantially in accordance with FIG. 30; is further characterized by a differential scanning calorimetry (DSC) thermogram substantially in accordance with FIG. 31; and is further characterized by a thermal gravimetric analysis (TGA) thermogram substantially in accordance with FIG. 32.

### III-7. Crystalline Form G

In one embodiment, the present disclosure provides crystalline Form G of a compound having Formula (I). In some embodiments, the X-ray powder diffraction pattern includes peaks at 5.5, 15.6, and 17.2 degrees 2θ (±0.2 degrees 2θ). In some embodiments, the X-ray powder diffraction pattern includes peaks at 5.5, 10.0, 15.6, and 17.2 degrees 2θ (0.2 degrees 2θ). In some embodiments, the X-ray powder diffraction pattern includes peaks at 5.5, 7.4, 10.0, 15.6, and 17.2 degrees 2θ (±0.2 degrees 2θ).

In one embodiment, the present disclosure provides crystalline Form G of a compound having Formula (I), characterized by an X-ray powder diffraction (XRPD) pattern including peaks at 5.5, 10.0, 15.6, 17.2, and 23.4 degrees 2θ (±0.2 degrees 2θ). In some embodiments, the X-ray powder diffraction pattern further includes peaks at 7.4, 7.8, 20.9, 24.5, and 27.7 degrees 2θ (±0.2 degrees 2θ). In some embodiments, crystalline Form G of a compound having Formula (I), characterized by an X-ray powder diffraction (XRPD) pattern including peaks (i.e., the first 10 peaks ranked according to relative peak intensity %) at 5.5, 7.4, 7.8, 10.0, 15.6, 17.2, 20.9, 23.4, 24.5, and 27.7 degrees 2θ (±0.2 degrees 2θ). In some embodiments, Form G is characterized by an X-ray powder diffraction (XRPD) pattern including one, two, three, four, five or more peaks listed in Table 12. In some embodiments, Form G is characterized by an X-ray powder diffraction (XRPD) pattern including at least five peaks listed in Table 12. In some embodiments, Form G is characterized by an X-ray powder diffraction (XRPD) pattern including at least four peaks listed in Table 12.

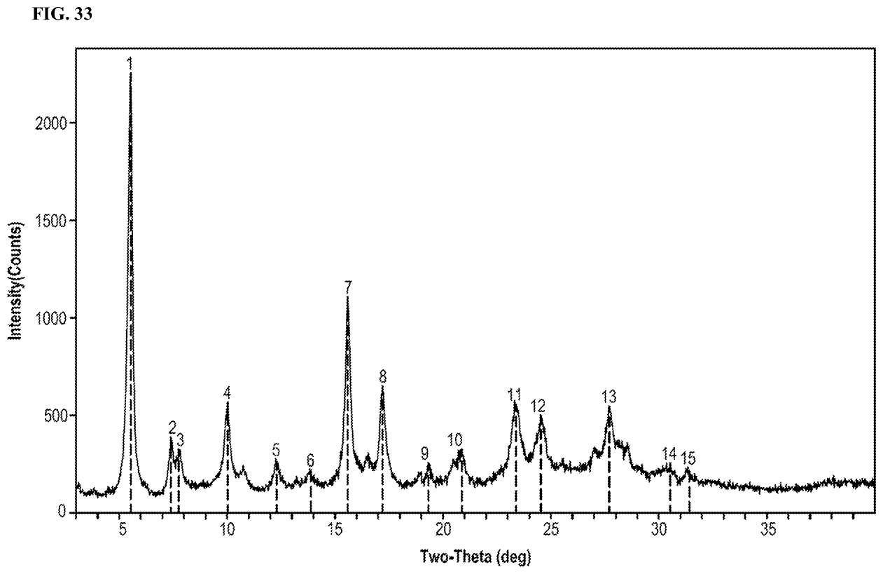

In some embodiments, crystalline Form G of a compound having Formula (I), is characterized by an X-ray powder diffraction pattern substantially in accordance with FIG. 33.

In some embodiments, crystalline Form G is substantially free of other crystalline or amorphous forms of the compound having Formula (I).

In some embodiments, crystalline Form G is further characterized by a differential scanning calorimetry (DSC) thermogram including an endothermic peaks at about 322.3° C. In some embodiments, crystalline Form G is further characterized by a differential scanning calorimetry (DSC) thermogram including an onset temperature of about 317.7° C. and an endothermic peak at about 322.8° C.

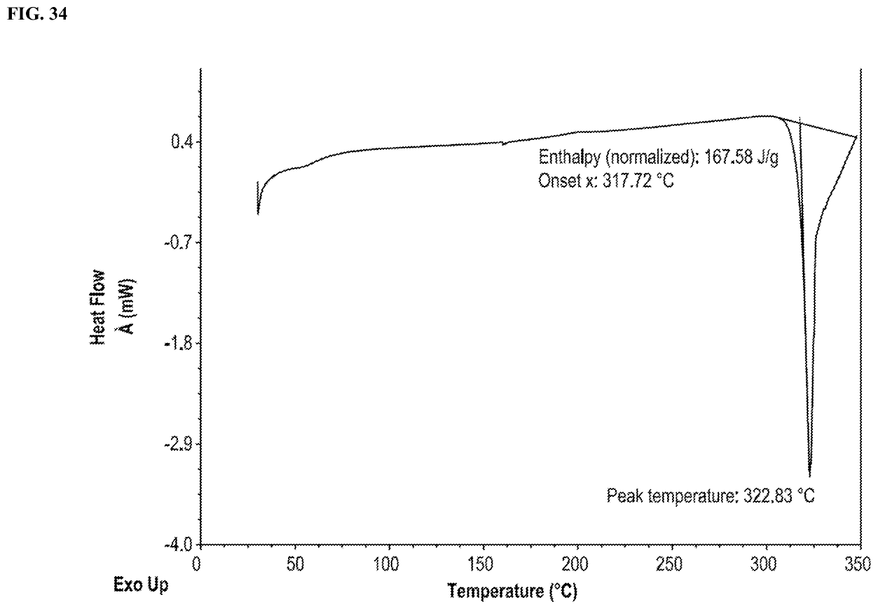

In some embodiments, crystalline Form G is further characterized by a differential scanning calorimetry (DSC) thermogram substantially in accordance with FIG. 34.

In some embodiments, crystalline Form G is further characterized by a first weight percent loss of 2.5% upon heating to about 160° C. and a second weight percent loss of about 0.9% upon heating to about 233° C., as measured by a thermal gravimetric analysis (TGA).

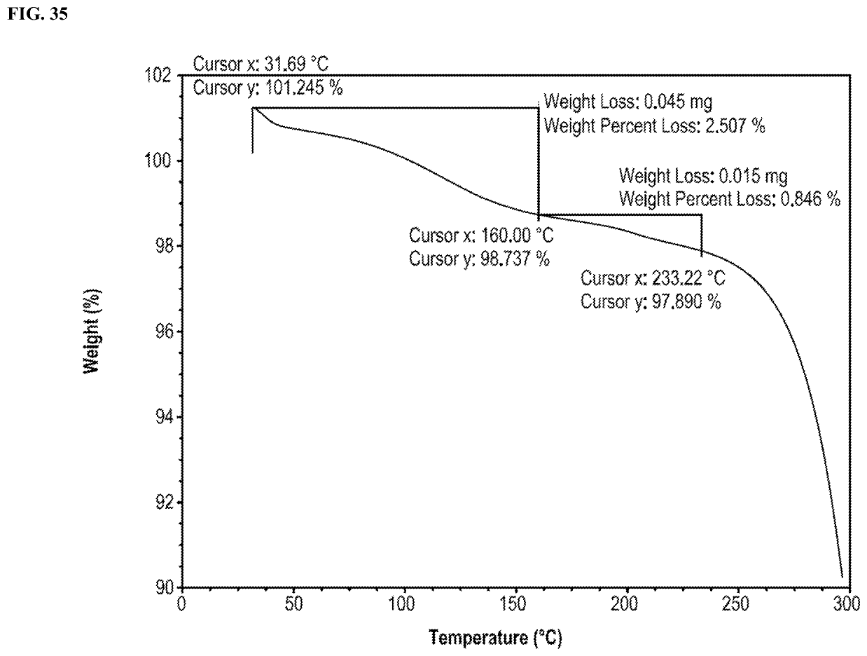

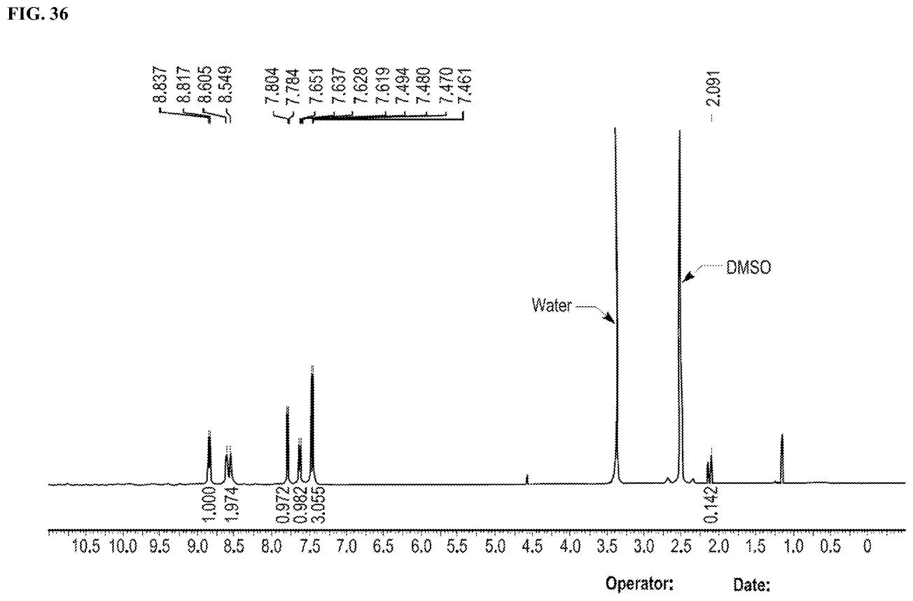

In some embodiments, crystalline Form G is further characterized by a thermal gravimetric analysis (TGA) thermogram substantially in accordance with FIG. 35.

In some embodiments, crystalline Form G is characterized by an X-ray powder diffraction pattern substantially in accordance with FIG. 33; and is further characterized by a differential scanning calorimetry (DSC) thermogram substantially in accordance with FIG. 34. In some embodiments, crystalline Form G is characterized by an X-ray powder diffraction pattern substantially in accordance with FIG. 33; is further characterized by a differential scanning calorimetry (DSC) thermogram substantially in accordance with FIG. 34; and is further characterized by a thermal gravimetric analysis (TGA) thermogram substantially in accordance with FIG. 35.

### III-8. Crystalline Form H

In one embodiment, the present disclosure provides crystalline Form H of a compound having Formula (I). In some embodiments, the X-ray powder diffraction pattern includes peaks at 5.1, 5.8, and 15.6 degrees 2θ (±0.2 degrees 2θ). In some embodiments, the X-ray powder diffraction pattern includes peaks at 5.1, 5.8, 8.8, and 15.6 degrees 2θ (±0.2 degrees 2θ). In some embodiments, the X-ray powder diffraction pattern includes peaks at 5.1, 5.8, 7.8, 8.8, and 15.6 degrees 2θ (±0.2 degrees 2θ).

In one embodiment, the present disclosure provides crystalline Form H of a compound having Formula (I), characterized by an X-ray powder diffraction (XRPD) pattern including peaks at 5.1, 5.8, 8.8, 15.6, and 23.4 degrees 2θ (±0.2 degrees 2θ). In some embodiments, the X-ray powder diffraction pattern further includes peaks at 7.8, 11.9, 17.3, and 28.3 degrees 2θ (±0.2 degrees 2θ). In some embodiments, crystalline Form H of a compound having Formula (I), characterized by an X-ray powder diffraction (XRPD) pattern including peaks (i.e., the first 9 peaks ranked according to relative peak intensity %) at 5.1, 5.8, 7.8, 8.8, 11.9, 15.6, 17.3, 23.4, and 28.3 degrees 2θ (±0.2 degrees 2θ). In some embodiments, Form H is characterized by an X-ray powder diffraction (XRPD) pattern including one, two, three, four, five or more peaks listed in Table 13. In some embodiments, Form H is characterized by an X-ray powder diffraction (XRPD) pattern including at least five peaks listed in Table 13. In some embodiments, Form H is characterized by an X-ray powder diffraction (XRPD) pattern including at least four peaks listed in Table 13.

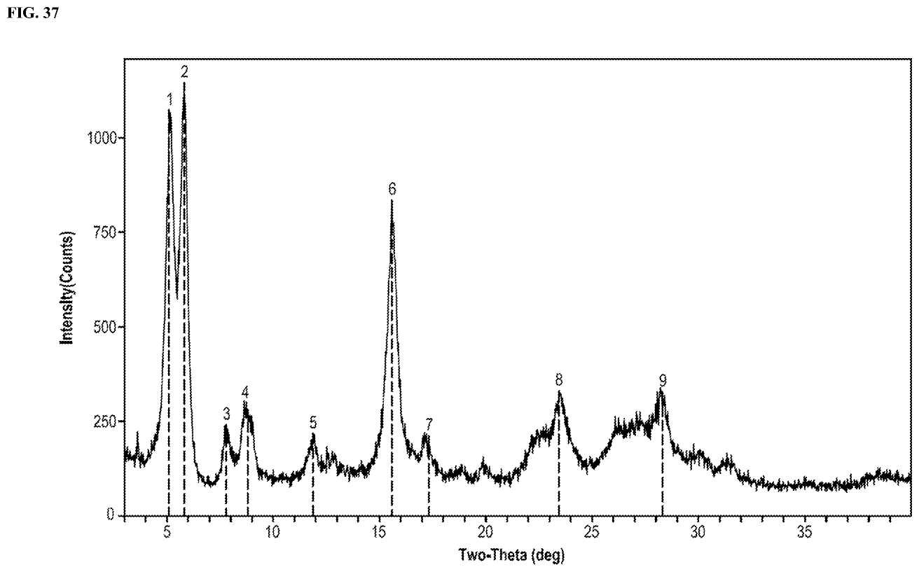

In some embodiments, crystalline Form H of a compound having Formula (I), is characterized by an X-ray powder diffraction pattern substantially in accordance with FIG. 37.

In some embodiments, crystalline Form H is substantially free of other crystalline or amorphous forms of the compound having Formula (I).

In some embodiments, crystalline Form H is further characterized by a differential scanning calorimetry (DSC) thermogram including one or more endothermic peaks at about 226.3° C. and about 322.6° C. In some embodiments, crystalline Form H is further characterized by a differential scanning calorimetry (DSC) thermogram including an endothermic peak at about 226.3° C. In some embodiments, crystalline Form H is further characterized by a differential scanning calorimetry (DSC) thermogram including an endothermic peak at about 322.6° C. In some embodiments, crystalline Form H is further characterized by a differential scanning calorimetry (DSC) thermogram including an onset temperature of about 210.5° C. and an endothermic peak at about 226.3° C. In some embodiments, crystalline Form H is further characterized by a differential scanning calorimetry (DSC) thermogram including an onset temperature of about 315.5° C. and an endothermic peak at about 322.6° C.

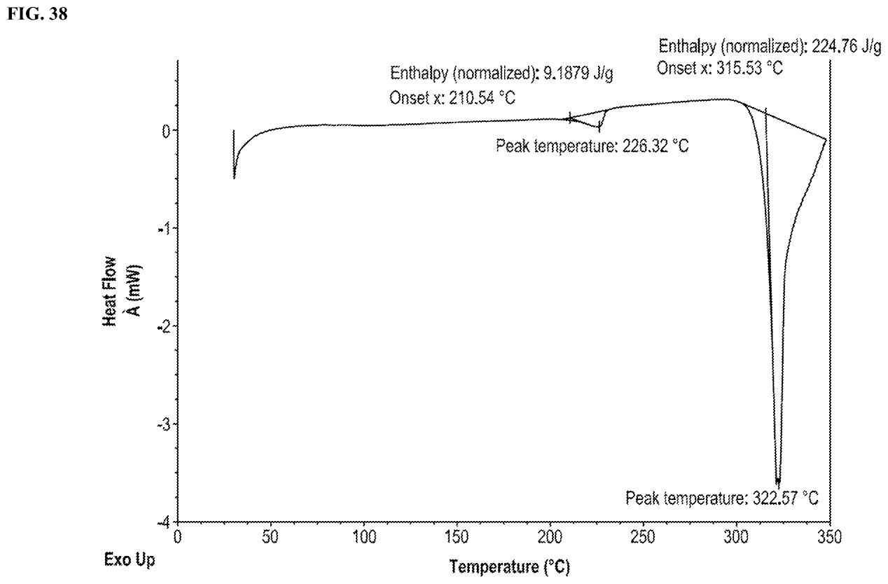

In some embodiments, crystalline Form H is further characterized by a differential scanning calorimetry (DSC) thermogram substantially in accordance with FIG. 38.

In some embodiments, crystalline Form H is further characterized by a weight percent loss of about 8.7% upon heating to about 238° C., as measured by a thermal gravimetric analysis (TGA).

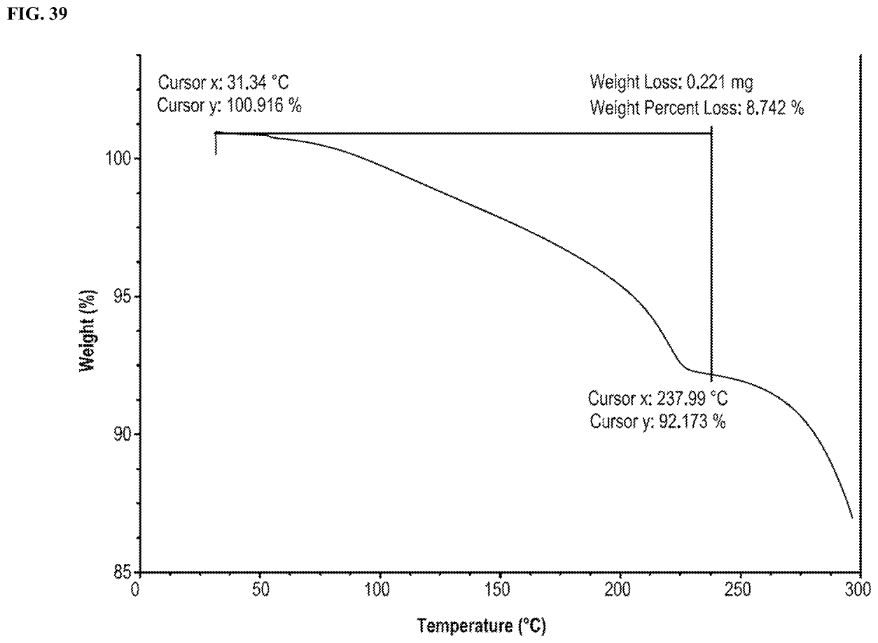

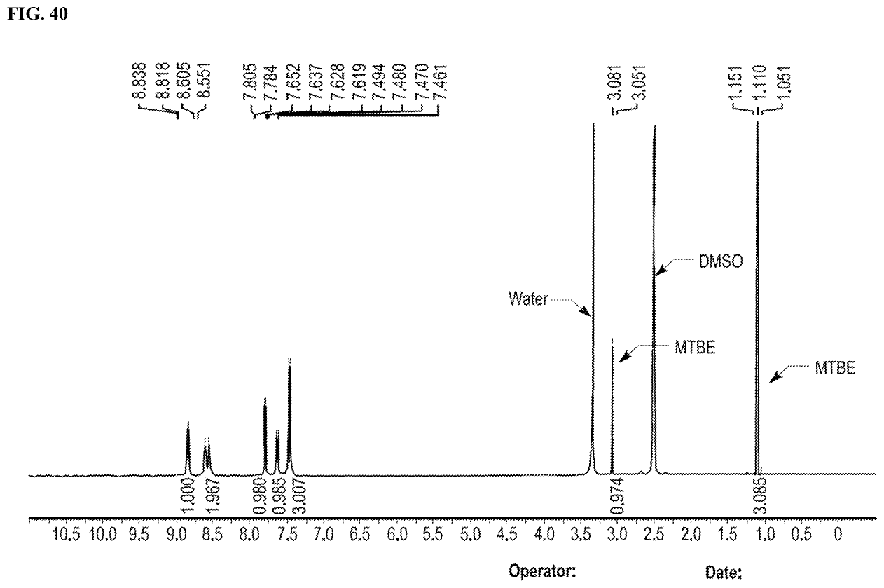

In some embodiments, crystalline Form H is further characterized by a thermal gravimetric analysis (TGA) thermogram substantially in accordance with FIG. 39.

Crystalline Form H is in a methyl t-butyl ether solvate form.

In some embodiments, crystalline Form H is characterized by an X-ray powder diffraction pattern substantially in accordance with FIG. 37; and is further characterized by a differential scanning calorimetry (DSC) thermogram substantially in accordance with FIG. 38. In some embodiments, crystalline Form H is characterized by an X-ray powder diffraction pattern substantially in accordance with FIG. 37; is further characterized by a differential scanning calorimetry (DSC) thermogram substantially in accordance with FIG. 38; and is further characterized by a thermal gravimetric analysis (TGA) thermogram substantially in accordance with FIG. 39.

### III-9. Crystalline Form I

In one embodiment, the present disclosure provides crystalline Form I of a compound having Formula (I). In some embodiments, the X-ray powder diffraction pattern includes peaks at 5.8 6.4, and 16.1 degrees 2θ (±0.2 degrees 2θ). In some embodiments, the X-ray powder diffraction pattern includes peaks at 5.8, 6.4, 16.1, and 16.5 degrees 2θ (±0.2 degrees 2θ). In some embodiments, the X-ray powder diffraction pattern includes peaks at 5.8, 6.4, 13.1, 16.1, and 16.5 degrees 2θ (±0.2 degrees 2θ).

In one embodiment, the present disclosure provides crystalline Form I of a compound having Formula (I), characterized by an X-ray powder diffraction (XRPD) pattern including peaks at 5.8, 6.4, 16.1, 16.5, and 27.7 degrees 2θ (±0.2 degrees 2θ). In some embodiments, the X-ray powder diffraction pattern further includes peaks at 10.0, 10.8, 13.1, 25.5, and 29.0 degrees 2θ (±0.2 degrees 2θ). In some embodiments, the X-ray powder diffraction pattern further includes peaks at 8.4, 11.9, 17.7, 19.6, and 23.3 degrees 2θ (±0.2 degrees 2θ). In some embodiments, crystalline Form I of a compound having Formula (I), characterized by an X-ray powder diffraction (XRPD) pattern including peaks (i.e., the first 10 peaks ranked according to relative peak intensity %) at 5.8, 6.4, 10.0, 10.8, 13.1, 16.1, 16.5, 25.5, 27.7, and 29.0 degrees 2θ (±0.2 degrees 2θ). In some embodiments, crystalline Form I of the compound having Formula (I), characterized by an X-ray powder diffraction (XRPD) pattern including peaks at 5.8, 6.4, 10.0, 10.8, 13.1, 16.1, and 16.5 degrees 2θ (±0.2 degrees 2θ). In some embodiments, crystalline Form I of the compound having Formula (I), characterized by an X-ray powder diffraction (XRPD) pattern comprising at least any of the two peaks selected from 5.8, 6.4, 10.0, 10.8, 13.1, 16.1, and 16.5 degrees 2θ (±0.2 degrees 2θ). In some embodiments, crystalline Form I of the compound having Formula (I), characterized by an X-ray powder diffraction (XRPD) pattern comprising at least any of the three peaks selected from 5.8, 6.4, 10.0, 10.8, 13.1, 16.1, and 16.5 degrees 2θ (±0.2 degrees 2θ). In some embodiments, crystalline Form I of the compound having Formula (I), characterized by an X-ray powder diffraction (XRPD) pattern comprising at least any of the four peaks selected from 5.8, 6.4, 10.0, 10.8, 13.1, 16.1, and 16.5 degrees 2θ (n 0.2 degrees 2θ). In some embodiments, crystalline Form I of the compound having Formula (I), characterized by an X-ray powder diffraction (XRPD) pattern comprising at least any of the five peaks selected from 5.8, 6.4, 10.0, 10.8, 13.1, 16.1, and 16.5 degrees 2θ (n 0.2 degrees 2θ). In some embodiments, crystalline Form I of the compound having Formula (I), characterized by an X-ray powder diffraction (XRPD) pattern comprising at least any of the six peaks selected from 5.8, 6.4, 10.0, 10.8, 13.1, 16.1, and 16.5 degrees 2θ (±0.2 degrees 2θ). In some embodiments, Form I is characterized by an X-ray powder diffraction (XRPD) pattern including one, two, three, four, five or more peaks listed in Table 15. In some embodiments, Form I is characterized by an X-ray powder diffraction (XRPD) pattern including at least five peaks listed in Table 15. In some embodiments, Form I is characterized by an X-ray powder diffraction (XRPD) pattern including at least four peaks listed in Table 15.

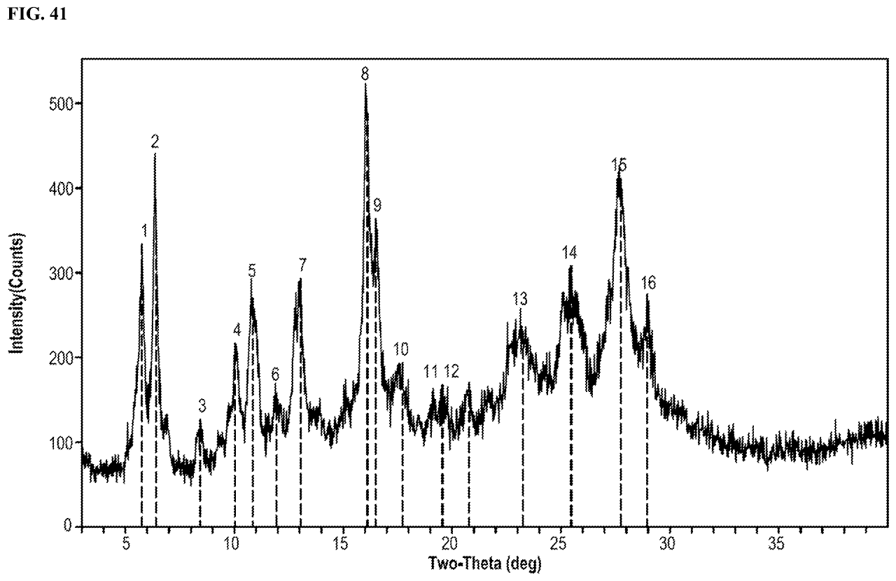

In some embodiments, crystalline Form I of a compound having Formula (I), is characterized by an X-ray powder diffraction pattern substantially in accordance with FIG. 41.

In some embodiments, crystalline Form I is substantially free of other crystalline or amorphous forms of the compound having Formula (I).

In some embodiments, crystalline Form I is further characterized by a differential scanning calorimetry (DSC) thermogram including one or more endothermic peaks at about 267.05° C. and about 323.7° C. In some embodiments, crystalline Form I is further characterized by a differential scanning calorimetry (DSC) thermogram including an endothermic peak at about 267.05° C. In some embodiments, crystalline Form I is further characterized by a differential scanning calorimetry (DSC) thermogram including an endothermic peak at about 323.7° C. In some embodiments, crystalline Form I is further characterized by a differential scanning calorimetry (DSC) thermogram including an onset temperature of about 261.6° C. and an endothermic peak at about 267.05° C. In some embodiments, crystalline Form I is further characterized by a differential scanning calorimetry (DSC) thermogram including an onset temperature of about 319.6° C. and an endothermic peak at about 323.7° C.

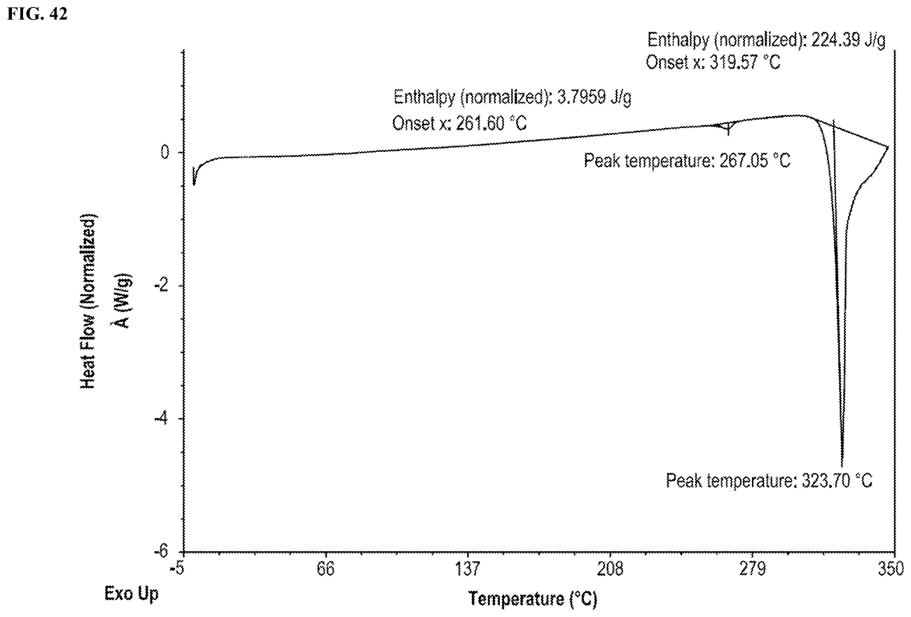

In some embodiments, crystalline Form I is further characterized by a differential scanning calorimetry (DSC) thermogram substantially in accordance with FIG. 42.

In some embodiments, crystalline Form I is further characterized by a weight percent loss of about 1.10% upon heating to about 220° C., as measured by a thermal gravimetric analysis (TGA).

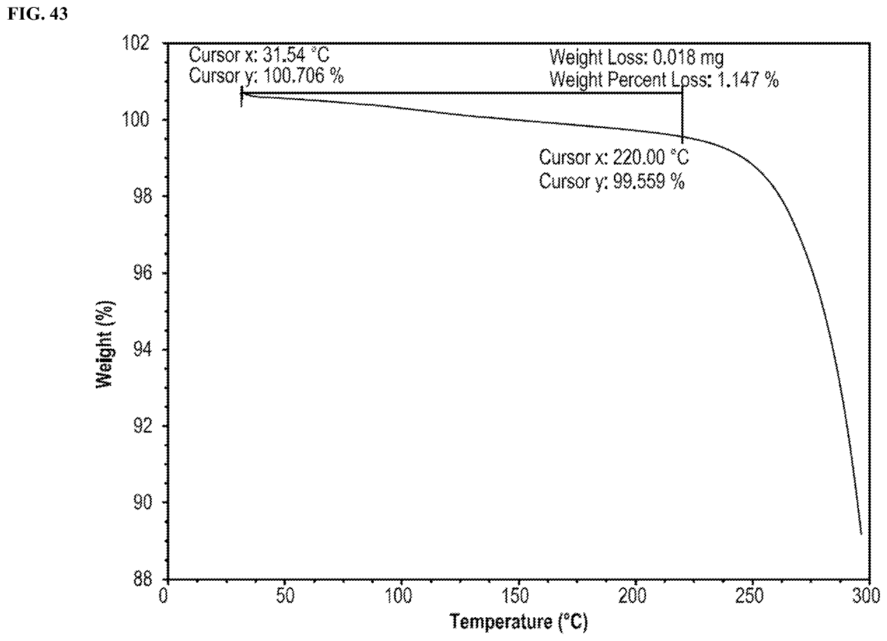

In some embodiments, crystalline Form I is further characterized by a thermal gravimetric analysis (TGA) thermogram substantially in accordance with FIG. 43.

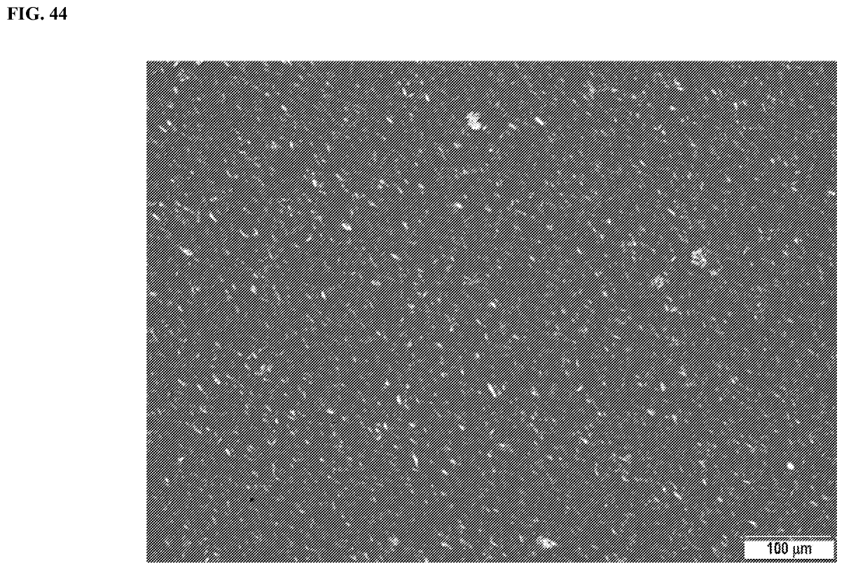

In some embodiments, crystalline Form I is further characterized by a polarized light microscope (PLM) profile substantially as shown in FIG. 44. In some embodiments, crystalline Form I is in an irregular particle characterized by a polarized light microscope (PLM) profile substantially as shown in FIG. 44.

Crystalline Form I is in a anhydrate form.

In some embodiments, crystalline Form I is characterized by an X-ray powder diffraction pattern substantially in accordance with FIG. 41; and is further characterized by a differential scanning calorimetry (DSC) thermogram substantially in accordance with FIG. 42. In some embodiments, crystalline Form I is characterized by an X-ray powder diffraction pattern substantially in accordance with FIG. 41; is further characterized by a differential scanning calorimetry (DSC) thermogram substantially in accordance with FIG. 42; and is further characterized by a thermal gravimetric analysis (TGA) thermogram substantially in accordance with FIG. 43.

### III-10. Crystalline Form J

In one embodiment, the present disclosure provides crystalline Form J of a compound having Formula (I). In some embodiments, the X-ray powder diffraction pattern includes peaks at 7.7, 12.9, and 14.6 degrees 2θ (±0.2 degrees 2θ). In some embodiments, the X-ray powder diffraction pattern includes peaks at 7.7, 12.9, 14.6 and 18.1 degrees 2θ (±0.2 degrees 2θ). In some embodiments, the X-ray powder diffraction pattern includes peaks at 7.7, 12.9, 14.6, 17.4, and 18.1 degrees 2θ (±0.2 degrees 2θ).

In one embodiment, the present disclosure provides crystalline Form J of a compound having Formula (I), characterized by an X-ray powder diffraction (XRPD) pattern including peaks at 7.7, 12.9, 14.6, 26.9, and 27.6 degrees 2θ (±0.2 degrees 2θ). In some embodiments, the X-ray powder diffraction pattern further includes peaks at 18.1, 22.2, 23.2, 25.3, and 27.4 degrees 2θ (±0.2 degrees 2θ). In some embodiments, the X-ray powder diffraction pattern further includes peaks at 17.4, 21.3, 23.8, 26.0, and 26.8 degrees 2θ (±0.2 degrees 2θ). In some embodiments, crystalline Form J of a compound having Formula (I), characterized by an X-ray powder diffraction (XRPD) pattern including peaks (i.e., the first 10 peaks ranked according to relative peak intensity %) at 7.7, 12.9, 14.6, 18.1, 22.2, 23.2, 25.3, 26.9, 27.4, and 27.6 degrees 2θ (±0.2 degrees 2θ). In some embodiments, Form J is characterized by an X-ray powder diffraction (XRPD) pattern including one, two, three, four, five or more peaks listed in Table 16. In some embodiments, Form J is characterized by an X-ray powder diffraction (XRPD) pattern including at least five peaks listed in Table 16. In some embodiments, Form J is characterized by an X-ray powder diffraction (XRPD) pattern including at least four peaks listed in Table 16.

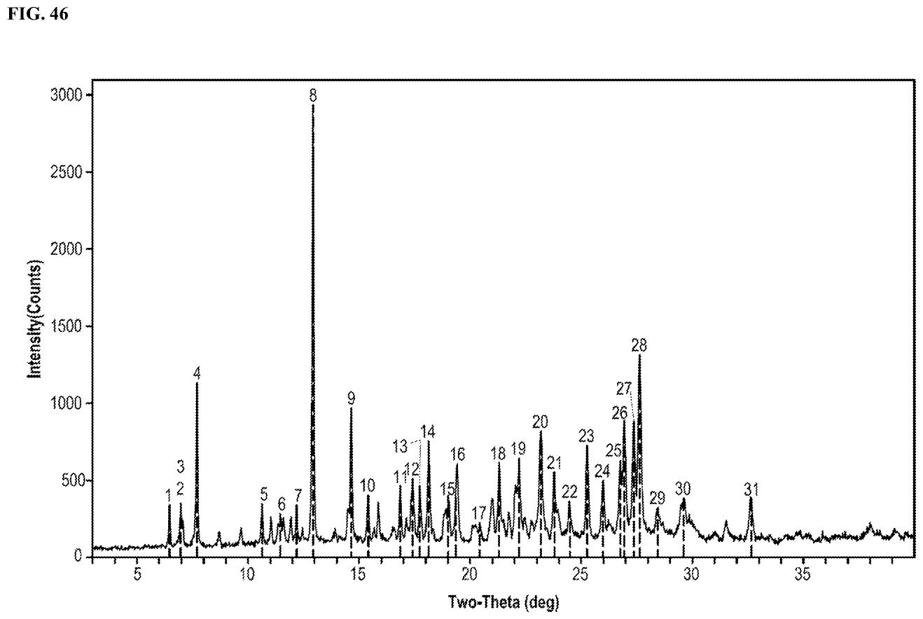

In some embodiments, crystalline Form J of a compound having Formula (I), is characterized by an X-ray powder diffraction pattern substantially in accordance with FIG. 46.

In some embodiments, crystalline Form J is substantially free of other crystalline or amorphous forms of the compound having Formula (I).

In some embodiments, crystalline Form J is further characterized by a differential scanning calorimetry (DSC) thermogram including one or more endothermic peaks at about 131.9° C., 212.0° C., and about 324.0° C. In some embodiments, crystalline Form J is further characterized by a differential scanning calorimetry (DSC) thermogram including an endothermic peak at about 131.9° C. In some embodiments, crystalline Form J is further characterized by a differential scanning calorimetry (DSC) thermogram including an endothermic peak at about 212.0° C. In some embodiments, crystalline Form J is further characterized by a differential scanning calorimetry (DSC) thermogram including an endothermic peak at about 324.0° C. In some embodiments, crystalline Form J is further characterized by a differential scanning calorimetry (DSC) thermogram including an onset temperature of about 84.2° C. and an endothermic peak at about 131.9° C. In some embodiments, crystalline Form J is further characterized by a differential scanning calorimetry (DSC) thermogram including an onset temperature of about 204.0° C. and an endothermic peak at about 212.0° C. In some embodiments, crystalline Form J is further characterized by a differential scanning calorimetry (DSC) thermogram including an onset temperature of about 319.4° C. and an endothermic peak at about 324.0° C.

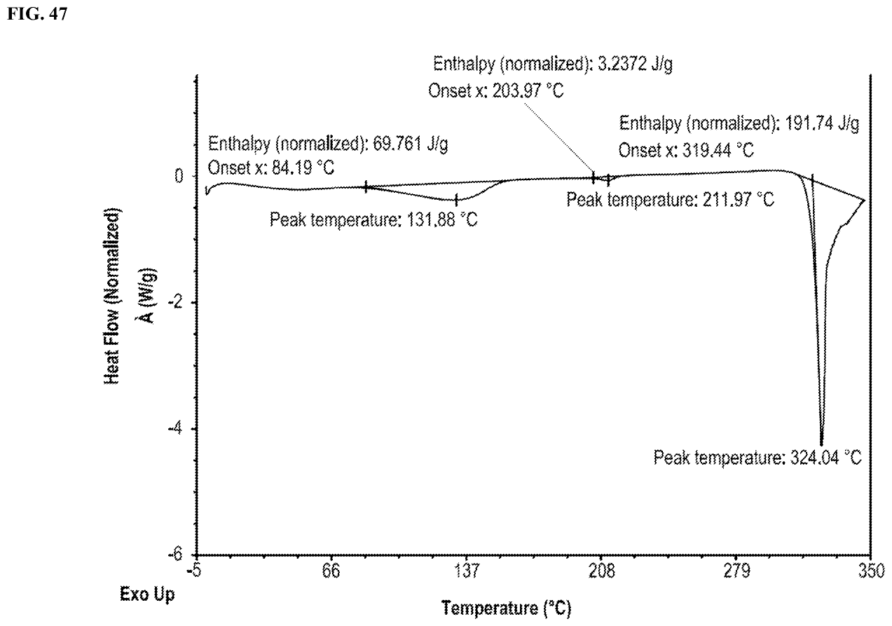

In some embodiments, crystalline Form J is further characterized by a differential scanning calorimetry (DSC) thermogram substantially in accordance with FIG. 47.

In some embodiments, crystalline Form J is further characterized by a weight percent loss of about 9.9% upon heating to about 200° C., as measured by a thermal gravimetric analysis (TGA).

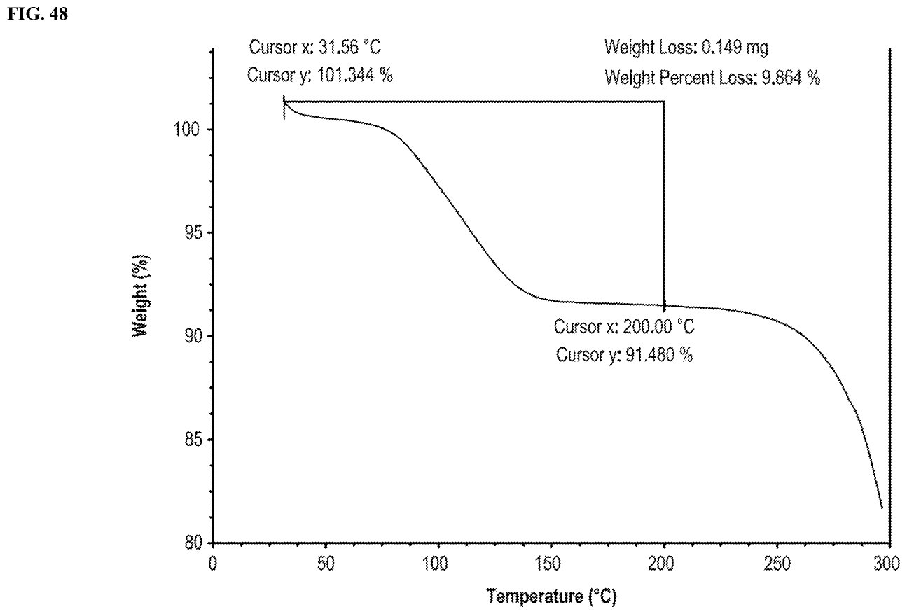

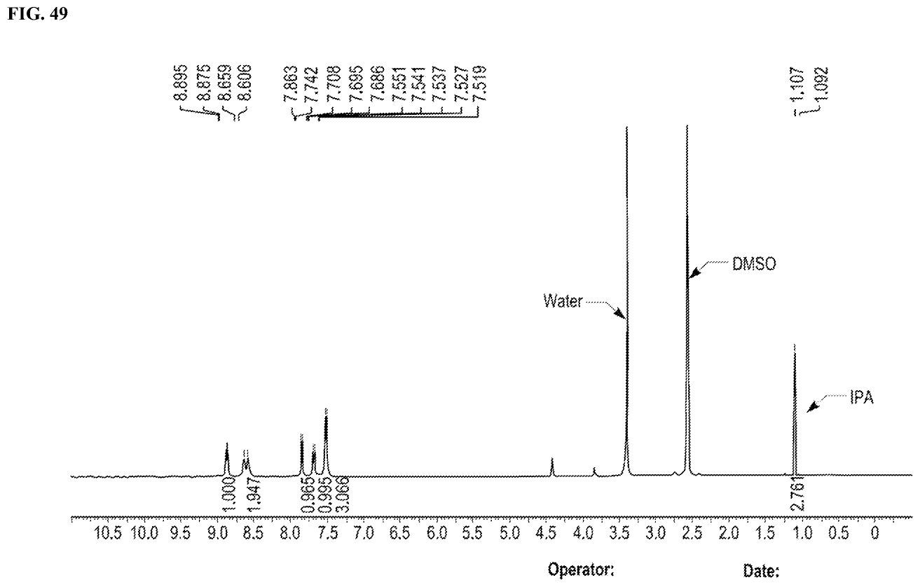

In some embodiments, crystalline Form J is further characterized by a thermal gravimetric analysis (TGA) thermogram substantially in accordance with FIG. 48.

Crystalline Form J is in an isopropyl solvate form.

In some embodiments, crystalline Form J is characterized by an X-ray powder diffraction pattern substantially in accordance with FIG. 46; and is further characterized by a differential scanning calorimetry (DSC) thermogram substantially in accordance with FIG. 47. In some embodiments, crystalline Form J is characterized by an X-ray powder diffraction pattern substantially in accordance with FIG. 46; is further characterized by a differential scanning calorimetry (DSC) thermogram substantially in accordance with FIG. 47; and is further characterized by a thermal gravimetric analysis (TGA) thermogram substantially in accordance with FIG. 48.

### III-11. Crystalline Form K

In one embodiment, the present disclosure provides crystalline Form K of the compound having Formula (I). In some embodiments, the X-ray powder diffraction pattern includes peaks at 5.5, 11.1, and 16.5 degrees 2θ (±0.2 degrees 2θ). In some embodiments, the X-ray powder diffraction pattern includes peaks at 5.5, 11.1, 13.8, and 16.5 degrees 2θ (0.2 degrees 2θ). In some embodiments, the X-ray powder diffraction pattern includes peaks at 5.5, 11.1, 13.8, 16.5, and 17.5 degrees 2θ (±0.2 degrees 2θ).

In one embodiment, the present disclosure provides crystalline Form K of the compound having Formula (I), characterized by an X-ray powder diffraction (XRPD) pattern including peaks at 5.5, 11.1, 16.5, 27.1, and 27.7 degrees 2θ (±0.2 degrees 2θ). In some embodiments, the X-ray powder diffraction pattern further includes peaks at 13.8, 17.5, 22.2, 25.2, and 28.9 degrees 2θ (±0.2 degrees 2θ). In some embodiments, crystalline Form K of the compound having Formula (I), characterized by an X-ray powder diffraction (XRPD) pattern including peaks (i.e., the first 10 peaks ranked according to relative peak intensity %) at 5.5, 11.1, 13.8, 16.5, 17.5, 22.2, 25.2, 27.1, 27.7, and 28.9 degrees 2θ (±0.2 degrees 2θ). In some embodiments, Form K is characterized by an X-ray powder diffraction (XRPD) pattern including one, two, three, four, five or more peaks listed in Table 17. In some embodiments, Form K is characterized by an X-ray powder diffraction (XRPD) pattern including at least five peaks listed in Table 17. In some embodiments, Form K is characterized by an X-ray powder diffraction (XRPD) pattern including at least four peaks listed in Table 17.

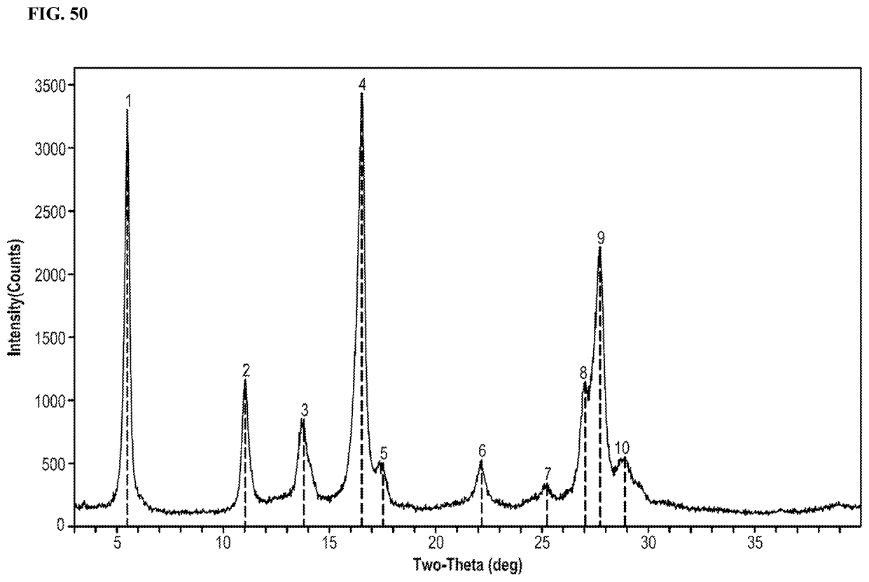

In some embodiments, crystalline Form K of the compound having Formula (I) is characterized by an X-ray powder diffraction (XRPD) pattern substantially in accordance with FIG. 50.

In some embodiments, crystalline Form K is substantially free of other crystalline or amorphous forms of the compound having Formula (I).

### III-12. Crystalline Form L

In one embodiment, the present disclosure provides crystalline Form L of a compound having Formula (I). In some embodiments, the X-ray powder diffraction pattern includes peaks at 11.7, 17.9, and 18.4 degrees 2θ (±0.2 degrees 2θ). In some embodiments, the X-ray powder diffraction pattern includes peaks at 11.7, 12.3, 17.9, and 18.4 degrees 2θ (±0.2 degrees 2θ). In some embodiments, the X-ray powder diffraction pattern includes peaks at 11.7, 12.3, 16.6, 17.9, and 18.4 degrees 2θ (±0.2 degrees 2θ).

In one embodiment, the present disclosure provides crystalline Form L of a compound having Formula (I), characterized by an X-ray powder diffraction (XRPD) pattern including peaks at 11.7, 17.9, 18.4, 26.3, and 27.0 degrees 2θ (±0.2 degrees 2θ). In some embodiments, the X-ray powder diffraction pattern further includes peaks at 10.8, 12.3, 16.3, 16.6, and 22.2 degrees 2θ (±0.2 degrees 2θ). In some embodiments, the X-ray powder diffraction pattern further includes peaks at 17.0, 19.7, 20.3, 23.3, and 28.1 degrees 2θ (±0.2 degrees 2θ). In some embodiments, crystalline Form L of a compound having Formula (I), characterized by an X-ray powder diffraction (XRPD) pattern including peaks (i.e., the first 10 peaks ranked according to relative peak intensity %) at 10.8, 11.7, 12.3, 16.3, 16.6, 17.9, 18.4, 22.2, 26.3, and 27.0 degrees 2θ (±0.2 degrees 2θ). In some embodiments, Form L is characterized by an X-ray powder diffraction (XRPD) pattern including one, two, three, four, five or more peaks listed in Table 18. In some embodiments, Form L is characterized by an X-ray powder diffraction (XRPD) pattern including at least five peaks listed in Table 18. In some embodiments, Form L is characterized by an X-ray powder diffraction (XRPD) pattern including at least four peaks listed in Table 18.

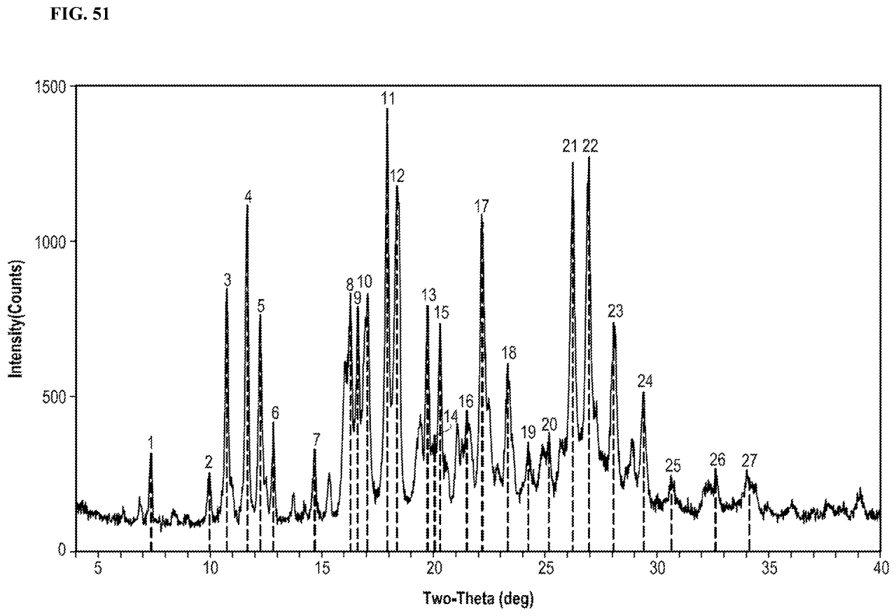

In some embodiments, crystalline Form L of a compound having Formula (I), is characterized by an X-ray powder diffraction pattern substantially in accordance with FIG. 51.

In some embodiments, crystalline Form L is substantially free of other crystalline or amorphous forms of the compound having Formula (I).

In some embodiments, crystalline Form L is further characterized by a differential scanning calorimetry (DSC) thermogram including one or more endothermic peaks at about 202.5° C. and about 326.6.0° C. In some embodiments, crystalline Form L is further characterized by a differential scanning calorimetry (DSC) thermogram including an endothermic peak at about 202.5° C. In some embodiments, crystalline Form L is further characterized by a differential scanning calorimetry (DSC) thermogram including an endothermic peak at about 326.6.0° C. In some embodiments, crystalline Form L is further characterized by a differential scanning calorimetry (DSC) thermogram including an onset temperature of about 195.6° C. and an endothermic peak at about 202.5° C. In some embodiments, crystalline Form L is further characterized by a differential scanning calorimetry (DSC) thermogram including an onset temperature of about 322.6° C. and an endothermic peak at about 326.6.0° C.

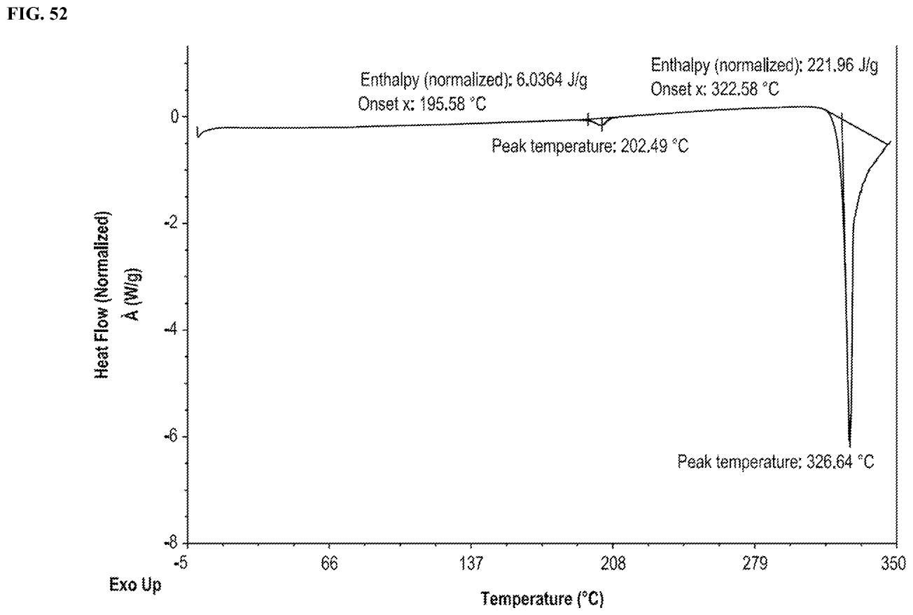

In some embodiments, crystalline Form L is further characterized by a differential scanning calorimetry (DSC) thermogram substantially in accordance with FIG. 52.

In some embodiments, crystalline Form L is further characterized by a weight percent loss of about 2.0% upon heating to about 220° C., as measured by a thermal gravimetric analysis (TGA).

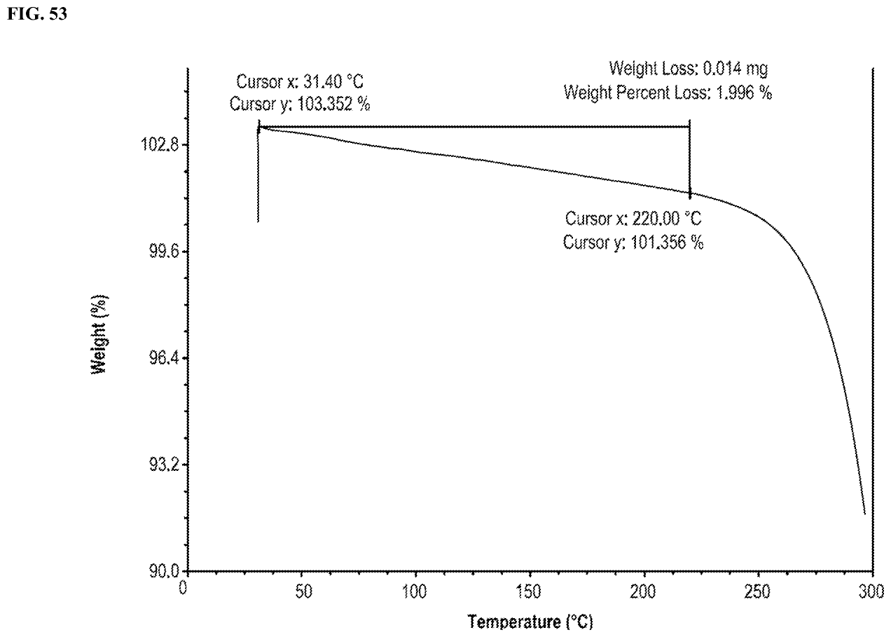

In some embodiments, crystalline Form L is further characterized by a thermal gravimetric analysis (TGA) thermogram substantially in accordance with FIG. 53.

Crystalline Form L is in an anhydrate form.

In some embodiments, crystalline Form L is characterized by an X-ray powder diffraction pattern substantially in accordance with FIG. 51; and is further characterized by a differential scanning calorimetry (DSC) thermogram substantially in accordance with FIG. 52. In some embodiments, crystalline Form L is characterized by an X-ray powder diffraction pattern substantially in accordance with FIG. 51; is further characterized by a differential scanning calorimetry (DSC) thermogram substantially in accordance with FIG. 52; and is further characterized by a thermal gravimetric analysis (TGA) thermogram substantially in accordance with FIG. 53.

### III-13. Crystalline Form M

In one embodiment, the present disclosure provides crystalline Form M of the compound having Formula (I). In some embodiments, the present disclosure provides crystalline Form M of the compound having Formula (I), characterized by an X-ray powder diffraction (XRPD) pattern including peaks at 6.2, 12.4, and 13.4 degrees 2θ (±0.2 degrees 2θ). In some embodiments, the present disclosure provides crystalline Form M of the compound having Formula (I), characterized by an X-ray powder diffraction (XRPD) pattern including peaks at 6.2, 10.5, 12.4, and 13.4 degrees 2θ (±0.2 degrees 2θ). In some embodiments, the present disclosure provides crystalline Form M of the compound having Formula (I), characterized by an X-ray powder diffraction (XRPD) pattern including peaks at 6.2, 6.7, 10.5, 12.4, and 13.4 degrees 2θ (±0.2 degrees 2θ).

In some embodiments, the X-ray powder diffraction pattern further includes peaks at 16.1, 17.6, 24.2, 27.1, and 28.3 degrees 2θ (±0.2 degrees 2θ). In some embodiments, the X-ray powder diffraction pattern further includes peaks at 17.0, 20.2, 22.7, 23.1, and 26.5 degrees 2θ (±0.2 degrees 2θ). In some embodiments, crystalline Form M of the compound having Formula (I), characterized by an X-ray powder diffraction (XRPD) pattern including peaks (i.e., the first 10 peaks ranked according to relative peak intensity %) at 6.2, 6.7, 10.5, 12.4, 13.4, 16.1, 17.6, 24.2, 27.1, and 28.3 degrees 2θ (±0.2 degrees 2θ). In some embodiments, Form M is characterized by an X-ray powder diffraction (XRPD) pattern including one, two, three, four, five or more peaks listed in Table 19. In some embodiments, Form M is characterized by an X-ray powder diffraction (XRPD) pattern including at least five peaks listed in Table 19. In some embodiments, Form M is characterized by an X-ray powder diffraction (XRPD) pattern including at least four peaks listed in Table 19.

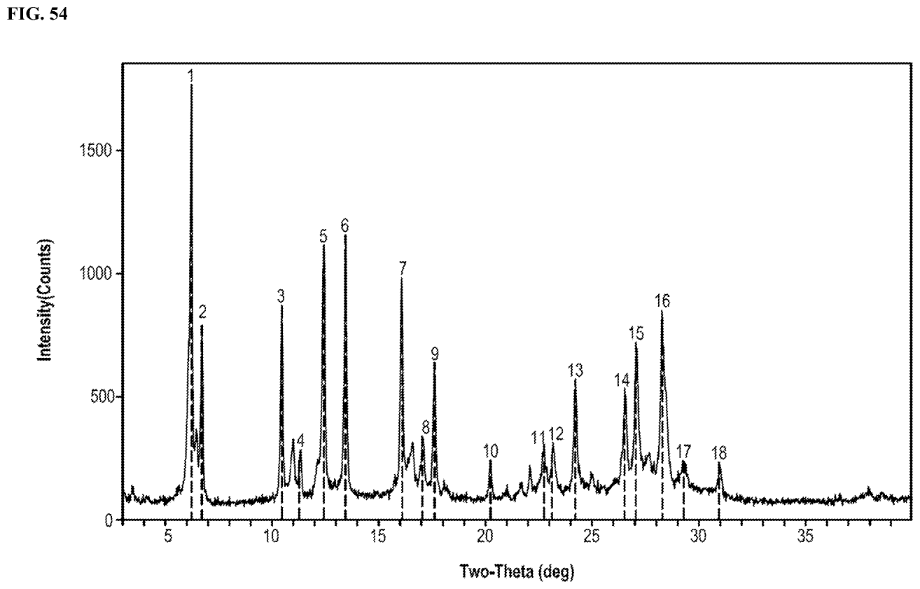

In some embodiments, crystalline Form M of the compound having Formula (I) is characterized by an X-ray powder diffraction (XRPD) pattern substantially in accordance with FIG. 54.

In some embodiments, crystalline Form M is substantially free of other crystalline or amorphous forms of the compound having Formula (I).

### III-14. Crystalline Form N

In one embodiment, the present disclosure provides crystalline Form N of the compound having Formula (I). In some embodiments, the X-ray powder diffraction pattern includes peaks at 5.4, 10.7, and 16.1 degrees 2θ (±0.2 degrees 2θ). In some embodiments, the X-ray powder diffraction pattern includes peaks at 5.4, 10.7, 14.6 and 16.1 degrees 2θ (±0.2 degrees 2θ). In some embodiments, the X-ray powder diffraction pattern includes peaks at 5.4, 10.7, 12.4, 14.6 and 16.1 degrees 2θ (±0.2 degrees 2θ).

In some embodiments, Form N is characterized by an X-ray powder diffraction (XRPD) pattern comprising peaks at 5.4, 10.7, 16.1, 27.0, and 28.0 degrees 2θ (±0.2 degrees 2θ). In some embodiments, the X-ray powder diffraction pattern further includes peaks at 14.6, 21.6, 24.9, 25.4, and 28.4 degrees 2θ (±0.2 degrees 2θ). In some embodiments, crystalline Form N of the compound having Formula (I), characterized by an X-ray powder diffraction (XRPD) pattern including peaks (i.e., the first 10 peaks ranked according to relative peak intensity %) at 5.4, 10.7, 16.1, 14.6, 21.6, 24.9, 25.4, 27.0, 28.0, and 28.4 degrees 2θ (±0.2 degrees 2θ). In some embodiments, Form N is characterized by an X-ray powder diffraction (XRPD) pattern including one, two, three, four, five or more peaks listed in Table 20. In some embodiments, Form N is characterized by an X-ray powder diffraction (XRPD) pattern including at least five peaks listed in Table 20. In some embodiments, Form N is characterized by an X-ray powder diffraction (XRPD) pattern including at least four peaks listed in Table 20.

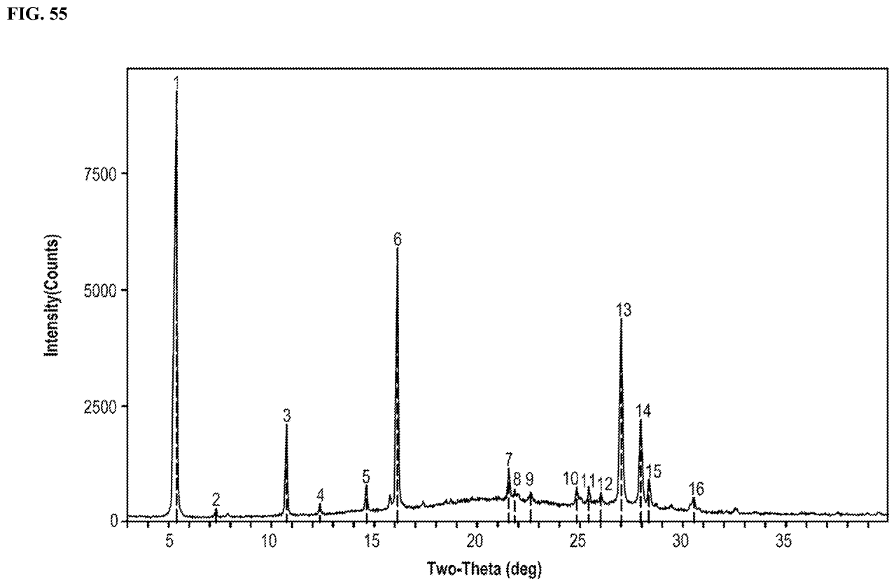

In some embodiments, crystalline Form N of the compound having Formula (I) is characterized by an X-ray powder diffraction (XRPD) pattern substantially in accordance with FIG. 55.

In some embodiments, crystalline Form N is substantially free of other crystalline or amorphous forms of the compound having Formula (I).

### III-15. Crystalline Form O

In one embodiment, the present disclosure provides crystalline Form O of the compound having Formula (I). In some embodiments, the X-ray powder diffraction pattern includes peaks at 5.0, 5.9, and 13.4 degrees 2θ (±0.2 degrees 2θ). In some embodiments, the X-ray powder diffraction pattern includes peaks at 5.0, 5.9, 13.4, and 17.8 degrees 2θ (±0.2 degrees 2θ). In some embodiments, the X-ray powder diffraction pattern includes peaks at 5.0, 5.9, 13.4, 15.3, and 17.8 degrees 2θ (±0.2 degrees 2θ).

In some embodiments, Form O is characterized by an X-ray powder diffraction (XRPD) pattern comprising peaks at 5.0, 5.9, 13.4, 17.8, and 25.7 degrees 2θ (±0.2 degrees 20). In some embodiments, the X-ray powder diffraction pattern further includes peaks at 12.8, 15.3, 26.9, 27.9, and 29.0 degrees 2θ (±0.2 degrees 2θ). In some embodiments, crystalline Form O of the compound having Formula (I), characterized by an X-ray powder diffraction (XRPD) pattern including peaks (i.e., the first 10 peaks ranked according to relative peak intensity %) at 5.0, 5.9, 12.8, 13.4, 15.3, 17.8, 25.7, 26.9, 27.9, and 29.0 degrees 2θ (±0.2 degrees 2θ). In some embodiments, Form O is characterized by an X-ray powder diffraction (XRPD) pattern including one, two, three, four, five or more peaks listed in Table 21. In some embodiments, Form O is characterized by an X-ray powder diffraction (XRPD) pattern including at least five peaks listed in Table 21. In some embodiments, Form O is characterized by an X-ray powder diffraction (XRPD) pattern including at least four peaks listed in Table 21.

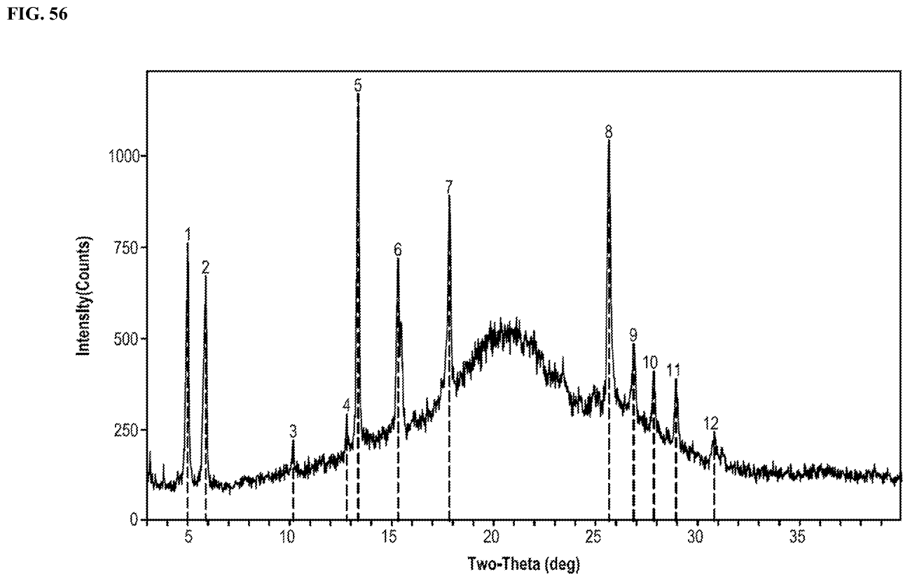

In some embodiments, crystalline Form O of the compound having Formula (I) is characterized by an X-ray powder diffraction (XRPD) pattern substantially in accordance with FIG. 56.

In some embodiments, crystalline Form O is substantially free of other crystalline or amorphous forms of the compound having Formula (I).

### III-16. Crystalline Form P

In one embodiment, the present disclosure provides crystalline Form P of the compound having Formula (I). In some embodiments, the X-ray powder diffraction pattern includes peaks at 5.6, 6.0, and 17.1 degrees 2θ (±0.2 degrees 2θ). In some embodiments, the X-ray powder diffraction pattern includes peaks at 5.6, 6.0, 10.8, and 17.1 degrees 2θ (±0.2 degrees 2θ). In some embodiments, the X-ray powder diffraction pattern includes peaks at 5.6, 6.0, 10.8, 12.0 and 17.1 degrees 2θ (±0.2 degrees 2θ).

In some embodiments, Form P is characterized by an X-ray powder diffraction (XRPD) pattern including peaks at 5.6, 6.0, 17.1, 26.6, and 27.9 degrees 2θ (±0.2 degrees 2θ). In some embodiments, the X-ray powder diffraction pattern further includes peaks at 10.8, 12.0, 14.3, 14.8, and 18.1 degrees 2θ (±0.2 degrees 2θ). In some embodiments, the X-ray powder diffraction pattern further includes peaks at 6.7, 9.5, 15.8, 16.8, 30.3 degrees 2θ (±0.2 degrees 2θ). In some embodiments, crystalline Form P of the compound having Formula (I), characterized by an X-ray powder diffraction (XRPD) pattern including peaks (i.e., the first 10 peaks ranked according to relative peak intensity %) at 5.6, 6.0, 10.8, 12.0, 14.3, 14.8, 17.1, 18.1, 26.6, and 27.9 degrees 2θ (±0.2 degrees 2θ). In some embodiments, crystalline Form P of the compound having Formula (I), characterized by an X-ray powder diffraction (XRPD) pattern including peaks at 5.6, 6.0, 10.8, 12.0, 14.3, 14.8, 17.1, and 18.1 degrees 2θ (±0.2 degrees 2θ). In some embodiments, Form P is characterized by an X-ray powder diffraction (XRPD) pattern including one, two, three, four, five or more peaks listed in Table 22. In some embodiments, Form P is characterized by an X-ray powder diffraction (XRPD) pattern including at least five peaks listed in Table 22. In some embodiments, Form P is characterized by an X-ray powder diffraction (XRPD) pattern including at least four peaks listed in Table 22.

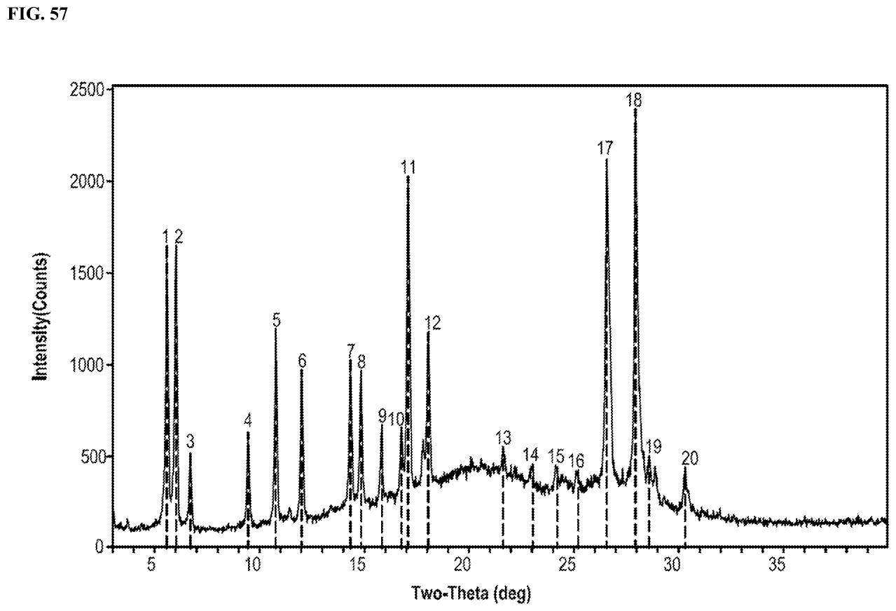

In some embodiments, crystalline Form P of the compound having Formula (I) is characterized by an X-ray powder diffraction (XRPD) pattern substantially in accordance with FIG. 57.

In some embodiments, crystalline Form O is substantially free of other crystalline or amorphous forms of the compound having Formula (I).

### III-17. Crystalline Form O

In one embodiment, the present disclosure provides crystalline Form Q of the compound having Formula (I). In some embodiments, the X-ray powder diffraction pattern includes peaks at 3.3, 5.4, and 16.1 degrees 2θ (±0.2 degrees 2θ). In some embodiments, the X-ray powder diffraction pattern includes peaks at 3.3, 5.4, 10.7, and 16.1 degrees 2θ (±0.2 degrees 2θ). In some embodiments, the X-ray powder diffraction pattern includes peaks at 3.3, 4.0, 5.4, 10.7, and 16.1 degrees 2θ (±0.2 degrees 2θ).

In some embodiments, Form Q is characterized by an X-ray powder diffraction (XRPD) pattern including peaks at 3.3, 5.4, 16.1, 27.0, and 31.7 degrees 2θ (±0.2 degrees 2θ). In some embodiments, the X-ray powder diffraction pattern includes peaks at 3.3, 5.4, and 16.1 degrees 2θ (±0.2 degrees 2θ). In some embodiments, the X-ray powder diffraction pattern further includes peaks at 4.0, 10.7, 25.0, 27.9, and 28.0 degrees 2θ (±0.2 degrees 2θ). In some embodiments, the X-ray powder diffraction pattern further includes peaks at 8.2, 14.5, 19.5, 22.6, and 24.9 degrees 2θ (±0.2 degrees 2θ). In some embodiments, crystalline Form Q of the compound having Formula (I), characterized by an X-ray powder diffraction (XRPD) pattern including peaks (i.e., the first 10 peaks ranked according to relative peak intensity %) at 3.3, 4.0, 5.4, 10.7, 16.1, 25.0, 27.0, 27.9, 28.0, and 31.7 degrees 2θ (±0.2 degrees 2θ). In some embodiments, Form Q is characterized by an X-ray powder diffraction (XRPD) pattern including one, two, three, four, five or more peaks listed in Table 23. In some embodiments, Form Q is characterized by an X-ray powder diffraction (XRPD) pattern including at least five peaks listed in Table 23. In some embodiments, Form Q is characterized by an X-ray powder diffraction (XRPD) pattern including at least four peaks listed in Table 23.

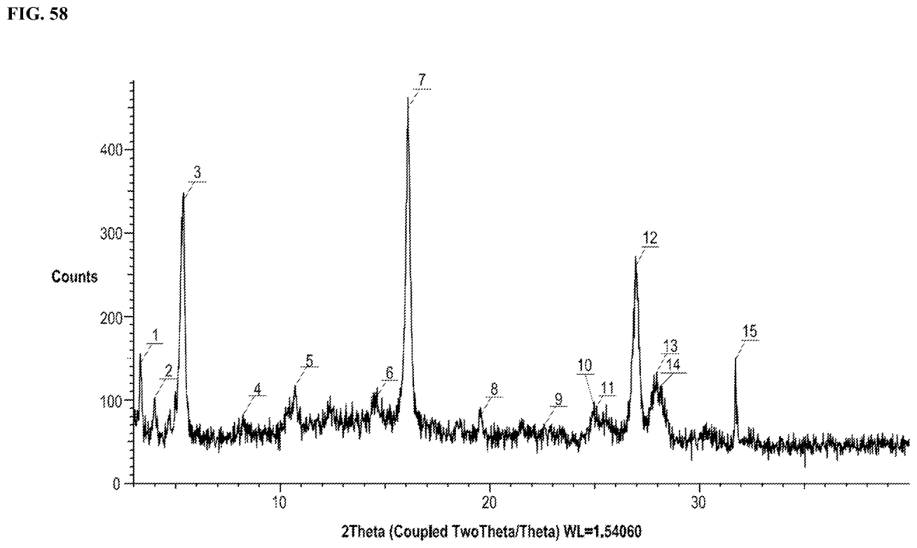

In some embodiments, crystalline Form Q of the compound having Formula (I) is characterized by an X-ray powder diffraction (XRPD) pattern substantially in accordance with FIG. 58.

In some embodiments, crystalline Form Q is substantially free of other crystalline or amorphous forms of the compound having Formula (I).

### III-18. Crystalline Form R

In one embodiment, the present disclosure provides crystalline Form R of the compound having Formula (I). In some embodiments, the X-ray powder diffraction pattern includes peaks at 3.2, 7.0, and 13.9 degrees 2θ (±0.2 degrees 2θ). In some embodiments, the X-ray powder diffraction pattern includes peaks at 3.2, 7.0, 13.9, and 18.9 degrees 2θ (±0.2 degrees 2θ). In some embodiments, the X-ray powder diffraction pattern includes peaks at 3.2, 7.0, 13.9, 18.5 and 18.9 degrees 2θ (±0.2 degrees 2θ).

In one embodiment, the present disclosure provides crystalline Form R of the compound having Formula (I). In some embodiments, Form R is characterized by an X-ray powder diffraction (XRPD) pattern including one, two, three, four, five or more peaks listed in Table 24. In some embodiments, Form R is characterized by an X-ray powder diffraction (XRPD) pattern including at least five peaks listed in Table 24. In some embodiments, Form R is characterized by an X-ray powder diffraction (XRPD) pattern including at least four peaks listed in Table 24.

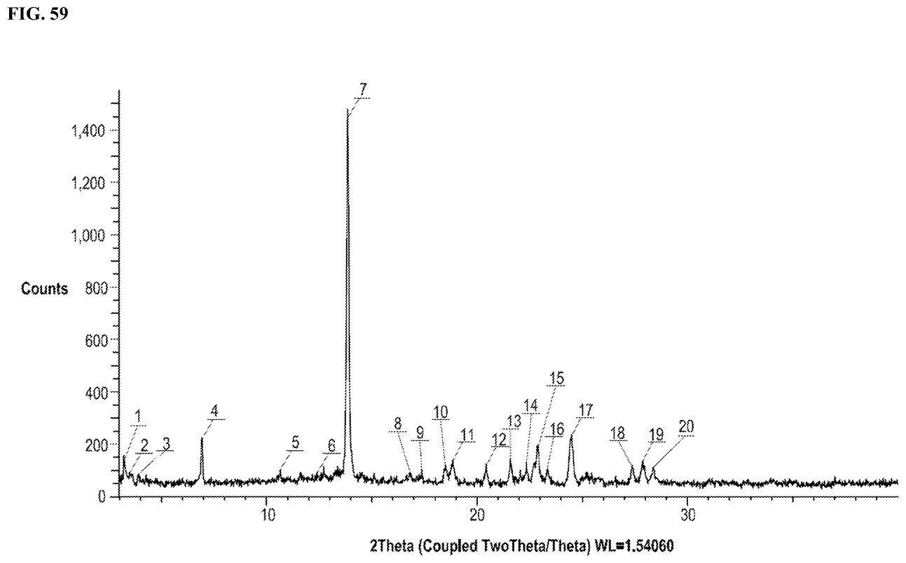

In some embodiments, crystalline Form R of the compound having Formula (I) is characterized by an X-ray powder diffraction (XRPD) pattern substantially in accordance with FIG. 59.

In some embodiments, crystalline Form R is substantially free of other crystalline or amorphous forms of the compound having Formula (I).

### III-19. Crystalline Form S

In one embodiment, the present disclosure provides crystalline Form S of the compound having Formula (I). In some embodiments, Form S is characterized by an X-ray powder diffraction (XRPD) pattern including peaks at 6.1, 13.5, and 16.5 degrees 2θ (±0.2 degrees 2θ). In some embodiments, Form S is characterized by an X-ray powder diffraction (XRPD) pattern including peaks at 6.1, 10.8, 13.5, and 16.5 degrees 2θ (±0.2 degrees 2θ). In some embodiments, Form S is characterized by an X-ray powder diffraction (XRPD) pattern including peaks at 6.1, 10.4, 10.8, 13.5, and 16.5 degrees 2θ (±0.2 degrees 2θ).

In some embodiments, the X-ray powder diffraction pattern further includes peaks at 12.3, 12.4, 14.0, 17.4, and 28.6 degrees 2θ (±0.2 degrees 2θ). In some embodiments, the X-ray powder diffraction pattern further includes peaks at 12.0, 16.0, 19.0, 26.8, and 29.3 degrees 2θ (±0.2 degrees 2θ). In some embodiments, crystalline Form S of the compound having Formula (I), characterized by an X-ray powder diffraction (XRPD) pattern including peaks (i.e., the first 10 peaks ranked according to relative peak intensity %) at 6.1, 10.4, 10.8, 12.3, 12.4, 13.5, 14.0, 16.5, 17.4, and 28.6 degrees 2θ (±0.2 degrees 2θ). In some embodiments, crystalline Form S of the compound having Formula (I), characterized by an X-ray powder diffraction (XRPD) pattern including peaks at 6.1, 10.4, 10.8, 13.5, and 16.5 degrees 2θ (±0.2 degrees 2θ). In some embodiments, Form S is characterized by an X-ray powder diffraction (XRPD) pattern including one, two, three, four, five or more peaks listed in Table 25. In some embodiments, Form S is characterized by an X-ray powder diffraction (XRPD) pattern including at least five peaks listed in Table 25. In some embodiments, Form S is characterized by an X-ray powder diffraction (XRPD) pattern including at least four peaks listed in Table 25.

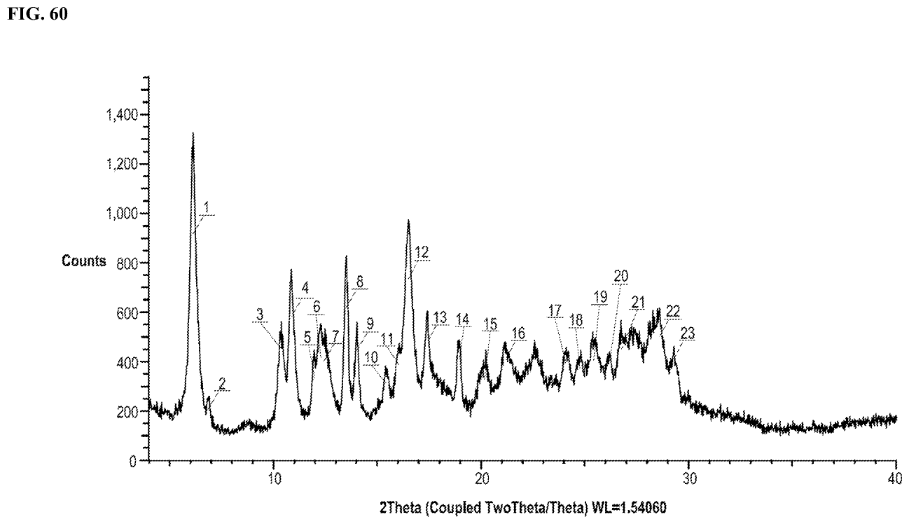

In some embodiments, crystalline Form S of the compound having Formula (I) is characterized by an X-ray powder diffraction (XRPD) pattern substantially in accordance with FIG. 60.

In some embodiments, crystalline Form S is substantially free of other crystalline or amorphous forms of the compound having Formula (I).

Crystalline Form S is in an anhydrate form.

### III-20. Crystalline Form T

In one embodiment, the present disclosure provides crystalline Form T of a compound having Formula (I). In some embodiments, Form T is characterized by an X-ray powder diffraction (XRPD) pattern including peaks at 6.5, 10.9, and 16.9 degrees 2θ (±0.2 degrees 2θ). In some embodiments, Form T is characterized by an X-ray powder diffraction (XRPD) pattern including peaks at 6.5, 10.9, 13.0, and 16.9 degrees 2θ (±0.2 degrees 2θ). In some embodiments, Form T is characterized by an X-ray powder diffraction (XRPD) pattern including peaks at 6.5, 10.9, 12.4, 13.0, and 16.9 degrees 2θ (±0.2 degrees 2θ).

In some embodiments, the X-ray powder diffraction pattern further includes peaks at 10.5, 16.6, 22.2, 27.5, and 27.9 degrees 2θ (±0.2 degrees 2θ). In some embodiments, the X-ray powder diffraction pattern further includes peaks at 17.6, 23.2, 24.0, 25.3, and 25.4 degrees 2θ (±0.2 degrees 2θ). In some embodiments, crystalline Form T of a compound having Formula (I), characterized by an X-ray powder diffraction (XRPD) pattern including peaks (i.e., the first 10 peaks ranked according to relative peak intensity %) at 6.5, 10.5, 10.9, 12.4, 13.0, 16.6, 16.9, 22.2, 27.5, and 27.9 degrees 2θ (±0.2 degrees 2θ). In some embodiments, crystalline Form T of the compound having Formula (I), characterized by an X-ray powder diffraction (XRPD) pattern including peaks at 6.5, 10.9, 12.4, 13.0, and 16.9 degrees 2θ (±0.2 degrees 2θ). In some embodiments, crystalline Form T of the compound having Formula (I), characterized by an X-ray powder diffraction (XRPD) pattern comprising at least any of the two peaks selected from 6.5, 10.9, 12.4, 13.0, and 16.9 degrees 2θ (±0.2 degrees 2θ). In some embodiments, crystalline Form T of the compound having Formula (I), characterized by an X-ray powder diffraction (XRPD) pattern comprising at least any of the three peaks selected from 6.5, 10.9, 12.4, 13.0, and 16.9 degrees 2θ (±0.2 degrees 2θ). In some embodiments, crystalline Form T of the compound having Formula (I), characterized by an X-ray powder diffraction (XRPD) pattern comprising at least any of the four peaks selected from 6.5, 10.9, 12.4, 13.0, and 16.9 degrees 2θ (±0.2 degrees 2θ). In some embodiments, Form T is characterized by an X-ray powder diffraction (XRPD) pattern including one, two, three, four, five or more peaks listed in Table 26. In some embodiments, Form T is characterized by an X-ray powder diffraction (XRPD) pattern including at least five peaks listed in Table 26. In some embodiments, Form T is characterized by an X-ray powder diffraction (XRPD) pattern including at least four peaks listed in Table 26.

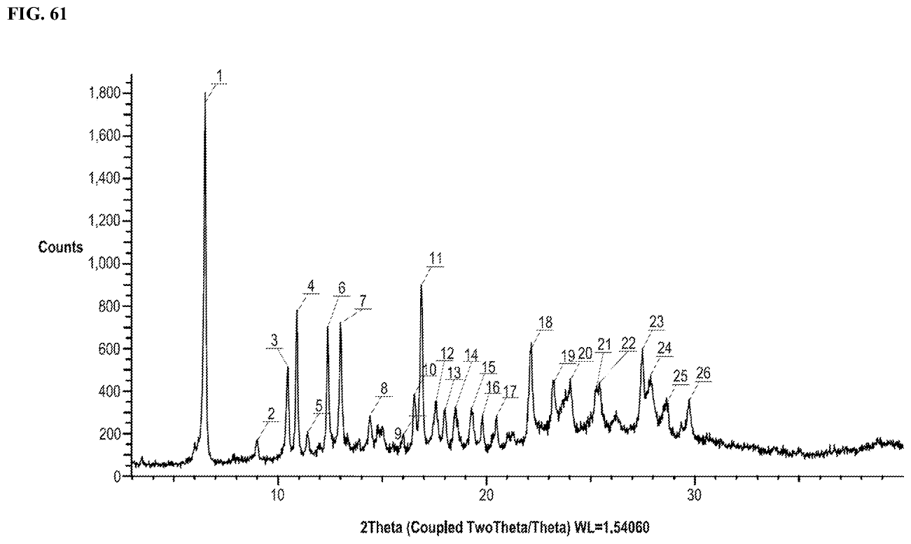

In some embodiments, crystalline Form T of a compound having Formula (I), is characterized by an X-ray powder diffraction pattern substantially in accordance with FIG. 61.

In some embodiments, crystalline Form T is substantially free of other crystalline or amorphous forms of the compound having Formula (I).

In some embodiments, crystalline Form T is further characterized by a differential scanning calorimetry (DSC) thermogram including an endothermic peaks at about 319.4° C. In some embodiments, crystalline Form T is further characterized by a differential scanning calorimetry (DSC) thermogram including an onset temperature of about 307.6° C. and an endothermic peak at about 319.4° C.

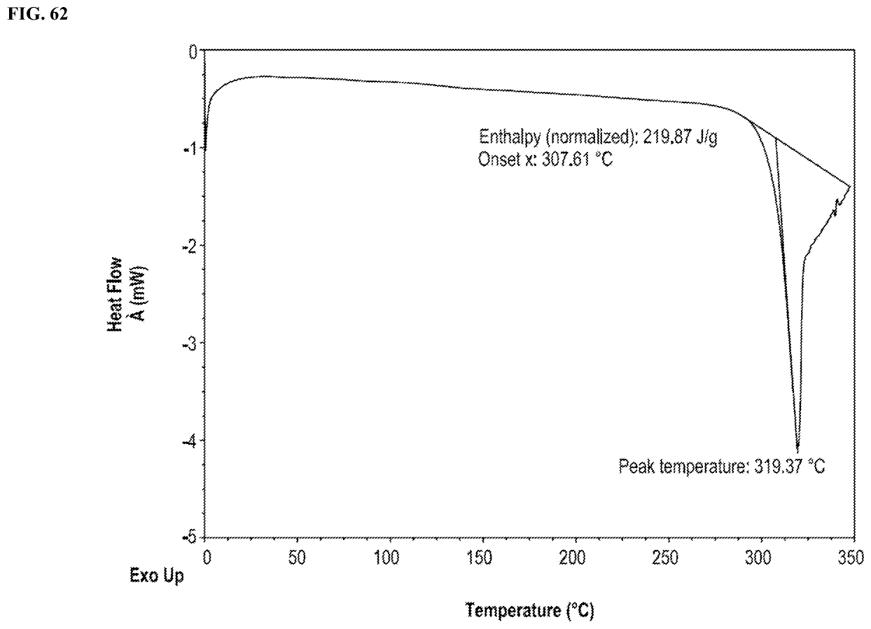

In some embodiments, crystalline Form T is further characterized by a differential scanning calorimetry (DSC) thermogram substantially in accordance with FIG. 62.

In some embodiments, crystalline Form T is further characterized by a weight percent loss of about 0.8% upon heating to about 220° C., as measured by a thermal gravimetric analysis (TGA).

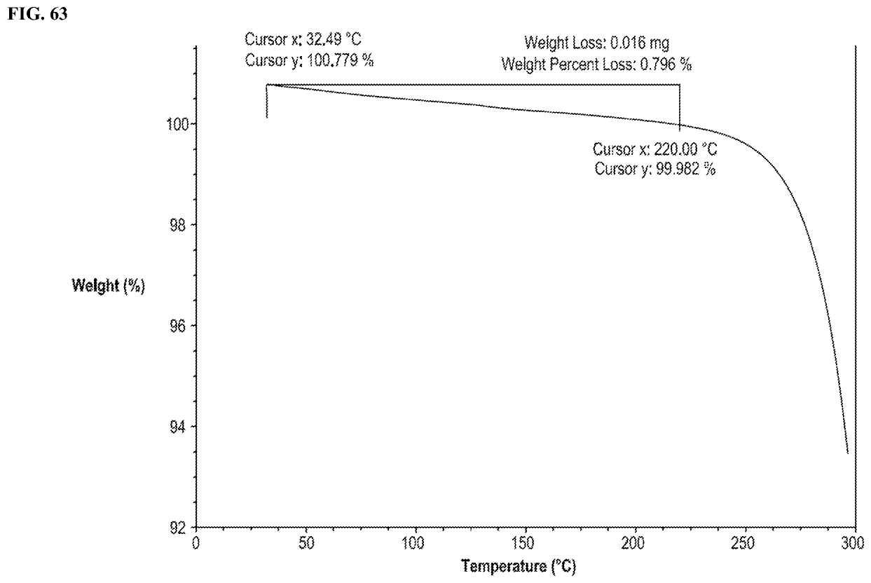

In some embodiments, crystalline Form T is further characterized by a thermal gravimetric analysis (TGA) thermogram substantially in accordance with FIG. 63.

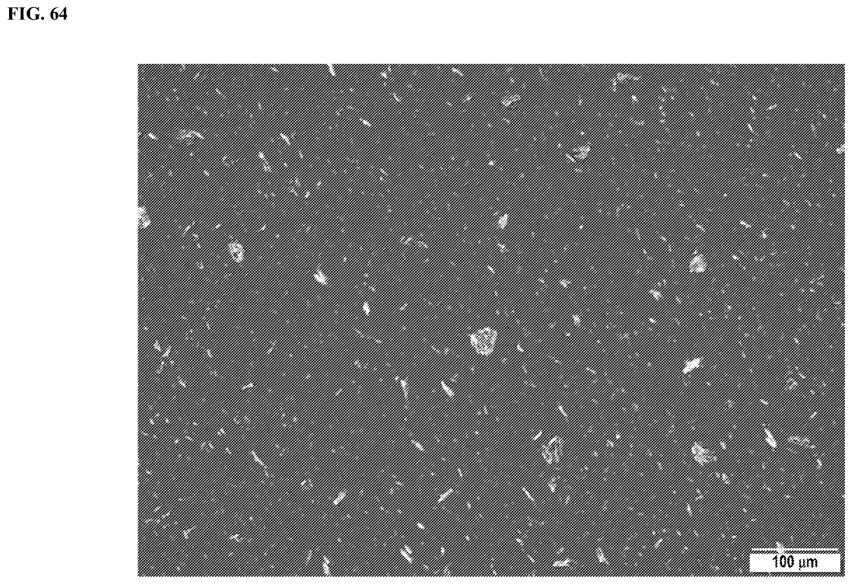

In some embodiments, crystalline Form T is further characterized by a polarized light microscope (PLM) profile substantially as shown in FIG. 64. In some embodiments, crystalline Form T is in an irregular particle characterized by a polarized light microscope (PLM) profile substantially as shown in FIG. 64.

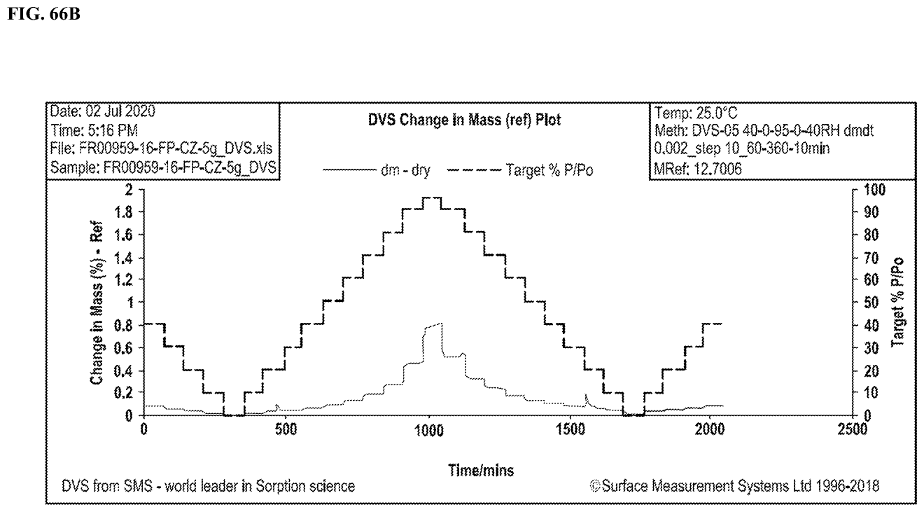

In some embodiments, crystalline Form T is further characterized by a dynamic vapor sorption (DVS) profile substantially as shown in FIG. 66.

Crystalline Form T is in an anhydrate form.

In some embodiments, crystalline Form T is characterized by an X-ray powder diffraction pattern substantially in accordance with FIG. 61; and is further characterized by a differential scanning calorimetry (DSC) thermogram substantially in accordance with FIG. 62. In some embodiments, crystalline Form T is characterized by an X-ray powder diffraction pattern substantially in accordance with FIG. 61; is further characterized by a differential scanning calorimetry (DSC) thermogram substantially in accordance with FIG. 62; and is further characterized by a thermal gravimetric analysis (TGA) thermogram substantially in accordance with FIG. 63.

### III-21. Crystalline Form U

In one embodiment, the present disclosure provides crystalline Form U of the compound having Formula (I). In some embodiments, Form U is characterized by an X-ray powder diffraction (XRPD) pattern including peaks at 6.2, 12.4, and 16.4 degrees 2θ (±0.2 degrees 2θ). In some embodiments, Form U is characterized by an X-ray powder diffraction (XRPD) pattern including peaks at 6.2, 10.3, 12.4, and 16.4 degrees 2θ (±0.2 degrees 2θ). In some embodiments, Form U is characterized by an X-ray powder diffraction (XRPD) pattern including peaks at 6.2, 10.3, 10.8, 12.4, and 16.4 degrees 2θ (±0.2 degrees 2θ).

In some embodiments, the X-ray powder diffraction pattern further includes peaks at 17.6, 21.3, 22.4, 27.2, and 28.5 degrees 2θ (±0.2 degrees 2θ). In some embodiments, the X-ray powder diffraction pattern further includes peaks at 8.8, 13.8, 18.3, 19.8, and 23.2 degrees 2θ (±0.2 degrees 2θ). In some embodiments, crystalline Form U of the compound having Formula (I), characterized by an X-ray powder diffraction (XRPD) pattern including peaks (i.e., the first 10 peaks ranked according to relative peak intensity %) at 6.2, 10.3, 10.8, 12.4, 16.4, 17.6, 21.3, 22.4, 27.2, and 28.5 degrees 2θ (±0.2 degrees 2θ). In some embodiments, Form U is characterized by an X-ray powder diffraction (XRPD) pattern including one, two, three, four, five or more peaks listed in Table 28. In some embodiments, Form U is characterized by an X-ray powder diffraction (XRPD) pattern including at least five peaks listed in Table 28. In some embodiments, Form U is characterized by an X-ray powder diffraction (XRPD) pattern including at least four peaks listed in Table 28.

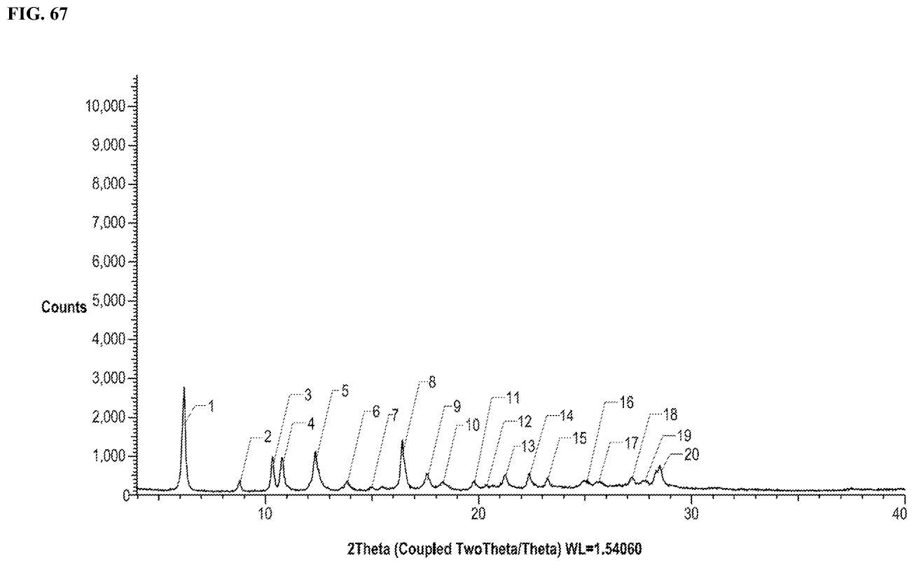

In some embodiments, crystalline Form U of the compound having Formula (I) is characterized by an X-ray powder diffraction (XRPD) pattern substantially in accordance with FIG. 67.

In some embodiments, crystalline Form U is substantially free of other crystalline or amorphous forms of the compound having Formula (I).

### III-22. Solid State Forms and Form V

In some embodiments, the present disclosure provides solid state forms of the compound having Formula (I). Solid state forms of the compound having Formula (I) includes embodiments where the solid state form is not a single, isolated crystalline form. In some embodiments, the solid state form of the compound having Formula (I) is substantially crystalline. In some embodiments, the solid state form of the compound having Formula (I) comprises at least 30 wt. % of a particular crystalline form. In some embodiments, the solid state form of the compound having Formula (I) comprises at least 35 wt. % of a particular crystalline form. In some embodiments, the solid state form of the compound having Formula (I) comprises at least 40 wt. % of a particular crystalline form. In some embodiments, the solid state form of the compound having Formula (I) comprises at least 45 wt. % of a particular crystalline form. In some embodiments, the solid state form of the compound having Formula (I) comprises at least 50 wt. % of a particular crystalline form. In some embodiments, the solid state form of the compound having Formula (I) comprises at least 55 wt. % of a particular crystalline form. In some embodiments, the solid state form of the compound having Formula (I) comprises at least 60 wt. % of a particular crystalline form. In some embodiments, the solid state form of the compound having Formula (I) comprises at least 65 wt. % of a particular crystalline form. In some embodiments, the solid state form of the compound having Formula (I) comprises at least 70 wt. % of a particular crystalline form. In some embodiments, the solid state form of the compound having Formula (I) comprises at least 75 wt. % of a particular crystalline form. In some embodiments, the solid state form of the compound having Formula (I) comprises at least 80 wt. % of a particular crystalline form. In some embodiments, the solid state form of the compound having Formula (I) comprises at least 85 wt. % of a particular crystalline form. In some embodiments, the solid state form of the compound having Formula (I) comprises at least 90 wt. % of a particular crystalline form. In some embodiments, the solid state form of the compound having Formula (I) comprises at least 95 wt. % of a particular crystalline form. In some embodiments, the solid state form of the compound having Formula (I) comprises at least 99 wt. % of a particular crystalline form.

In some embodiments, the particular crystalline form in the solid state forms described herein is Form A. In some embodiments, the particular crystalline form in the solid state forms described herein is Form B. In some embodiments, the particular crystalline form in the solid state forms described herein is Form C. In some embodiments, the particular crystalline form in the solid state forms described herein is Form D. In some embodiments, the particular crystalline form in the solid state forms described herein is Form E. In some embodiments, the particular crystalline form in the solid state forms described herein is Form F. In some embodiments, the particular crystalline form in the solid state forms described herein is Form G. In some embodiments, the particular crystalline form in the solid state forms described herein is Form H. In some embodiments, the particular crystalline form in the solid state forms described herein is Form I. In some embodiments, the particular crystalline form in the solid state forms described herein is Form J. In some embodiments, the particular crystalline form in the solid state forms described herein is Form K. In some embodiments, the particular crystalline form in the solid state forms described herein is Form L. In some embodiments, the particular crystalline form in the solid state forms described herein is Form M. In some embodiments, the particular crystalline form in the solid state forms described herein is Form N. In some embodiments, the particular crystalline form in the solid state forms described herein is Form O. In some embodiments, the particular crystalline form in the solid state forms described herein is Form P. In some embodiments, the particular crystalline form in the solid state forms described herein is Form Q. In some embodiments, the particular crystalline form in the solid state forms described herein is Form R. In some embodiments, the particular crystalline form in the solid state forms described herein is Form S. In some embodiments, the particular crystalline form in the solid state forms described herein is Form T. In some embodiments, the particular crystalline form in the solid state forms described herein is Form U.

In some embodiments, the particular crystalline form is the solid state forms described herein is Form A.

In some embodiments, the solid state form of the compound having Formula (I) comprises at least 30 wt. % crystalline Form A. In some embodiments, the solid state form of the compound having Formula (I) comprises at least 35 wt. % crystalline Form A. In some embodiments, the solid state form of the compound having Formula (I) comprises at least 40 wt. % crystalline Form A. In some embodiments, the solid state form of the compound having Formula (I) comprises at least 45 wt. % crystalline Form A. In some embodiments, the solid state form of the compound having Formula (I) comprises at least 50 wt. % crystalline Form A In some embodiments, the solid state form of the compound having Formula (I) comprises at least 55 wt. % crystalline Form A. In some embodiments, the solid state form of the compound having Formula (I) comprises at least 60 wt. % crystalline Form A. In some embodiments, the solid state form of the compound having Formula (I) comprises at least 65 wt. % crystalline Form A. In some embodiments, the solid state form of the compound having Formula (I) comprises at least 70 wt. % crystalline Form A. In some embodiments, the solid state form of the compound having Formula (I) comprises at least 75 wt. % crystalline Form A. In some embodiments, the solid state form of the compound having Formula (I) comprises at least 80 wt. % crystalline Form A. In some embodiments, the solid state form of the compound having Formula (I) comprises at least 85 wt. % crystalline Form A. In some embodiments, the solid state form of the compound having Formula (I) comprises at least 90 wt. % crystalline Form A. In some embodiments, the solid state form of the compound having Formula (I) comprises at least 95 wt. % crystalline Form A. In some embodiments, the solid state form of the compound having Formula (I) comprises at least 99 wt. % crystalline Form A.

In some embodiments, the solid state form of the compound having Formula (I) comprises Formula (I) as a free base. In some embodiments, the solid state form is of Formula (I) as a free base.

In some embodiments, the present disclosure provides solid state Form V of the compound having Formula (I).

In one embodiment, the present disclosure provides solid state Form V of a compound having Formula (I), characterized by an X-ray powder diffraction (XRPD) pattern including peaks at 6.2, 10.9, and 16.5 degrees 2θ (±0.2 degrees 2θ). In one embodiment, the present disclosure provides solid state Form V of a compound having Formula (I), characterized by an X-ray powder diffraction (XRPD) pattern including peaks at 6.2, 10.4, 10.9, and 16.5 degrees 2θ (±0.2 degrees 2θ). In one embodiment, the present disclosure provides solid state Form V of a compound having Formula (I), characterized by an X-ray powder diffraction (XRPD) pattern including peaks at 6.2, 10.4, 10.9, 12.3, and 16.5 degrees 2θ (±0.2 degrees 2θ).

In some embodiments, the X-ray powder diffraction pattern further includes peaks at 12.5, 21.3, 26.1, 27.5, and 28.6 degrees 2θ (±0.2 degrees 2θ). In some embodiments, the X-ray powder diffraction pattern further includes peaks at 8.9, 19.9, 22.6, and 23.4 degrees 2θ (±0.2 degrees 2θ). In some embodiments, solid state Form V of a compound having Formula (I) is characterized by an X-ray powder diffraction (XRPD) pattern including peaks (i.e., the first 10 peaks ranked according to relative peak intensity %) at 6.2, 10.4, 10.9, 12.3, 12.5, 16.5, 21.3, 26.1, 27.5, and 28.6 degrees 2θ (±0.2 degrees 2θ). In some embodiments, solid state Form V of the compound having Formula (I) is characterized by an X-ray powder diffraction (XRPD) pattern including peaks at 6.2, 10.4, 10.9, 12.3, and 16.5 degrees 2θ (0.2 degrees 2θ). In some embodiments, solid state Form V of the compound having Formula (I), characterized by an X-ray powder diffraction (XRPD) pattern comprising at least any of the two peaks selected from 6.2, 10.4, 10.9, 12.3, and 16.5 degrees 2θ (±0.2 degrees 2θ). In some embodiments, solid state Form V of the compound having Formula (I) is characterized by an X-ray powder diffraction (XRPD) pattern comprising at least any of the three peaks selected from 6.2, 10.4, 10.9, 12.3, and 16.5 degrees 2θ (±0.2 degrees 2θ). In some embodiments, solid state Form V of the compound having Formula (I) is characterized by an X-ray powder diffraction (XRPD) pattern comprising at least any of the four peaks selected from 6.2, 10.4, 10.9, 12.3, and 16.5 degrees 2θ (±0.2 degrees 2θ). In some embodiments, solid state Form V is characterized by an X-ray powder diffraction (XRPD) pattern including one, two, three, four, five or more peaks listed in Table 2. In some embodiments, solid state Form V is characterized by an X-ray powder diffraction (XRPD) pattern including at least five peaks listed in Table 2. In some embodiments, solid state Form V is characterized by an X-ray powder diffraction (XRPD) pattern including at least four peaks listed in Table 2. In some embodiments, solid state Form V is characterized by an X-ray powder diffraction (XRPD) pattern including at least three peaks listed in Table 2.

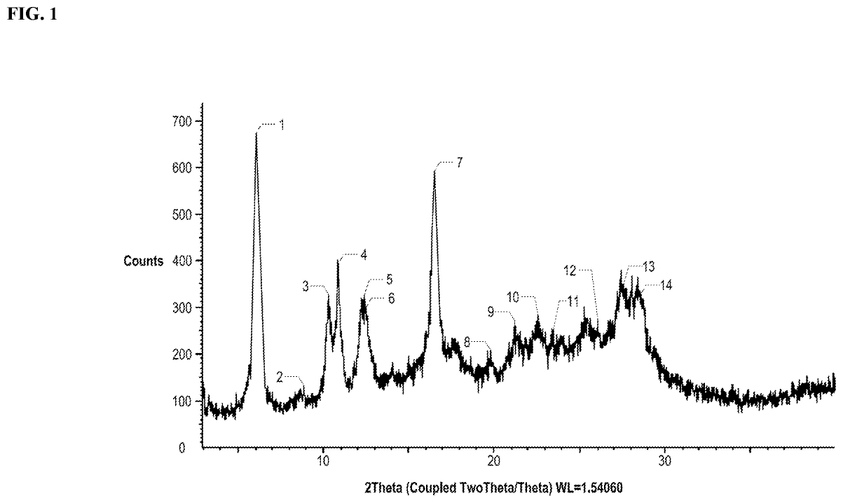

In some embodiments, solid state Form V of a compound having Formula (I) is characterized by an X-ray powder diffraction pattern substantially in accordance with FIG. 1.

In some embodiments, solid state Form V is further characterized by a differential scanning calorimetry (DSC) thermogram including an endothermic peak at about 321.6° C. In some embodiments, solid state Form V is further characterized by a differential scanning calorimetry (DSC) thermogram including an onset temperature of about 316.3° C. and an endothermic peak at about 321.6° C.

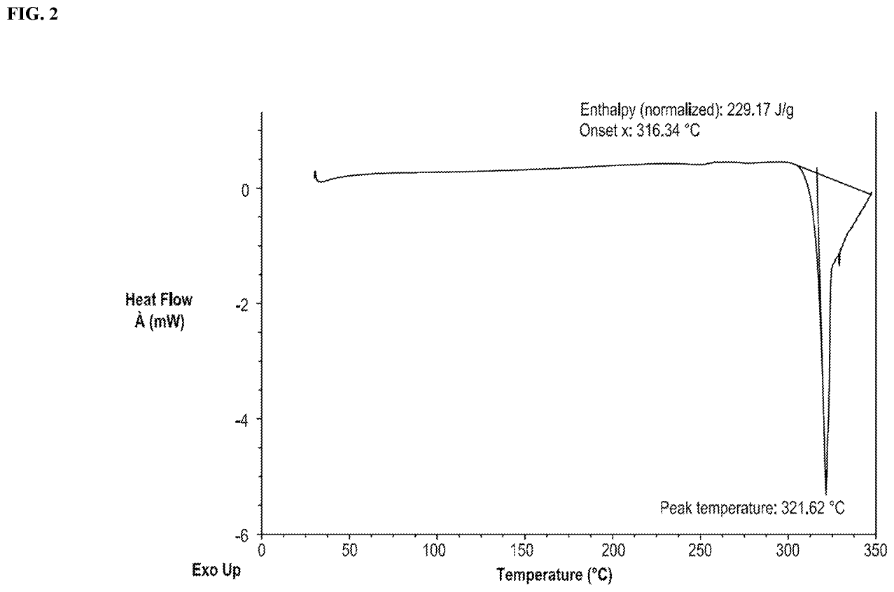

In some embodiments, solid state Form V is further characterized by a differential scanning calorimetry (DSC) thermogram substantially in accordance with FIG. 2.

In some embodiments, solid state Form V is further characterized by a weight percent loss of about 0.7% upon heating to about 200° C., as measured by a thermal gravimetric analysis (TGA).

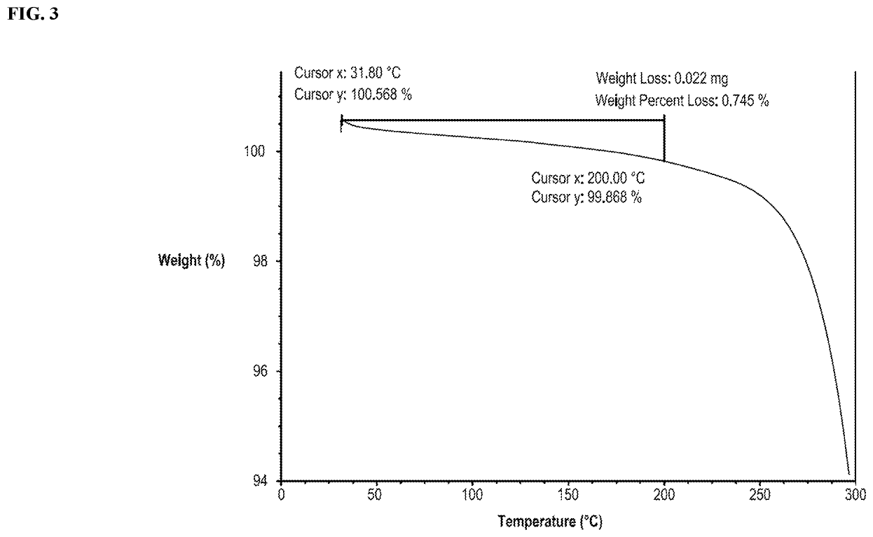

In some embodiments, solid state Form V is further characterized by a thermal gravimetric analysis (TGA) thermogram substantially in accordance with FIG. 3.

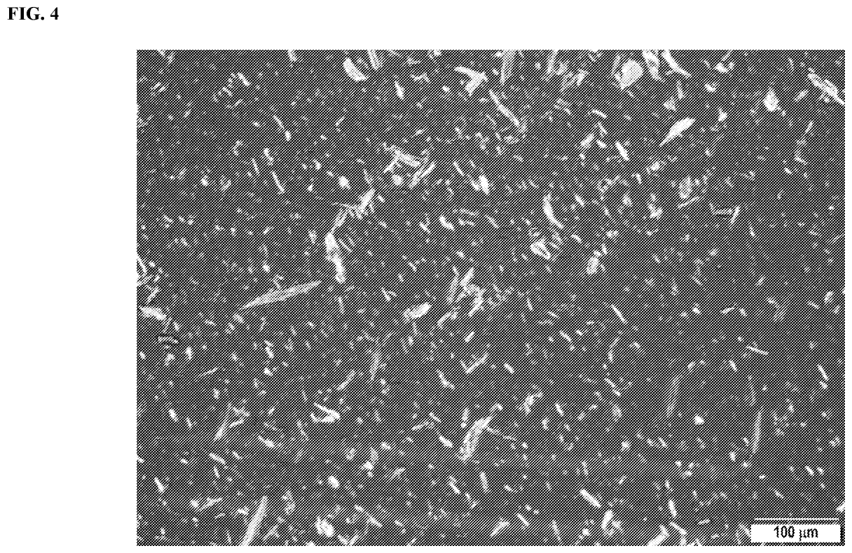

In some embodiments, solid state Form V is further characterized by a polarized light microscope (PLM) profile substantially as shown in FIG. 4. In some embodiments, solid state Form V is in a needle-like particle characterized by a polarized light microscope (PLM) profile substantially as shown in FIG. 4.

Solid state Form V is in an anhydrous form.

In some embodiments, solid state Form V is characterized by an X-ray powder diffraction pattern substantially in accordance with FIG. 1; and is further characterized by a differential scanning calorimetry (DSC) thermogram substantially in accordance with FIG. 2. In some embodiments, solid state Form V is characterized by an X-ray powder diffraction pattern substantially in accordance with FIG. 1; is further characterized by a differential scanning calorimetry (DSC) thermogram substantially in accordance with FIG. 2; and is further characterized by a thermal gravimetric analysis (TGA) thermogram substantially in accordance with FIG. 3.

### IV. Substantially Amorphous Form

In a second aspect, the present disclosure provides a substantially amorphous form of the compound having Formula (I). In some embodiments, the substantially amorphous form is substantially free of other crystalline forms of the compound having Formula (I). In some embodiments, the substantially amorphous form includes no more than about 10% of other crystalline forms of the compound having Formula (I). In some embodiments, the substantially amorphous form includes no more than about 8%, 7%, 6%, 5%, 4%, 3%, 2%, 1%, or 0.5% of other crystalline forms of the compound having Formula (I). In some embodiments, the substantially amorphous form includes no more than about 5% of other crystalline forms of the compound having Formula (I).

In some embodiments, the substantially amorphous form is prepared by grinding any one of crystalline Forms A to U, each of which is as defined and described herein. In some embodiments, any one of crystalline Forms A to U is ground in the absence of a solvent or water (e.g., dry grinding), thereby providing the substantially amorphous form. In some embodiments, any one of crystalline Forms A to U is ground in the presence of a solvent (e.g., ethanol) (e.g., wet grinding), thereby providing the substantially amorphous form. In some embodiments, the substantially amorphous form is prepared by grinding solid state Form V (e.g., dry grinding or wet grinding in ethanol). In some embodiments, the substantially amorphous form is prepared by grinding Form A (e.g., dry grinding or wet grinding in ethanol). In some embodiments, the substantially amorphous form is prepared by grinding Form D (e.g., dry grinding or wet grinding in ethanol). In some embodiments, the substantially amorphous form is prepared by grinding Form T (e.g., dry grinding or wet grinding in ethanol). In some embodiments, the substantially amorphous form is prepared by grinding Form T in the absence of a solvent or water (e.g., dry grinding).

In general, the dry or wet grinding can be conducted using methods known in the art, for example manually or mechanically. In some embodiments, the dry or wet grinding is conducted manually.

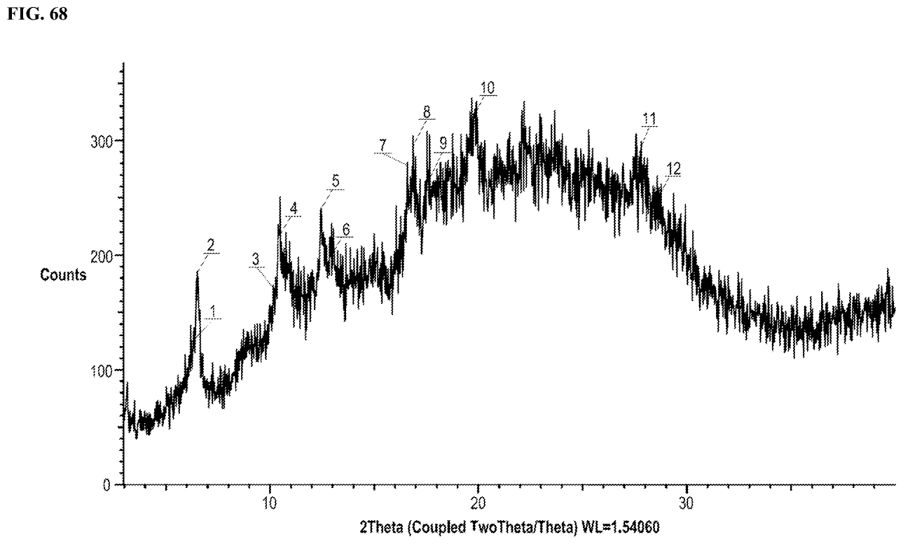

In some embodiments, the substantially amorphous form of the compound having Formula (I) is characterized by an X-ray powder diffraction (XRPD) pattern substantially in accordance with FIG. 68.

In some embodiments, the present disclosure provides a substantially amorphous form of the compound having Formula (I), wherein the substantially amorphous form is prepared by grinding any one of crystalline Forms A to U (e.g., dry grinding or wet grinding in ethanol); and is characterized by an X-ray powder diffraction (XRPD) pattern substantially in accordance with FIG. 68.

In some embodiments, the present disclosure provides a substantially amorphous form of the compound having Formula (I), wherein the substantially amorphous form is prepared by grinding Form T in the absence of a solvent or water (e.g., dry grinding); and is characterized by an X-ray powder diffraction (XRPD) pattern substantially in accordance with FIG. 68.

### V. Composition

The crystalline forms of the compound of Formula (I) as described herein may be in the form of compositions suitable for administration to a subject. In general, such compositions are pharmaceutical compositions comprising a crystalline form of the compound of Formula (I) as described herein and one or more pharmaceutically acceptable or physiologically acceptable excipients.

The substantially amorphous from of the compound of Formula (I) as described herein may be in the form of compositions suitable for administration to a subject. In general, such compositions are pharmaceutical compositions comprising a substantially amorphous form of the compound of Formula (I) as described herein and one or more pharmaceutically acceptable or physiologically acceptable excipients.

In some embodiments, the crystalline form of the compound of Formula (I) as described herein is in a pharmaceutical composition. In some embodiments, the pharmaceutical composition includes any one of crystalline Forms A to U of the compound of Formula (I) as described herein or a solid state form of the compound of Formula (I) as described herein and one or more pharmaceutically acceptable or physiologically acceptable excipients. In some embodiments, the pharmaceutical composition includes crystalline Form A of the compound of Formula (I) as described herein and one or more pharmaceutically acceptable or physiologically acceptable excipients. In some embodiments, the pharmaceutical composition includes crystalline Form B of the compound of Formula (I) as described herein and one or more pharmaceutically acceptable or physiologically acceptable excipients. In some embodiments, the pharmaceutical composition includes crystalline Form C of the compound of Formula (I) as described herein and one or more pharmaceutically acceptable or physiologically acceptable excipients. In some embodiments, the pharmaceutical composition includes crystalline Form D of the compound of Formula (I) as described herein and one or more pharmaceutically acceptable or physiologically acceptable excipients. In some embodiments, the pharmaceutical composition includes crystalline Form E of the compound of Formula (I) as described herein and one or more pharmaceutically acceptable or physiologically acceptable excipients. In some embodiments, the pharmaceutical composition includes crystalline Form F of the compound of Formula (I) as described herein and one or more pharmaceutically acceptable or physiologically acceptable excipients. In some embodiments, the pharmaceutical composition includes crystalline Form G of the compound of Formula (I) as described herein and one or more pharmaceutically acceptable or physiologically acceptable excipients. In some embodiments, the pharmaceutical composition includes crystalline Form H of the compound of Formula (I) as described herein and one or more pharmaceutically acceptable or physiologically acceptable excipients. In some embodiments, the pharmaceutical composition includes crystalline Form I of the compound of Formula (I) as described herein and one or more pharmaceutically acceptable or physiologically acceptable excipients. In some embodiments, the pharmaceutical composition includes crystalline Form J of the compound of Formula (I) as described herein and one or more pharmaceutically acceptable or physiologically acceptable excipients. In some embodiments, the pharmaceutical composition includes crystalline Form K of the compound of Formula (I) as described herein and one or more pharmaceutically acceptable or physiologically acceptable excipients. In some embodiments, the pharmaceutical composition includes crystalline Form L of the compound of Formula (I) as described herein and one or more pharmaceutically acceptable or physiologically acceptable excipients. In some embodiments, the pharmaceutical composition includes crystalline Form M of the compound of Formula (I) as described herein and one or more pharmaceutically acceptable or physiologically acceptable excipients. In some embodiments, the pharmaceutical composition includes crystalline Form N of the compound of Formula (I) as described herein and one or more pharmaceutically acceptable or physiologically acceptable excipients. In some embodiments, the pharmaceutical composition includes crystalline Form O of the compound of Formula (I) as described herein and one or more pharmaceutically acceptable or physiologically acceptable excipients. In some embodiments, the pharmaceutical composition includes crystalline Form P of the compound of Formula (I) as described herein and one or more pharmaceutically acceptable or physiologically acceptable excipients. In some embodiments, the pharmaceutical composition includes crystalline Form Q of the compound of Formula (I) as described herein and one or more pharmaceutically acceptable or physiologically acceptable excipients. In some embodiments, the pharmaceutical composition includes crystalline Form S of the compound of Formula (I) as described herein and one or more pharmaceutically acceptable or physiologically acceptable excipients. In some embodiments, the pharmaceutical composition includes crystalline Form T of the compound of Formula (I) as described herein and one or more pharmaceutically acceptable or physiologically acceptable excipients. In some embodiments, the pharmaceutical composition includes crystalline Form U of the compound of Formula (I) as described herein and one or more pharmaceutically acceptable or physiologically acceptable excipients. In some embodiments, the pharmaceutical composition includes a solid state form of a compound of Formula (I) as described herein and one or more pharmaceutically acceptable or physiologically acceptable excipients. In some embodiments, the pharmaceutical composition includes solid state Form V as described herein and one or more pharmaceutically acceptable or physiologically acceptable excipients.

In some embodiments, the pharmaceutical composition includes the substantially amorphous from of the compound of Formula (I) as described herein and one or more pharmaceutically acceptable or physiologically acceptable excipients.

The pharmaceutical compositions may be used in the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., a crystalline form or a substantially amorphous form of the compound of Formula (I) as described herein) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a crystalline form of the compound of Formula (I) as described herein, or a salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a compound of Formula (I) or a subembodiment described herein, or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound of Formula (I) as described herein, or a salt thereof over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

A crystalline form or a substantially amorphous form of the compound of Formula (I) as described herein may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

### VI. Methods for Preparing Crystalline Forms

In a third aspect, the present disclosure provides a method for preparing solid state Form V of a compound having Formula (I). The method includes:
a) forming a first slurry comprising a crude compound of Formula (I) and a first organic solvent;
b) isolating a first precipitate from step a);
c) forming a second slurry comprising the first precipitate of step b) and a second organic solvent;
d) isolating a second precipitate; and
e) drying the second precipitate to provide the solid state Form V of Formula (I),
   wherein the first organic solvent is a C₁₋₄ alkyl alcohol and the second organic solvent is a chlorinated aprotic solvent.

In some embodiments, the first organic solvent is methanol, ethanol, isopropyl, or mixtures thereof. In some embodiments, the first organic solvent is ethanol.

In some embodiments, the second organic solvent is dichloromethane.

In some embodiments, the first organic solvent is ethanol; and the second organic solvent is dichloromethane.

Steps a) to d) can be conducted at any temperature, for example at a temperature of from 10° C. to 50° C. In some embodiments, steps a) to d) are conducted at a temperature of from 20° C. to 30° C. In some embodiments, steps a) to d) are conducted at a temperature of about 25° C. In some embodiments, steps a) to d) are conducted at room temperature.

In some embodiments, the first slurry of step a) is formed at a temperature of about 25° C. and maintained for a period of from 30 minutes to 2 hours. In some embodiments, the first slurry of step a) is formed at a temperature of about 25° C. and maintained for a period of about one hour.

In some embodiments, the second slurry of step c) is formed at a temperature of about 25° C. and maintained for a period of from 30 minutes to 2 hours. In some embodiments, the second slurry of step c) is formed at a temperature of about 25° C. and maintained for a period of about one hour.

The isolating of step b) and/or step d) can be conducted by any method known in the art. In some embodiments, isolating of step b) and/or step d) is conducted by filtration.

The drying of step e) can be conducted by any method known in the art, for example under a vacuum at a temperature of from room temperature to 80° C. In some embodiments, the drying of step e) is conducted at a temperature of from 30° C. to 50° C. In some embodiments, the drying of step e) is conducted at a temperature of about 40° C. In some embodiments, the drying of step e) is conducted under a vacuum at a temperature of from 30° C. to 50° C. In some embodiments, the drying of step e) is conducted under a vacuum at a temperature of about 40° C. In some embodiments, the drying of step e) is conducted under a vacuum at a temperature of about 40° C. for a period of from 1-5 hours. In some embodiments, the drying of step e) is conducted under a vacuum at a temperature of about 40° C. for a period of about 3 hours.

In some embodiments, the crude compound of Formula (I) is present in the first slurry and/or the second slurry in an amount of from about 25 g/L to 100 g/L. In some embodiments, the crude compound of Formula (I) is present in the first slurry in an amount of from about 25 g/L to 100 g/L. In some embodiments, the crude compound of Formula (I) is present in the first slurry in an amount of from about 50 g/L to 90 g/L. In some embodiments, the crude compound of Formula (I) is present in the first slurry in an amount of from about 50 g/L to 70 g/L. In some embodiments, the crude compound of Formula (I) is present in the first slurry in an amount of about 60 g/L. In some embodiments, the compound of Formula (I) from step b) is present in the second slurry in an amount of from about 25 g/L to 100 g/L. In some embodiments, the compound of Formula (I) from step b) is present in the second slurry in an amount of from about 50 g/L to 70 g/L. In some embodiments, the compound of Formula (I) from step b) is present in the second slurry in an amount of about 60 g/L.

In some embodiments, prior to step a), the method further includes:
a-1) suspending a first crude compound of Formula (I) in water and stirring the suspension for a period of from 6 to 24 hours;
a-2) removing a precipitate by filtration to provide a filtrate comprising a crude compound of Formula (I); and
a-3) concentrating the filtrate to provide the crude compound of Formula (I).

In a fourth aspect, the present disclosure provides a method for preparing crystalline Form A of a compound having Formula (I). The method includes:
a) forming a first mixture comprising a crude compound of Formula (I), ACN, and water;
b) solvent exchanging with water at a temperature of no more than about 65° C. to form a second mixture;
c) cooling the second mixture and stirring to form a third precipitate;
d) isolating the third precipitate;
e) forming a slurry comprising the third precipitate, methyl ethyl ketone (MEK), and water;
f) isolating a fourth precipitate from step e);
g) drying the fourth precipitate to provide the crystalline Form A of Formula (I).

In step a), in some embodiments, a ratio of ACN to water is from about 5:1 to about 2:1 by volume. In some embodiments, a ratio of ACN to water is about 4:1 by volume.

Step a) can be conducted at an elevated temperature, for example at a temperature of from about 75° C. to 80° C. In some embodiments, step a) is conducted at a temperature of from about 75° C. to 80° C.

In step b), in some embodiments, the solvent exchanging with water is conducted by 1) concentrating the first mixture of step a); 2) adding water; 3) concentrating; and 4) adding a final portion of water.

In some embodiments, the method further includes adding a crystalline seed of the compound of Formula (I) (e.g., Form A) to the first mixture of step a) prior to or during the solvent exchanging of step b). In some embodiments, a crystalline seed of the compound of Formula (I) (e.g., Form A) is added after concentrating the first mixture of step a). In some embodiments, the crystalline seed of the compound of Formula (I) is Form A.

Step b) can be conducted at an elevated temperature of no more than about 65° C. In some embodiments, step b) is conducted at a temperature of no more than about 65° C.

Step c) can be conducted at a chilled temperature, for example at a temperature of from about 0° C. to 5° C. In some embodiments, step c) is conducted at a temperature of from about 0° C. to 5° C. The stirring of step c) can be conducted for a period of from about 5 hours to 24 hours. In some embodiments, the stirring of step c) is conducted for a period of from about 10 to 18 hours. In some embodiments, the stirring of step c) is conducted for a period of about 13 hours.

In some embodiments, the third precipitate of step c) is formed at a temperature of from about 0° C. to 5° C. and stirred for a period of from 10 to 18 hours. In some embodiments, the third precipitate of step c) is formed at a temperature of from about 0° C. to 5° C. and stirred for a period of about 13 hours.

In step e), in some embodiments, a ratio of methyl ethyl ketone (MEK) to water is from about 20:1 to 5:1 by volume. In step e), in some embodiments, a ratio of methyl ethyl ketone (MEK) to water is from about 12:1 to 8:1 by volume. In step e), in some embodiments, a ratio of methyl ethyl ketone (MEK) to water is from about 11:1 to 9:1 by volume. In some embodiments, a ratio of methyl ethyl ketone (MEK) to water is about 10:1 by volume. In some embodiments, a mixture of step e) has a water content of from about 9 to 10% by weight. In some embodiments, a mixture of step e) has a water content of from about 8% to about 110% by weight.

In some embodiments, the slurry of step e) is formed at a temperature of from about 60° C. to 65° C. and stirred for a period of from about 15 to 24 hours. In some embodiments, the slurry of step e) is formed at a temperature of from about 60° C. to 65° C. and stirred for a period of from about 17 to 22 hours. In some embodiments, the slurry of step e) is further cooled to a temperature of from about 0° C. to 5° C. In some embodiments, the slurry of step e) is further cooled to a temperature of from about 0° C. to 5° C. and stirred for a period of from about 15 to 24 hours. In some embodiments, the slurry of step e) is further cooled to a temperature of about 0° C. and stirred for a period of about 4 hours; and further stirred at a temperature of from about 0° C. to 5° C. for a period of about 16 hours.

The isolating of step d) and/or step f) can be conducted by any method known in the art. In some embodiments, isolating of step d) and/or step f) is conducted by filtration.

The drying of step g) can be conducted by any method known in the art, for example under a vacuum at a temperature of from room temperature to 80° C. In some embodiments, the drying of step e) is conducted at a temperature of from 65° C. to 70° C. In some embodiments, the drying of step e) is conducted under a vacuum at a temperature of from about 65° C. to 70° C. for a period of from 1 to 4 days. In some embodiments, the drying of step e) is conducted under a vacuum at a temperature of from about 65° C. to 70° C. for a period of about 3 days.

In some embodiments, the crude compound of Formula (I) is present in the first mixture in an amount of from about 10 g/L to 100 g/L, from about 20 g/L to 100 g/L, from about 30 g/L to 100 g/L, from about 10 g/L to 50 g/L, from about 20 g/L to 50 g/L, or from about 30 g/L to 50 g/L. In some embodiments, the crude compound of Formula (I) is present in the first mixture in an amount of from about 20 g/L to 100 g/L. In some embodiments, the crude compound of Formula (I) is present in the first mixture in an amount of from about 20 g/L to 50 g/L. In some embodiments, the crude compound of Formula (I) is present in the first mixture in an amount of about 30 g/L.

The first crude compound of Formula (I) can be obtained from an amination reaction as shown below:

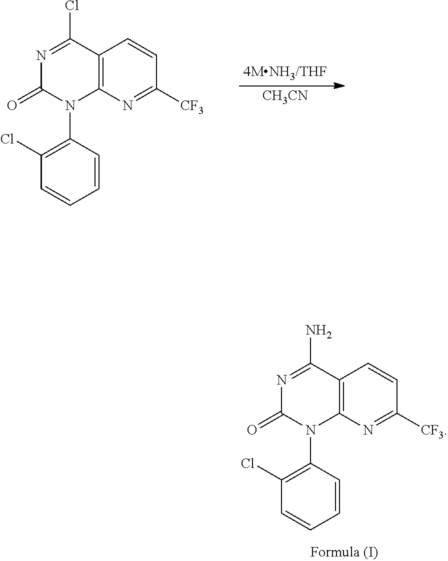 [SMILES: O=c1nc(Cl)c2ccc(C(F)(F)F)nc2n1-c1ccccc1Cl ; "CH3CN" ; "4M·NH3/THF" ; Nc1nc(=O)n(-c2ccccc2Cl)c2nc(C(F)(F)F)ccc12]

In some embodiments, the first crude compound of Formula (I) is obtained by concentrating a reaction mixture of the above amination reaction.

The first crude compound of Formula (I) can be obtained from the two-step reaction as shown below:

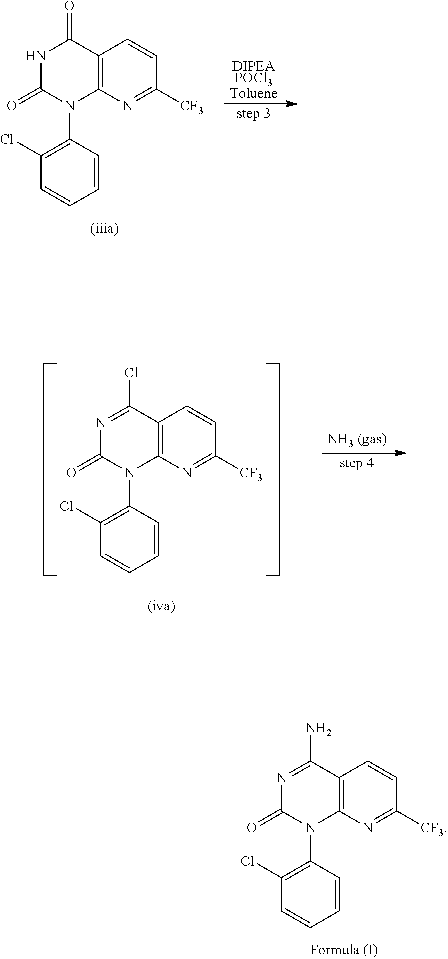

The crystalline form of the present disclosure can be prepared from the compound of Formula (I) by any one of methods as described herein, wherein the crystalline form is any one of crystalline Form A to U.

In some embodiments, the present disclosure provides a method for preparing a crystalline form of a compound having Formula (I), wherein crystalline form is any one of crystalline Form A to U, and the method is selected from the group consisting of:
a) by equilibration with a solvent (e.g., stirring a suspension of the compound of Formula (I) in a solvent at 25° C. for 14 days or at 50° C. for 7 days);
b) by temperature cycling (e.g., equilibrating the compound of Formula (I) with a solvent under a temperature cycle of from 5° C. to 50° C. at a heating/cooling rate of 0.2° C./min for 6 cycles);
d) by crystallization at room temperature by slow evaporation (e.g., evaporating a solution including the compound of Formula (I) under an ambient condition);
e) by crystallization at room temperature by fast evaporation (e.g., evaporating a solution including the compound of Formula (I) under a nitrogen flow);
f) by precipitation with an addition of anti-solvent (e.g., dissolving the compound of Formula (I) in a solvent and adding an anti-solvent slowly to provide a precipitate);
g) by crystallization from a hot saturated solution by slow cooling (e.g., dissolving the compound of Formula (I) in a solvent at 50° C. and cooling the filtered solution to 5° C. at 0.1° C./min);
h) crystallization from a hot saturated solution by fast cooling (e.g., dissolving the compound of Formula (I) in a solvent at 50° C. and cooling the filtered solution in an ice bath);
i) by vapor diffusion (e.g., placing a clear solution of the compound of Formula (I) in a small glass vial without a lid; placing the lidless small vial into a large glass vial containing an anti-solvent in the bottom; and placing the capped large vial at room temperature); and
j) by competitive equilibration (e.g., placing two crystalline forms of the compound of Formula (I) in a solvent; and stirring the resulting suspension at 25° C. and/or 50° C. for 7 days).

In a fifth aspect, the present disclosure provides a method for preparing a crystalline Form of a compound having Formula (I), including:
a) forming a slurry comprising solid state Form V of Formula (I) and a solvent;
b) stirring the slurry for a period of at least a day; and
c) isolating a precipitate; and
d) drying the precipitate to provide the crystalline Form of Formula (I),
wherein the crystalline Form is crystalline Form C, D, E, I, or A-1; and the solvent is methanol, water, a mixture of methanol and water, a mixture of acetone and water, or a mixture of acetonitrile and water.

In some embodiments, the crystalline Form is crystalline Form C; and the solvent is methanol.

In some embodiments, the crystalline Form is crystalline Form D; and the solvent is water.

In some embodiments, the crystalline Form is crystalline Form E; and the solvent is a mixture of methanol and water. In some embodiments, a ratio of methanol to water is about 1:1 by volume.

In some embodiments, the crystalline Form is crystalline Form I; and the solvent is a mixture of acetone and water. In some embodiments, a ratio of acetone to water is about 1:1 by volume.

In some embodiments, the crystalline Form is crystalline Form A; and the solvent is a mixture of acetonitrile and water. In some embodiments, a ratio of acetonitrile to water is about 1:1 by volume.

In some embodiments, the stirring of step b) is conducted at a temperature of about 25° C. and/or about 50° C. In some embodiments, the stirring of step b) is conducted at a temperature of about 25° C. In some embodiments, the stirring of step b) is conducted at a temperature of about 50° C. In some embodiments, the stirring of step b) is conducted at a temperature of about 25° C. and about 50° C.

In some embodiments, the stirring of step b) is conducted for a period of from 3 to 6 days. In some embodiments, the stirring of step b) is conducted at a first temperature of about 50° C. for a first period of 3 days and at a second temperature of about 25° C. for a second period of 3 days. In some embodiments, the stirring of step b) is conducted at a temperature of about 50° C. for a period of 6 days. In some embodiments, the stirring of step b) is conducted at a temperature of about 50° C. for a period of 3 days. In some embodiments, the stirring of step b) is conducted at a temperature of about 25° C. for a period of 6 days.

The isolating of step c) can be conducted by any methods known in the art. In some embodiments, the isolating of step c) is conducted by filtration.

The drying of step d) can be conducted by any methods known in the art. In some embodiments, the drying of step d) is conducted at room temperature. In some embodiments, the drying of step d) is conducted at room temperature for a period of from 10 hours to 72 hours. In some embodiments, the drying of step d) is conducted at room temperature for a period of from 20 hours to 72 hours. In some embodiments, the drying of step d) is conducted at room temperature for a period of about 20 hours. In some embodiments, the drying of step d) is conducted at room temperature for a period of about 65 hours.

In some embodiments, the solid state Form V of Formula (I) is present in the slurry in an amount of from 50 mg/mL to 120 mg/mL. In some embodiments, the solid state Form V of Formula (I) is present in the slurry in an amount of from 60 mg/mL to 100 mg/mL. In some embodiments, the solid state Form V of Formula (I) is present in the slurry in an amount of about 70 mg/mL in methanol. In some embodiments, the solid state Form V of Formula (I) is present in the slurry in an amount of about 100 mg/mL in water. In some embodiments, the solid state Form V of Formula (I) is present in the slurry in an amount of about 83 mg/mL in a mixture of methanol and water, wherein a ratio of methanol to water is about 1:1 by volume. In some embodiments, the solid state Form V of Formula (I) is present in the slurry in an amount of about 63 mg/mL in a mixture of acetone and water, wherein a ratio of acetone to water is about 1:1 by volume. In some embodiments, the solid state Form V of Formula (I) is present in the slurry in an amount of about 65 mg/mL in a mixture of acetonitrile and water, wherein a ratio of acetonitrile to water is about 1:1 by volume.

In some embodiments, the present disclosure provides a method for preparing crystalline Form C of a compound having Formula (I), the method including:
a) forming a slurry comprising solid state Form V of Formula (I) and methanol;
b) stirring the slurry for at a first temperature of about 50° C. for a first period of 3 days and at a second temperature of about 25° C. for a second period of 3 days; and
c) isolating a precipitate by filtration; and
d) drying the precipitate at room temperature to provide crystalline Form C of Formula (I).

In some embodiments of the method for preparing crystalline Form C, the solid state Form V of Formula (I) is present in the slurry in an amount of about 70 mg/mL in methanol. In some embodiments, the drying of step d) is conducted at room temperature for a period of about 20 hours.

In some embodiments, the present disclosure provides a method for preparing crystalline Form D of a compound having Formula (I), the method including:
a) forming a slurry comprising solid state Form V of Formula (I) and water;
b) stirring the slurry for at a temperature of about 50° C. for a period of 6 days; and
c) isolating a precipitate by filtration; and
d) drying the precipitate at room temperature to provide crystalline Form D of Formula (I).

In some embodiments of the method for preparing crystalline Form D, the solid state Form V of Formula (I) is present in the slurry in an amount of about 100 mg/mL in water. In some embodiments, the drying of step d) is conducted at room temperature for a period of about 20 hours.

In some embodiments, the present disclosure provides a method for preparing crystalline Form E of a compound having Formula (I), the method including:
a) forming a slurry comprising solid state Form V of Formula (I) and a mixture of methanol and water;
b) stirring the slurry for at a first temperature of about 50° C. for a first period of 3 days and at a second temperature of about 25° C. for a second period of 3 days; and
c) isolating a precipitate by filtration; and
d) drying the precipitate at room temperature to provide crystalline Form E of Formula (I),
   wherein a ratio of methanol to water is about 1:1 by volume.

In some embodiments of the method for preparing crystalline Form E, the solid state Form V of Formula (I) is present in the slurry in an amount of about 83 mg/mL in a mixture of methanol and water, wherein a ratio of methanol to water is about 1:1 by volume. In some embodiments, the drying of step d) is conducted at room temperature for a period of about 20 hours.

In some embodiments, the present disclosure provides a method for preparing crystalline Form I of a compound having Formula (I), the method including:
a) forming a slurry comprising solid state Form V of Formula (I) and a mixture of acetone and water;
b) stirring the slurry for at a temperature of about 50° C. for a period of 3 days; and
c) isolating a precipitate by filtration; and
d) drying the precipitate at room temperature to provide crystalline Form I of Formula (I),
   wherein a ratio of acetone to water is about 1:1 by volume.

In some embodiments of the method for preparing crystalline Form I, the solid state Form V of Formula (I) is present in the slurry in an amount of about 63 mg/mL in a mixture of acetone and water, wherein a ratio of acetone to water is about 1:1 by volume. In some embodiments, the drying of step d) is conducted at room temperature for a period of about 65 hours.

In some embodiments, the present disclosure provides a method for preparing crystalline Form A of a compound having Formula (I), the method including:
a) forming a slurry comprising solid state Form V of Formula (I) and a mixture of acetonitrile and water;
b) stirring the slurry for at a temperature of about 25° C. for a period of 6 days; and
c) isolating a precipitate by filtration; and
d) drying the precipitate at room temperature to provide crystalline Form A of Formula (I),
   wherein a ratio of acetonitrile to water is about 1:1 by volume.

In some embodiments of the method for preparing crystalline Form A, the solid state Form V of Formula (I) is present in the slurry in an amount of about 68 mg/mL in a mixture of acetonitrile and water, wherein a ratio of acetonitrile to water is about 1:1 by volume. In some embodiments, the drying of step d) is conducted at room temperature for a period of from about 20 to 72 hours.

### VII. Methods for the Treatment

In a sixth aspect, the present disclosure provides a method for treating a disease mediated by MAT2A in a patient, the method including administering to the patient a therapeutically effective amount of a crystalline form or a substantially amorphous form of the compound of Formula (I) as described herein or a pharmaceutical composition thereof as described herein. In some embodiments, the disease is cancer.

Overexpression of the enzyme MAT2A has been demonstrated to mediate certain cancers. In an embodiment, the cancer is neuroblastoma, intestine carcinoma (such as rectum carcinoma, colon carcinoma, familiary adenomatous polyposis carcinoma and hereditary non-polyposis colorectal cancer), esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors (such as glioblastoma, astrocytoma, meningioma, medulloblastoma and peripheral neuroectodermal tumors), Hodgkin lymphoma, esophagogastric cancer, gastrointestinal (GI) cancer, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

In another embodiment, the cancer is lung cancer, non-small cell lung (NSLC) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the vagina, carcinoma of the vulva, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers.

Methylthioadenosine phosphorylase (MTAP) is an enzyme found in all normal tissues that catalyzes the conversion of methylthioadenosine (MTA) into adenine and 5-methylthio-ribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

Many human and murine malignant cells lack MTAP activity. MTAP deficiency is not only found in tissue culture cells but the deficiency is also present in primary leukemias, gliomas, melanomas, pancreatic cancers, non-small cell lung cancers (NSLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas. It has been reported by K. Marjon et al., Cell Reports 15 (2016) 574-587, incorporated herein by reference, that proliferation of cancer cells that are MTAP null is inhibited by knocking down MAT2A expression with shRNA. An MTAP null cancer is a cancer in which the MTAP gene has been deleted or lost or otherwise deactivated or a cancer in which the MTAP protein has a reduced or impaired function.

In a seventh aspect, the present disclosure provides a method of treating a MTAP null cancer in a patient, the method including administering to the patient a therapeutically effective amount of a crystalline form or a substantially amorphous form of the compound of Formula (I) as described herein or a pharmaceutical composition thereof as described herein. Accordingly, in an embodiment of the present disclosure there is provided a method for treating an MTAP null cancer in a patient wherein said cancer is characterized by a reduction or absence of MTAP expression or absence of the MTAP gene, reduced level of MTAP protein, reduced function of MTAP protein, or a combination thereof, as compared to cancers where the MTAP gene is present and fully functioning, said method comprising administering to the patient in need thereof a therapeutically effective amount of a crystalline form or a substantially amorphous form of the compound of Formula (I) as described herein or a pharmaceutical composition thereof as described herein. In an embodiment, the MTAP null cancer is leukemia, glioma, melanoma, pancreatic, non-small cell lung cancer (NSLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma or mesothelioma. In another embodiment, the MTAP null cancer is pancreatic cancer. In yet another embodiment, the MTAP null cancer is bladder cancer, melanoma, brain cancer, lung cancer, pancreatic cancer, breast cancer, esophageal cancer, head and neck cancer, kidney cancer, colon cancer, diffuse large B cell lymphoma (DLBCL), acute lymphoblastic leukemia (ALL) or mantle cell lymphoma (MCL). In yet another embodiment, the MTAP null cancer is gastric cancer. In yet another embodiment, the cancer is colon cancer. In yet another embodiment, the MTAP null cancer is liver cancer. In yet another embodiment, the MTAP null cancer is glioblastoma multiforme (GBM). In yet another embodiment, the MTAP null cancer is bladder cancer. In yet another embodiment, the MTAP null cancer is esophageal cancer. In yet another embodiment, the MTAP null cancer is breast cancer. In yet another embodiment, the MTAP null cancer is NSLCC. In yet another embodiment, the MTAP null cancer is MCL. In yet another embodiment, the MTAP null cancer is DLBCL. In yet another embodiment, the MTAP null cancer is ALL.

In yet another embodiment, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, esophageal cancer, malignant peripheral nerve sheath tumor, and mesothelioma. In an embodiment, the cancer is mesothelioma. In an embodiment, the cancer is non-small cell lung cancer. In another embodiment, the cancer is nonsquamous non-small cell lung cancer. In one embodiment, the cancer is cancer of the colon or rectum. In an embodiment, the cancer is adenocarcinoma of the colon or rectum. In an embodiment, the cancer is breast cancer. In an embodiment, the cancer is adenocarcinoma of the breast. In an embodiment, the cancer is gastric cancer. In an embodiment, the cancer is gastric adenocarcinoma. In an embodiment, the cancer is pancreatic cancer. In an embodiment, the cancer is pancreatic adenocarcinoma. In an embodiment, the cancer is bladder cancer. In an embodiment, the cancer is characterized as being MTAP-null. In an embodiment, the cancer is characterized as being MTAP-deficient. In still another embodiment, the cancer is a solid tumor. In still another embodiment, the cancer is a MTAP-deleted solid tumor. In still another embodiment, the cancer is a metastatic MTAP-deleted solid tumor. In still another embodiment, the cancer is metastatic. In still another embodiment, the cancer is a solid malignant tumor. In still another embodiment, the cancer is a solid tumor. In still another embodiment, the cancer is MTAP-deficient lung or MTAP-deficient pancreatic cancer, including MTAP-deficient NSCLC or MTAP-deficient pancreatic ductal adenocarcinoma (PDAC) or MTAP-deficient esophageal cancer. In another embodiment, the cancer is a tumor having an MTAP gene deletion. In any one of the embodiments herein, the cancer is a solid tumor or a haematological cancer. In one embodiment, the tumor is deficient in MTAP. In another embodiment, the tumor is normal in its expression of MTAP. In still another embodiment, the cancer is NSCLC, mesothelioma, squamous carcinoma of the head and neck, salivary gland tumors, urothelial cancers, sarcomas, or ovarian cancer. In still another embodiment, the cancer is NSCLC, esophagogastric and pancreatic cancers. In still another embodiment, the cancer is characterized by a reduction or absence of MTAP gene expression, absence of MTAP gene, reduced function of MTAP protein, reduced level or absence of MTAP protein, MTA accumulation, or combination thereof. In still another embodiment, the cancer is characterized by a reduction or absence of MTAP gene expression. In still another embodiment, the cancer is characterized by reduced function of MTAP protein. In still another embodiment, the cancer is characterized reduced level or absence of MTAP protein. In still another embodiment, the cancer is characterized by MTA accumulation.

Genomic analysis of MTAP null cell lines has shown that cell lines that also incorporate a KRAS mutation or a p53 mutation were sensitive to MAT2A inhibition.

In an eighth aspect, the present disclosure provides a method for treating a cancer in a patient, wherein the cancer is characterized by a reduction or absence of MTAP gene expression, the absence of the MTAP gene, reduced level of MTAP protein, reduced function of MTAP protein, absence of MTAP protein, or a combination thereof, the method includes administering to the subject a therapeutically effective amount of a crystalline form or a substantially amorphous form of the compound of Formula (I) as described herein or a pharmaceutical composition thereof as described herein. Accordingly, also provided is a method for treating a cancer in a patient wherein said cancer is characterized by reduction or absence of MTAP expression or absence of the MTAP gene, reduced level of MTAP protein, reduced function of MTAP protein, absence of MTAP protein, (i.e., MTAP null), or a combination thereof, and further characterized by the presence of mutant KRAS and/or mutant p53, said method comprising administering to the patient a therapeutically effective amount of a crystalline form or a substantially amorphous form of the compound of Formula (I) as described herein. In one embodiment, the cancer is MTAP null and KRAS mutant. In another embodiment, the cancer is MTAP null and p53 mutant. In yet another embodiment, the cancer is MTAP null, KRAS mutant and p53 mutant.

The term “mutant KRAS” or “KRAS mutation” refers to KRAS protein (or gene encoding said protein) incorporating an activating mutation that alters its normal function. For example, a mutant KRAS protein may incorporate a single amino acid substitution at position 12 or 13. In a particular embodiment, the KRAS mutant incorporates a G12X or G13X substitution, wherein X represents any amino acid change at the indicated position. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. In an embodiment, said p53 mutation is, Y126_splice, K132Q, M133K, R174fs, R175H, R196*, C238S, C242Y, G245S, R248W, R248Q, 1255T, D259V, S261 splice, R267P, R273C, R282W, A159V or R280K. In an embodiment, the foregoing cancer is non-small cell lung cancer (NSLCC), pancreatic cancer, head and neck cancer, gastric cancer, breast cancer, colon cancer or ovarian cancer.

In some embodiments, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma and mesothelioma.

### VII. Embodiments

Embodiment 1. Crystalline Form of a compound having Formula (I):

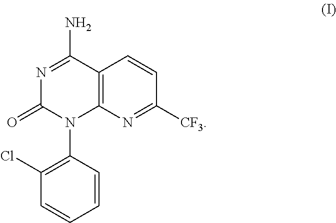 [SMILES: Nc1nc(=O)n(-c2ccccc2Cl)c2nc(C(F)(F)F)ccc12]

Embodiment 2. Crystalline Form A of a compound having Formula (i):

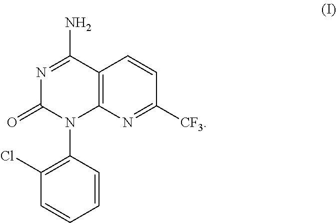 [SMILES: Nc1nc(=O)n(-c2ccccc2Cl)c2nc(C(F)(F)F)ccc12]

Embodiment 3. The crystalline Form A of embodiment 2, characterized by an X-ray powder diffraction (XRPD) pattern comprising peaks at 6.1, 11.1, and 16.6 degrees 2θ (±0.2 degrees 2θ).

Embodiment 4. The crystalline Form A of embodiment 2, which is characterized by an X-ray powder diffraction (XRPD) pattern comprising any of the three peaks selected from 6.1, 6.6, 10.0, 11.1, 12.1, 15.6, and 16.6 degrees 2θ (±0.2 degrees 2θ).

Embodiment 5. The crystalline Form A of embodiment 2, which is characterized by an X-ray powder diffraction (XRPD) pattern comprising any of the four peaks selected from 6.1, 6.6, 10.0, 11.1, 12.1, 15.6, and 16.6 degrees 2θ (±n 0.2 degrees 2θ).

Embodiment 6. The crystalline Form A of embodiment 2, which is characterized by an X-ray powder diffraction (XRPD) pattern comprising any of the five peaks selected from 6.1, 6.6, 10.0, 11.1, 12.1, 15.6, and 16.6 degrees 2θ (±0.2 degrees 2θ).

Embodiment 7. The crystalline Form A of embodiment 2, which is characterized by an X-ray powder diffraction pattern comprising peaks at 6.6, 15.6, 22.4, 27.4, and 28.4 degrees 2θ (±0.2 degrees 2θ).

Embodiment 8. The crystalline Form A of any of embodiments 2 to 7, which is characterized by an X-ray powder diffraction pattern comprising peaks at 10.0, 18.5, 20.8, 25.3, and 25.7 degrees 2θ (±0.2 degrees 2θ).

Embodiment 9. The crystalline Form A of embodiment 2, characterized by an X-ray powder diffraction pattern substantially in accordance with FIG. 6.

Embodiment 10. The crystalline Form A of any of embodiments 2 to 9, which is substantially free of other crystalline or amorphous forms of the compound having Formula (I).

Embodiment 11. The crystalline Form A of any of embodiments 2 to 10, further characterized by a differential scanning calorimetry (DSC) thermogram comprising an endothermic peak at about 323.5° C.

Embodiment 12. The crystalline Form A of embodiment 11, wherein the DSC thermogram is substantially in accordance with FIG. 7.

Embodiment 13. The crystalline Form A of any of embodiments 2 to 12, further characterized by a weight percent loss of about 1.0% upon heating to about 220° C., as measured by a thermal gravimetric analysis (TGA).

Embodiment 14. The crystalline Form A of any of embodiments 2 to 13, further characterized by a thermal gravimetric analysis (TGA) thermogram substantially in accordance with FIG. 8.

Embodiment 15. The crystalline Form A of any of embodiments 2 to 14, further characterized by a polarized light microscope (PLM) profile substantially as shown in FIG. 9.

Embodiment 16. Crystalline Form B of a compound having Formula (I):

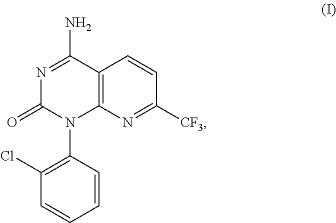 [SMILES: Nc1nc(=O)n(-c2ccccc2Cl)c2nc(C(F)(F)F)ccc12]

characterized by an X-ray powder diffraction (XRPD) pattern comprising peaks at 10.6, 16.6, 18.1, 26.6, and 27.3 degrees 2θ (±0.2 degrees 2θ).

Embodiment 17. The crystalline Form B of embodiment 16, wherein the X-ray powder diffraction pattern further comprises peaks at 11.5, 11.8, 12.0, 19.7, and 28.3 degrees 2θ (±0.2 degrees 2θ).

Embodiment 18. The crystalline Form B of embodiment 16 or 17, wherein the X-ray powder diffraction pattern further comprises peaks at 17.3, 20.3, 22.1, 24.2, and 29.6 degrees 2θ (±0.2 degrees 2θ).

Embodiment 19. The crystalline Form B of embodiment 16, wherein the X-ray powder diffraction pattern is substantially in accordance with FIG. 12.

Embodiment 20. The crystalline Form B of any of embodiments 16 to 19, which is substantially free of other crystalline or amorphous forms of the compound having Formula (I).

Embodiment 21. Crystalline Form C of a compound having Formula (I):

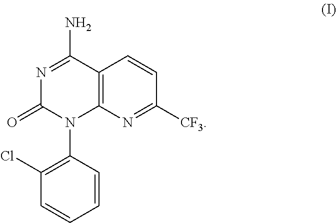 [SMILES: Nc1nc(=O)n(-c2ccccc2Cl)c2nc(C(F)(F)F)ccc12]

Embodiment 22. The crystalline Form C of embodiment 21, characterized by an X-ray powder diffraction (XRPD) pattern comprising peaks at 11.8, 16.6, 17.5, 27.2, and 28.2 degrees 2θ (±0.2 degrees 2θ).

Embodiment 23. The crystalline Form C of embodiment 21, wherein the X-ray powder diffraction pattern further comprises peaks at 19.7, 20.3, 23.7, 24.5, and 29.8 degrees 2θ (±0.2 degrees 2θ).

Embodiment 24. The crystalline Form C of any of embodiments 21 to 23, wherein the X-ray powder diffraction pattern further comprises peaks at 10.9, 17.8, 21.8, 26.0, and 26.6 degrees 2θ (±0.2 degrees 2θ).

Embodiment 25. The crystalline Form C of embodiment 21, wherein the X-ray powder diffraction pattern is substantially in accordance with FIG. 13.

Embodiment 26. The crystalline Form C of any of embodiments 21 to 25, which is substantially free of other crystalline or amorphous forms of the compound having Formula (I).

Embodiment 27. The crystalline Form C of any of embodiments 21 to 26, further characterized by a differential scanning calorimetry (DSC) thermogram comprising one or more endothermic peaks at about 69.5° C., about 197.5° C., and about 326.6° C.

Embodiment 28. The crystalline Form C of embodiment 27, wherein the DSC thermogram is substantially in accordance with FIG. 14.

Embodiment 29. The crystalline Form C of any of embodiments 21 to 28, further characterized by a weight percent loss of about 3.9% upon heating to about 220° C., as measured by a thermal gravimetric analysis (TGA).

Embodiment 30. The crystalline Form C of any of embodiments 21 to 29, further characterized by a thermal gravimetric analysis (TGA) thermogram substantially in accordance with FIG. 15.

Embodiment 31. The crystalline Form C of any of embodiments 21 to 30, further characterized by a polarized light microscope (PLM) profile substantially as shown in FIG. 16.

Embodiment 32. The crystalline Form C of any of embodiments 21 to 31, further characterized by a water content of about 2.6% by weight, as measured by a Karl Fischer (KF) method.

Embodiment 33. The crystalline Form C of any of embodiments 21 to 32, in a hydrate form.

Embodiment 34. Crystalline Form D of a compound having Formula (I):

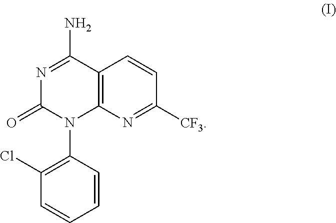 [SMILES: Nc1nc(=O)n(-c2ccccc2Cl)c2nc(C(F)(F)F)ccc12]

Embodiment 35. The crystalline Form D of embodiment 34, characterized by an X-ray powder diffraction (XRPD) pattern comprising peaks at 6.1, 12.4, 13.7, 16.5, 27.6 degrees 2θ (±0.2 degrees 2θ).

Embodiment 36. The crystalline Form D of embodiment 34, wherein the X-ray powder diffraction pattern further comprises peaks at 10.9, 14.8, 25.2, 26.7, and 27.9 degrees 2θ (±0.2 degrees 2θ).

Embodiment 37. The crystalline Form D of any of embodiments 34 to 36, wherein the X-ray powder diffraction pattern further comprises peaks at 10.3, 19.3, 21.2, 24.1, and 29.9 degrees 2θ (±0.2 degrees 2θ).

Embodiment 38. The crystalline Form D of embodiment 34, wherein the X-ray powder diffraction pattern is substantially in accordance with FIG. 19.

Embodiment 39. The crystalline Form D of any of embodiments 34 to 38, which is substantially free of other crystalline or amorphous forms of the compound having Formula (I).

Embodiment 40. The crystalline Form D of any of embodiments 34 to 39, further characterized by a differential scanning calorimetry (DSC) thermogram comprising one or more endothermic peaks at about 66.8° C. and about 322.0° C.

Embodiment 41. The crystalline Form D of embodiment 40, wherein the DSC thermogram is substantially in accordance with FIG. 20.

Embodiment 42. The crystalline Form D of any of embodiments 34 to 41, further characterized by a weight percent loss of about 2.1% upon heating to about 220° C., as measured by a thermal gravimetric analysis (TGA).

Embodiment 43. The crystalline Form D of any of embodiments 34 to 42, further characterized by a thermal gravimetric analysis (TGA) thermogram substantially in accordance with FIG. 21.

Embodiment 44. The crystalline Form D of any of embodiments 34 to 43, further characterized by a polarized light microscope (PLM) profile substantially as shown in FIG. 22.

Embodiment 45. The crystalline Form D of any of embodiments 34 to 44, further characterized by a water content of about 2.3% by weight, as measured by a Karl Fischer (KF) method.

Embodiment 46. The crystalline Form D of any of embodiments 34 to 45, in a hydrate form.

Embodiment 47. Crystalline Form E of a compound having Formula (I):

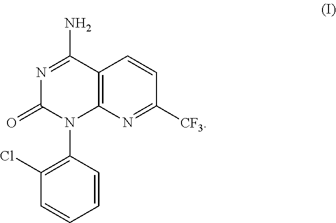 [SMILES: Nc1nc(=O)n(-c2ccccc2Cl)c2nc(C(F)(F)F)ccc12]

Embodiment 48. The crystalline Form E of embodiment 47, characterized by an X-ray powder diffraction (XRPD) pattern comprising peaks at 12.3, 13.7, 19.4, 26.7, and 27.6 degrees 2θ (±0.2 degrees 2θ).

Embodiment 49. The crystalline Form E of embodiment 47, wherein the X-ray powder diffraction pattern further comprises peaks at 20.8, 24.5, 25.3, 28.1, and 30.0 degrees 2θ (±0.2 degrees 2θ).

Embodiment 50. The crystalline Form E of any of embodiments 47 to 49, wherein the X-ray powder diffraction pattern further comprises peaks at 7.2, 14.6, 14.9, 18.2, and 22.5 degrees 2θ (±0.2 degrees 2θ).

Embodiment 51. The crystalline Form E of embodiment 47, wherein the X-ray powder diffraction pattern is substantially in accordance with FIG. 25.

Embodiment 52. The crystalline Form E of any of embodiments 47 to 51, which is substantially free of other crystalline or amorphous forms of the compound having Formula (I).

Embodiment 53. The crystalline Form E of any of embodiments 47 to 52, further characterized by a differential scanning calorimetry (DSC) thermogram comprising one or more endothermic peaks at about 85.9° C. and about 325.0° C.

Embodiment 54. The crystalline Form E of embodiment 53, wherein the DSC thermogram is substantially in accordance with FIG. 26.

Embodiment 55. The crystalline Form E of any of embodiments 47 to 54, further characterized by a weight percent loss of about 4.3% upon heating to about 195° C., as measured by a thermal gravimetric analysis (TGA).

Embodiment 56. The crystalline Form E of any of embodiments 47 to 55, further characterized by a thermal gravimetric analysis (TGA) thermogram substantially in accordance with FIG. 27.

Embodiment 57. The crystalline Form E of any of embodiments 47 to 56, further characterized by a polarized light microscope (PLM) profile substantially as shown in FIG. 28.

Embodiment 58. The crystalline Form E of any of embodiments 47 to 57, further characterized by a water content of about 4.4% by weight, as measured by a Karl Fischer (KF) method.

Embodiment 59. The crystalline Form E of any of embodiments 47 to 58, in a hydrate form.

Embodiment 60. Crystalline Form F of a compound having Formula (I):

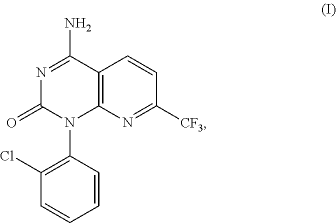 [SMILES: Nc1nc(=O)n(-c2ccccc2Cl)c2nc(C(F)(F)F)ccc12]

characterized by an X-ray powder diffraction (XRPD) pattern comprising peaks at 7.8, 18.2, 23.8, 27.5, and 27.8 degrees 2θ (±0.2 degrees 2θ).

Embodiment 61. The crystalline Form F of embodiment 60, wherein the X-ray powder diffraction pattern further comprises peaks at 14.7, 15.7, 17.8, 24.9, and 25.4 degrees 2θ (±0.2 degrees 2θ).

Embodiment 62. The crystalline Form F of embodiment 60 or 61, wherein the X-ray powder diffraction pattern further comprises peaks at 12.4, 13.1, 17.3, 19.7, and 21.3 degrees 2θ (±0.2 degrees 2θ).

Embodiment 63. The crystalline Form F of embodiment 52, wherein the X-ray powder diffraction pattern is substantially in accordance with FIG. 30.

Embodiment 64. The crystalline Form F of any of embodiments 60 to 63, which is substantially free of other crystalline or amorphous forms of the compound having Formula (I).

Embodiment 65. The crystalline Form F of any of embodiments 60 to 64, further characterized by a differential scanning calorimetry (DSC) thermogram comprising one or more endothermic peaks at about 74.9° C., 212.0° C., and about 325.8° C.

Embodiment 66. The crystalline Form F of embodiment 65, wherein the DSC thermogram is substantially in accordance with FIG. 31.

Embodiment 67. The crystalline Form F of any of embodiments 60 to 66, further characterized by a weight percent loss of about 3.2% upon heating to about 200° C., as measured by a thermal gravimetric analysis (TGA).

Embodiment 68. The crystalline Form F of any of embodiments 60 to 67, further characterized by a thermal gravimetric analysis (TGA) thermogram substantially in accordance with FIG. 32.

Embodiment 69. The crystalline Form F of any of embodiments 60 to 68, further characterized by a water content of about 2.4% by weight, as measured by a Karl Fischer (KF) method.

Embodiment 70. Crystalline Form G of a compound having Formula (I):

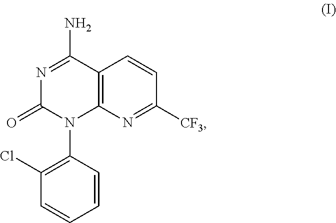 [SMILES: Nc1nc(=O)n(-c2ccccc2Cl)c2nc(C(F)(F)F)ccc12]

characterized by an X-ray powder diffraction (XRPD) pattern comprising peaks at 5.5, 10.0, 15.6, 17.2, and 23.4 degrees 2θ (±0.2 degrees 2θ).

Embodiment 71. The crystalline Form G of embodiment 70, wherein the X-ray powder diffraction pattern further comprises peaks at 7.4, 7.8, 20.9, 24.5, and 27.7 degrees 2θ (0.2 degrees 2θ).

Embodiment 72. The crystalline Form G of embodiment 70, wherein the X-ray powder diffraction pattern is substantially in accordance with FIG. 33.

Embodiment 73. The crystalline Form G of any of embodiments 70 to 72, which is substantially free of other crystalline or amorphous forms of the compound having Formula (I).

Embodiment 74. The crystalline Form G of any of embodiments 70 to 73, further characterized by a differential scanning calorimetry (DSC) thermogram comprising an endothermic peaks at about 322.3° C.

Embodiment 75. The crystalline Form G of embodiment 74, wherein the DSC thermogram is substantially in accordance with FIG. 34.

Embodiment 76. The crystalline Form G of any of embodiments 70 to 75, further characterized by a first weight percent loss of 2.5% upon heating to about 160° C. and a second weight percent loss of about 0.9% upon heating to about 233° C., as measured by a thermal gravimetric analysis (TGA).

Embodiment 77. The crystalline Form G of any of embodiments 70 to 76, further characterized by a thermal gravimetric analysis (TGA) thermogram substantially in accordance with FIG. 35.

Embodiment 78. Crystalline Form H of a compound having Formula (I):

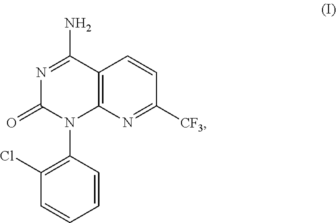 [SMILES: Nc1nc(=O)n(-c2ccccc2Cl)c2nc(C(F)(F)F)ccc12]

characterized by an X-ray powder diffraction (XRPD) pattern comprising peaks at 5.1, 5.8, 8.8, 15.6, and 23.4 degrees 2θ (±0.2 degrees 2θ).

Embodiment 79. The crystalline Form H of embodiment 78, wherein the X-ray powder diffraction pattern further comprises peaks at 7.8, 11.9, 17.3, and 28.3 degrees 2θ (0.2 degrees 2θ).

Embodiment 80. The crystalline Form H of embodiment 78, wherein the X-ray powder diffraction pattern is substantially in accordance with FIG. 37.

Embodiment 81. The crystalline Form H of any of embodiments 78 to 80, which is substantially free of other crystalline or amorphous forms of the compound having Formula (I).

Embodiment 82. The crystalline Form H of any of embodiments 78 to 81, further characterized by a differential scanning calorimetry (DSC) thermogram comprising one or more endothermic peaks at about 226.3° C. and about 322.6° C.

Embodiment 83. The crystalline Form H of embodiment 82, wherein the DSC thermogram is substantially in accordance with FIG. 38.

Embodiment 84. The crystalline Form H of any of embodiments 78 to 83, further characterized by a weight percent loss of about 8.7% upon heating to about 238° C., as measured by a thermal gravimetric analysis (TGA).

Embodiment 85. The crystalline Form H of any of embodiments 78 to 84, further characterized by a thermal gravimetric analysis (TGA) thermogram substantially in accordance with FIG. 39.

Embodiment 86. Crystalline Form I of a compound having Formula (I):

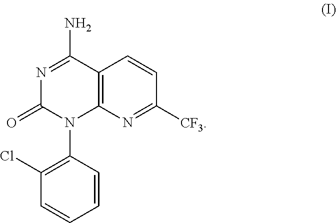 [SMILES: Nc1nc(=O)n(-c2ccccc2Cl)c2nc(C(F)(F)F)ccc12]

Embodiment 87. The crystalline Form I of embodiment 86, characterized by an X-ray powder diffraction (XRPD) pattern comprising peaks at 5.8, 6.4, 16.1, 16.5, and 27.7 degrees 2θ (±0.2 degrees 2θ).

Embodiment 88. The crystalline Form I of embodiment 86, wherein the X-ray powder diffraction pattern further comprises peaks at 10.0, 10.8, 13.1, 25.5, and 29.0 degrees 2θ (±0.2 degrees 2θ).

Embodiment 89. The crystalline Form I of embodiment 86 or 88, wherein the X-ray powder diffraction pattern further comprises peaks at 8.4, 11.9, 17.7, 19.6, and 23.3 degrees 2θ (±0.2 degrees 2θ).

Embodiment 90. The crystalline Form I of embodiment 86, wherein the X-ray powder diffraction pattern is substantially in accordance with FIG. 41.

Embodiment 91. The crystalline Form I of any of embodiments 86 to 90, which is substantially free of other crystalline or amorphous forms of the compound having Formula (I).

Embodiment 92. The crystalline Form I of any of embodiments 86 to 91, further characterized by a differential scanning calorimetry (DSC) thermogram comprising one or more endothermic peaks at about 267.05° C. and about 323.7° C.

Embodiment 93. The crystalline Form I of embodiment 92, wherein the DSC thermogram is substantially in accordance with FIG. 42.

Embodiment 94. The crystalline Form I of any of embodiments 86 to 93, further characterized by a weight percent loss of about 1.1% upon heating to about 220° C., as measured by a thermal gravimetric analysis (TGA).

Embodiment 95. The crystalline Form I of any of embodiments 86 to 94, further characterized by a thermal gravimetric analysis (TGA) thermogram substantially in accordance with FIG. 43.

Embodiment 96. The crystalline Form I of any of embodiments 86 to 95, further characterized by a polarized light microscope (PLM) profile substantially as shown in FIG. 44.

Embodiment 97. The crystalline Form I of any of embodiments 86 to 96, further characterized by a water content of about 1.4% by weight, as measured by a Karl Fischer (KF) method.

Embodiment 98. Crystalline Form J of a compound having Formula (I):

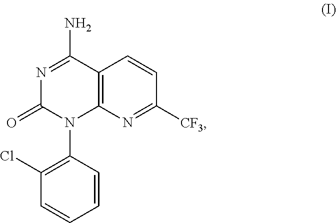 [SMILES: Nc1nc(=O)n(-c2ccccc2Cl)c2nc(C(F)(F)F)ccc12]

characterized by an X-ray powder diffraction (XRPD) pattern comprising peaks at 7.7, 12.9, 14.6, 26.9, and 27.6 degrees 2θ (±0.2 degrees 2θ).

Embodiment 99. The crystalline Form J of embodiment 98, wherein the X-ray powder diffraction pattern further comprises peaks at 18.1, 22.2, 23.2, 25.3, and 27.4 degrees 2θ (±0.2 degrees 2θ).

Embodiment 100. The crystalline Form J of embodiment 98 or 99, wherein the X-ray powder diffraction pattern further comprises peaks at 17.4, 21.3, 23.8, 26.0, and 26.8 degrees 2θ (±0.2 degrees 2θ).

Embodiment 101. The crystalline Form J of embodiment 98, wherein the X-ray powder diffraction pattern is substantially in accordance with FIG. 46.

Embodiment 102. The crystalline Form J of any of embodiments 98 to 101, which is substantially free of other crystalline or amorphous forms of the compound having Formula (I).

Embodiment 103. The crystalline Form J of any of embodiments 98 to 102, further characterized by a differential scanning calorimetry (DSC) thermogram comprising one or more endothermic peaks at about 131.9° C., 212.0° C., and about 324.0° C.

Embodiment 104. The crystalline Form J of embodiment 103, wherein the DSC thermogram is substantially in accordance with FIG. 47.

Embodiment 105. The crystalline Form J of any of embodiments 98 to 104, further characterized by a weight percent loss of about 9.9% upon heating to about 200° C., as measured by a thermal gravimetric analysis (TGA).

Embodiment 106. The crystalline Form J of any of embodiments 98 to 105, further characterized by a thermal gravimetric analysis (TGA) thermogram substantially in accordance with FIG. 48.

Embodiment 107. The crystalline Form J of any of embodiments 98 to 106, in an isopropanol solvate form.

Embodiment 108. Crystalline Form K of a compound having Formula (I):

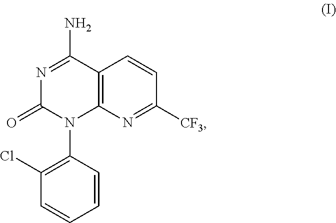 [SMILES: Nc1nc(=O)n(-c2ccccc2Cl)c2nc(C(F)(F)F)ccc12]

characterized by an X-ray powder diffraction (XRPD) pattern comprising peaks at 5.5, 11.1, 16.5, 27.1, and 27.7 degrees 2θ (±0.2 degrees 2θ).

Embodiment 109. The crystalline Form K of embodiment 108, wherein the X-ray powder diffraction pattern further comprises peaks at 13.8, 17.5, 22.2, 25.2, and 28.9 degrees 2θ (±0.2 degrees 2θ).

Embodiment 110. The crystalline Form K of embodiment 108, wherein the X-ray powder diffraction pattern is substantially in accordance with FIG. 50.

Embodiment 111. The crystalline Form K of any of embodiments 108 to 110, which is substantially free of other crystalline or amorphous forms of the compound having Formula (I).

Embodiment 112. Crystalline Form L of a compound having Formula (I):

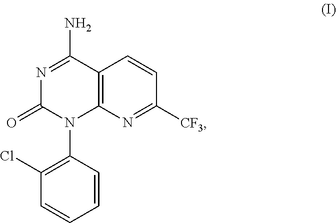 [SMILES: Nc1nc(=O)n(-c2ccccc2Cl)c2nc(C(F)(F)F)ccc12]

characterized by an X-ray powder diffraction (XRPD) pattern comprising peaks at 11.7, 17.9, 18.4, 26.3, and 27.0 degrees 2θ (±0.2 degrees 2θ).

Embodiment 113. The crystalline Form L of embodiment 112, wherein the X-ray powder diffraction pattern further comprises peaks at 10.8, 12.3, 16.3, 16.6, and 22.2 degrees 2θ (±0.2 degrees 2θ).

Embodiment 114. The crystalline Form L of embodiment 112 or 113, wherein the X-ray powder diffraction pattern further comprises peaks at 17.0, 19.7, 20.3, 23.3, and 28.1 degrees 2θ (±0.2 degrees 2θ).

Embodiment 115. The crystalline Form L of embodiment 112, wherein the X-ray powder diffraction pattern is substantially in accordance with FIG. 51.

Embodiment 116. The crystalline Form L of any of embodiments 112 to 115, which is substantially free of other crystalline or amorphous forms of the compound having Formula (I).

Embodiment 117. The crystalline Form L of any of embodiments 112 to 116, further characterized by a differential scanning calorimetry (DSC) thermogram comprising one or more endothermic peaks at about 202.5° C. and about 326.6.0° C.

Embodiment 118. The crystalline Form L of embodiment 117, wherein the DSC thermogram is substantially in accordance with FIG. 52.

Embodiment 119. The crystalline Form L of any of embodiments 112 to 118, further characterized by a weight percent loss of about 2.0% upon heating to about 220° C., as measured by a thermal gravimetric analysis (TGA).

Embodiment 120. The crystalline Form L of any of embodiments 112 to 119, further characterized by a thermal gravimetric analysis (TGA) thermogram substantially in accordance with FIG. 53.

Embodiment 121. Crystalline Form M of a compound having Formula (I):

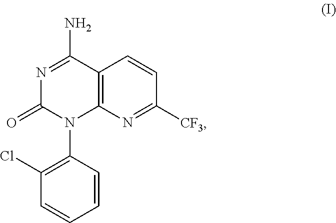 [SMILES: Nc1nc(=O)n(-c2ccccc2Cl)c2nc(C(F)(F)F)ccc12]

characterized by an X-ray powder diffraction (XRPD) pattern comprising peaks at 6.2, 6.7, 10.5, 12.4, and 13.4 degrees 2θ (±0.2 degrees 2θ).

Embodiment 122. The crystalline Form M of embodiment 121, wherein the X-ray powder diffraction pattern further comprises peaks at 16.1, 17.6, 24.2, 27.1, and 28.3 degrees 2θ (±0.2 degrees 2θ).

Embodiment 123. The crystalline Form M of embodiment 121 or 122, wherein the X-ray powder diffraction pattern further comprises peaks at 17.0, 20.2, 22.7, 23.1, and 26.5 degrees 2θ (±0.2 degrees 2θ).

Embodiment 124. The crystalline Form M of embodiment 121, wherein the X-ray powder diffraction pattern is substantially in accordance with FIG. 54.

Embodiment 125. The crystalline Form M of any of embodiments 121 to 124, which is substantially free of other crystalline or amorphous forms of the compound having Formula (I).

Embodiment 126. Crystalline Form N of a compound having Formula (I):

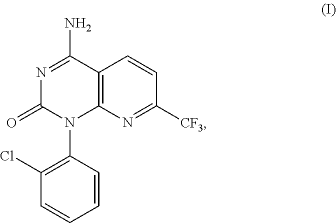 [SMILES: Nc1nc(=O)n(-c2ccccc2Cl)c2nc(C(F)(F)F)ccc12]

characterized by an X-ray powder diffraction (XRPD) pattern comprising peaks at 5.4, 10.7, 16.1, 27.0, and 28.0 degrees 2θ (±0.2 degrees 2θ).

Embodiment 127. The crystalline Form N of embodiment 126, wherein the X-ray powder diffraction pattern is substantially in accordance with FIG. 55.

Embodiment 128. The crystalline Form N of any of embodiments 126 to 127, which is substantially free of other crystalline or amorphous forms of the compound having Formula (I).

Embodiment 129. Crystalline Form O of a compound having Formula (I):

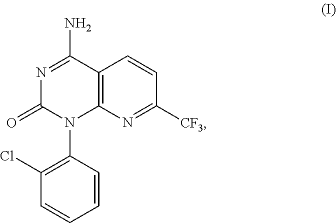 [SMILES: Nc1nc(=O)n(-c2ccccc2Cl)c2nc(C(F)(F)F)ccc12]

characterized by an X-ray powder diffraction (XRPD) pattern comprising peaks at 5.0, 5.9, 13.4, 17.8, and 25.7 degrees 2θ (±0.2 degrees 2θ).

Embodiment 130. The crystalline Form O of embodiment 129, wherein the X-ray powder diffraction pattern further comprises peaks at 12.8, 15.3, 26.9, 27.9, and 29.0 degrees 2θ (±0.2 degrees 2θ).

Embodiment 131. The crystalline Form O of embodiment 129, wherein the X-ray powder diffraction pattern is substantially in accordance with FIG. 56.

Embodiment 132. The crystalline Form O of any of embodiments 129 to 131, which is substantially free of other crystalline or amorphous forms of the compound having Formula (I).

Embodiment 133. Crystalline Form P of a compound having Formula (I):

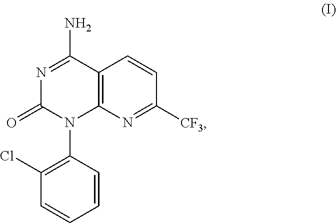 [SMILES: Nc1nc(=O)n(-c2ccccc2Cl)c2nc(C(F)(F)F)ccc12]

characterized by an X-ray powder diffraction (XRPD) pattern comprising peaks at 5.6, 6.0, 17.1, 26.6, and 27.9 degrees 2θ (±0.2 degrees 2θ).

Embodiment 134. The crystalline Form P of embodiment 133, wherein the X-ray powder diffraction pattern further comprises peaks at 10.8, 12.0, 14.3, 14.8, and 18.1 degrees 2θ (±0.2 degrees 2θ).

Embodiment 135. The crystalline Form P of embodiment 133, wherein the X-ray powder diffraction pattern is substantially in accordance with FIG. 57.

Embodiment 136. The crystalline Form P of any of embodiments 133 to 135, which is substantially free of other crystalline or amorphous forms of the compound having Formula (I).

Embodiment 137. Crystalline Form Q of a compound having Formula (I):

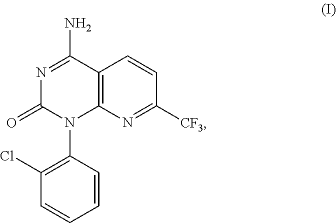 [SMILES: Nc1nc(=O)n(-c2ccccc2Cl)c2nc(C(F)(F)F)ccc12]

characterized by an X-ray powder diffraction (XRPD) pattern comprising peaks at 3.3, 5.4, 16.1, 27.0, and 31.7 degrees 2θ (±0.2 degrees 2θ).

Embodiment 138. The crystalline Form Q of embodiment 137, wherein the X-ray powder diffraction pattern further comprises peaks at 4.0, 10.7, 25.0, 27.9, 28.0 degrees 2θ (0.2 degrees 2θ).

Embodiment 139. The crystalline Form Q of embodiment 137, wherein the X-ray powder diffraction pattern is substantially in accordance with FIG. 58.

Embodiment 140. The crystalline Form Q of any of embodiments 137 to 139, which is substantially free of other crystalline or amorphous forms of the compound having Formula (I).

Embodiment 141. Crystalline Form R of a compound having Formula (I):

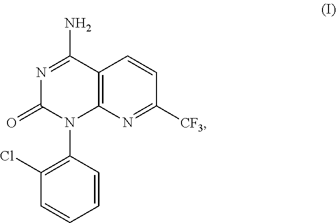 [SMILES: Nc1nc(=O)n(-c2ccccc2Cl)c2nc(C(F)(F)F)ccc12]

characterized by an X-ray powder diffraction (XRPD) pattern comprising three, four, five, or more peaks at 3.2, 3.5, 3.9, 6.9, 10.7, 12.4, 13.9, 16.8, 17.4, 18.5, 18.9, 20.4, 21.6, 22.4, 22.9, 23.4, 24.5, 27.4, 27.9, and 28.4 degrees 2θ (±0.2 degrees 2θ).

Embodiment 142. The crystalline Form R of embodiment 141, wherein the X-ray powder diffraction pattern is substantially in accordance with FIG. 59.

Embodiment 143. The crystalline Form R of embodiment 141 or 142, which is substantially free of other crystalline or amorphous forms of the compound having Formula (I).

Embodiment 144. Crystalline Form S of a compound having Formula (I):

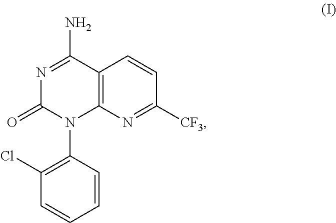 [SMILES: Nc1nc(=O)n(-c2ccccc2Cl)c2nc(C(F)(F)F)ccc12]

characterized by an X-ray powder diffraction (XRPD) pattern comprising peaks at 6.1, 10.4, 10.8, 13.5, and 16.5 degrees 2θ (±0.2 degrees 2θ).

Embodiment 145. The crystalline Form S of embodiment 144, wherein the X-ray powder diffraction pattern further comprises peaks at 12.3, 12.4, 14.0, 17.4, and 28.6 degrees 2θ (±0.2 degrees 2θ).

Embodiment 146. The crystalline Form S of 144, wherein the X-ray powder diffraction pattern is substantially in accordance with FIG. 60.

Embodiment 147. The crystalline Form S of any of embodiments 144 to 146, which is substantially free of other crystalline or amorphous forms of the compound having Formula (I).

Embodiment 148. Crystalline Form T of a compound having Formula (I):

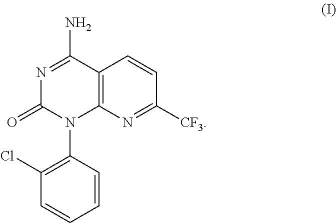 [SMILES: Nc1nc(=O)n(-c2ccccc2Cl)c2nc(C(F)(F)F)ccc12]

Embodiment 149. The crystalline Form T of embodiment 148, characterized by an X-ray powder diffraction (XRPD) pattern comprising peaks at 6.5, 10.9, 12.4, 13.0, and 16.9 degrees 2θ (±0.2 degrees 2θ).

Embodiment 150. The crystalline Form T of embodiment 148, wherein the X-ray powder diffraction pattern further comprises peaks at 10.5, 16.6, 22.2, 27.5, and 27.9 degrees 2θ (±0.2 degrees 2θ).

Embodiment 151. The crystalline Form T of embodiment 148, wherein the X-ray powder diffraction pattern is substantially in accordance with FIG. 61.

Embodiment 152. The crystalline Form T of any of embodiments 148 to 151, which is substantially free of other crystalline or amorphous forms of the compound having Formula (I).

Embodiment 153. The crystalline Form T of any of embodiments 148 to 152, further characterized by a differential scanning calorimetry (DSC) thermogram comprising an endothermic peaks at about 319.4° C.

Embodiment 154. The crystalline Form T of embodiment 153, wherein the DSC thermogram is substantially in accordance with FIG. 62.

Embodiment 155. The crystalline Form T of any of embodiments 148 to 154, further characterized by a weight percent loss of about 0.8% upon heating to about 220° C., as measured by a thermal gravimetric analysis (TGA).

Embodiment 156. The crystalline Form T of any of embodiments 148 to 155, further characterized by a thermal gravimetric analysis (TGA) thermogram substantially in accordance with FIG. 63.

Embodiment 157. The crystalline Form T of any of embodiments 148 to 156, further characterized by a polarized light microscope (PLM) profile substantially as shown in FIG. 64.

Embodiment 158. The crystalline Form T of any of embodiments 148 to 157, further characterized by a water content of about 1.7% by weight, as measured by a Karl Fischer (KF) method.

Embodiment 159. Crystalline Form U of a compound having Formula (I):

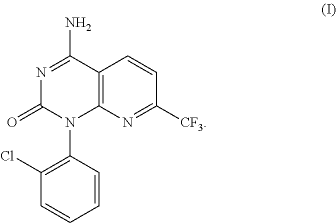 [SMILES: Nc1nc(=O)n(-c2ccccc2Cl)c2nc(C(F)(F)F)ccc12]

characterized by an X-ray powder diffraction (XRPD) pattern comprising peaks at 6.2, 10.3, 10.8, 12.4, and 16.4 degrees 2θ (±0.2 degrees 2θ).

Embodiment 160. The crystalline Form U of embodiment 159, wherein the X-ray powder diffraction pattern further comprises peaks at 17.6, 21.3, 22.4, 27.2, and 28.5 degrees 2θ (±0.2 degrees 2θ).

Embodiment 161. The crystalline Form U of embodiment 159, wherein the X-ray powder diffraction pattern is substantially in accordance with FIG. 67.

Embodiment 162. The crystalline Form U of any of embodiments 159 to 161, which is substantially free of other crystalline or amorphous forms of the compound having Formula (I).

Embodiment 163. A solid state form of a compound having Formula (I),

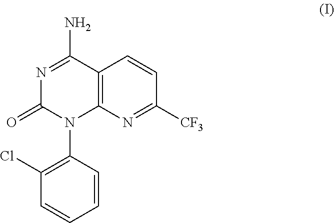 [SMILES: Nc1nc(=O)n(-c2ccccc2Cl)c2nc(C(F)(F)F)ccc12]

or a pharmaceutically acceptable salt thereof.

Embodiment 164. The solid state form of embodiment 163, wherein the solid state form is substantially crystalline.

Embodiment 165. The solid state form of embodiment 163 or 164, wherein the solid state form is of Formula (I) as a free base.

Embodiment 166. The solid state form of embodiment 163 or 164, wherein the solid state form is of Formula (I) as a pharmaceutically acceptable salt thereof.

Embodiment 167. The solid state form of any of embodiments 163 to 166, which is characterized by an X-ray powder diffraction (XRPD) pattern comprising peaks at 6.2, 10.9, and 16.5 degrees 2θ (±0.2 degrees 2θ.

Embodiment 168. The solid form of any of embodiments 163 to 166, which is characterized by an X-ray powder diffraction (XRPD) pattern substantially in accordance with FIG. 1.

Embodiment 169. The solid state form of any of embodiments 163 to 166, wherein the solid state form comprises at least 30 wt. % of a particular crystalline form, at least 40 wt. % of a particular crystalline form, at least 50 wt. % of a particular crystalline form, at least 60 wt. % of a particular crystalline form, at least 70 wt. % of a particular crystalline form, at least 80 wt. % of a particular crystalline form, at least 90 wt. % of a particular crystalline form, at least 95 wt. % of a particular crystalline form, or at least 99 wt. % of a particular crystalline form.

Embodiment 170. The solid state form of any of embodiments 163 to 166, wherein the solid state form comprises at least 50 wt. % of a particular crystalline form, at least 60 wt. % of a particular crystalline form, at least 70 wt. % of a particular crystalline form, at least 80 wt. % of a particular crystalline form, at least 90 wt. % of a particular crystalline form, at least 95 wt. % of a particular crystalline form, or at least 99 wt. % of a particular crystalline form.

Embodiment 171. The solid state form of above two embodiments, wherein the particular crystalline form is according to any of embodiments 1 to 162.

Embodiment 172. The solid state form of above two embodiments, wherein the particular crystalline form is crystalline Form A.

Embodiment 173. A pharmaceutical composition comprising a crystalline form of any of embodiments 2 to 162, and at least one pharmaceutically acceptable excipient.

Embodiment 174. A pharmaceutical composition comprising the solid state form of any of embodiments 163 to 172 and at least one pharmaceutically acceptable excipient.

Embodiment 175. The pharmaceutical composition of embodiment 174, wherein the particular crystalline form is crystalline Form A.

Embodiment 176. A pharmaceutical composition comprising crystalline Form A.

Embodiment 177. A pharmaceutical composition comprising crystalline Form A and at least one pharmaceutically acceptable excipient.

Embodiment 178. A pharmaceutical composition comprising a crystalline form of any of embodiments 2 to 162; and one or more pharmaceutically acceptable or physiologically acceptable excipients.

Embodiment 179. A method for treating a disease treatable by inhibition of MAT2A in a patient comprising administering to the patient a therapeutically effective amount of a crystalline form of any of embodiments 2 to 162, the solid state form of any of embodiments 163 to 172, or a pharmaceutical composition of any of embodiments 173-178.

Embodiment 180. The method of embodiment 179, wherein the disease is cancer.

Embodiment 181. A method of treating a MTAP null cancer in a patient comprising administering to the patient a therapeutically effective amount of a crystalline form of any of embodiments 2 to 162, the solid state form of any of embodiments 163 to 172, or a pharmaceutical composition of any of embodiments 173-178.

Embodiment 182. A method for treating a cancer in a patient, wherein the cancer is characterized by a reduction or absence of MTAP gene expression, the absence of the MTAP gene, reduced level of MTAP protein, reduced function of MTAP protein, absent of MTAP protein, or a combination thereof, comprising administering to the subject a therapeutically effective amount of a crystalline form of any of embodiments 2 to 162, the solid state form of any of embodiments 163 to 172, or a pharmaceutical composition of any of embodiments 173-178.

Embodiment 183. The method of any of embodiments 180 to 182, wherein the cancer is selected from the group consisting of cancer is leukemia, glioma, melanoma, pancreatic, non-small cell lung cancer (NSCLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophagogastric cancer, gastrointestinal (GI) cancer, or mesothelioma.

Embodiment 184. A method of making a pharmaceutical composition comprising mixing crystalline Form A with at least one pharmaceutically acceptable excipient.

Embodiment 185. A method of making a pharmaceutical composition comprising mixing the solid state form of any of embodiments 163 to 172 with at least one pharmaceutically acceptable excipient.

Embodiment 186. A pharmaceutical composition prepared by combining crystalline Form A with at least one pharmaceutically acceptable excipient.

Embodiment 187. A pharmaceutical composition prepared by combining the solid state form of any of embodiments 163 to 172 with at least one pharmaceutically acceptable excipient.

Embodiment 188. A method for preparing crystalline Form A of a compound having Formula (I):

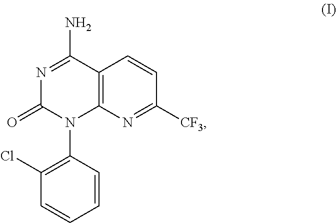 [SMILES: Nc1nc(=O)n(-c2ccccc2Cl)c2nc(C(F)(F)F)ccc12]

comprising:
a) forming a first mixture comprising a crude compound of Formula (I), ACN, and water;
b) solvent exchanging with water at a temperature of no more than about 65° C. to form a second mixture;
c) cooling the second mixture and stirring to form a third precipitate; d) isolating the third precipitate;
e) forming a slurry comprising the third precipitate, methyl ethyl ketone (MEK), and water;
f) isolating a fourth precipitate from step e);
g) drying the fourth precipitate to provide the crystalline Form A of Formula (I).

Embodiment 189. The method of embodiment 188, wherein step a) is conducted at a temperature of from about 75° C. to 80° C.

Embodiment 190. The method of embodiment 188 or 190, wherein, in step a), a ratio of ACN to water is about 4:1 by volume.

Embodiment 191. The method of any of embodiments 188 to 190, wherein step c) is conducted at a temperature of from about 0° C. to 5° C. and stirred for a period of from about 12 to 16 hours.

Embodiment 192. The method of any of embodiments 188 to 191, wherein, in step e), a ratio of MEK to water is about 10:1 by volume.

Embodiment 193. The method of any of embodiments 188 to 192, wherein step e) is conducted at a temperature of from about 60° C. to 65° C. and stirred for a period of from about 17 to 22 hours; and further cooled to a temperature of from about 0° C. to 5° C. and stirred for a period of from about 15 to 24 hours.

Embodiment 194. The method of any of embodiments 188 to 193, wherein the isolating of step d) and/or step e) is conducted by filtration.

Embodiment 195. The method of any of embodiments 188 to 193, wherein the drying of step g) is conducted at a temperature of from 65° C. to 70° C.

Embodiment 196. The method of any of embodiments 188 to 195, wherein the crude compound of Formula (I) is present in the first mixture in an amount of from about 20 g/L to 100 g/L.

Embodiment 197. A method for preparing a crystalline Form of a compound having Formula (I):

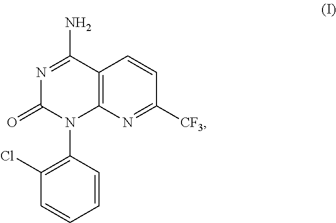 [SMILES: Nc1nc(=O)n(-c2ccccc2Cl)c2nc(C(F)(F)F)ccc12]

comprising:
a) forming a slurry comprising solid state Form V of Formula (I) and a solvent;
b) stirring the slurry for a period of at least a day; and
c) isolating a precipitate; and
d) drying the precipitate to provide the crystalline Form of Formula (I),
   wherein the crystalline Form is crystalline Form C, D, E, I, or A-1; and
the solvent is methanol, water, a mixture of methanol and water, a mixture of acetone and water, or a mixture of acetonitrile and water.

Embodiment 198. The method of embodiment 197, wherein the crystalline Form is crystalline Form C; and the solvent is methanol.

Embodiment 199. The method of embodiment 197, wherein the crystalline Form is crystalline Form D; and the solvent is water.

Embodiment 200. The method of embodiment 197, wherein the crystalline Form is crystalline Form E; and the solvent is a mixture of methanol and water.

Embodiment 201. The method of embodiment 200, wherein a ratio of methanol to water is about 1:1 by volume.

Embodiment 202. The method of embodiment 197, wherein the crystalline Form is crystalline Form I; and the solvent is a mixture of acetone and water.

Embodiment 203. The method of embodiment 202, wherein a ratio of acetone to water is about 1:1 by volume.

Embodiment 204. The method of embodiment 197, wherein the crystalline Form is crystalline Form A; and the solvent is a mixture of acetonitrile and water.

Embodiment 205. The method of embodiment 204, wherein a ratio of acetonitrile to water is about 1:1 by volume.

Embodiment 206. The method of any of embodiments 197 to 205, wherein the stirring of step b) is conducted at a temperature of about 25° C. and/or about 50° C.

Embodiment 207. The method of any of embodiments 197 to 206, wherein the stirring of step b) is conducted for a period of from 3 to 6 days.

Embodiment 208. The method of any of embodiments 197 to 207, wherein the stirring of step b) is conducted at a first temperature of about 50° C. for a first period of 3 days and at a second temperature of about 25° C. for a second period of 3 days.

Embodiment 209. The method of any of embodiments 197 to 207, wherein the stirring of step b) is conducted at a temperature of about 50° C. for a period of 6 days.

Embodiment 210. The method of any of embodiments 197 to 207, wherein the stirring of step b) is conducted at a temperature of about 50° C. for a period of 3 days.

Embodiment 211. The method of any of embodiments 197 to 207, wherein the stirring of step b) is conducted at a temperature of about 25° C. for a period of 6 days.

Embodiment 212. The method of any of embodiments 197 to 211, wherein the isolating of step c) is conducted by filtration.

Embodiment 213. The method of any of embodiments 197 to 212, wherein the drying of step d) is conducted at room temperature.

Embodiment 214. The method of any of embodiments 197 to 213, wherein the crystalline solid state Form V of Formula (I) is present in the slurry in an amount of from 50 mg/mL to 120 mg/mL.

### IX. Examples

The following examples are provided to illustrate, but not limit the current description.

Standard abbreviations are used, including the following: d=doublet; dd=doublet of doublets; DIPEA=N,N-diisopropylethylamine; DMSO=dimethylsulfoxide; EDTA=ethylenediaminetetraacetic acid; EtOH=ethanol; g=gram; mg=milligram; μg=microgram; ng=nanogram; μM=micromolar; mM=millimolar; nM=nanomolar; h or hr=hour(s); min=minute(s); kDa=kilodalton; kg=kilogram; l or L=liter; ml or mL=milliliter; μl or μL=microliter; LC=liquid chromatography; LCMS=liquid chromatography and mass spectrometry; m/z=mass to charge ratio; m=multiplet; MeCN/ACN=acetonitrile; MS=mass spectrometry; N=normal; NMR=nuclear magnetic resonance; PE=petroleum ether; rac=racemic; Rt=retention time; sat.=saturated; t=triplet; THF=tetrahydrofuran; TLC=thin layer chromatography; TsOH=p-toluenesulfonic acid; TsOH=p-toluenesulfonic acid; MsOH=methanesulfonic acid; TfOH=triflic acid; (COCl)₂=oxalyl chloride; t-BuOH=tert-butyl alcohol; MTBE=methyl tert-butyl ether.

### Instrumental Methods

X-ray Powder Diffractometer (XRPD)
XRPD method 1 (screening, scale up and evaluation of polymorphs except variable
temperature and humidity XRPD study)

| | |
|---|---|
| Instrument | Bruker D8 Advance |
| Detector | LYNXEYE_XE_T(1D mode) |
| Open angle | 2.94° |
| Scan mode | Continuous PSD fast |
| Radiation | Cu/K-Alpha1 (λ = 1.5418 \acute{Å}) |
| X-ray generator power | 40 kV, 40 mA |
| Step size | 0.02° |
| Time per step | 0.12 second per step |
| Scan range | 3° to 40° |
| Primary beam path slits | Twin_Primary motorized slit 10.0 mm by sample length; |
| | SollerMount axial soller 2.5° |
| Secondary beam path | Detector OpticsMount soller slit 2.5°; Twin_Secondary motorized |
| slits | slit 5.2 mm |
| Sample rotation speed | 15 rpm |

XRPD method 2 (variable temperature and humidity XRPD study)

| | |
|---|---|
| Instrument | Bruker D8 Advance |
| Detector | PSD LynxEye (1D mode) |
| Open angle | 2.948° |
| Scan mode | Continuous scan |
| Radiation | Cu/K-Alpha1 (λ = 1.54060 \acute{Å}) |
| X-ray generator power | 40 kV, 40 mA |
| Step size | 0.02° |
| Time per step | 0.6 second per step |
| Scan range | 4° to 40° |
| slits | Fixed incident beam optics: primary Soller slit 2.5 deg; divergence |
| | slit 0.6 mm; secondary Soller slit 2.5 deg; detesctor slit 10.50 mm; |
| | antiscattering slit 7.100 mm |
| Sample rotation speed | 0 rpm |

XRPD method 3 (solubility testing of Form T and almost amorphous form in FaSSIF
and FeSSIF; Form T after DVS)

| | |
|---|---|
| Instrument | Bruker D8 Advance |
| Detector | PSD LynxEye (1D mode) |
| Open angle | 2.94° |
| Scan mode | Continuous PSD fast |
| Radiation | Cu/K-Alpha1 (λ = 1.5418 \acute{Å}) |
| X-ray generator power | 40 kV, 40 mA |
| Step size | 0.02° |
| Time per step | 0.12 second per step |
| Scan range | 3° to 40° |
| Primary beam path slits | Twin_Primary motorized slit 10.0 mm by sample length; |
| | SollerMount axial soller 2.5° |
| Secondary beam path | Detector OpticsMount soller slit 2.5°; Twin_Secondary motorized |
| slits | slit 7.1 mm |
| Sample rotation speed | 15 rpm |

Differential Scanning Calorimetric (DSC)

| | |
|---|---|
| Instrument | TA Discovery 2500 |
| Sample pan | Tzero pan and Tzero hermetic lid with a pin hole |
| Temperature range | 30 to 350° C. or before decomposition |
| Heating rate | 10° C./min |
| Nitrogen flow | 50 mL/min |
| Sample mass | ~1-2 mg |

Thermal Gravimetric Analysis (TGA)

| | |
|---|---|
| Instrument | Discovery 5500 |
| Sample pan | Aluminum, open |
| Nitrogen flow | Balance 10 mL/min; sample 25 mL/min |
| Start temperature | Ambient condition (below 35° C.) |
| Final temperature | 300° C. or abort next segment if weight <80% (w/w) |
| | (The weight percent loss of the compound is no more than 20% |
| | (w/w)) |
| Heating rate | 10° C./min |
| Sample mass | ~2-10 mg |

Karl Fischer

| | |
|---|---|
| Instrument | Mettler Toledo Coulometric KF Titrator C30 |
| Method | Coulometric |

Polarized Light Microscope (PLM)

| | |
|---|---|
| Instrument | OLYMPUS |
| Method | Crossed polarizer, silicone oil added |
| temperature | 25° C. |
| dm/dt | 0.002%/min |

Nuclear Magnetic Resonance (NMR)

| | |
|---|---|
| Instrument | Bruker Avance-AV 400M |
| Frequency | 400 MHz |
| Probe | 5 mm PABBO BB-1H/D |
| Number of scan | 8 |
| Temperature | 297.6 K |
| Relaxation delay | 1 second |

Dynamic Vapor Sorption (DVS)

| | |
|---|---|
| Instrument | Advantage and Intrinsic |
| Total gas flow | 200 sccm |
| Oven temperature | 25° C. |
| Solvent | Water |
| Method | Cycle: 40-0-95-0-40% RH |
| | Stage Step: 10% |
| | Equilibrium: 0.002 dm/dt (%/min) |
| | Minimum dm/dt stability duration: 60 min |
| | Maximum dm/dt stage time: 360 min |

High Performance Liquid Chromatograph (HPLC)
HPLC method 1

| Instrument | Shimadzu LC-20ADXR | | |
|---|---|---|---|
| HPLC method | Wave length: 220 nm | | |
| | Column: Zorbax SB-C 18, 150 × 4.6 mm, 5 μm | | |
| | Detector: UV 220 nm | | |
| | Column temperature: 40° C. | | |
| | Flow rate: 1.2 mL/min | | |
| | Mobile phase A: 0.1% TFA in Water | | |
| | Mobile phase B: Acetonitrile | | |
| | Diluent: Acetonitrile or Acetonitrile/water (80:20/V/V) | | |
| | Injection volume: 5 uL | | |
| | Gradient: | | |
| | Time (min) | Mobile Phase A (%) | Mobile Phase B (%) |
| | 0.0 | 95 | 5 |
| | 0.01 | 95 | 4 |
| | 9.0 | 5 | 95 |
| | 13.0 | 5 | 95 |
| | 13.1 | 95 | 5 |
| | 17.0 | 95 | 5 |

HPLC method 2

| Instrument | Shimadzu LC-20ADXR | | |
|---|---|---|---|
| HPLC method | Wave length: 230 nm | | |
| | Column: Waters Xbridge phenyl (150 mm*4.6 mm, 3.5 μm | | |
| | PN: 186003335) | | |
| | Detector: UV 220 nm | | |
| | Column temperature: 30° C. | | |
| | Flow rate: 1.0 mL/min | | |
| | Mobile phase A: 0.05% TFA in Water | | |
| | Mobile phase B: 0.05% TFA in Acetonitrile | | |
| | Diluent: Acetonitrile or Acetonitrile/water (80:20/V/V) | | |
| | Injection volume: 5 uL | | |
| | Gradient: | | |
| | Time (min) | Mobile Phase A (%) | Mobile Phase B (%) |
| | 0.00 | 95 | 5 |
| | 7.50 | 40 | 60 |
| | 10.00 | 5 | 95 |
| | 15.00 | 5 | 95 |
| | 16.00 | 95 | 5 |
| | 21.00 | 95 | 5 |

### Example 1: Preparation of 4-Amino-1-(2-chlorophenyl)-7-(trifluoromethyl)pyrido[2,3-d]pyrimidin-2(1H)-one (the compound of Formula (I))

### Step 1:

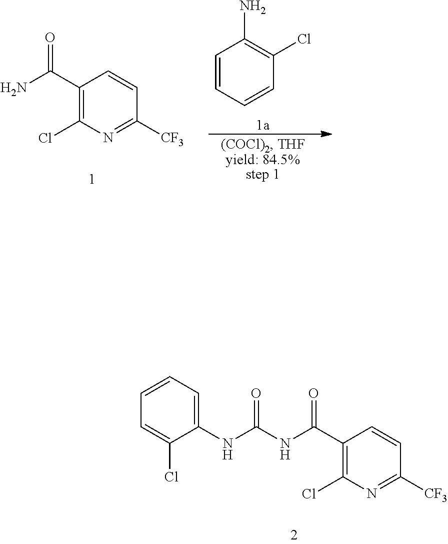

To a solution of compound 1 (300 g, 1.34 mol, 1.00 eq) in THF (2.40 L) was drop-wise added (COCl)₂ (178 g, 1.41 mol, 123 mL, 1.05 eq) at 25° C. over 10 mins. The reaction mixture was heated to 70° C. over 1 h, and then the mixture was stirred at 70° C. for 4.5 h. The reaction mixture was cooled to 30° C. over 0.5 h, and Compound 1a (180 g, 1.41 mol, 149 mL, 1.05 eq) was added drop-wised to the reaction mixture over 15 mins. The reaction mixture was stirred at 30° C. for 2.5 h. The two reaction mixtures were concentrated in vacuum to give an off-white solid, which was triturated with methanol (1.20 L) for 0.5 h. Then the mixture was filtered and the solid dried under vacuum to give Compound 2 (900 g, 2.27 mol. 84.5% yield, 95.1% purity) as a white solid. LCMS: RT=0.982 min, m/z=377.9 (M+H)⁺. HPLC: RT=3.164 min, 95.1% purity under 220 nm. ¹H NMR: (400 MHz DMSO-d₆) δ 11.69 (s, 1H), 10.73 (s, 1H), 8.42 (d, J=8.0 Hz, 1H), 8.21 (d, J=4.0 Hz, 1H), 8.10 (d, J=7.2 Hz, 1H), 7.55 (d, J=6.4 Hz, 1H), 7.40-7.20 (m, 1H), 7.18-7.15 (m, 1H).

### Step 2:

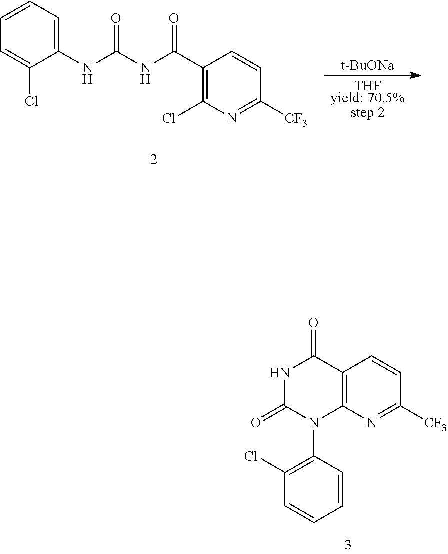 [SMILES: "t-BuONa" ; O=C(NC(=O)c1ccc(C(F)(F)F)nc1Cl)Nc1ccccc1Cl ; "THFyield: 70.5%step 2" ; O=c1[nH]c(=O)n(-c2ccccc2Cl)c2nc(C(F)(F)F)ccc12]

To a solution of compound 2 (292 g, 734 mmol, 1.00 eq) in THF (3.00 L) was added t-BuONa (155 g, 1.61 mol, 2.20 eq) slowly at 25° C.~50° C. over 0.5 h (with adding t-BuONa, the temperature increased from 25° C. to 50° C., the mixture became clear and soon turned to a yellow suspension). After adding t-BuONa, the mixture was stirred at 25° C.~50° C. for another 0.5 h. The four reaction mixtures were poured into 1 N HCl (10.0 L) with stirring and extracted with ethyl acetate (5.00 L*2). The combined organic layers were dried over Na₂SO₄, filtered and concentrated in vacuo at 50° C. to give a yellow solid which was triturated with MTBE (2.00 L) at 25° C. for 0.5 h, filtered and dried in vacuo at 50° C. to yield compound 3 (750 g, 2.07 mol, 70.5% yield, 94.2% purity) an off-white solid. LCMS: RT=1.100 min, m/z=342.0 (M+H)⁺. HPLC: RT=2.692 min, 94.2% purity under 220 nm. ¹H NMR: (400 MHz DMSO-d₆) δ 12.31 (s, 1H), 8.68 (d, J=8.0 Hz, 1H), 7.79 (d, J=8.0 Hz, 1H), 7.70-7.67 (m, 1H), 7.58-7.52 (m, 3H).

### Step 3:

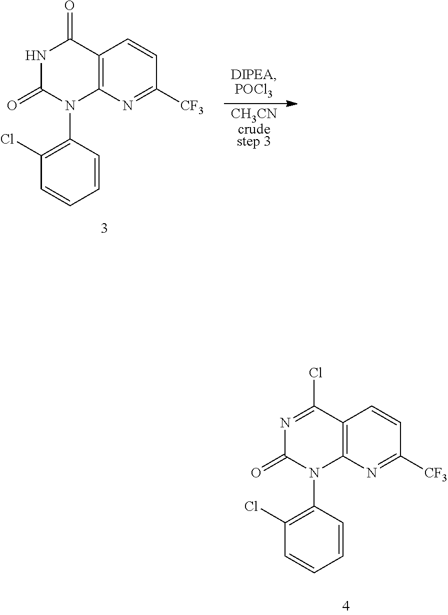 [SMILES: O=c1[nH]c(=O)n(-c2ccccc2Cl)c2nc(C(F)(F)F)ccc12 ; "CH3CNcrudestep 3" ; "DIPEA,POCl3" ; O=c1nc(Cl)c2ccc(C(F)(F)F)nc2n1-c1ccccc1Cl]

To the mixture of compound 3 (300 g, 878 mmol, 1.00 eq) and DIPEA (227 g, 1.76 mol, 305 mL, 2.00 eq) in CH₃CN (3.00 L) was drop-wise added POCl₃ (202 g, 1.32 mol, 122 mL, 1.50 eq) at 25° C. Then the mixture was stirred at 80° C. for 2 h. The reaction mixture was concentrated by distillation at 50° C. to give the crude compound 4 as brown oil (640 g).

### Step 4:

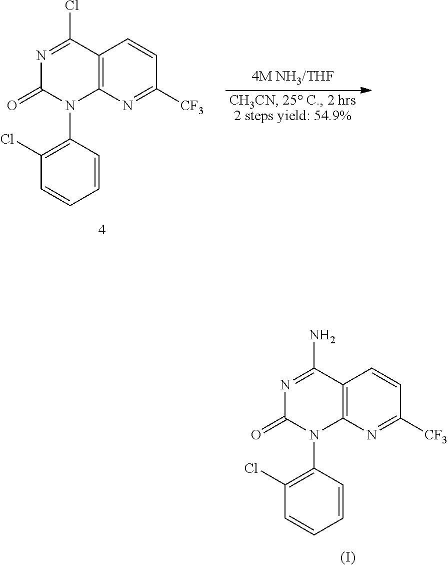

To a solution of compound 4 (99.35 g, 275 mmol) in CH₃CN (1.00 L) was drop-wise added the solution of NH₃/THF (4 M, 689 mL, 10.0 eq) at 0° C. (a lot of yellow solid precipitated out). The mixture was warmed to 25° C. and stirred at 25° C. for 2 hrs. The reaction mixture was concentrated in vacuum to give a yellow solid. 1.00 L water was added to the yellow solid with stirring for 12 hrs. The suspension was filtered and the resulting yellow solid was slurried with EtOH (800 mL) at 25° C. for 1 hr and then filtered. The yellow solid was slurried with DCM (1.00 L) at 25° C. for 1 h, filtered and then dried at 40° C. by oil pump for 3 hrs to yield (I) (52.0 g, 151 mmol, 54.9% yield, 99.2% purity) as yellow solid. LCMS: Rt=1.909 mins, m/z=341.2 (M+H)⁺. HPLC: Rt=2.415 mins, 99.2% purity in 220 nm. ¹H NMR: (400 MHz, DMSO-d₆) δ 8.83 (d, J=8.0 Hz, 1H), 8.57 (br. s, 2H), 7.79 (d, J=8.0 Hz, 1H), 7.65-7.62 (m, 1H), 7.49-7.46 (m, 3H).

### Example 2: Recrystallization of the Compound of Formula (I) To Provide Form A

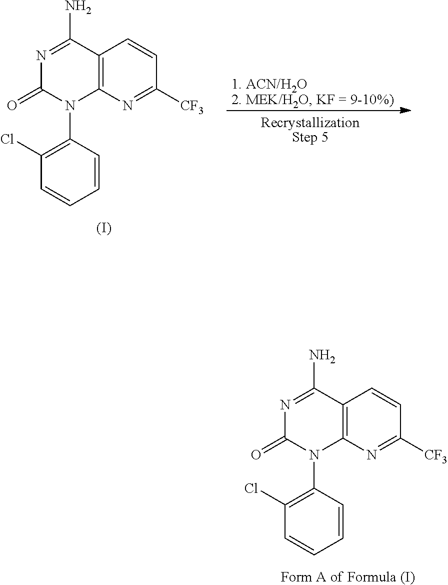

Crude compound of Formula (I) (100 g, 0.29 mol, 1.0 eq.) was dissolved in MeCN (2.18 kg, 28 vol, 21.8×) and H₂O (700 g, 7 vol, 7×) at 75-80° C. for 1-3 h. Adjust the mixture to 60-65° C. and concentrate to 30 vol below 65° C. Then charge Form A seed crystal (2 g, 0.02×) and stir the mixture at 60-65° C. for 1.5 h. Concentrate it to 20 vol below 65° C. Charge 1 kg (10 vol, 10×) water into the mixture. Concentrate it to 20 vol below 65° C. again. Charge 1 kg (10 vol, 10×) water into the mixture again. Cool it to 0-5° C. in 1.5 h. Then stir it at 0-5° C. for 13 h. Filter and wash the wet cake with 156 g (2 vol, 1-2×) of MeCN three times. Then charge the wet cake into the reactor and charge MEK (405 g, 5 vol, 4.05×) and H₂O (48 g, 0.48 vol, 0.48×) to adjust the KF: 9.0-10.0 wt % (9.4%). Then stir it at 60-65° C. for 17-22 h. Cool it to 0° C. in 4 h and stir it at 0-5° C. for 16 h. A sample was taken to check the form (Form A) and supernatant residual final product (FP) (1.3 wt %). Filter and wash the wet cake with MEK (156 g, 2 vol, 1.62×) two times. Dry the wet cake at 65-70° C. for 69 h to obtain Form A product (89.45 g, 99.8% purity, 100% assay) in 89% yield.

### Example 3: Phosphate Sensor Fluorescence Assay of the Compound of Formula (I)

The ability of the compound of Formula (I) described in Example 1 to inhibit MAT2A enzyme was determined using a Phosphate Sensor Fluorescence Assay described below.

MAT2A enzyme is incubated with a test compound in DMSO or DMSO and its substrates (L-methionine and ATP) in a microtiter plate. The enzymatic reaction is stopped by the addition of Working Phosphate Sensor Mixture. The plate is analyzed for fluorescence at 450 nm. The high control (DMSO with enzyme and its substrates) gives high fluorescence which represents no inhibition of enzymatic activity while the low control (DMSO with MAT2A substrates and no enzyme) gives low fluorescence which represents full inhibition of enzymatic activity.

### Materials:

Human MAT2A: Cepter, amino acids 1-395
Tris, pH 7.5: Invitrogen cat #15567-027
KCl: Ambion cat #AM9640G
MgCl₂: Ambion cat #AM9530G
Brij-35: Sigma cat B4184-10ML
DTT: Goldbio cat #DTT100
BGG: Sigma cat #G5009-25G
PNP: Novus Biologicals cat #NBP1-50872
7-MEG: Cayman Chemical cat #15988
L-Methionine: Alfa Aesar cat #J61904
ATP: Alfa Aesar cat #J60336
Phosphate Sensor: Thermo Fisher cat #PV4407
EDTA: Life Tech cat #15575-038
Assay plate: 384-well black polypropylene plate: Thomas Scientific cat #1149Q35

### Final Assay Conditions:

Assay Buffer: 50 mM Tris, pH 7.5/50 mM KCl/10 mM MgCl₂/0.01% Brij-35/1 mM DTT/0.1% BGG/40 nM PNP/6 μM 7-MEG
MAT2A: 10 nM for Cepter clone ID 329, lot 00023-123 before the addition of Working Phosphate Sensor Mixture
5 nM for Cepter clone ID 334, lot 00023-148 before the addition of Working Phosphate Sensor Mixture
L-methionine: 500 μM before the addition of Working Phosphate Sensor Mixture
ATP: 500 μM before the addition of Working Phosphate Sensor Mixture

### Procedure:

For the assay, a mixture of 1 mM L-methionine/1 mM ATP (2× final pre-stopped concentration) in assay buffer; MAT2A (2× final pre-stopped concentration) in Assay Buffer and Working Phosphate Sensor Mixture (1.5 μM Phosphate Sensor/30 mM EDTA in Assay Buffer, which is 3× final concentrations) were prepared. Test compounds or DMSO were added to the appropriate well suing D300e digital dispenser. 5 μl/well of Assay Buffer was added to the wells corresponding to the negative control and 5 μl/well of MAT2A was added to all the wells except for those corresponding to the negative control. After incubating the plate at room temperature for 15 minutes, 5 μl/well of the 1 mM L-methionine/1 mM ATP mixture was added to all wells. The plate was centrifuged at 1000 rpm for 1 minute and then incubated at room temperature for 1 hour. 5 μl of the Working Phosphate Sensor Mixture was added to all wells and the plate was centrifuged at 1000 rpm for 1 minute. The plate was read for fluorescence at 450 nm after exciting at 430 nm.

### Data Analysis:

Percent inhibition was calculated in Chemical and Biological Information System (CBIS), (ChemInnovation Software Inc.). Curves were fitted by CBIS as % inhibition vs. log [compound concentration] using a 4-parameter inhibition model.

$$Fit = ( A + ( ( B - A ) / ( 1 + ( ( C / x ) \hat{} D ) ) ) )$$

$$Res = ( y - fit )$$

The IC₅₀ of the compound of Formula (I) is listed as ++++, wherein “++++” represents IC₅₀<200 nM.

### Example 4: Solid State Form V

Solid state Form V is a solid form in medium crystallinity.

Solid state Form V was prepared from the compound of Formula (I) (~100 g). The starting material was the yellow solid precipitating after amination and vacuum concentration, as described in step 4 of Example 1. 1.00 L water was added to the yellow solid with stirring for 12 hrs. The suspension was filtered and concentrated to give a second yellow solid. The second yellow solid was slurried with EtOH (800 mL) at 25° C. for 1 hr. Then the mixture was filtered hard to give a third yellow solid and then the third yellow solid was slurried with DCM (1.0 L) at 25° C. for 1 h. Then the mixture was filtered hard to give the final yellow solid. The final yellow solid was dried under 40° C. by oil pump for 3 hrs to provide solid state Form V of the compound of Formula (I) (52.0 g, 99.3% purity).

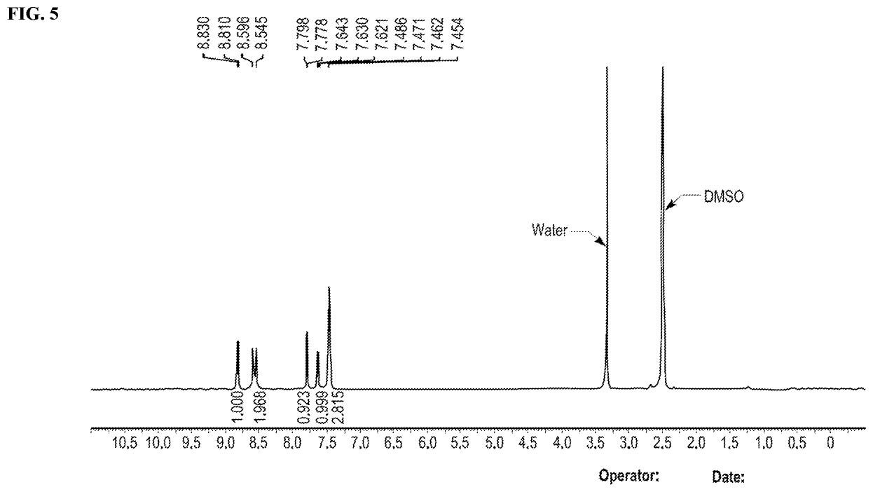

TABLE 1
Characterization of solid state Form V

| Parameter | Method | Result | FIG. |
|---|---|---|---|
| Stoichiometry | ¹H-NMR | No solvent residue | FIG. 5 |
| X-ray diffraction | XRPD, 3-40° (2 | Medium crystallinity | FIG. 1 |
| | theta) | | |
| Melting onset | DSC, 10° C./min | Onset: 316.3° C. | FIG. 2 |
| and enthalpy | | | |
| Thermogravimetry | TGA, 10° C./min | 0.7% @ 200.0° C. | FIG. 3 |
| Water content | KF | 0.5% by weight | — |
| Particle size | PLM | Needle-like particles | FIG. 4 |
| and morphology | | | |

TABLE 2
Peak Position of XRPD pattern as shown in FIG. 1
Peak Search Report (14 Peaks)

| No. | 2-Theta | d(A) | Relative Intensity (%) | Intensity |
|---|---|---|---|---|
| 1 | 6.15 | 14.36 | 100.00 | 664.17 |
| 2 | 8.91 | 9.92 | 5.94 | 130.40 |
| 3 | 10.36 | 8.53 | 34.81 | 315.66 |
| 4 | 10.88 | 8.13 | 45.76 | 386.06 |
| 5 | 12.29 | 7.19 | 31.02 | 312.19 |
| 6 | 12.50 | 7.07 | 27.99 | 295.49 |
| 7 | 16.54 | 5.35 | 75.41 | 587.05 |
| 8 | 19.87 | 4.46 | 6.45 | 189.03 |
| 9 | 21.29 | 4.17 | 13.31 | 249.82 |
| 10 | 22.56 | 3.94 | 8.66 | 241.92 |
| 11 | 23.42 | 3.80 | 8.04 | 247.10 |
| 12 | 26.06 | 3.42 | 9.92 | 265.98 |
| 13 | 27.54 | 3.24 | 25.23 | 343.46 |
| 14 | 28.64 | 3.11 | 25.40 | 328.58 |

### Example 5: Crystalline Form A

About 1.5 g of solid state Form V was equilibrated in 22 mL of ACN/water (1/1, V/V) with magnetic stirring at 25° C. for about 6 days. Precipitates were filtrated and dried at room temperature for about 18 hours. 1.36 g off-white solids were obtained in yield of 90%. Crystalline Form A was characterized by an X-ray powder diffraction pattern substantially in accordance with FIG. 6.

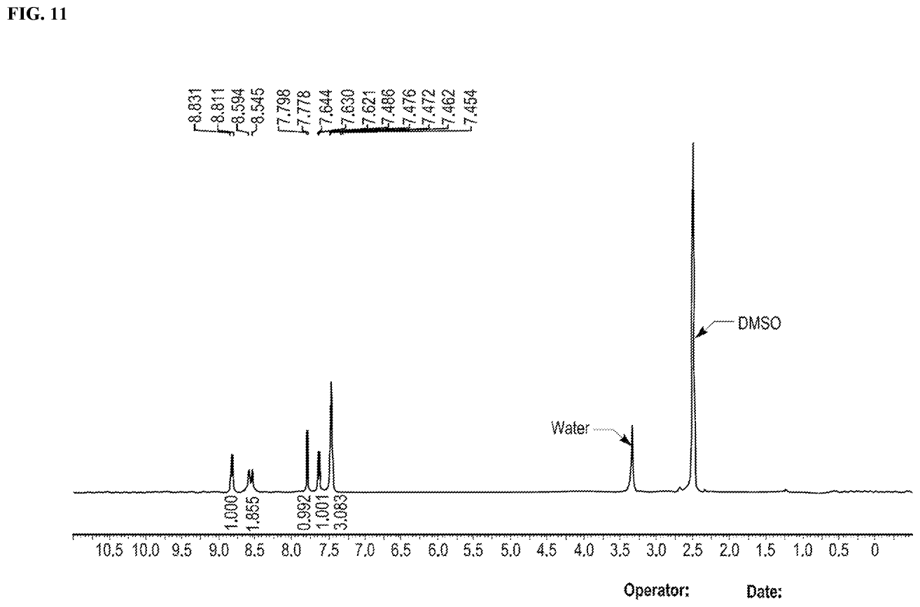

TABLE 3
Characterization of Crystalline Form A

| Parameter | Method | Result | FIG. |
|---|---|---|---|
| Yield | — | 90% | — |
| Purity | HPLC | 99.5% | — |
| X-ray diffraction | XRPD, 3-40° | High crystallinity | FIG. 6 |
| | (2 theta) | | |
| DSC melting onset | DSC, 10° C./min | Onset: 318.1° C. | FIG. 7 |
| Thermogravimetry | TGA, 10° C./min | 1.0% @ 220.0° C. | FIG. 8 |
| Stoichiometry | ¹H-NMR | No solvent residue | FIG. 11 |
| Water content | KF | 0.9% by weight | — |
| Morphology | PLM | Irregular particles | FIG. 9 |
| Hygroscopicity | DVS | See below | FIG. 10 |

Characterized by a dynamic vapor sorption (DVS), Form A is slightly hygroscopic with 0.7% water uptake in 95% RH at 25° C. No form change was observed after the DVS test.

TABLE 4
Peak Position of XRPD pattern as shown in FIG. 6
Peak Search Report (25 Peaks)

| No. | 2-Theta | d(A) | Relative Intensity (%) | Intensity |
|---|---|---|---|---|
| 1 | 6.06 | 14.58 | 99.14 | 955.74 |
| 2 | 6.57 | 13.45 | 32.70 | 354.45 |
| 3 | 7.14 | 12.37 | 4.72 | 98.98 |
| 4 | 8.33 | 10.61 | 4.79 | 102.46 |
| 5 | 10.04 | 8.80 | 15.36 | 209.80 |
| 6 | 11.07 | 7.99 | 47.59 | 503.13 |
| 7 | 12.08 | 7.32 | 43.69 | 469.37 |
| 8 | 13.09 | 6.76 | 9.53 | 162.33 |
| 9 | 14.24 | 6.22 | 3.65 | 110.44 |
| 10 | 15.58 | 5.68 | 16.37 | 234.97 |
| 11 | 16.59 | 5.34 | 100.00 | 990.82 |
| 12 | 18.50 | 4.79 | 11.22 | 174.81 |
| 13 | 20.27 | 4.38 | 4.34 | 115.51 |
| 14 | 20.78 | 4.27 | 15.25 | 219.05 |
| 15 | 21.71 | 4.09 | 7.88 | 156.97 |
| 16 | 22.44 | 3.96 | 18.08 | 252.90 |
| 17 | 22.78 | 3.90 | 3.80 | 125.17 |
| 18 | 23.57 | 3.77 | 7.35 | 163.06 |
| 19 | 25.33 | 3.51 | 10.73 | 205.87 |
| 20 | 25.74 | 3.46 | 10.36 | 202.51 |
| 21 | 26.28 | 3.39 | 9.71 | 195.00 |
| 22 | 27.43 | 3.25 | 18.55 | 273.79 |
| 23 | 27.69 | 3.22 | 40.46 | 471.11 |
| 24 | 28.42 | 3.14 | 38.60 | 449.74 |
| 25 | 31.42 | 2.84 | 4.29 | 122.30 |

### Example 6: Crystalline Form B

Crystalline Form B was obtained from any one of equilibration, temperature cycling, slow cooling, and fast cooling experiments in methanol, methanol/water, or ethanol/water solvent system, as described herein. Crystalline Form B was prepared by a temperature cycling experiment in methanol, and characterized by an X-ray powder diffraction pattern substantially in accordance with FIG. 12.

TABLE 5
Peak Position of XRPD pattern as shown in FIG. 12
Peak Search Report (23 Peaks)

| No. | 2-Theta | d(A) | BG | Height | I% | Area | I% | FWHM |
|---|---|---|---|---|---|---|---|---|
| 1 | 7.305 | 12.0918 | 57 | 89 | 21 | 820 | 6.8 | 0.16 |
| 2 | 9.989 | 8.8474 | 71 | 57 | 13.5 | 455 | 3.8 | 0.139 |
| 3 | 10.617 | 8.3255 | 80 | 208 | 49.2 | 1681 | 14 | 0.132 |
| 4 | 10.883 | 8.1224 | 86 | 125 | 29.6 | 1064 | 8.9 | 0.139 |
| 5 | 11.479 | 7.7024 | 95 | 180 | 42.6 | 1492 | 12.5 | 0.144 |
| 6 | 11.829 | 7.475 | 97 | 188 | 44.4 | 3241 | 27 | 0.282 |
| 7 | 12.01 | 7.3631 | 89 | 170 | 40.2 | 3895 | 32.5 | 0.398 |
| 8 | 15.881 | 5.5759 | 100 | 49 | 11.6 | 405 | 3.4 | 0.144 |
| 9 | 16.643 | 5.3223 | 109 | 279 | 66 | 6199 | 51.7 | 0.386 |
| 10 | 17.298 | 5.1222 | 135 | 133 | 31.4 | 1744 | 14.6 | 0.228 |
| 11 | 18.115 | 4.893 | 123 | 399 | 94.3 | 6681 | 55.8 | 0.291 |
| 12 | 19.708 | 4.5009 | 105 | 191 | 45.2 | 2091 | 17.5 | 0.19 |
| 13 | 20.28 | 4.3752 | 117 | 137 | 32.4 | 1717 | 14.3 | 0.218 |
| 14 | 21.249 | 4.178 | 139 | 98 | 23.2 | 1152 | 9.6 | 0.204 |
| 15 | 22.102 | 4.0184 | 151 | 145 | 34.3 | 921 | 7.7 | 0.11 |
| 16 | 22.699 | 3.9141 | 136 | 56 | 13.2 | 351 | 2.9 | 0.109 |
| 17 | 23.735 | 3.7455 | 116 | 73 | 17.3 | 1229 | 10.3 | 0.292 |
| 18 | 24.185 | 3.6769 | 111 | 140 | 33.1 | 2401 | 20 | 0.298 |
| 19 | 25.206 | 3.5302 | 109 | 53 | 12.5 | 960 | 8 | 0.296 |
| 20 | 26.555 | 3.3539 | 118 | 423 | 100 | 11982 | 100 | 0.492 |
| 21 | 27.271 | 3.2675 | 180 | 397 | 93.9 | 7871 | 65.7 | 0.344 |
| 22 | 28.269 | 3.1543 | 169 | 187 | 44.2 | 3334 | 27.8 | 0.31 |
| 23 | 29.62 | 3.0134 | 134 | 144 | 34 | 2596 | 21.7 | 0.313 |

### Example 7: Crystalline Form C

Crystalline Form C was obtained from any one of equilibration, temperature cycling, slow cooling and fast cooling experiments in methanol, methanol/water or ethanol/water system, as described herein.

Crystalline Form C was scaled up according to the procedure as follows: About 500 mg of solid state Form V was equilibrated in 7 mL of MeOH with magnetic stirring at 50° C. for about 3 days. Then it was cooled to 25° C. naturally and stirred at 25° C. for about 3 days. Precipitates were filtrated and dried at room temperature for about 20 hours. 366.6 mg white solids was obtained in 73% yield.

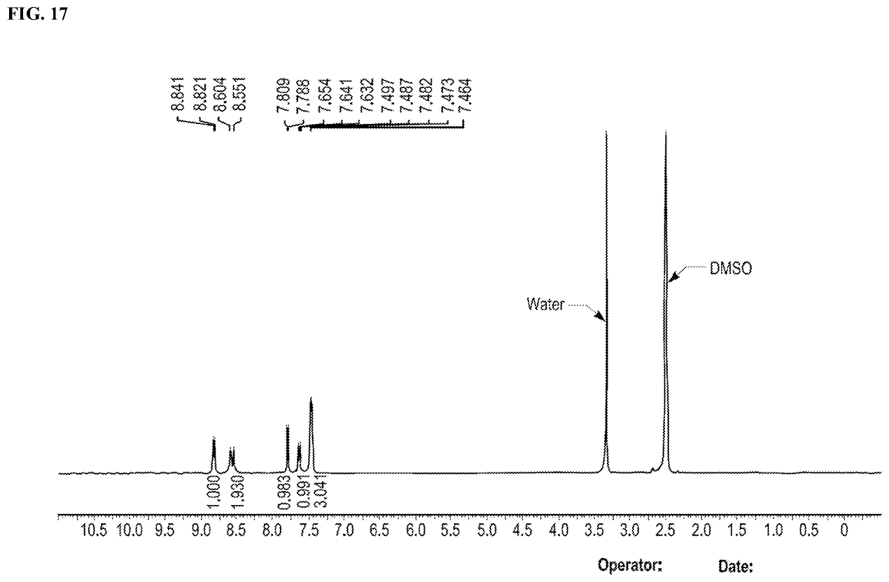

TABLE 6
Characterization of Crystalline Form C

| Parameters | Method | Results | FIG. |
|---|---|---|---|
| Yield | — | 73% | — |
| Purity | HPLC | 99.7% | — |
| X-ray diffraction | 3-40° (2 theta) | See Table below | FIG. 13 |
| DSC melting onset | DSC, | Onset: | FIG. 14 |
| | 10° C./min | 43.8° C. (66.1 J/g); | |
| | | 184.5° C. (5.7 J/g); | |
| | | 321.3° C. | |
| Thermogravimetry | TGA, | 3.9%@220° C. | FIG. 15 |
| | 10° C./min | | |
| Stoichiometry | ¹H-NMR | No solvent residue | FIG. 17 |
| Water content | Karl Fisher | 2.6% by weight | — |
| Morphology | PLM | Irregular particles | FIG. 16 |
| Hygroscopicity | DVS | See below | FIG. 18 |

Characterized by a dynamic vapor sorption (DVS), crystalline Form C has consistent water content between 30% RH and 95% RH. However, Form C starts to loss water and converts to Form L when humidity is below 30% RH. Form L absorbs water and converts back to Form C in 80% RH and above.

TABLE 7
Peak Position of XRPD pattern as shown in FIG. 13
Peak Search Report (24 Peaks)

| No. | 2-Theta | d(A) | BG | Height | I% | Area | I% | FWHM |
|---|---|---|---|---|---|---|---|---|
| 1 | 9.125 | 9.6835 | 63 | 142 | 9.1 | 1087 | 8.9 | 0.133 |
| 2 | 10.065 | 8.7808 | 67 | 125 | 8 | 993 | 8.1 | 0.138 |
| 3 | 10.88 | 8.1249 | 81 | 348 | 22.3 | 1898 | 15.6 | 0.095 |
| 4 | 11.825 | 7.4777 | 92 | 1558 | 100 | 12198 | 100 | 0.136 |
| 5 | 16.6 | 5.336 | 84 | 689 | 44.2 | 6039 | 49.5 | 0.152 |
| 6 | 17.466 | 5.0732 | 94 | 651 | 41.8 | 5366 | 44 | 0.143 |
| 7 | 17.79 | 4.9816 | 89 | 337 | 21.6 | 7800 | 63.9 | 0.378 |
| 8 | 18.097 | 4.8977 | 92 | 176 | 11.3 | 4572 | 37.5 | 0.451 |
| 9 | 19.691 | 4.5048 | 111 | 505 | 32.4 | 5374 | 44.1 | 0.185 |
| 10 | 20.32 | 4.3667 | 104 | 420 | 27 | 4822 | 39.5 | 0.199 |
| 11 | 21.039 | 4.2191 | 119 | 175 | 11.2 | 1583 | 13 | 0.157 |
| 12 | 21.794 | 4.0747 | 98 | 233 | 15 | 2868 | 23.5 | 0.214 |
| 13 | 23.737 | 3.7453 | 101 | 544 | 34.9 | 6924 | 56.8 | 0.221 |
| 14 | 24.55 | 3.6231 | 113 | 402 | 25.8 | 5951 | 48.8 | 0.257 |
| 15 | 25.266 | 3.522 | 113 | 109 | 7 | 1008 | 8.3 | 0.161 |
| 16 | 25.97 | 3.4282 | 137 | 213 | 13.7 | 1918 | 15.7 | 0.156 |
| 17 | 26.638 | 3.3437 | 184 | 260 | 16.7 | 1882 | 15.4 | 0.118 |
| 18 | 27.187 | 3.2774 | 186 | 675 | 43.3 | 11109 | 91.1 | 0.286 |
| 19 | 27.556 | 3.2343 | 227 | 158 | 10.1 | 730 | 6 | 0.076 |
| 20 | 28.169 | 3.1653 | 181 | 748 | 48 | 10262 | 84.1 | 0.238 |
| 21 | 29.822 | 2.9935 | 119 | 415 | 26.6 | 8474 | 69.5 | 0.355 |
| 22 | 33.011 | 2.7112 | 97 | 129 | 8.3 | 3017 | 24.7 | 0.406 |
| 23 | 34.622 | 2.5887 | 89 | 61 | 3.9 | 1211 | 9.9 | 0.345 |
| 24 | 37.377 | 2.4039 | 99 | 205 | 13.2 | 1822 | 14.9 | 0.154 |

### Example 8: Crystalline Form D

Crystalline Form D was obtained from any one of equilibration experiments in water at 50° C. and antisolvent experiments in methanol/water system, as described herein.

Crystalline Form D was scaled up according to the procedure as follows: About 500 mg of solid state Form V was equilibrated in 5 mL of water with magnetic stirring at 50° C. for about 6 days. Precipitates were filtrated and dried at room temperature for about 20 hours. 410.8 mg off-white solids was obtained in 82% yield.

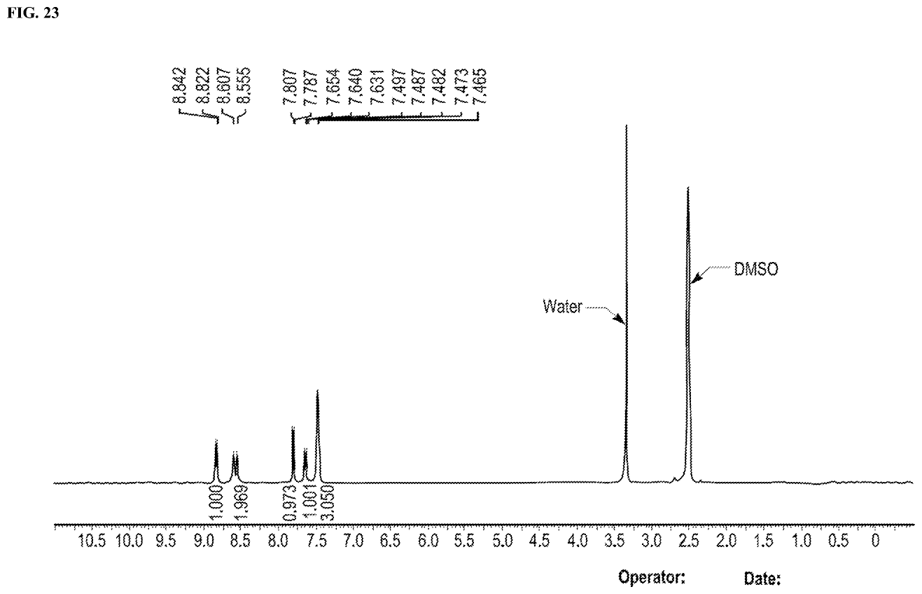

TABLE 7
Characterization of Crystalline Form D

| Parameters | Method | Results | FIG. |
|---|---|---|---|
| Yield | — | 82% | — |
| Purity | HPLC | 99.6% | — |
| X-ray diffraction | 3-40° (2 theta) | Pattern D | FIG. 19 |
| DSC melting onset | DSC, 10° C./min | Onset: | FIG. 20 |
| | | 45.8° C. (30.0 J/g); | |
| | | 317.1° C. | |
| Thermogravimetry | TGA, 10° C./min | 2.1%@220° C. | FIG. 21 |
| Stoichiometry | ¹H-NMR | No solvent residue | FIG. 23 |
| Water content | Karl Fisher | 2.3% by weight | — |
| Morphology | PLM | Irregular particles | FIG. 22 |
| Hygroscopicity | DVS | See below | FIG. 24 |

Characterized by a dynamic vapor sorption (DVS), crystalline Form D maintains its water content between 10% RH and 95% RH, but is slightly hygroscopic with about 0.9% water uptake from 40% to 95% RH. Form D starts to loss water when humidity is lower than 20% RH and restores water content when relative humidity is higher than 20% RH. No form change was observed after the DVS test.

TABLE 8
Peak Position of XRPD pattern as shown in FIG. 19
Peak Search Report (18 Peaks)

| No. | 2-Theta | d(A) | BG | Height | I% | Area | I% | FWHM |
|---|---|---|---|---|---|---|---|---|
| 1 | 6.147 | 14.3667 | 105 | 682 | 66.5 | 13432 | 78.2 | 0.342 |
| 2 | 10.315 | 8.5691 | 89 | 219 | 21.4 | 5499 | 32 | 0.436 |
| 3 | 10.925 | 8.0919 | 139 | 346 | 33.8 | 6586 | 38.3 | 0.331 |
| 4 | 12.357 | 7.1572 | 154 | 1025 | 100 | 17181 | 100 | 0.291 |
| 5 | 13.722 | 6.4478 | 151 | 504 | 49.2 | 3263 | 19 | 0.112 |
| 6 | 14.845 | 5.9624 | 161 | 245 | 23.9 | 2395 | 13.9 | 0.17 |
| 7 | 16.544 | 5.354 | 217 | 534 | 52.1 | 12327 | 71.7 | 0.401 |
| 8 | 18.05 | 4.9104 | 214 | 94 | 9.2 | 1517 | 8.8 | 0.28 |
| 9 | 19.257 | 4.6052 | 180 | 222 | 21.7 | 3331 | 19.4 | 0.261 |
| 10 | 21.165 | 4.1943 | 228 | 145 | 14.1 | 3611 | 21 | 0.432 |
| 11 | 22.563 | 3.9375 | 274 | 80 | 7.8 | 1374 | 8 | 0.298 |
| 12 | 24.124 | 3.6861 | 272 | 193 | 18.8 | 3019 | 17.6 | 0.272 |
| 13 | 25.205 | 3.5304 | 281 | 336 | 32.8 | 7571 | 44.1 | 0.391 |
| 14 | 26.743 | 3.3307 | 321 | 374 | 36.5 | 2351 | 13.7 | 0.109 |
| 15 | 27.611 | 3.228 | 391 | 504 | 49.2 | 7203 | 41.9 | 0.248 |
| 16 | 27.903 | 3.1948 | 355 | 285 | 27.8 | 8396 | 48.9 | 0.481 |
| 17 | 28.517 | 3.1274 | 379 | 117 | 11.4 | 2498 | 14.5 | 0.349 |
| 18 | 29.906 | 2.9853 | 225 | 202 | 19.7 | 1839 | 10.7 | 0.158 |

### Example 9: Crystalline Form E

Crystalline Form E was obtained from any one of equilibration experiments in methanol/water system, as described herein.

Crystalline Form E was scaled up according to the procedure as follows: About 500 mg of solid state Form V was equilibrated in 6 mL of mixture solvent of MeOH/water (1/1, V/V) with magnetic stirring at 50° C. for about 3 days. Then it was cooled to 25° C. naturally and stirred at 25° C. for about 3 days. Precipitates were filtrated and dried at room temperature for about 20 hours. 475.1 mg off-white solids was obtained in 95% yield.

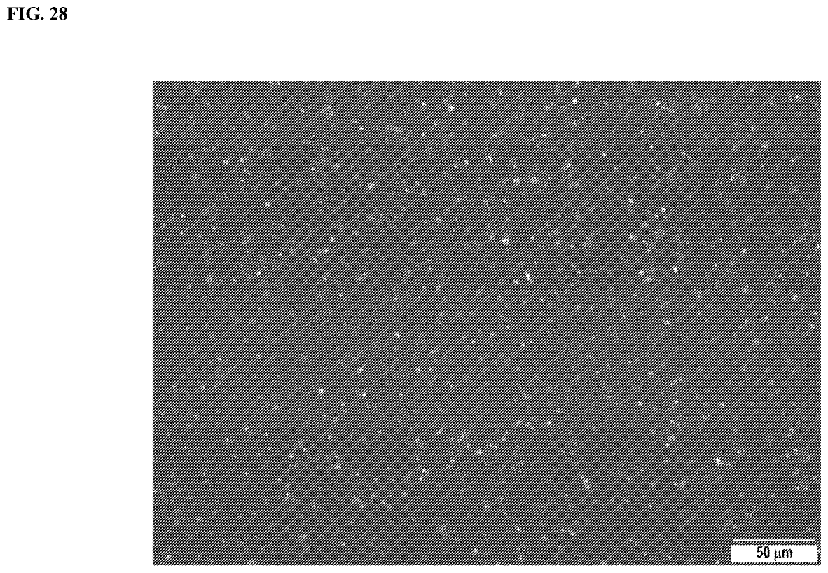

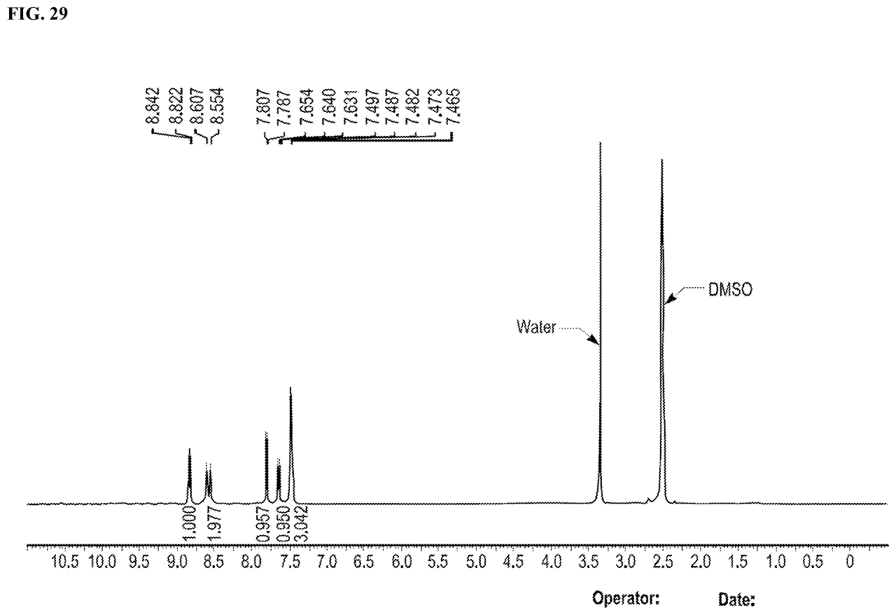

TABLE 9
Characterization of Crystalline Form E

| Parameters | Method | Results | FIG. |
|---|---|---|---|
| Yield | | 95% | — |
| Purity | HPLC | 99.6% | — |
| X-ray diffraction | 3-40° (2 theta) | Pattern E | FIG. 25 |
| DSC melting onset | DSC, 10° C./min | Onset: | FIG. 26 |
| | | 58.9° C. (103.8 J/g); | |
| | | 195.4° C. (3.8 J/g); | |
| | | 318.3° C. | |
| Thermogravimetry | TGA, 10° C./min | 4.3%@195.3° C. | FIG. 27 |
| Stoichiometry | ¹H-NMR | No solvent residue | FIG. 29 |
| Water content | Karl Fisher | 4.4% by weight | — |
| Morphology | PLM | Irregular particles | FIG. 28 |

TABLE 10
Peak Position of XRPD pattern as shown in FIG. 25
Peak Search Report (20 Peaks)

| No. | 2-Theta | d(A) | BG | Height | I% | Area | I% | FWHM |
|---|---|---|---|---|---|---|---|---|
| 1 | 7.204 | 12.261 | 57 | 146 | 11.8 | 1870 | 8.7 | 0.222 |
| 2 | 12.252 | 7.2179 | 112 | 1241 | 100 | 21390 | 100 | 0.299 |
| 3 | 13.743 | 6.4382 | 111 | 714 | 57.5 | 6055 | 28.3 | 0.147 |
| 4 | 14.566 | 6.0761 | 111 | 226 | 18.2 | 2822 | 13.2 | 0.204 |
| 5 | 14.91 | 5.9369 | 108 | 360 | 29 | 4844 | 22.6 | 0.234 |
| 6 | 15.723 | 5.6316 | 99 | 90 | 7.3 | 1321 | 6.2 | 0.255 |
| 7 | 17.282 | 5.127 | 132 | 122 | 9.8 | 3128 | 14.6 | 0.445 |
| 8 | 18.239 | 4.86 | 139 | 211 | 17 | 3180 | 14.9 | 0.262 |
| 9 | 19.385 | 4.5752 | 116 | 691 | 55.7 | 17814 | 83.3 | 0.448 |
| 10 | 20.819 | 4.2632 | 112 | 501 | 40.4 | 4915 | 23 | 0.16 |
| 11 | 21.733 | 4.086 | 218 | 144 | 11.6 | 2906 | 13.6 | 0.35 |
| 12 | 22.488 | 3.9504 | 193 | 337 | 27.2 | 4218 | 19.7 | 0.217 |
| 13 | 23.469 | 3.7874 | 200 | 93 | 7.5 | 1176 | 5.5 | 0.22 |
| 14 | 24.491 | 3.6316 | 253 | 502 | 40.5 | 10518 | 49.2 | 0.364 |
| 15 | 25.266 | 3.522 | 239 | 376 | 30.3 | 8682 | 40.6 | 0.401 |
| 16 | 26.698 | 3.3363 | 224 | 984 | 79.3 | 9723 | 45.5 | 0.172 |
| 17 | 27.617 | 3.2273 | 254 | 676 | 54.5 | 12849 | 60.1 | 0.311 |
| 18 | 28.046 | 3.1789 | 236 | 508 | 40.9 | 13611 | 63.6 | 0.438 |
| 19 | 29.946 | 2.9814 | 191 | 458 | 36.9 | 8892 | 41.6 | 0.337 |
| 20 | 31.069 | 2.8761 | 190 | 80 | 6.4 | 1025 | 4.8 | 0.222 |

### Example 10: Crystalline Form F

Crystalline Form F was obtained from any one of temperature cycling, slow cooling, and anti-solvent experiments in ethanol or ethanol/water solvent system, as described herein. Crystalline Form F was prepared by a temperature cycling experiment in ethanol-water, and characterized by an X-ray powder diffraction pattern substantially in accordance with FIG. 30, a DSC thermogram substantially in accordance with FIG. 31, and a thermal gravimetric analysis (TGA) thermogram substantially in accordance with FIG. 32. It contains about 0.2 equivalent of (2.7% by weight) ethanol based on ¹H-NMR result and about 0.5 (2.4% by weight) equivalent of water based on KF result. It exhibits a desolvation/dehydration peak at Tₒₙₛₑₜ of 45.2° C. followed by an endothermic solid-solid transition peak at Tₒₙₛₑₜ of 205.2° C. with enthalpy of about 3 J/g. It finally melts at 319.7° C. combined with decomposition. TGA shows about 3.2% weight loss at about 200° C.

TABLE 11
Peak Position of XRPD pattern as shown in FIG. 30
Peak Search Report (24 Peaks)

| No. | 2-Theta | d(A) | BG | Height | I% | Area | I% | FWHM |
|---|---|---|---|---|---|---|---|---|
| 1 | 7.834 | 11.2754 | 71 | 797 | 100 | 4672 | 54.3 | 0.102 |
| 2 | 10.677 | 8.2787 | 91 | 127 | 15.9 | 1960 | 22.8 | 0.268 |
| 3 | 11.808 | 7.4883 | 109 | 74 | 9.3 | 2226 | 25.9 | 0.522 |
| 4 | 12.41 | 7.1265 | 120 | 171 | 21.5 | 1632 | 19 | 0.166 |
| 5 | 13.089 | 6.7583 | 110 | 179 | 22.5 | 1118 | 13 | 0.108 |
| 6 | 14.729 | 6.0091 | 106 | 230 | 28.9 | 2393 | 27.8 | 0.181 |
| 7 | 15.664 | 5.6525 | 121 | 221 | 27.7 | 1746 | 20.3 | 0.129 |
| 8 | 16.646 | 5.3212 | 146 | 102 | 12.8 | 1790 | 20.8 | 0.305 |
| 9 | 17.322 | 5.1151 | 156 | 139 | 17.4 | 2110 | 24.5 | 0.248 |
| 10 | 17.792 | 4.9809 | 153 | 199 | 25 | 2992 | 34.8 | 0.246 |
| 11 | 18.242 | 4.8593 | 149 | 236 | 29.6 | 3190 | 37.1 | 0.235 |
| 12 | 19.283 | 4.5992 | 138 | 94 | 11.8 | 1476 | 17.2 | 0.257 |
| 13 | 19.695 | 4.5039 | 138 | 151 | 18.9 | 1815 | 21.1 | 0.209 |
| 14 | 20.3 | 4.3709 | 139 | 102 | 12.8 | 1176 | 13.7 | 0.188 |
| 15 | 21.327 | 4.1628 | 153 | 147 | 18.4 | 1690 | 19.7 | 0.2 |
| 16 | 22.365 | 3.9718 | 158 | 105 | 13.2 | 1684 | 19.6 | 0.278 |
| 17 | 23.78 | 3.7387 | 158 | 423 | 53.1 | 4521 | 52.6 | 0.186 |
| 18 | 24.921 | 3.57 | 165 | 219 | 27.5 | 2935 | 34.1 | 0.219 |
| 19 | 25.41 | 3.5024 | 165 | 190 | 23.8 | 2053 | 23.9 | 0.177 |
| 20 | 26.697 | 3.3364 | 274 | 87 | 10.9 | 1316 | 15.3 | 0.263 |
| 21 | 27.45 | 3.2465 | 258 | 659 | 82.7 | 8600 | 100 | 0.227 |
| 22 | 27.798 | 3.2067 | 265 | 319 | 40 | 3431 | 39.9 | 0.176 |
| 23 | 28.224 | 3.1593 | 247 | 75 | 9.4 | 783 | 9.1 | 0.171 |
| 24 | 29.762 | 2.9993 | 153 | 102 | 12.8 | 1173 | 13.6 | 0.2 |

### Example 11: Crystalline Form G

Crystalline Form G was obtained from any one of equilibration, temperature cycling, slow evaporation, and fast cooling experiments in acetone or acetone-water system, as described herein. Crystalline Form G was also obtained from any one of slow evaporation and fast evaporation experiments in MEK, slow cooling experiments in ethanol/acetone system, and anti-solvent experiment in ethanol/heptane. Crystalline Form G was prepared by a temperature cycling experiment in acetone-water, and characterized by an X-ray powder diffraction pattern substantially in accordance with FIG. 33, a DSC thermogram substantially in accordance with FIG. 34, and a thermal gravimetric analysis (TGA) thermogram substantially in accordance with FIG. 35.

TABLE 12
Peak Position of XRPD pattern as shown in FIG. 33
Peak Search Report (15 Peaks)

| No. | 2-Theta | d(A) | BG | Height | I% | Area | I% | FWHM |
|---|---|---|---|---|---|---|---|---|
| 1 | 5.533 | 15.9605 | 105 | 2158 | 100 | 32169 | 100 | 0.259 |
| 2 | 7.394 | 11.9454 | 110 | 273 | 12.7 | 5043 | 15.7 | 0.302 |
| 3 | 7.763 | 11.3794 | 113 | 207 | 9.6 | 5909 | 18.4 | 0.467 |
| 4 | 10.026 | 8.8152 | 146 | 417 | 19.3 | 8488 | 26.4 | 0.353 |
| 5 | 12.293 | 7.1941 | 125 | 132 | 6.1 | 2294 | 7.1 | 0.302 |
| 6 | 13.885 | 6.3726 | 136 | 78 | 3.6 | 1545 | 4.8 | 0.344 |
| 7 | 15.585 | 5.6813 | 190 | 916 | 42.4 | 14266 | 44.3 | 0.27 |
| 8 | 17.198 | 5.1516 | 181 | 466 | 21.6 | 8279 | 25.7 | 0.309 |
| 9 | 19.321 | 4.5901 | 152 | 98 | 4.5 | 886 | 2.8 | 0.157 |
| 10 | 20.874 | 4.2521 | 155 | 165 | 7.6 | 4228 | 13.1 | 0.445 |
| 11 | 23.388 | 3.8004 | 238 | 314 | 14.6 | 7466 | 23.2 | 0.413 |
| 12 | 24.534 | 3.6254 | 251 | 245 | 11.4 | 5123 | 15.9 | 0.363 |
| 13 | 27.7 | 3.2178 | 262 | 282 | 13.1 | 7984 | 24.8 | 0.492 |
| 14 | 30.536 | 2.9251 | 167 | 83 | 3.8 | 2027 | 6.3 | 0.424 |
| 15 | 31.413 | 2.8454 | 158 | 51 | 2.4 | 714 | 2.2 | 0.243 |

### Example 12: Crystalline Form H

Crystalline Form H was obtained from any one of equilibration, temperature cycling experiments in MTBE and MTBE/water system; or any one of slow cooling, fast cooling, and anti-solvent experiments in methanol/MTBE system, as described herein. Crystalline Form H was prepared by a temperature cycling experiment in MTBE-water, and characterized by an X-ray powder diffraction pattern substantially in accordance with FIG. 37, a DSC thermogram substantially in accordance with FIG. 38, and a thermal gravimetric analysis (TGA) thermogram substantially in accordance with FIG. 39. It has an endothermic peak at Tₒₙₛₑₜ of 210.5° C. with enthalpy of about 9 J/g followed by a melting peak at Tₒₙₛₑₜ of 315.5° C., which is combined with decomposition. TGA shows about 8.7% weight loss at about 240° C. It contains about 0.3 equivalent of (8.1% by weight) MTBE based on ¹H-NMR result. Crystalline Form H is a MTBE solvate.

TABLE 13
Peak Position of XRPD pattern as shown in FIG. 37
Peak Search Report (9 Peaks)

| No. | 2-Theta | d(A) | BG | Height | I% | Area | I% | FWHM |
|---|---|---|---|---|---|---|---|---|
| 1 | 5.106 | 17.2931 | 153 | 922 | 86.7 | 26133 | 89.5 | 0.492 |
| 2 | 5.819 | 15.1751 | 84 | 1064 | 100 | 29201 | 100 | 0.477 |
| 3 | 7.798 | 11.3284 | 128 | 110 | 10.3 | 1399 | 4.8 | 0.221 |
| 4 | 8.802 | 10.0378 | 131 | 148 | 13.9 | 4010 | 13.7 | 0.47 |
| 5 | 11.869 | 7.4502 | 123 | 92 | 8.6 | 1430 | 4.9 | 0.27 |
| 6 | 15.584 | 5.6814 | 154 | 682 | 64.1 | 18166 | 62.2 | 0.463 |
| 7 | 17.324 | 5.1145 | 130 | 79 | 7.4 | 1437 | 4.9 | 0.316 |
| 8 | 23.432 | 3.7933 | 179 | 150 | 14.1 | 4534 | 15.5 | 0.525 |
| 9 | 28.309 | 3.1499 | 202 | 124 | 11.7 | 3023 | 10.4 | 0.423 |

### Example 13: Crystalline Form I

Crystalline Form I was obtained from an equilibration experiment in acetone/water system at 50° C., as described herein.

Crystalline Form I was scaled up according to the procedure as follows: About 100 mg of solid state Form V was equilibrated in 1.6 mL of mixture solvent of Acetone/water (1/1, V/V) with magnetic stirring at 50° C. for about 3 days. Precipitates were filtrated and dried at room temperature for about 65 hours. 71.6 mg white solids were obtained in 71% yield.

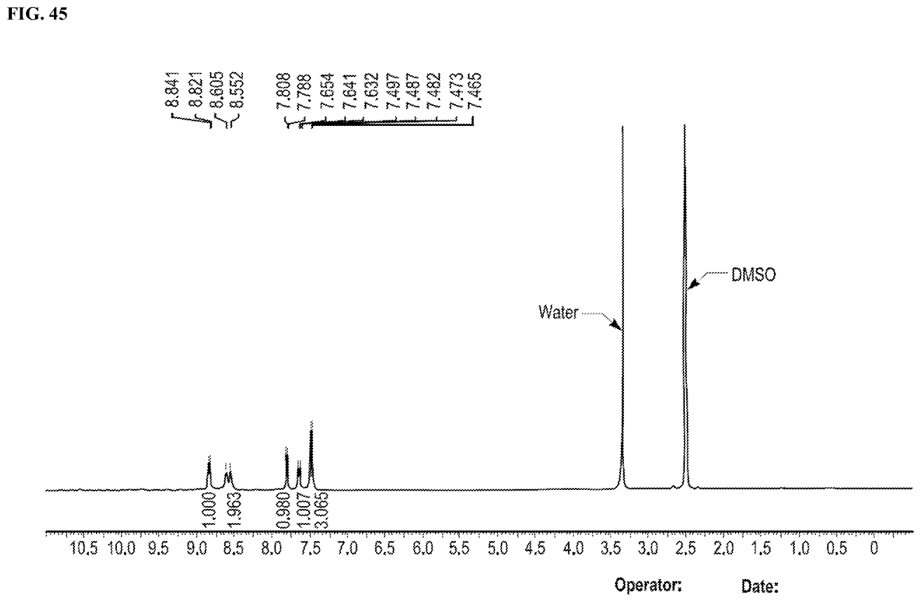

TABLE 14
Characterization of Crystalline Form I

| Parameters | Method | Results | FIG. |
|---|---|---|---|
| Yield | | 71% | — |
| Purity | HPLC | 99.6% | — |
| X-ray diffraction | 3-40° (2 theta) | Form I | FIG. 41 |
| DSC melting onset | DSC, 10° C./min | Onset: 261.6° C. | FIG. 42 |
| | | (4.0 J/g); 319.6° C. | |
| Thermogravimetry | TGA, 10° C./min | 1.1%@220.0° C. | FIG. 43 |
| Stoichiometry | ¹H-NMR | No solvent residue | FIG. 45 |
| Water content | Karl Fisher | 1.4% by weight | — |
| Morphology | PLM | Irregular particles | FIG. 44 |

TABLE 15
Peak Position of XRPD pattern as shown in FIG. 41
Peak Search Report (16 Peaks)

| No. | 2-Theta | d(A) | BG | Height | I% | Area | I% | FWHM |
|---|---|---|---|---|---|---|---|---|
| 1 | 5.759 | 15.3334 | 89 | 245 | 72.1 | 5004 | 52.9 | 0.355 |
| 2 | 6.407 | 13.7838 | 69 | 282 | 82.9 | 6070 | 64.2 | 0.374 |
| 3 | 8.441 | 10.4668 | 73 | 53 | 15.6 | 608 | 6.4 | 0.187 |
| 4 | 10.028 | 8.8132 | 110 | 106 | 31.2 | 1857 | 19.6 | 0.304 |
| 5 | 10.845 | 8.1515 | 119 | 151 | 44.4 | 3176 | 33.6 | 0.365 |
| 6 | 11.923 | 7.4162 | 111 | 46 | 13.5 | 658 | 7 | 0.248 |
| 7 | 13.051 | 6.7781 | 118 | 175 | 51.5 | 4159 | 44 | 0.413 |
| 8 | 16.113 | 5.496 | 149 | 340 | 100 | 9456 | 100 | 0.483 |
| 9 | 16.485 | 5.3729 | 155 | 209 | 61.5 | 6330 | 66.9 | 0.495 |
| 10 | 17.726 | 4.9995 | 135 | 58 | 17.1 | 1121 | 11.9 | 0.336 |
| 11 | 19.554 | 4.5361 | 115 | 52 | 15.3 | 1139 | 12 | 0.38 |
| 12 | 20.785 | 4.2702 | 128 | 42 | 12.4 | 136 | 1.4 | 0.056 |
| 13 | 23.263 | 3.8204 | 164 | 66 | 19.4 | 3258 | 34.5 | 0.807 |
| 14 | 25.474 | 3.4937 | 170 | 138 | 40.6 | 5200 | 55 | 0.616 |
| 15 | 27.739 | 3.2133 | 206 | 206 | 60.6 | 6914 | 73.1 | 0.583 |
| 16 | 28.949 | 3.0818 | 201 | 74 | 21.8 | 420 | 4.4 | 0.093 |

### Example 14: Crystalline Form J

Crystalline Form J was obtained from slow evaporation or fast cooling experiment in isopropanol, as described herein. Crystalline Form J was prepared by fast cooling in isopropanol, and characterized by an X-ray powder diffraction pattern substantially in accordance with FIG. 46, a DSC thermogram substantially in accordance with FIG. 47, and a thermal gravimetric analysis (TGA) thermogram substantially in accordance with FIG. 48. It exhibits a desolvation peak at Tₒₙₛₑₜ of 84.2° C. followed by an endothermic solid-solid transition peak at Tₒₙₛₑₜ of 204.0° C. It finally melts at 319.4° C. combined with decomposition. TGA shows about 9.9% weight loss at about 200° C. It contains about 2.3 equivalent of (28.5% by weight) IPA based on ¹H-NMR result. Crystalline Form J is in an isopropanol solvate form.

TABLE 16
Peak Position of XRPD pattern as shown in FIG. 46
Peak Search Report (31 Peaks)

| No. | 2-Theta | d(A) | BG | Height | I% | Area | I% | FWHM |
|---|---|---|---|---|---|---|---|---|
| 1 | 6.472 | 13.6461 | 65 | 264 | 9.3 | 1170 | 8.8 | 0.072 |
| 2 | 6.956 | 12.6981 | 68 | 173 | 6.1 | 1979 | 14.9 | 0.199 |
| 3 | 6.981 | 12.6524 | 69 | 272 | 9.6 | 1969 | 14.8 | 0.126 |
| 4 | 7.702 | 11.4696 | 69 | 1060 | 37.4 | 5072 | 38.1 | 0.083 |
| 5 | 10.636 | 8.3108 | 86 | 253 | 8.9 | 1171 | 8.8 | 0.08 |
| 6 | 11.442 | 7.7271 | 101 | 173 | 6.1 | 1354 | 10.2 | 0.136 |
| 7 | 12.191 | 7.2539 | 109 | 221 | 7.8 | 927 | 7 | 0.073 |
| 8 | 12.927 | 6.8429 | 106 | 2837 | 100 | 13313 | 100 | 0.081 |
| 9 | 14.647 | 6.043 | 105 | 860 | 30.3 | 5649 | 42.4 | 0.114 |
| 10 | 15.415 | 5.7435 | 99 | 296 | 10.4 | 1612 | 12.1 | 0.089 |
| 11 | 16.852 | 5.2567 | 128 | 328 | 11.6 | 1569 | 11.8 | 0.083 |
| 12 | 17.403 | 5.0915 | 136 | 366 | 12.9 | 2769 | 20.8 | 0.131 |
| 13 | 17.733 | 4.9974 | 121 | 333 | 11.7 | 1906 | 14.3 | 0.094 |
| 14 | 18.138 | 4.8867 | 127 | 620 | 21.9 | 3762 | 28.3 | 0.105 |
| 15 | 19.019 | 4.6623 | 102 | 291 | 10.3 | 3691 | 27.7 | 0.207 |
| 16 | 19.356 | 4.5821 | 101 | 272 | 9.6 | 7287 | 54.7 | 0.465 |
| 17 | 20.429 | 4.3438 | 110 | 106 | 3.7 | 1717 | 12.9 | 0.265 |
| 18 | 21.293 | 4.1694 | 138 | 360 | 12.7 | 3609 | 27.1 | 0.174 |
| 19 | 22.202 | 4.0006 | 154 | 484 | 17.1 | 5518 | 41.4 | 0.198 |
| 20 | 23.184 | 3.8334 | 136 | 676 | 23.8 | 7067 | 53.1 | 0.182 |
| 21 | 23.792 | 3.7368 | 131 | 413 | 14.6 | 4151 | 31.2 | 0.175 |
| 22 | 24.503 | 3.6299 | 131 | 159 | 5.6 | 2023 | 15.2 | 0.221 |
| 23 | 25.267 | 3.5219 | 129 | 590 | 20.8 | 4277 | 32.1 | 0.126 |
| 24 | 25.982 | 3.4266 | 139 | 352 | 12.4 | 3288 | 24.7 | 0.162 |
| 25 | 26.76 | 3.3287 | 212 | 410 | 14.5 | 2903 | 21.8 | 0.123 |
| 26 | 26.942 | 3.3066 | 164 | 719 | 25.3 | 8680 | 65.2 | 0.197 |
| 27 | 27.369 | 3.256 | 159 | 719 | 25.3 | 7284 | 54.7 | 0.166 |
| 28 | 27.636 | 3.2251 | 200 | 1112 | 39.2 | 9740 | 73.2 | 0.152 |
| 29 | 28.451 | 3.1346 | 155 | 159 | 5.6 | 1850 | 13.9 | 0.19 |
| 30 | 29.601 | 3.0154 | 132 | 244 | 8.6 | 5626 | 42.3 | 0.4 |
| 31 | 32.657 | 2.7398 | 107 | 264 | 9.3 | 3257 | 24.5 | 0.214 |

### Example 15: Crystalline Form K

Crystalline Form K was obtained from slow evaporation or fast evaporation experiment in THF, as described herein. Crystalline Form K was also prepared by competitive equilibration in THF, and characterized by an X-ray powder diffraction pattern substantially in accordance with FIG. 50.

TABLE 17
Peak Position of XRPD pattern as shown in FIG. 50
Peak Search Report (10 Peaks)

| No. | 2-Theta | d(A) | BG | Height | I% | Area | I% | FWHM |
|---|---|---|---|---|---|---|---|---|
| 1 | 5.493 | 16.0767 | 147 | 3164 | 100 | 52353 | 78.7 | 0.287 |
| 2 | 11.048 | 8.0017 | 148 | 1012 | 32 | 21007 | 31.6 | 0.36 |
| 3 | 13.806 | 6.4088 | 224 | 619 | 19.6 | 17275 | 26 | 0.485 |
| 4 | 16.524 | 5.3602 | 386 | 3055 | 96.6 | 66534 | 100 | 0.378 |
| 5 | 17.524 | 5.0567 | 151 | 346 | 10.9 | 5275 | 7.9 | 0.265 |
| 6 | 22.162 | 4.0077 | 173 | 345 | 10.9 | 8747 | 13.1 | 0.44 |
| 7 | 25.244 | 3.525 | 209 | 123 | 3.9 | 2656 | 4 | 0.375 |
| 8 | 27.045 | 3.2943 | 252 | 889 | 28.1 | 29978 | 45.1 | 0.586 |
| 9 | 27.739 | 3.2134 | 481 | 1736 | 54.9 | 55336 | 83.2 | 0.554 |
| 10 | 28.904 | 3.0865 | 360 | 182 | 5.8 | 5667 | 8.5 | 0.541 |

### Example 16: Crystalline Form L

Crystalline Form L was prepared by heating Form C to 120° C. Crystalline Form L was characterized by an X-ray powder diffraction pattern substantially in accordance with FIG. 51, a DSC thermogram substantially in accordance with FIG. 52, and a thermal gravimetric analysis (TGA) thermogram substantially in accordance with FIG. 53.

TABLE 18
Peak Position of XRPD pattern as shown in FIG. 51
Peak Search Report (27 Peaks)

| No. | 2-Theta | d(A) | BG | Height | I% | Area | I% | FWHM |
|---|---|---|---|---|---|---|---|---|
| 1 | 7.369 | 11.986 | 85 | 227 | 18.1 | 2106 | 11.4 | 0.146 |
| 2 | 9.976 | 8.8588 | 99 | 150 | 12 | 1139 | 6.2 | 0.127 |
| 3 | 10.747 | 8.2253 | 105 | 741 | 59 | 7963 | 43.3 | 0.18 |
| 4 | 11.67 | 7.5769 | 118 | 998 | 79.5 | 9472 | 51.5 | 0.159 |
| 5 | 12.245 | 7.2223 | 106 | 655 | 52.2 | 8235 | 44.8 | 0.211 |
| 6 | 12.837 | 6.8903 | 122 | 291 | 23.2 | 1765 | 9.6 | 0.096 |
| 7 | 14.69 | 6.0253 | 106 | 220 | 17.5 | 2102 | 11.4 | 0.16 |
| 8 | 16.288 | 5.4375 | 153 | 678 | 54 | 17928 | 97.4 | 0.443 |
| 9 | 16.623 | 5.3287 | 156 | 633 | 50.4 | 10587 | 57.5 | 0.264 |
| 10 | 17.039 | 5.1994 | 181 | 630 | 50.2 | 12247 | 66.6 | 0.326 |
| 11 | 17.927 | 4.944 | 175 | 1255 | 100 | 15870 | 86.3 | 0.2 |
| 12 | 18.361 | 4.828 | 169 | 1010 | 80.5 | 18400 | 100 | 0.287 |
| 13 | 19.74 | 4.4937 | 169 | 621 | 49.5 | 11227 | 61 | 0.303 |
| 14 | 20.056 | 4.4237 | 176 | 194 | 15.5 | 6875 | 37.4 | 0.594 |
| 15 | 20.31 | 4.3689 | 167 | 566 | 45.1 | 9583 | 52.1 | 0.284 |
| 16 | 21.496 | 4.1304 | 207 | 246 | 19.6 | 5328 | 29 | 0.363 |
| 17 | 22.184 | 4.0038 | 230 | 832 | 66.3 | 12797 | 69.5 | 0.258 |
| 18 | 23.329 | 3.8099 | 220 | 383 | 30.5 | 5287 | 28.7 | 0.231 |
| 19 | 24.238 | 3.6689 | 194 | 155 | 12.4 | 1770 | 9.6 | 0.191 |
| 20 | 25.167 | 3.5357 | 205 | 155 | 12.4 | 3051 | 16.6 | 0.33 |
| 21 | 26.248 | 3.3924 | 336 | 918 | 73.1 | 11617 | 63.1 | 0.212 |
| 22 | 26.976 | 3.3026 | 316 | 956 | 76.2 | 13792 | 75 | 0.242 |
| 23 | 28.065 | 3.1768 | 256 | 481 | 38.3 | 6387 | 34.7 | 0.223 |
| 24 | 29.405 | 3.035 | 177 | 333 | 26.5 | 4439 | 24.1 | 0.21 |
| 25 | 30.646 | 2.9148 | 141 | 95 | 7.6 | 1555 | 8.5 | 0.274 |
| 26 | 32.638 | 2.7414 | 134 | 128 | 10.2 | 2990 | 16.3 | 0.392 |
| 27 | 34.139 | 2.6242 | 135 | 98 | 7.8 | 2704 | 14.7 | 0.463 |

### Example 17: Crystalline Form M

Crystalline Form M was obtained from water activity experiments in acetonitrile/water system at 50° C. or from an equilibration experiment with solid state Form V in acetonitrile at 50° C., as described herein. Crystalline Form M was prepared from ACN-water, and characterized by an X-ray powder diffraction pattern substantially in accordance with FIG. 54.

TABLE 19
Peak Position of XRPD pattern as shown in FIG. 54
Peak Search Report (18 Peaks)

| No. | 2-Theta | d(A) | BG | Height | I% | Area | I% | FWHM |
|---|---|---|---|---|---|---|---|---|
| 1 | 6.226 | 14.184 | 68 | 1703 | 100 | 18919 | 100 | 0.193 |
| 2 | 6.718 | 13.1471 | 85 | 704 | 41.3 | 4957 | 26.2 | 0.115 |
| 3 | 10.457 | 8.4528 | 96 | 774 | 45.4 | 5206 | 27.5 | 0.11 |
| 4 | 11.286 | 7.8335 | 107 | 130 | 7.6 | 1279 | 6.8 | 0.171 |
| 5 | 12.436 | 7.1118 | 117 | 999 | 58.7 | 9795 | 51.8 | 0.17 |
| 6 | 13.442 | 6.5815 | 124 | 1035 | 60.8 | 7291 | 38.5 | 0.122 |
| 7 | 16.106 | 5.4986 | 126 | 701 | 41.2 | 8165 | 43.2 | 0.202 |
| 8 | 17.041 | 5.199 | 144 | 190 | 11.2 | 1705 | 9 | 0.147 |
| 9 | 17.624 | 5.0282 | 122 | 513 | 30.1 | 3991 | 21.1 | 0.127 |
| 10 | 20.239 | 4.3839 | 76 | 161 | 9.5 | 1628 | 8.6 | 0.176 |
| 11 | 22.753 | 3.905 | 104 | 198 | 11.6 | 3712 | 19.6 | 0.306 |
| 12 | 23.132 | 3.8419 | 115 | 144 | 8.5 | 2704 | 14.3 | 0.326 |
| 13 | 24.225 | 3.671 | 116 | 451 | 26.5 | 4918 | 26 | 0.189 |
| 14 | 26.521 | 3.3582 | 192 | 272 | 16 | 2757 | 14.6 | 0.176 |
| 15 | 27.067 | 3.2916 | 176 | 542 | 31.8 | 8041 | 42.5 | 0.242 |
| 16 | 28.29 | 3.152 | 179 | 639 | 37.5 | 8796 | 46.5 | 0.239 |
| 17 | 29.29 | 3.0466 | 133 | 102 | 6 | 1617 | 8.5 | 0.275 |
| 18 | 30.954 | 2.8866 | 105 | 124 | 7.3 | 831 | 4.4 | 0.116 |

### Example 18: Crystalline Form N

Crystalline Form N was prepared from any one of equilibration experiments of solid state Form V in acetonitrile at 30° C., 40° C. and 45° C., as described herein. Crystalline Form N was prepared from an equilibration experiment of solid state Form V in acetonitrile at 30° C., and characterized by an X-ray powder diffraction pattern substantially in accordance with FIG. 55.

TABLE 20
Peak Position of XRPD pattern as shown in FIG. 55
Peak Search Report (16 Peaks)

| No. | 2-Theta | d(A) | BG | Height | I% | Area | I% | FWHM |
|---|---|---|---|---|---|---|---|---|
| 1 | 5.387 | 16.3918 | 102 | 9190 | 100 | 55530 | 100 | 0.105 |
| 2 | 7.322 | 12.0639 | 95 | 173 | 1.9 | 958 | 1.7 | 0.096 |
| 3 | 10.744 | 8.2278 | 132 | 1957 | 21.3 | 10623 | 19.1 | 0.094 |
| 4 | 12.371 | 7.1486 | 146 | 224 | 2.4 | 1399 | 2.5 | 0.108 |
| 5 | 14.623 | 6.0525 | 227 | 542 | 5.9 | 3678 | 6.6 | 0.118 |
| 6 | 16.134 | 5.4891 | 275 | 5626 | 61.2 | 34743 | 62.6 | 0.107 |
| 7 | 21.55 | 4.1202 | 474 | 662 | 7.2 | 4478 | 8.1 | 0.117 |
| 8 | 21.817 | 4.0704 | 463 | 228 | 2.5 | 4048 | 7.3 | 0.29 |
| 9 | 22.597 | 3.9315 | 432 | 183 | 2 | 1715 | 3.1 | 0.163 |
| 10 | 24.857 | 3.5791 | 381 | 350 | 3.8 | 3071 | 5.5 | 0.152 |
| 11 | 25.431 | 3.4995 | 377 | 367 | 4 | 3574 | 6.4 | 0.169 |
| 12 | 26.022 | 3.4214 | 410 | 199 | 2.2 | 1003 | 1.8 | 0.088 |
| 13 | 27.007 | 3.2987 | 398 | 3976 | 43.3 | 27936 | 50.3 | 0.122 |
| 14 | 27.963 | 3.1881 | 362 | 1828 | 19.9 | 15130 | 27.2 | 0.144 |
| 15 | 28.355 | 3.1449 | 337 | 566 | 6.2 | 4871 | 8.8 | 0.149 |
| 16 | 30.536 | 2.9251 | 219 | 286 | 3.1 | 3666 | 6.6 | 0.223 |

### Example 19: Crystalline Form O

Crystalline Form O was prepared from any one of equilibration experiments of solid state Form V in ethanol at 30-50° C., as described herein. Crystalline Form O was prepared from an equilibration experiment of solid state Form V in ethanol at 30° C., and characterized by an X-ray powder diffraction pattern substantially in accordance with FIG. 56.

TABLE 21
Peak Position of XRPD pattern as shown in FIG. 56
Peak Search Report (12 Peaks)

| No. | 2-Theta | d(A) | BG | Height | I% | Area | I% | FWHM |
|---|---|---|---|---|---|---|---|---|
| 1 | 5.002 | 17.6516 | 100 | 662 | 68.2 | 5172 | 64.1 | 0.136 |
| 2 | 5.882 | 15.0132 | 100 | 571 | 58.9 | 5003 | 62 | 0.152 |
| 3 | 10.182 | 8.6806 | 134 | 85 | 8.8 | 310 | 3.8 | 0.063 |
| 4 | 12.803 | 6.9088 | 196 | 95 | 9.8 | 178 | 2.2 | 0.033 |
| 5 | 13.359 | 6.6223 | 203 | 970 | 100 | 7043 | 87.3 | 0.126 |
| 6 | 15.318 | 5.7796 | 246 | 474 | 48.9 | 5750 | 71.3 | 0.211 |
| 7 | 17.834 | 4.9695 | 352 | 540 | 55.7 | 5348 | 66.3 | 0.172 |
| 8 | 25.657 | 3.4692 | 311 | 734 | 75.7 | 8063 | 100 | 0.191 |
| 9 | 26.866 | 3.3157 | 290 | 194 | 20 | 2040 | 25.3 | 0.183 |
| 10 | 27.86 | 3.1996 | 249 | 160 | 16.5 | 1182 | 14.7 | 0.128 |
| 11 | 28.949 | 3.0818 | 206 | 181 | 18.7 | 1615 | 20 | 0.155 |
| 12 | 30.827 | 2.8981 | 155 | 86 | 8.9 | 1333 | 16.5 | 0.269 |

### Example 20: Crystalline Form P

Crystalline Form P was prepared from an equilibration experiment of solid state Form V in ACN at 50° C., as described herein. Crystalline Form P was characterized by an X-ray powder diffraction pattern substantially in accordance with FIG. 57.

TABLE 22
Peak Position of XRPD pattern as shown in FIG. 57
Peak Search Report (20 Peaks)

| No. | 2-Theta | d(A) | BG | Height | I% | Area | I% | FWHM |
|---|---|---|---|---|---|---|---|---|
| 1 | 5.578 | 15.8317 | 120 | 1530 | 75 | 12921 | 60.5 | 0.147 |
| 2 | 6.018 | 14.6744 | 115 | 1536 | 75.3 | 12312 | 57.7 | 0.139 |
| 3 | 6.7 | 13.1827 | 113 | 399 | 19.5 | 2739 | 12.8 | 0.119 |
| 4 | 9.457 | 9.3439 | 109 | 518 | 25.4 | 3941 | 18.5 | 0.132 |
| 5 | 10.782 | 8.1983 | 129 | 1065 | 52.2 | 8165 | 38.3 | 0.133 |
| 6 | 12.011 | 7.3622 | 151 | 820 | 40.2 | 5706 | 26.7 | 0.121 |
| 7 | 14.338 | 6.1724 | 220 | 802 | 39.3 | 6594 | 30.9 | 0.143 |
| 8 | 14.848 | 5.9613 | 233 | 732 | 35.9 | 6141 | 28.8 | 0.146 |
| 9 | 15.841 | 5.5899 | 256 | 411 | 20.1 | 2708 | 12.7 | 0.114 |
| 10 | 16.786 | 5.2772 | 296 | 358 | 17.5 | 3336 | 15.6 | 0.152 |
| 11 | 17.094 | 5.1829 | 321 | 1710 | 83.8 | 12630 | 59.2 | 0.128 |
| 12 | 18.055 | 4.909 | 352 | 826 | 40.5 | 7763 | 36.4 | 0.163 |
| 13 | 21.631 | 4.105 | 392 | 156 | 7.6 | 2358 | 11 | 0.262 |
| 14 | 23.037 | 3.8574 | 340 | 112 | 5.5 | 764 | 3.6 | 0.118 |
| 15 | 24.2 | 3.6747 | 320 | 118 | 5.8 | 1978 | 9.3 | 0.291 |
| 16 | 25.199 | 3.5312 | 318 | 102 | 5 | 852 | 4 | 0.145 |
| 17 | 26.577 | 3.3512 | 344 | 1778 | 87.1 | 20629 | 96.7 | 0.201 |
| 18 | 27.944 | 3.1902 | 358 | 2041 | 100 | 21340 | 100 | 0.182 |
| 19 | 28.585 | 3.1202 | 369 | 131 | 6.4 | 927 | 4.3 | 0.116 |
| 20 | 30.312 | 2.9462 | 193 | 243 | 11.9 | 3049 | 14.3 | 0.218 |

### Example 21: Crystalline Form Q

Crystalline Form Q was obtained from solubility study with Form A in a simulated gastric fluid (SGF, pH 2.0), as described herein. Crystalline Form Q was characterized by an X-ray powder diffraction pattern substantially in accordance with FIG. 58.

TABLE 23
Peak Position of XRPD pattern as shown in FIG. 58
Peak Search Report (15 Peaks)

| | | | Relative | |
|---|---|---|---|---|
| No. | 2-Theta | d(A) | Intensity (%) | Intensity |
| 1 | 3.34 | 26.47 | 21.03 | 144.19 |
| 2 | 4.00 | 22.08 | 12.81 | 102.27 |
| 3 | 5.36 | 16.48 | 73.98 | 338.49 |
| 4 | 8.23 | 10.74 | 6.89 | 81.98 |
| 5 | 10.70 | 8.26 | 14.55 | 116.26 |
| 6 | 14.54 | 6.09 | 9.33 | 100.41 |
| 7 | 16.09 | 5.50 | 100.00 | 449.00 |
| 8 | 19.53 | 4.54 | 7.28 | 80.64 |
| 9 | 22.60 | 3.93 | 5.30 | 68.68 |
| 10 | 24.97 | 3.56 | 10.85 | 92.95 |
| 11 | 24.91 | 3.57 | 8.18 | 82.39 |
| 12 | 26.98 | 3.30 | 53.01 | 260.92 |
| 13 | 27.88 | 3.20 | 13.47 | 108.81 |
| 14 | 27.96 | 3.19 | 19.62 | 132.22 |
| 15 | 31.72 | 2.82 | 26.87 | 148.54 |

### Example 22: Crystalline Form R

Crystalline Form R was obtained from solubility study with Form A in 0.1 N HCl solution and contained about 68.5% impurity from HPLC result. Crystalline Form R was characterized by an X-ray powder diffraction pattern substantially in accordance with FIG. 59.

TABLE 24
Peak Position of XRPD pattern as shown in FIG. 59
Peak Search Report (20 Peaks)

| | | | Relative | |
|---|---|---|---|---|
| No. | 2-Theta | d(A) | Intensity (%) | Intensity |
| 1 | 3.24 | 27.29 | 5.85 | 138.81 |
| 2 | 3.49 | 25.28 | 2.67 | 90.30 |
| 3 | 3.90 | 22.62 | 2.81 | 86.01 |
| 4 | 6.93 | 12.75 | 12.14 | 219.50 |
| 5 | 10.65 | 8.30 | 2.96 | 100.98 |
| 6 | 12.41 | 7.13 | 2.07 | 90.53 |
| 7 | 13.86 | 6.39 | 100.00 | 1446.45 |
| 8 | 16.83 | 5.26 | 2.31 | 87.74 |
| 9 | 17.38 | 5.10 | 3.40 | 101.11 |
| 10 | 18.50 | 4.79 | 4.30 | 109.72 |
| 11 | 18.85 | 4.70 | 5.96 | 133.24 |
| 12 | 20.44 | 4.34 | 4.92 | 116.75 |
| 13 | 21.59 | 4.11 | 6.35 | 138.33 |
| 14 | 22.35 | 3.97 | 4.86 | 120.49 |
| 15 | 22.89 | 3.88 | 9.38 | 182.44 |
| 16 | 23.35 | 3.81 | 4.01 | 105.79 |
| 17 | 24.48 | 3.63 | 13.09 | 226.26 |
| 18 | 27.39 | 3.25 | 4.07 | 108.25 |
| 19 | 27.88 | 3.20 | 5.88 | 134.80 |
| 20 | 28.39 | 3.14 | 4.17 | 110.78 |

### Example 23: Crystalline Form S

Crystalline Form S was obtained from any one of variable temperature XRPD experiments by heating Form D to 130° C. or from any one of variable humidity XRPD experiments of Form D when humidity was lower than 100% RH, as described herein. Crystalline Form S was prepared by heating Form D to 130° C., and characterized by an X-ray powder diffraction pattern substantially in accordance with FIG. 60. Form S is an anhydrous form.

TABLE 25
Peak Position of XRPD pattern as shown in FIG. 60
Peak Search Report (23 Peaks)

| | | | Relative | |
|---|---|---|---|---|
| No. | 2-Theta | d(A) | Intensity (%) | Intensity |
| 1 | 6.11 | 14.44 | 100.00 | 916.74 |
| 2 | 6.84 | 12.91 | 9.29 | 206.53 |
| 3 | 10.37 | 8.52 | 39.31 | 451.07 |
| 4 | 10.84 | 8.15 | 54.99 | 580.16 |
| 5 | 11.99 | 7.37 | 20.13 | 331.27 |
| 6 | 12.28 | 7.20 | 30.78 | 414.98 |
| 7 | 12.40 | 7.13 | 28.45 | 398.24 |
| 8 | 13.50 | 6.55 | 56.83 | 617.98 |
| 9 | 14.01 | 6.31 | 34.35 | 445.45 |
| 10 | 15.44 | 5.73 | 15.14 | 311.63 |
| 11 | 16.04 | 5.52 | 23.83 | 387.54 |
| 12 | 16.50 | 5.37 | 68.56 | 734.05 |
| 13 | 17.39 | 5.10 | 33.35 | 472.46 |
| 14 | 18.95 | 4.68 | 25.18 | 407.92 |
| 15 | 20.16 | 4.40 | 11.68 | 327.19 |
| 16 | 21.20 | 4.19 | 18.46 | 408.55 |
| 17 | 24.09 | 3.69 | 10.60 | 392.49 |
| 18 | 24.76 | 3.59 | 7.74 | 373.43 |
| 19 | 25.43 | 3.50 | 11.80 | 403.78 |
| 20 | 26.20 | 3.40 | 11.79 | 399.07 |
| 21 | 26.78 | 3.33 | 19.22 | 449.18 |
| 22 | 28.59 | 3.12 | 30.13 | 496.62 |
| 23 | 29.29 | 3.05 | 21.56 | 411.24 |

### Example 24: Crystalline Form T

Crystalline Form T was characterized by an X-ray powder diffraction pattern substantially in accordance with FIG. 61. This Form T further characterized according to Table 26. Crystalline Form T was also obtained from any one of variable temperature XRPD experiments by heating solid state Form V to 250° C. then cooling to 25° C., as described herein.

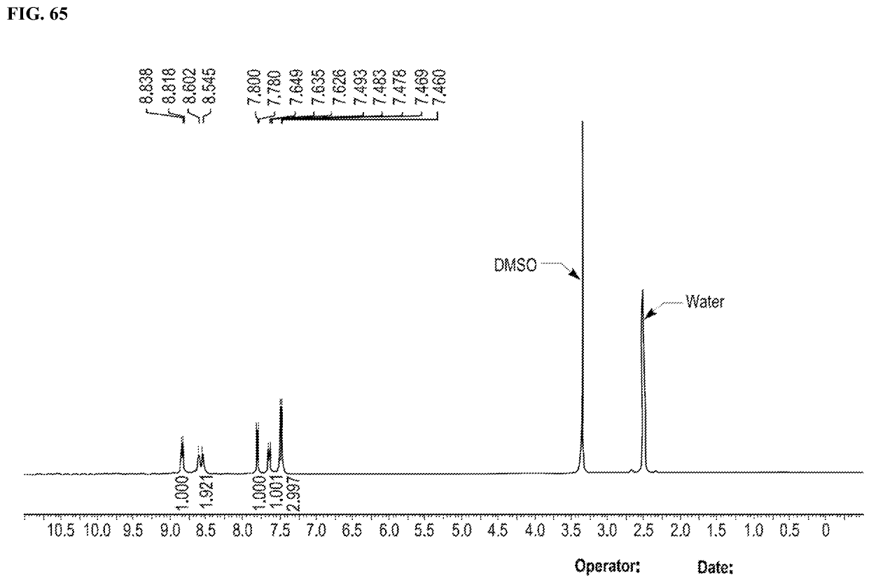

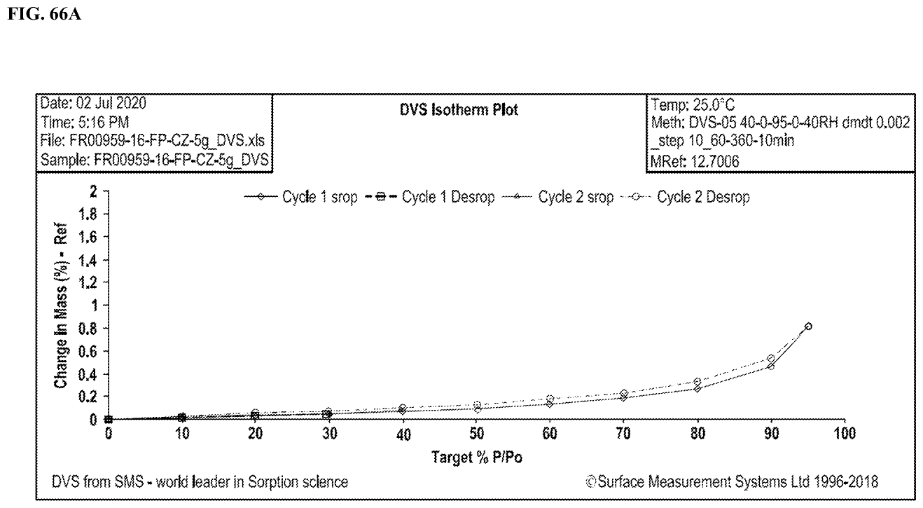

TABLE 26
Characterization of Crystalline Form T

| Parameter | Method | Result | FIG. |
|---|---|---|---|
| Purity | HPLC | 99.1% | — |
| X-ray diffraction | XRPD, 3-40° | High crystallinity, | FIG. 61 |
| | (2 theta) | see Table below | |
| DSC melting onset | DSC, 10° C./min | Onset: 306.6° C. | FIG. 62 |
| Thermogravimetry | TGA, 10° C./min | 0.8% @ 220.0° C. | FIG. 63 |
| Stoichiometry | ¹H-NMR | No solvent residue | FIG. 65 |
| Water content | KF | 1.7% by weight | — |
| Morphology | PLM | Irregular particles | FIG. 64 |
| Hygroscopicity | DVS | See below | FIG. 66 |

Characterized by a dynamic vapor sorption (DVS), Form T is slightly hygroscopic with 0.8% water uptake in 95% RH at 25° C. No form change was observed after the DVS test.

TABLE 27
Peak Position of XRPD pattern as shown in FIG. 61
Peak Search Report (26 Peaks)

| | | | Relative | |
|---|---|---|---|---|
| No. | 2-Theta | d(A) | Intensity (%) | Intensity |
| 1 | 6.51 | 13.57 | 100.00 | 1754.59 |
| 2 | 9.00 | 9.82 | 5.26 | 165.66 |
| 3 | 10.48 | 8.44 | 24.66 | 510.55 |
| 4 | 10.91 | 8.10 | 39.43 | 767.24 |
| 5 | 11.43 | 7.73 | 5.88 | 204.22 |
| 6 | 12.40 | 7.13 | 34.50 | 702.15 |
| 7 | 13.01 | 6.80 | 35.02 | 717.80 |
| 8 | 14.42 | 6.14 | 9.15 | 284.11 |
| 9 | 16.03 | 5.53 | 3.81 | 192.13 |
| 10 | 16.55 | 5.35 | 14.64 | 382.21 |
| 11 | 16.89 | 5.25 | 44.68 | 894.37 |
| 12 | 17.59 | 5.04 | 12.44 | 351.46 |
| 13 | 18.02 | 4.92 | 9.66 | 304.43 |
| 14 | 18.53 | 4.78 | 10.82 | 322.00 |
| 15 | 19.29 | 4.60 | 10.91 | 316.46 |
| 16 | 19.80 | 4.48 | 8.93 | 275.32 |
| 17 | 20.49 | 4.33 | 8.65 | 278.20 |
| 18 | 22.15 | 4.01 | 24.70 | 597.71 |
| 19 | 23.22 | 3.83 | 13.84 | 437.03 |
| 20 | 24.03 | 3.70 | 13.96 | 450.28 |
| 21 | 25.31 | 3.52 | 11.84 | 420.71 |
| 22 | 25.43 | 3.50 | 12.94 | 439.20 |
| 23 | 27.50 | 3.24 | 22.45 | 596.22 |
| 24 | 27.88 | 3.20 | 15.32 | 471.94 |
| 25 | 28.64 | 3.11 | 8.13 | 339.48 |
| 26 | 29.73 | 3.00 | 10.20 | 354.20 |

### Example 25: Crystalline Form U

Crystalline Form S was obtained from by heating Form A to 250° C. Form U converted to Form T after cooling to 25° C. There is no obvious thermal event in DSC curve of Form U. Crystalline Form U was characterized by an X-ray powder diffraction pattern substantially in accordance with FIG. 67.

TABLE 28
Peak Position of XRPD pattern as shown in FIG. 67
Peak Search Report (20 Peaks)

| | | | Relative | |
|---|---|---|---|---|
| No. | 2-Theta | d(A) | Intensity (%) | Intensity |
| 1 | 6.18 | 14.28 | 100.00 | 1807.30 |
| 2 | 8.80 | 10.04 | 8.92 | 241.90 |
| 3 | 10.34 | 8.55 | 34.47 | 690.00 |
| 4 | 10.79 | 8.20 | 33.27 | 675.37 |
| 5 | 12.35 | 7.16 | 39.90 | 797.24 |
| 6 | 13.84 | 6.39 | 8.58 | 264.12 |
| 7 | 14.98 | 5.91 | 3.01 | 168.51 |
| 8 | 16.43 | 5.39 | 52.14 | 1020.24 |
| 9 | 17.59 | 5.04 | 14.80 | 391.12 |
| 10 | 18.33 | 4.84 | 8.15 | 272.37 |
| 11 | 19.81 | 4.48 | 8.85 | 291.28 |
| 12 | 20.39 | 4.35 | 3.45 | 215.32 |
| 13 | 21.26 | 4.18 | 13.69 | 404.84 |
| 14 | 22.38 | 3.97 | 14.01 | 423.01 |
| 15 | 23.24 | 3.82 | 9.12 | 345.06 |
| 16 | 25.07 | 3.55 | 5.04 | 283.16 |
| 17 | 25.64 | 3.47 | 4.36 | 280.52 |
| 18 | 27.19 | 3.28 | 9.52 | 374.48 |
| 19 | 27.80 | 3.21 | 6.64 | 321.44 |
| 20 | 28.53 | 3.13 | 21.50 | 562.24 |

### Example 26: Substantially Amorphous Form

A substantially amorphous form was obtained by dry grinding Form T manually. An X-ray powder diffraction pattern of a substantially amorphous form is shown in FIG. 68.

### Example 27: Approximate Solubility of Solid State Form V at 25° C. and 50° C.

About 5 mg of solid state Form V was weighed to a 2 mL-glass vial and aliquot of 20 μL of each solvent was added and stirred for about 10 min to determine solubility at 25° C. About 10 mg of solid state Form V was weighed to a 2 mL-glass vial and aliquot of 20 μL of each solvent was added and stirred for about 10 min to determine solubility at 50° C. Maximum volume of each solvent added was 1 mL. Approximate solubility was determined by visual observation.

TABLE 29
Approximate solubility of solid state Form V at 25° C. and 50° C.

Solubility (mg/mL)

| Exp. ID | Solvent | 25° C. | 50° C. |
|---|---|---|---|
| SL1 | Water | <5 | <5 |
| SL2 | Methanol | 15-18 | 30-35 |
| SL3 | Ethanol | 7-8 | 15-17 |
| SL4 | 2-Propanol | <5 | 5-10 |
| SL5 | Ethyl acetate | <5 | <5 |
| SL6 | Isopropyl acetate | <5 | <5 |
| SL7 | Acetone | <5 | 5-10 |
| SL8 | Methyl ethyl ketone | <5 | <5 |
| SL9 | t-Butyl methyl ether | <5 | <5 |
| SL10 | Tetrahydrofuran | <5 | <5 |
| SL11 | 2-methyl-THF | <5 | <5 |
| SL12 | Acetonitrile | <5 | <5 |
| SL13 | Heptane | <5 | <5 |
| SL14 | Dichloromethane | <5 | <5 |
| SL15 | Toluene | <5 | <5 |

### Example 28: Equilibration with Solvents at 25° C. for 2 Weeks

About 50 mg of solid state Form V was equilibrated in 0.8 mL of solvent at 25° C. for 2 weeks with a stirring plate. Obtained suspension was filtered. The solid part (wet cake) was investigated by XRPD. When differences were observed, additional investigations were performed (e.g. DSC, TGA etc.).

TABLE 30
Crystalline Forms Formed by Equilibration
of solid state Form V at 25° C. for 2 Weeks

| Exp. | | |
|---|---|---|
| ID | Solvent/Concentration (mg/mL) | Crystalline Form |
| EQ2 | Methanol (65) | Form C (hemi-hydrate) |
| EQ3 | Methanol/water = 1/1 (v/v) (64) | Form E (hemi-hydrate) |
| EQ5 | Ethanol/water = 1/1 (v/v) (64) | Form C (hemi-hydrate) |
| EQ7 | Acetone (63) | Form G |
| EQ8 | Acetone/water = 1/1 (v/v) (63) | Form G |
| EQ10 | t-Butyl methyl ether (65) | Form H (MTBE solvate) |
| EQ11 | t-Butyl methyl ether/water = 95/5(v/v) | Form H (MTBE solvate) |
| | (63) | |
| EQ16 | Acetonitrile/water = 1/1 (v/v) (62) | Form A |

### Example 29: Equilibration with Solvents at 50° C. for 1 Week

About 50 mg of solid state Form V was equilibrated in 0.8 mL of solvent at 50° C. for 1 week with a stirring plate. Obtained suspension was filtered. The solid part (wet cake) was investigated by XRPD. When differences were observed, additional investigations were performed (e.g. DSC, TGA etc.).

TABLE 31
Crystalline Forms Formed by Equilibration
of solid state Form V at 50° C. for 1 Week

| Exp. | Solvent/Concentration | |
|---|---|---|
| ID | (mg/mL) | Crystalline Form |
| EQ20 | Water (64) | Form D (mono hydrate) |
| EQ21 | Methanol (63) | Form C (hemi-hydrate) |
| EQ22 | Methanol/water = 1/1 (v/v) (64) | Form E (hemi-hydrate) |
| EQ24 | Ethanol/water = 1/1 (v/v) (63) | Form C (hemi-hydrate) |
| EQ26 | Acetone (65) | Form G |
| EQ27 | Acetone/water = 1/1 (v/v) (63) | Form I |
| EQ29 | t-Butyl methyl ether (65) | Form H (MTBE solvate) |
| EQ30 | t-Butyl methyl ether/water = 95/5(v/v) | Form H (MTBE solvate) |
| | (63) | |

### Example 30: Temperature Cycling Experiments

About 50 mg of solid state Form V was equilibrated in 0.8 mL of solvents under a temperature cycle between 5° C. to 50° C. at a heating/cooling rate of 0.2° C./min for 6 cycles. After 6 cycles, precipitates were collected at 5° C. by centrifugation. The solid part (wet cake) was investigated by XRPD. When differences were observed, additional investigations were performed (e.g. DSC, TGA etc.).

TABLE 32
Crystalline Forms Formed by Temperature
Cycling of solid state Form V

| | Solvent/ | | |
|---|---|---|---|
| Exp. | Concentration | Crystalline | |
| ID | (mg/mL) | Form | Comments |
| TC2 | Methanol (63) | Form B then | Form B changed to Form C |
| | | changed to | after exposure to ambient |
| | | Form C | condition for about 2 days |
| TC3 | Methanol/water = | A mixture of | Form D changed to a |
| | 1/1 (v/v) (63) | Form D and | mixture of Form C and |
| | | Form C | Form D after exposure |
| | | | to ambient condition |
| | | | for about 2 days |
| TC5 | Ethanol/water = 1/1 | Form B then | Form B changed to Form F |
| | (v/v) (64) | changed to | after exposure to ambient |
| | | Form F | condition for about 2 days |
| TC8 | Acetone/water = 1/1 | Form G | — |
| | (v/v) (62) | | |
| TC10 | t-Butyl methyl ether | Form H | — |
| | (64) | (MTBE | |
| | | solvate) | |
| TC11 | t-Butyl methyl | Form H | — |
| | ether/water = | (MTBE | |
| | 95/5(v/v) (63) | solvate) | |

### Example 31: Crystallization at Room Temperature by Slow Evaporation

Combined with approximate solubility experiment, solubility samples were filtered by 0.45 μm nylon filter. Obtained solutions were slow evaporated at ambient condition. Solid residues were examined for their polymorphic form.

TABLE 33
Crystalline Forms Formed by Slow Evaporation

| Exp. | | |
|---|---|---|
| ID | Solvent/Concentration (mg/mL) | Crystalline Form |
| SE1 | Methanol (15) | Solid state Form V |
| SE2 | Ethanol (7) | Form F with low crystalline |
| SE3 | 2-Propanol (5) | Form J (IPA solvate) |
| SE6 | Acetone (5) | Form G |
| SE7 | Methyl ethyl ketone (5) | Form G |
| SE9 | Tetrahydrofuran (5) | Form K |
| SE12 | Methanol/Ethyl acetate = 1/1 | Solid state Form V |
| | (v/v) (7) | |
| SE13 | Methanol/Acetonitrile = 1/1 | Solid state Form V |
| | (v/v) (11) | |
| SE14 | Ethanol/t-Butyl methyl ether = 1/1 | A mixture of Form F and |
| | (v/v) (6) | Form G |
| SE15 | Ethanol/Dichloromethane = 1/1 | A mixture of Form F and |
| | (v/v) (6) | Form G |

### Example 32: Crystallization at Room Temperature by Fast Evaporation

About 10 mg of solid state Form V was equilibrated in 0.8-3.0 mL of solvent at 25° C. The samples are filtered by 0.45 μm nylon filter. Obtained solutions were fast evaporated under nitrogen flow. Solid residues were examined for their polymorphic form.

TABLE 34
Crystalline Forms Formed by Fast Evaporation

| Exp. | | |
|---|---|---|
| ID | Solvent/Concentration (mg/mL) | Crystalline Form |
| FE1 | Methanol (14) | Form B |
| FE2 | Ethanol (7) | Solid state Form V |
| FE3 | 2-Propanol (4) | Solid state Form V |
| FE6 | Acetone (3) | Form G |
| FE7 | Methyl ethyl ketone (3) | Form G |
| FE9 | Tetrahydrofuran (4) | Form K |
| FE10 | Acetonitrile (3) | Solid state Form V |
| FE12 | Methanol/Ethyl acetate = 1/1 (v/v) (5) | Solid state Form V |
| FE13 | Methanol/Acetonitrile = 1/1 (v/v) (5) | Solid state Form V |
| FE14 | Ethanol/t-Butyl methyl ether = 1/1 (v/v) (5) | Solid state Form V |
| FE15 | Ethanol/Dichloromethane = 1/1 (v/v) (5) | Solid state Form V |

### Example 33: Precipitation by Addition of Anti-Solvent

About 30-50 mg of solid state Form V was dissolved in a good solvent. Anti-solvent was added into the obtained solutions slowly. Precipitates were collected by filtration. The solid part (wet cake) was investigated by XRPD. When differences were observed, additional investigations will be performed (e.g. GH-NMR etc.).

TABLE 35
Crystalline Forms Formed by Addition of Anti-solvent

| Exp. | | | Solvent:Anti- | Crystalline |
|---|---|---|---|---|
| ID | Solvent | Anti-solvent | solvent (v:v) | Form |
| AS3 | Methanol | Water | 1/5 | Form D |
| | (15) | | | (monohydrate) |
| AS4 | Methanol | t-Butyl methyl | 1/5 | Form H (MTBE |
| | (15) | ether | | solvate) |
| AS5 | Ethanol (7) | Heptane | 1/5 | Form G |
| AS8 | Ethanol (7) | Water | 1/5 | Form F |

### Example 34: Crystallization From Hot Saturated Solutions by Slow Cooling

Approximate 50 mg of solid state Form V was dissolved in 1.7-6.0 mL of selected solvents at 50° C. Obtained solution or suspension was filtered by a 0.45 μm syringe membrane filter. Clear solutions were cooled to 5° C. at 0.1° C./min. Precipitates were collected by filtration. The solid part (wet cake) was investigated by XRPD. When differences were observed, additional investigations were performed (e.g. DSC, TGA, etc.).

TABLE 36
Crystalline Forms Formed by Slow Cooling
of Hot Saturated Solutions

| | Solvent/ | | |
|---|---|---|---|
| Exp. | Concentration | Crystalline | |
| ID | (mg/mL) | Form | Comments |
| SC1 | Methanol (30) | Form B then | Form B changed to |
| | | changed to | Form C after exposure |
| | | Form C | to ambient condition |
| | | | for about 2 days |
| SC2 | Ethanol (14) | Form F | — |
| SC3 | 2-Propanol (7) | Solid state | — |
| | | Form V | |
| SC4 | Acetone (7) | Form G | — |
| SC5 | Methanol/water = | A mixture of Form | — |
| | 1/1 (v/v) (8) | C and Form D | |
| SC6 | Ethanol/water = 1/1 | Form F | A mixture of Form L |
| | (v/v) (8) | | and Form C (after |
| | | | heating to 120° C.) |
| SC7 | Methanol/MTBE = | Form H (MTBE | — |
| | 1/1(v/v) (18) | solvate) | |
| SC8 | Ethanol/acetone = | Form G | — |
| | 1/1 (v/v) (16) | | |

### Example 35: Crystallization From Hot Saturated Solutions by Fast Cooling

About 50 mg of solid state Form V was dissolved in 1.7-8.0 mL of selected solvents at 50° C. Obtained solution or suspension was filtered by a 0.45 μm syringe membrane filter. Clear solutions were put into an ice bath and agitated. Precipitates were collected by filtration. The solid part (wet cake) was investigated by XRPD. When differences were observed, additional investigations were performed (e.g. DSC, TGA, etc.).

TABLE 37
Crystalline Forms Formed by Fast Cooling
of Hot Saturated Solutions

| | Solvent/Concentration | |
|---|---|---|
| Exp. ID | (mg/mL) | Crystalline Form |
| FC1 | Methanol (30) | Form C |
| FC2 | Ethanol (13) | Solid state Form V |
| FC3 | 2-Propanol (7) | Form J (IPA solvate) |
| FC4 | Acetone (7) | Form G |
| FC5 | Methanol/water = 1/1 (v/v) (6) | Form C |
| FC6 | Ethanol/water = 1/1 (v/v) (8) | Form F |
| FC7 | Methanol/MTBE = 1/1(v/v) (13) | Form H (MTBE solvate) |
| FC8 | Ethanol/acetone = 1/1 (v/v) (16) | A mixture of Form G and |
| | | Form F |

### Example 36: Vapor Diffusion Experiments

About 25-50 mg of solid state Form V was added to a glass vial and dissolved with 3 mL of good solvent. After sonication for about 30 seconds, obtained solution or suspension is filtered by a 0.45 μm syringe membrane filter. A clear solution is obtained. A suitable volume of the clear solution is transferred into a small glass vial (8 mL) without a lid and the lidless small vial was placed into a large glass vial (40 mL) containing anti-solvents in the bottom (solvent: anti-solvent=1:4, v/v). The large vials are tightly capped and placed at room temperature. Precipitates were collected by filtration. Solid part (wet cake) was investigated by XRPD.

TABLE 38
Crystalline Forms Formed by Vapor Diffusion

| Exp. | | | Solvent:Anti- | Crystalline |
|---|---|---|---|---|
| ID | Solvent | Anti-solvent | solvent | Form |
| VD2 | Methanol | Tetrahydrofuran | 1:4 | Solid state Form |
| | (16) | | | V |
| VD4 | Ethanol | Heptane | 1:4 | Solid state Form |
| | (8) | | | V |
| VD5 | Ethanol | Ethyl acetate | 1:4 | Solid state Form |
| | (8) | | | V |
| VD6 | Ethanol | t-Butyl methyl | 1:4 | Form H |
| | (8) | ether | | |

### Example 37: Water Activity Study

In a first study, mixtures of about 5 mg solid state Form V, 5 mg Form C, 5 mg Form D, and 5 mg Form E were equilibrated in 0.5 mL ACN/water mixtures to evaluate water activities at 5° C., 25° C. and 50° C. These solvent mixtures were saturated with the compound of Formula (I) before addition of the polymorph seeds. In a second study, about 30 mg solid state Form V was equilibrated in 0.5 mL in EtOH/water and MeOH/water mixtures to evaluate water activities at 25° C. These solvent mixtures were saturated with the compound of Formula (I) before addition of the polymorph seeds. Obtained suspension was filtered. Solid part (wet cake) was investigated by XRPD.

TABLE 39
Water activity study of solid state Form V and Forms C, D and
E at 5° C., 25° C. and 50° C.

| | | Solid state Form V and Forms C, D, and E | | |
|---|---|---|---|---|
| Exp. | | 5° C. | 25° C. | 50° C. |
| ID | Solvents | (6 days) | (6 days) | (6 days) |
| AW1 | ACN/water = | Form A | Form A | Form M |
| | 0.986/0.014 | | | |
| | (v/v) aw = 0.29 | | | |
| AW2 | ACN/water = | Form A | Form A | Form M then |
| | 0.959/0.041 | | | changed to |
| | (v/v) aw = 0.61 | | | solid state Form |
| | | | | V after exposure |
| | | | | to ambient |
| | | | | condition for |
| | | | | about 1 day |
| AW3 | ACN/water = | Form A | Form A | Form M |
| | 0.881/0.119 | | | |
| | (v/v) aw = 0.90 | | | |

TABLE 40
Water activity study of solid state Form V in
MeOH/water and EtOH/water systems at 25° C.

| Exp. | | Solid state Form V |
|---|---|---|
| ID | Solvents | 25° C. (4 days) |
| AW4 | EtOH/water = 0.4/0.6 (v/v) aw = 0.91 | Form F (protected by kapton film) |
| AW5 | EtOH/water = 0.3/0.7 (v/v) aw = 0.93 | Form F (protected by kapton film) |
| AW6 | EtOH/water = 0.2/0.8 (v/v) aw = 0.95 | Form F (protected by kapton film)^{a} |
| AW7 | EtOH/water = 0.1/0.9 (v/v) aw = 0.97 | Form E (protected by kapton film) |
| AW8 | MeOH aw = 0.0 | Form C (protected by kapton film) |
| AW9 | MeOH/water = 0.970/0.030 (v/v) | Form F (protected by kapton film) |
| | aw = 0.1 | |
| AW10 | MeOH/water = 0.930/0.070 (v/v) | Form F (protected by kapton film) |
| | aw = 0.2 | |
| AW11 | MeOH/water = 0.893/0.107 (v/v) | Form F (protected by kapton film) |
| | aw = 0.3 | |
| AW12 | MeOH/water = 0.775/0.225 (v/v) | Form F (protected by kapton film) |
| | aw = 0.5 | |
| AW13 | MeOH/water = 0.580/0.420 (v/v) | Form F (protected by kapton film) |
| | aw = 0.7 | |
| AW14 | MeOH/water = 0.4/0.6 (v/v) aw = 0.81 | Form E (protected by kapton film) |
| AW15 | MeOH/water = 0.3/0.7 (v/v) aw = 0.86 | Form E (protected by kapton film) |
| AW16 | MeOH/water = 0.2/0.8 (v/v) aw = 0.91 | Form E (protected by kapton film) |
| AW17 | MeOH/water = 0.1/0.9 (v/v) aw = 0.96 | Form E (protected by kapton film) |
| AW18 | Water aw = 1.0 | Form E (protected by kapton film)^{b} |

^{a}Form F with low crystallinity (suspension and dry solids were tested without kapton film); and
^{b}Form E (suspension and dry solids were tested without kapton film)

### Example 38: Competitive Equilibration Experiments

Mixtures of about 5 mg solid state Form V and 5 mg Form I were added to 0.5 mL selected solvents. These solvents were saturated with the compound of Formula (I) before addition of the polymorph seeds. Obtained suspension was stirred at 25° C. or 50° C. for 5 days. Solid part (wet cake) was isolated by filtration and investigated by XRPD.

TABLE 41
Competitive Equilibration of solid state Form V and Form I

| Exp. | Sample | Form A and Form I | |
|---|---|---|---|
| ID | Solvent | 25° C. (5 days) | 50° C. (5 days) |
| CE1 | Ethanol | Form A | Solid state Form V |
| CE2 | Acetone | Form G | Form G |
| CE3 | Acetonitrile | Form A | Solid state Form V |
| CE4 | Ethyl acetate | Solid state Form V | Solid state Form V |
| CE5 | Tetrahydrofuran | Form K | Form K |
| | | Changed to solid state | |
| | | Form V after put at | |
| | | ambient condition for | |
| | | about 18 hours | |

### Example 39: Investigation of Changes of Form A

About 30 mg of Form A was equilibrated in 0.5 mL of ACN or EtOH at 30° C., 40° C., 45° C. and 50° C. for 3 days with a stirring plate. Obtained suspension was filtered. Solid part (wet cake) was investigated by XRPD.

TABLE 42
Investigation of Changes of Form A

| | | | | | 50° C. |
|---|---|---|---|---|---|
| Exp. | Sol- | 30° C. | 40° C. | 45° C. | (3 days |
| ID | vents | (3 days) | (3 days) | (3 days) | or 6 days) |
| FC1 | ACN | Form N | Form N | Form N | Form P |
| | | (protected by | (protected by | (protected by | (protected by |
| | | kapton film), | kapton film), | kapton film), | kapton film) |
| | | changed to | changed to | changed to | Form M |
| | | Form A at | Form A at | Form A at | (without |
| | | ambient | ambient | ambient | kapton film) |
| | | condition for | condition for | condition for | Form M |
| | | about 3 days | about 3 days | about 3 days | changed to |
| | | | | | solid state |
| | | | | | Form V after |
| | | | | | slightly |
| | | | | | grinding |
| FC2 | EtOH | Form O | Form O | Form O | Form O |
| | | (protected by | (protected by | (protected by | (protected by |
| | | kapton film), | kapton film), | kapton film), | kapton film), |
| | | changed to | changed to | changed to | changed to |
| | | Form A at | Form A at | Form A at | Form A at |
| | | ambient | ambient | ambient | ambient |
| | | condition for | condition for | condition for | condition for |
| | | about 3 days | about 3 days | about 3 days | about 3 days |

### Example 40: Hygroscopicity—Water Sorption and Desorption Experiments

Water sorption and desorption behaviors of Form A, Form C, Form D, and Form T were investigated by DVS at 25° C. with a cycle of 40-0-95-0-40% RH. Dm/dt was 0.002. Minimum equilibration time was 60 min. Maximum equilibration time was 360 min. XRPD was measured after DVS test to determine potential form change.

Form A is slightly hygroscopic with 0.7% water uptake in 95% RH at 25° C. No form change was observed after the DVS test. The dynamic vapor sorption (DVS) profile of Form A is substantially as shown in FIG. 10.

Form C shows consistent water content between 30% RH and 95% RH. Form C starts to loss water when humidity is below 30% RH. Form L is the dehydration product of Form C according to variable humidity XRPD analysis. Form L absorbs water and converts back to Form C in 80% RH and above according to DVS and variable humidity XRPD analysis. A mixture of Form L and Form C was obtained after DVS test, as the DVS test was set to stop at 40% RH during the second sorption cycle. The dynamic vapor sorption (DVS) profile of Form C is substantially as shown in FIG. 18.

Form D is slightly hygroscopic with about 0.9% water uptake from 40% to 95% RH. Form D starts to loss water when humidity is lower than 20% RH. Form S is the dehydration product of Form D according to variable humidity XRPD analysis. Form S absorbs water and converts back to Form D when relative humidity is higher than 20% RH. No form change was observed after the DVS test. The dynamic vapor sorption (DVS) profile of Form D is substantially as shown in FIG. 24.

Form T is slightly hygroscopic with about 0.8% water uptake in 95% RH at 25° C. No form change was observed after the DVS test. The dynamic vapor sorption (DVS) profile of Form T is substantially as shown in FIG. 66.

### Example 41: Variable Humidity and Temperature XRPD Experiments

Based on DVS and DSC results of Form C, Form C starts to loss water when humidity is below 30% or when temperature is higher than about 44° C. In addition, the DSC curve shows a solid-solid transition at about 184° C. In order to determine identity of potential form changes after the dehydration and upon the solid-solid transition, variable humidity and variable temperature XRPD techniques were applied. XRPD patterns of the sample were measured online under 25° C./ambient environment (initial)-120° C./N₂ protection-25° C./ambient environment-25° C./80% RH (40 min)-25° C./0% RH (15 h), and 25° C./ambient environment (initial)-250° C./N₂ protection-25° C./ambient environment. The results show that Form C converts to Form L after dehydration by controlling relative humidity to 0% or temperature above 120° C. Form L coverts back to Form C after exposure to 25° C./80% RH for 40 min. When temperature is higher than 250° C., Form L converts to solid state Form V, indicating that the solid-solid transition in the DSC curve should be the phase transition from Form L to A and the two polymorphs are enantiotropically related.

TABLE 43
Variable Humidity and Temperature
XRPD Experiments of Form C

Form C

| Exp. | Temperature/Relative humidity | XRPD |
|---|---|---|
| ID | (holding time) | (XRPD method 2) |
| VT1 | 25° C./60% RH (initial) | Form C |
| VT2 | 120° C./60% RH (10 min) | Form L |
| VT3 | 25° C./60% RH (10 min) | A mixture of Form L |
| | | and Form C |
| VT4 | 25° C./80% RH (40 min) | Form C |
| VT5 | 25° C./0% RH (15 h) | A mixture of Form L |
| | | and Form C |

TABLE 44
Variable Temperature XRPD Experiments of Form C

| | Form C | |
|---|---|---|
| Exp. | Temperature/Relative humidity | |
| ID | (holding time) | XRPD (XRPD method 2) |
| VT6 | 25° C./60% RH (initial) | Form C |
| VT7 | 250° C./60% RH (10 min) | Solid state Form V |
| VT8 | 25° C./60% RH (final) | Solid state Form V |

Based on DVS and DSC results of Form D, Form D starts to loss water when humidity is below 10% or when temperature is higher than 46° C. In order to determine identity of the dehydration product, variable humidity and variable temperature XRPD techniques were applied. XRPD patterns of the sample were measured online with a temperature cycle of 25° C./ambient environment (initial)-130° C./N₂ protection (10 min)-25° C./N₂ protection-25° C./ambient environment (3h) and two water sorption and desorption cycles of 50% RH (initial)-90% RH (3h)-60% RH (2h)-40% RH (2h)-20% RH (3h)-10% RH (3h)-0% RH (6h)-10% RH (3h)-20% RH (3h)-50% RH at 25° C. The results show that Form D converts to Form S when humidity is 10% and below, or temperature is 130° C. (higher than dehydration temperature). Form S converts back to Form D after exposure to ambient condition for 3 h.

TABLE 45
Variable Humidity XRPD Experiments of Form D

| | Form D | |
|---|---|---|
| Exp. | Temperature/Relative humidity | |
| ID | (holding time) | XRPD (XRPD method 2) |
| VRH1 | 21° C./50% RH (initial) | Form D |
| VRH2 | 21° C./90% RH (3 h) | Form D |
| VRH3 | 21° C./60% RH (2 h) | Form D |
| VRH4 | 21° C./40% RH (2 h) | Form D |
| VRH5 | 21° C./20% RH (3 h) | Form D |
| VRH6 | 21° C./10% RH (3 h) | Form S |
| VRH7 | 21° C./0% RH (6 h) | Form S |
| VRH8 | 21° C./10% RH (3 h) | Form S |
| VRH9 | 21° C./20% RH (3 h) | Form S |
| VRH10 | 21° C./50% RH (final) | Form D |

TABLE 46
Variable Temperature XRPD Experiments of Form D

Form D

| Exp. | Temperature/Relative | |
|---|---|---|
| ID | humidity(holding time) | XRPD (XRPD method 2) |
| VT9 | 25° C./55% RH (initial) | Form D |
| VT10 | 130° C./55% RH (10 min) | Form S |
| VT11 | 25° C./N₂ protection (10 min) | Form S |
| VT12 | 25° C./55% RH (3 h) | Form D |
| VT13 | 25° C./55% RH (final) | Form D |

In order to investigate potential form change of Form A upon heating, variable temperature XRPD technique was applied. XRPD patterns of the sample were measured online with a temperature cycle of 25° C. (initial)-50° C./N₂ protection-70° C./N₂ protection-250° C./N₂ protection-25° C./N₂ protection-25° C./ambient condition (3h). The results show that there is no form change after heating to 70° C. Form A converts to Form U when temperature is 250° C., and Form U converts to Form T after cooling to 25° C.

TABLE 47
Variable Temperature XRPD Experiments of Form A

Form A

| Exp. | Temperature/Relative | |
|---|---|---|
| ID | humidity(holding time) | XRPD (XRPD method 2) |
| VT14 | 25° C./37% RH (initial) | Form A |
| VT15 | 50° C./37% RH (10 min) | Form A |
| VT16 | 70° C./37% RH (10 min) | Form A |
| VT17 | 250° C./37% RH (10 min) | Form U |
| VT18 | 25° C./N₂ protection (10 min) | Form T |
| VT19 | 25° C./37% RH (3 h) | Form T |

### Example 42: Behavior Under Compression

About 50-55 mg of solid state Form V, Form A, Form D, Form T, and an substantially amorphous form (obtained from grinding Form T) were compressed under 4 MPa or 2 MPa for 2 minutes with a hydraulic press. XRPD characterization was performed to investigate polymorphic behavior under compression.

TABLE 48
Behavior under compression

| Exp. | | | |
|---|---|---|---|
| ID | polymorph | Pressure/Time | XRPD |
| BUC1 | Solid state | 4 Mpa/2 min | No form change, crystallinity |
| | Form V | | slightly decreased |
| BUC2 | Solid state | 2 Mpa/2 min | No form change |
| | Form V | | |
| BUC3 | Form A | 4 Mpa/2 min | No form change, crystallinity |
| | | | decreased |
| BUC4 | Form A | 2 Mpa/2 min | No form change, crystallinity |
| | | | decreased slightly |
| BUC5 | Form D | 2 Mpa/2 min | No form change, crystallinity |
| | | | decreased |
| BUC6 | Form T | 2 Mpa/2 min | No form change, crystallinity |
| | | | decreased |
| BUC7 | Substantially | 2 Mpa/2 min | No form change |
| | amorphous | | |
| | form | | |

### Example 43: Grinding Simulation Experiments—Dry Grinding

About 20 mg of Form A, Form D, and Form T were ground manually with a mortar and a pestle for 5 min or 1 min. Potential from transition and degree of crystallinity were evaluated by XRPD. All the three forms (i.e., Forms A, D, and T) showed poor tolerance to dry grinding. A substantially amorphous form was obtained after drying grinding.

TABLE 49
Grinding Simulation Experiments - Dry Grinding

| Exp. ID | polymorph | | XRPD |
|---|---|---|---|
| GSE1 | Form A | Dry grinding for 5 min | substantially amorphous |
| GSE2 | Form A | Dry grinding for 1 min | substantially amorphous |
| GSE3 | Form D | Dry grinding for 5 min | substantially amorphous |
| GSE4 | Form D | Dry grinding for 1 min | substantially amorphous |
| GSE5 | Form T | Dry grinding for 5 min | substantially amorphous |

### Example 44: Grinding Simulation Experiments—Wet Grinding

Water or ethanol was added drop wise to about 20 mg of Form A, Form D, or Form T until solid was wetted sufficiently. Vortex was applied between each addition. Samples were dried under ambient condition for 10 min. Potential form transition and degree of crystallinity were evaluated by XRPD.

Form A and Form D showed poor tolerance to granulation with ethanol or water. A substantially amorphous form was obtained after granulation with ethanol. A mixture of solid state Form V and Form D was obtained after granulation with water. Form D and Form T showed good tolerance to granulation with water with no form change.

TABLE 50
Grinding Simulation Experiments - Wet Grinding

| Exp. ID | polymorph | Solvents | XRPD |
|---|---|---|---|
| GNSE1 | Form A | Ethanol | substantially amorphous |
| GNSE2 | | Water | Solid state Form V of low |
| | | | crystallinity + Form D |
| GNSE3 | Form D | Ethanol | substantially amorphous |
| GNSE4 | | Water | No form change |
| GNSE5 | Form T | Water | No form change |

### Example 45: Bulk Stability of Crystalline Forms

Solid state Form V, Form A, Form D, Form T, and the substantially amorphous form (from grinding of Form T) were stressed under 25° C./92 RH, 40% C/75% RH and 60° C. Solids obtained after bulk stability study were characterized by XRPD and HPLC.

Solid state Form V, Form A, Form D, and Form T showed good chemical stability and physical stability. The substantially amorphous form is physically stable, but showed slight degradation after exposure to 40° C./75 RH in an open container and 60° C. in a tight container.

TABLE 51
Bulk stability of solid state Form V, Forms A and D

| | | Parameter | | | | | |
|---|---|---|---|---|---|---|---|
| | | Solid state Form V | | Form A | | Form D | |
| | | Initial purity | | | | | |
| | | 98.9% | | 99.0% | | 98.0% | |
| Exp. ID | | Purity/Color | | Purity | | Purity | |
| | Solid state, 25° C./92% RH, open container | | | | | | |
| | | 1 week | 2 weeks | 1 week | 2 weeks | 1 week | 2 weeks |
| BS1 | Bulk | 98.9%/A | 98.9%/A | 99.0%/A | 99.2%/A | 98.0%/A | 98.0%/A |
| | (HPLC) | | | | | | |
| | Solid state, 40° C./75% RH, open container | | | | | | |
| | | 1 week | 2 weeks | 1 week | 2 weeks | 1 week | 2 weeks |
| BS2 | Bulk | 98.9%/A | 98.7%/A | 99.0%/A | 99.1%/A | 98.1%/A | 98.2%/A |
| | (HPLC) | | | | | | |
| | Solid state, 60° C., tight container | | | | | | |
| | | 1 week | 2 weeks | 1 week | 2 weeks | 1 week | 2 weeks |
| BS3 | Bulk | 98.8%/A | 98.9%/A | 99.1%/A | 99.2%/A | 98.1%/A | 97.8%/A |
| | (HPLC) | | | | | | |

Color A: All No change of color

TABLE 52
Bulk stability of Form T and Substantially Amorphous Form

| | | Parameter | | | | | |
|---|---|---|---|---|---|---|---|
| | | | | | Substantially | | |
| | | Form T | | | Amorphous Form | | |
| | | Initial purity | | | | | |
| | | 99.1% | | | 98.7% | | |
| Exp. ID | | Purity/Color | | | Purity/color | | |
| | Solid state, 25° C./92% RH, open container | | | | | | |
| | | 1 week | 2 weeks | 4 week | 1 weeks | 2 week | 4 weeks |
| BS1 | Bulk | 99.1%/A | 99.0%/A | 98.9%/A | 98.5%/A | 98.1%/A | 97.2%/A |
| | (HPLC) | | | | | | |
| | Solid state, 40° C./75% RH, open container | | | | | | |
| | | 1 week | 2 weeks | 4 week | 1 weeks | 2 week | 4 weeks |
| BS2 | Bulk | 98.9%/A | 98.7%/A | 98.3%/A | 97.5%/A | 96.1%/A | 93.5%/A |
| | (HPLC) | | | | | | |
| | Solid state, 60° C., tight container | | | | | | |
| | | 1 week | 2 weeks | 4 week | 1 weeks | 2 week | 4 weeks |
| BS3 | Bulk | 99.1%/A | 99.0%/A | 98.7%/A | 98.4%/A | 97.8%/B | 97.8%/A |
| | (HPLC) | | | | | | |

Color A: All No change of color;
Color B: Slightly discoloration

### Example 46: Equilibrium Solubility of Crystalline Forms

About 5 mg of solid state Form V, Form A, Form C, Form D, Form E, Form F, Form T, and the substantially amorphous form (from dry grinding Form T) was equilibrated in about 5 mL of Fed State Simulated Intestinal Fluid (FeSSIF) and Fasted State Simulated Intestinal Fluid (FaSSIF) separately at 37° C. for 24 hours with a stirring plate. Solid part and liquid part were separated by filtration. Solubility was measured by HPLC. Solid part (wet cake) was investigated by XRPD.

About 5 mg of solid state Form V and Form A were equilibrated in about 5 mL of simulated gastric fluid (SGF) (pH=2.0), 0.1 N HCl (pH=1.2) and phosphate buffer (pH=8.0) separately at 37° C. for 24 hours with a stirring plate. Solid part and liquid part were separated by filtration. Solubility was measured by HPLC. Solid part (wet cake) was investigated by XRPD.

The results indicate that all the forms showed comparable solubility in tested media. XRPD analysis showed no form change after the solubility tests, except that in 0.1 N HCl and SGF, two new patterns were obtained from the Form A sample. Additionally, it is found the impurity (compound 3) increased significantly in 0.1 N HCl (pH 1.2) and in SGF (pH 2.0), the acidic conditions.

TABLE 53
Solubility of solid state Form V, Forms A, C, D, E, F, T,
and substantially amorphous form in FeSSIF and FaSSIF

| | | | |
|---|---|---|---|
| Exp. | | Solubility (mg/mL)/(final | |
| ID | Medium | pH) | XRPD |

Solid state Form V, Target concentration: 1 mg/mL

| | | | |
|---|---|---|---|
| ES1^{a} | FaSSIF, pH 6.5 | 0.10 (6.5), no impurity | Solid state Form V |
| ES2^{a} | FeSSIF, pH 5.0 | 0.26 (5.0), no impurity | +NaCl |
| ES3^{b} | SGF, pH 2.0 | 0.07 (2.3), impurity: 0.05 | Solid state Form |
| | | | V + NaCl |
| ES4^{b} | pH 1.2 (0.1N | 0.15 (1.3), impurity: 0.06 | A mixture of |
| | HCl) | | impurity and solid |
| | | | state Form V |
| ES5^{b} | pH 8 (phosphate | 0.04 (8.0), no impurity | Solid state Form V |
| | buffer) | | |

Form A, Target concentration: 1 mg/mL

| | | | |
|---|---|---|---|
| ES6^{a} | FaSSIF, pH 6.5 | 0.09 (6.5), no impurity | Form A + NaCl |
| ES7^{a} | FeSSIF, pH 5.0 | 0.24 (5.0), no impurity | Form A + NaCl + |
| | | | one extra peak |
| ES8^{b} | SGF, pH 2.0 | 0.06 (2.2), impurity: 0.05 | Form Q |
| ES9^{b} | pH 1.2 (0.1N | 0.10 (1.3), impurity: 0.05 | Form R |
| | HCl) | | |
| ES10^{b} | pH 8 (phosphate | 0.03 (8.0), no impurity | Form A |
| | buffer) | | |

Form C, Target concentration: 1 mg/mL

| | | | |
|---|---|---|---|
| ES11^{a} | FaSSIF (6.5) | 0.06 (6.6), no impurity | Form C |
| ES12^{a} | FeSSIF(5.0) | 0.17 (5.2), no impurity | |

Form D, Target concentration: 1 mg/mL

| | | | |
|---|---|---|---|
| ES13^{a} | FaSSIF (6.5) | 0.09 (6.6), no impurity | Form D |
| ES14^{a} | FeSSIF(5.0) | 0.21 (5.2), no impurity | |

Form E, Target concentration: 1 mg/mL

| | | | |
|---|---|---|---|
| ES15^{a} | FaSSIF (6.5) | 0.06 (6.6), no impurity | Form E |
| ES16^{a} | FeSSIF(5.0) | 0.14 (5.2), no impurity | |

Form F, Target concentration: 1 mg/mL

| | | | |
|---|---|---|---|
| ES17^{b} | FaSSIF, pH 6.5 | 0.08 (6.6), impurity: 0.0008 | Form F |
| ES18^{b} | FeSSIF, pH 5.0 | 0.24 (5.0), impurity: 0.006 | |

Form T, Target concentration: 1 mg/mL

| | | | |
|---|---|---|---|
| ES19^{b} | FaSSIF, pH 6.5 | 0.08 (6.5), impurity: 0.0006 | Form T |
| ES20^{b} | FeSSIF, pH 5.0 | 0.16 (5.0), impurity: 0.003 | |

Substantially amorphous Form, Target concentration: 1 mg/mL

| | | | |
|---|---|---|---|
| ES21^{b} | FaSSIF, pH 6.5 | 0.12 (6.5), impurity: 0.002 | Substantially |
| ES22^{b} | FeSSIF, pH 5.0 | 0.23 (5.0), impurity: 0.005 | amorphous Form |

^{a}Solubility was tested by HPLC method 1;
^{b}Solubility was tested by HPLC method 2

### Example 47: Intrinsic Dissolution Rate Evaluation

Intrinsic dissolution rate (IDR) of solid state Form V, Form A, Form T, and the substantially amorphous form (from grinding of Form T) was compared in FaSSIF, pH 6.5.

About 100 mg of solid state Form V, Form A, Form T, and the substantially amorphous form (from grinding of Form T) were compressed under 2 Mpa for 2 min to form a compact disc of 8 mm in diameter. Only one surface of the pellet was exposed (surface area=0.50 cm²) to 900 mL FaSSIF, the dissolution medium. Temperature of chamber medium was set as 37±0.5° C. Shaft rotation as 100 rpm and sampling time points as 2 min, 5 min, 10 min, 15 min, 20 min, 25 min, 30 min, 35 min, 40 min, 50 min, 60 min, and 120 min. 2 mL sample solution was withdrawn at each time point and centrifuged at 14,000 rpm for 5 min to isolate supernatant. Then concentration of the supernatant was analyzed by HPLC. After 120 min, XRPD of disc surface was measured.

All the forms show no dissolution in FaSSIF. No form change was observed after IDR test. The concentration detected for solid state Form V and Form A should be caused by the amorphous content generated upon disc compression.

TABLE 54
Intrinsic Dissolution Rate Evaluation in FaSSIF at 37° C.

| | Parameter | | | | | | | |
|---|---|---|---|---|---|---|---|---|
| | Solid state | | | | | | Substantially | |
| | Form V | | Form A | | Form T | | amorphous form | |
| Group | 1 | 2 | 1 | 2 | 1 | 2 | 1 | 2 |

| IDR | No intrinsic | No intrinsic | No intrinsic | No intrinsic |
|---|---|---|---|---|
| (mg · cm⁻² · min⁻¹) | dissolution rate | dissolution rate | dissolution rate | dissolution rate |
| XRPD after IDR | No form change | No form change | No form change | No form change |
| Comments | Concentration | Concentration | <LOQ (0.0003 | <LOQ (0.0008 |
| | (0.006 mg/mL) | (0.006 mg/mL) | mg/mL) | mg/mL) |
| | remains same | remains same | | |
| | during the whole | during the whole | | |
| | process. | process. | | |

Form A, Form C, Form D, Form E, and Form I are relatively stable anhydrates or hydrates. Their relationships were investigated by competitive equilibration, water activity, and VT-XRPD experiments to allocate transition temperature or critical water activity at which form conversion occurs. Relative stability of Forms A, Form C, Form D, and Form E was investigated by water activity experiments in acetonitrile/water system at different temperatures. At 5° C. and 25° C., highly crystalline Form A was obtained in all the solvent mixtures. Relative stability of Form A and Form I was investigated by competitive equilibration experiments at 25° C. and 50° C. Form A were obtained from all the experiments. Feasibility of formulation processes of each form was also evaluated with compression, grinding and granulation simulation experiments. Form A was found to be a stable polymorph and suitable for further development.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. Crystalline Form A of a compound having Formula (I):

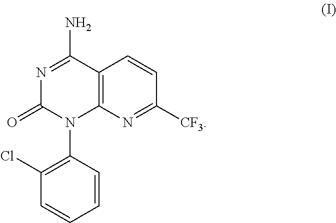 [SMILES: Nc1nc(=O)n(-c2ccccc2Cl)c2nc(C(F)(F)F)ccc12]

2. The crystalline Form A of claim 1, which is characterized by an X-ray powder diffraction (XRPD) pattern comprising 6.1, 11.1, and 16.6 degrees 2θ (±0.2 degrees 2θ).

3. The crystalline Form A of claim 1, which is characterized by an X-ray powder diffraction (XRPD) pattern comprising any of the three peaks selected from 6.1, 6.6, 10.0, 11.1, 12.1, 15.6, and 16.6 degrees 2θ (0.2 degrees 2θ).

4. The crystalline Form A of claim 1, which is characterized by an X-ray powder diffraction (XRPD) pattern comprising any of the four peaks selected from 6.1, 6.6, 10.0, 11.1, 12.1, 15.6, and 16.6 degrees 2θ (±0.2 degrees 2θ).

5. The crystalline Form A of claim 1, which is characterized by an X-ray powder diffraction (XRPD) pattern comprising any of the five peaks selected from 6.1, 6.6, 10.0, 11.1, 12.1, 15.6, and 16.6 degrees 2θ (±0.2 degrees 2θ).

6. The crystalline Form A of claim 1, which is characterized by an X-ray powder diffraction pattern comprising peaks at 6.1, 6.6, 11.1, 12.1, and 16.6 degrees 2θ (±0.2 degrees 2θ).

7. The crystalline Form A of claims 1, 2, or 6, which is characterized by an X-ray powder diffraction pattern comprising peaks at 10.0, 18.5, 20.8, 25.3, and 25.7 degrees 2θ (0.2 degrees 2θ).

8. The crystalline Form A of claim 1, characterized by an X-ray powder diffraction pattern substantially in accordance with FIG. 6.

9. The crystalline Form A of any one of claims 1 to 8, further characterized by a differential scanning calorimetry (DSC) thermogram comprising an endothermic peak at about 323.5° C.

10. The crystalline Form A of claim 9, wherein the DSC thermogram is substantially in accordance with FIG. 7.

11. The crystalline Form A of any one of claims 1 to 10, further characterized by a weight percent loss of about 1.0% upon heating to about 220° C., as measured by a thermal gravimetric analysis (TGA).

12. The crystalline Form A of any one of claims 1 to 11, further characterized by a thermal gravimetric analysis (TGA) thermogram substantially in accordance with FIG. 8.

13. The crystalline Form A of any one of claims 1 to 12, further characterized by a polarized light microscope (PLM) profile substantially as shown in FIG. 9.

14. Crystalline Form C of a compound having Formula (I):

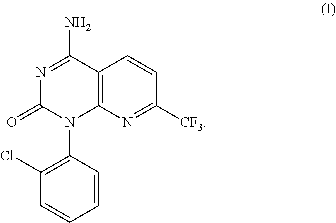 [SMILES: Nc1nc(=O)n(-c2ccccc2Cl)c2nc(C(F)(F)F)ccc12]

15. The crystalline Form C of claim 14, characterized by an X-ray powder diffraction (XRPD) pattern comprising peaks at 11.8, 16.6, 17.5, 27.2, and 28.2 degrees 2θ (0.2 degrees 2θ).

16. The crystalline Form C of claim 14, wherein the X-ray powder diffraction pattern further comprises peaks at 19.7, 20.3, 23.7, 24.5, and 29.8 degrees 2θ (±0.2 degrees 2θ).

17. The crystalline Form C of claim 14 or 16, wherein the X-ray powder diffraction pattern further comprises peaks at 10.9, 17.8, 21.8, 26.0, and 26.6 degrees 2θ (±0.2 degrees 2θ).

18. The crystalline Form C of claim 14, wherein the X-ray powder diffraction pattern is substantially in accordance with FIG. 13.

19. The crystalline Form C of any one of claims 14 to 18, wherein the DSC thermogram is substantially in accordance with FIG. 14.

20. The crystalline Form C of any one of claims 14 to 19, further characterized by a thermal gravimetric analysis (TGA) thermogram substantially in accordance with FIG. 15.

21. The crystalline Form C of any one of claims 14 to 20, further characterized by a polarized light microscope (PLM) profile substantially as shown in FIG. 16.

22. Crystalline Form D of a compound having Formula (I):

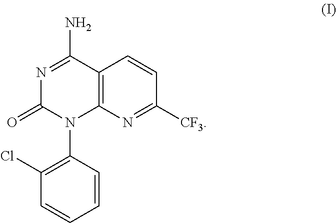 [SMILES: Nc1nc(=O)n(-c2ccccc2Cl)c2nc(C(F)(F)F)ccc12]

23. The crystalline Form D of claim 22, characterized by an X-ray powder diffraction (XRPD) pattern comprising peaks at 6.1, 12.4, 13.7, 16.5, 27.6 degrees 2θ (±0.2 degrees 2θ).

24. The crystalline Form D of claim 22, wherein the X-ray powder diffraction pattern further comprises peaks at 10.9, 14.8, 25.2, 26.7, and 27.9 degrees 2θ (±0.2 degrees 2θ).

25. The crystalline Form D of claim 22 or 24, wherein the X-ray powder diffraction pattern further comprises peaks at 10.3, 19.3, 21.2, 24.1, and 29.9 degrees 2θ (±0.2 degrees 2θ).

26. The crystalline Form D of claim 22, wherein the X-ray powder diffraction pattern is substantially in accordance with FIG. 19.

27. The crystalline Form D of any one of claims 22 to 26, wherein the DSC thermogram is substantially in accordance with FIG. 20.

28. The crystalline Form D of any one of claims 22 to 27, further characterized by a thermal gravimetric analysis (TGA) thermogram substantially in accordance with FIG. 21.

29. The crystalline Form D of any one of claims 22 to 28, further characterized by a polarized light microscope (PLM) profile substantially as shown in FIG. 22.

30. Crystalline Form E of a compound having Formula (I):

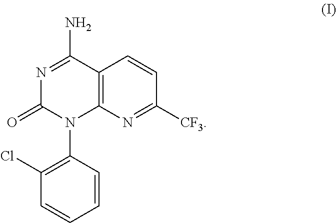 [SMILES: Nc1nc(=O)n(-c2ccccc2Cl)c2nc(C(F)(F)F)ccc12]

31. The crystalline Form E of claim 30, characterized by an X-ray powder diffraction (XRPD) pattern comprising peaks at 12.3, 13.7, 19.4, 26.7, and 27.6 degrees 2θ (0.2 degrees 2θ).

32. The crystalline Form E of claim 30, wherein the X-ray powder diffraction pattern further comprises peaks at 20.8, 24.5, 25.3, 28.1, and 30.0 degrees 2θ (±0.2 degrees 2θ).

33. The crystalline Form E of claim 30 or 32, wherein the X-ray powder diffraction pattern further comprises peaks at 7.2, 14.6, 14.9, 18.2, and 22.5 degrees 2θ (±0.2 degrees 2θ).

34. The crystalline Form E of claim 30, wherein the X-ray powder diffraction pattern is substantially in accordance with FIG. 25.

35. The crystalline Form E of any one of claims 30 to 34, wherein the DSC thermogram is substantially in accordance with FIG. 26.

36. The crystalline Form E of any one of claims 30 to 35, further characterized by a thermal gravimetric analysis (TGA) thermogram substantially in accordance with FIG. 27.

37. The crystalline Form E of any one of claims 30 to 36, further characterized by a polarized light microscope (PLM) profile substantially as shown in FIG. 28.

38. Crystalline Form I of a compound having Formula (I):

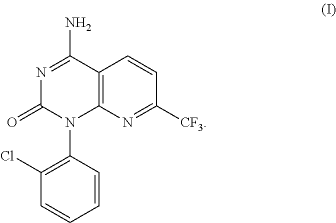 [SMILES: Nc1nc(=O)n(-c2ccccc2Cl)c2nc(C(F)(F)F)ccc12]

39. The crystalline Form I of claim 38, characterized by an X-ray powder diffraction (XRPD) pattern comprising peaks at 5.8, 6.4, 16.1, 16.5, and 27.7 degrees 2θ (0.2 degrees 2θ).

40. The crystalline Form I of claim 38, wherein the X-ray powder diffraction pattern further comprises peaks at 10.0, 10.8, 13.1, 25.5, and 29.0 degrees 2θ (±0.2 degrees 2θ).

41. The crystalline Form I of claim 38 or 40, wherein the X-ray powder diffraction pattern further comprises peaks at 8.4, 11.9, 17.7, 19.6, and 23.3 degrees 2θ (±0.2 degrees 2θ).

42. The crystalline Form I of claim 38, wherein the X-ray powder diffraction pattern is substantially in accordance with FIG. 41.

43. The crystalline Form I of any one of claims 38 to 42, wherein the DSC thermogram is substantially in accordance with FIG. 42.

44. The crystalline Form I of any one of claims 38 to 43, further characterized by a thermal gravimetric analysis (TGA) thermogram substantially in accordance with FIG. 43.

45. The crystalline Form I of any one of claims 38 to 44, further characterized by a polarized light microscope (PLM) profile substantially as shown in FIG. 44.

46. Crystalline Form T of a compound having Formula (I):

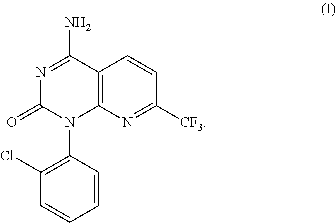 [SMILES: Nc1nc(=O)n(-c2ccccc2Cl)c2nc(C(F)(F)F)ccc12]

47. The crystalline Form T of claim 46, characterized by an X-ray powder diffraction (XRPD) pattern comprising peaks at 6.5, 10.9, 12.4, 13.0, and 16.9 degrees 2θ (0.2 degrees 2θ).

48. The crystalline Form T of claim 46, wherein the X-ray powder diffraction pattern further comprises peaks at 10.5, 16.6, 22.2, 27.5, and 27.9 degrees 2θ (±0.2 degrees 2θ).

49. The crystalline Form T of claim 46, wherein the X-ray powder diffraction pattern is substantially in accordance with FIG. 61.

50. The crystalline Form T of any one of claims 46 to 49, wherein the DSC thermogram is substantially in accordance with FIG. 62.

51. The crystalline Form T of any one of claims 46 to 50, further characterized by a thermal gravimetric analysis (TGA) thermogram substantially in accordance with FIG. 63.

52. The crystalline Form T of any one of claims 46 to 51, further characterized by a polarized light microscope (PLM) profile substantially as shown in FIG. 64.

53. A solid state form of a compound having Formula (I): 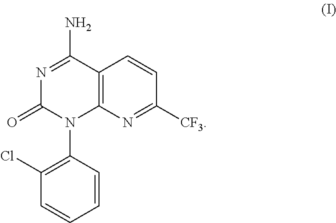 [SMILES: Nc1nc(=O)n(-c2ccccc2Cl)c2nc(C(F)(F)F)ccc12]
or a pharmaceutically acceptable salt thereof, wherein
the solid state form comprises at least 50 wt. % of a particular crystalline form, at least 60 wt. % of a particular crystalline form, at least 70 wt. % of a particular crystalline form, at least 80 wt. % of a particular crystalline form, at least 90 wt. % of a particular crystalline form, at least 95 wt. % of a particular crystalline form, or at least 99 wt. % of a particular crystalline form, wherein
the particular crystalline form is according to any one of claims 2 to 52.

54. The solid state form of claim 53, wherein the particular crystalline form is crystalline Form A.

55. A pharmaceutical composition comprising a crystalline form of any one of claims 2 to 52, and at least one pharmaceutically acceptable excipient.

56. A pharmaceutical composition comprising the solid state form of claim 53 or 54, and at least one pharmaceutically acceptable excipient.

57. A method for treating a disease treatable by inhibition of MAT2A in a patient comprising administering to the patient a therapeutically effective amount of a crystalline form of any one of claims 2 to 52, a solid state form of claim 53 or 54, or a pharmaceutical composition of claim 55 or 56.

58. The method of claim 57, wherein the disease is cancer.

59. A method of treating a MTAP null cancer in a patient comprising administering to the patient a therapeutically effective amount of a crystalline form of any one of claims 2 to 52, a solid state form of claim 53 or 54, or a pharmaceutical composition of claim 55 or 56.

60. A method for treating a cancer in a patient, wherein the cancer is characterized by a reduction or absence of MTAP gene expression, the absence of the MTAP gene, reduced level of MTAP protein, reduced function of MTAP protein, absence of MTAP protein, or a combination thereof, comprising administering to the subject a therapeutically effective amount of a crystalline form of any one of claims 2 to 52, a solid state form of claim 53 or 54, or a pharmaceutical composition of 55 or 56.

61. The method of claim 59 or 60, wherein the cancer is selected from the group consisting of cancer is leukemia, glioma, melanoma, pancreatic, non-small cell lung cancer (NSCLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophagogastric cancer, gastrointestinal (ii) cancer, or mesothelioma.

62. A method of making a pharmaceutical composition comprising mixing crystalline Form A with at least one pharmaceutically acceptable excipient.

63. A method of making a pharmaceutical composition comprising mixing the solid state form of claim 53 or 54 with at least one pharmaceutically acceptable excipient.

64. A pharmaceutical composition prepared by combining crystalline Form A with at least one pharmaceutically acceptable excipient.

65. A pharmaceutical composition prepared by combining the solid state form of claim 53 or 54 with at at least one pharmaceutically acceptable excipient.

66. A method for preparing crystalline Form A of a compound having Formula (I): 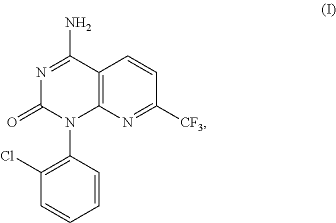 [SMILES: Nc1nc(=O)n(-c2ccccc2Cl)c2nc(C(F)(F)F)ccc12]
comprising:
a) forming a first mixture comprising a crude compound of Formula (I), ACN, and water;
b) solvent exchanging with water at a temperature of no more than about 65° C. to form a second mixture;
c) cooling the second mixture and stirring to form a third precipitate;
d) isolating the third precipitate;
e) forming a slurry comprising the third precipitate, methyl ethyl ketone (MEK), and water;
f) isolating a fourth precipitate from step e);
g) drying the fourth precipitate to provide the crystalline Form A of Formula (I).

67. The method of claim 66, wherein step a) is conducted at a temperature of from about 75° C. to 80° C.

68. The method of claim 66 or 67, wherein, in step a), a ratio of ACN to water is about 4:1 by volume.

69. The method of any one of claims 66 to 68, wherein step c) is conducted at a temperature of from about 0° C. to 5° C. and stirred for a period of from about 12 to 16 hours.

70. The method of any one of claims 66 to 69, wherein, in step e), a ratio of MEK to water is about 10:1 by volume.

71. The method of any one of claims 66 to 70, wherein step e) is conducted at a temperature of from about 60° C. to 65° C. and stirred for a period of from about 17 to 22 hours; and further cooled to a temperature of from about 0° C. to 5° C. and stirred for a period of from about 15 to 24 hours.

72. The method of any one of claims 66 to 71, wherein the isolating of step d) and/or step e) is conducted by filtration.

73. The method of any one of claims 66 to 71, wherein the drying of step g) is conducted at a temperature of from 65° C. to 70° C.

74. The method of any one of claims 66 to 73, wherein the crude compound of Formula (I) is present in the first mixture in an amount of from about 20 g/L to 100 g/L.

75. A method for preparing a crystalline Form of a compound having Formula (I): 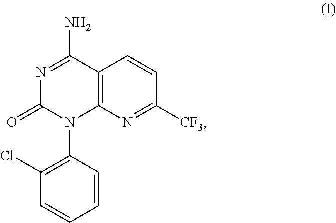 [SMILES: Nc1nc(=O)n(-c2ccccc2Cl)c2nc(C(F)(F)F)ccc12]
comprising:
a) forming a slurry comprising solid state Form V of Formula (I) and a solvent;
b) stirring the slurry for a period of at least a day; and
c) isolating a precipitate; and
d) drying the precipitate to provide the crystalline Form of Formula (I),
wherein the crystalline Form is crystalline Form C, D, E, I, or A-1; and
the solvent is methanol, water, a mixture of methanol and water, a mixture of acetone and water, or a mixture of acetonitrile and water.
